(19) United States
(12) Patent Application Publication
(10) Pub. No.: US 2010/0234364 A1
(43) Pub. Date: Sep. 16, 2010

(54) CCR2 INHIBITORS AND METHODS OF USE THEREOF

(76) Inventors: Arindrajit Basak, Groton, CT (US); Jeff Jin, Fremont, CA (US); Jimmie Moore, Redwood City, CA (US); Andrew M.K. Pennell, San Francisco, CA (US); Sreenlvas Punna, Sunnyvale, CA (US); Solomon Ungashe, Fremont, CA (US); Zheng Wei, Union City, CA (US)

Correspondence Address: BRINKS HOFER GILSON & LIONE, P.O. BOX 10395, CHICAGO, IL, 60610, US

(21) Appl. No.: 12/309,329

(22) PCT Filed: Jul. 10, 2007
(86) PCT No.: PCT/US2007/015785

§ 371 Date: May 21, 2010

(51) Int. Cl. A61K 31/54; C07C 311/21; A61K 31/18; C07D 213/78; A61K 31/4409; C07D 265/30; C07D 263/32; A61K 31/5375; A61K 31/421; C07D 213/42; A61K 31/4418; C07D 207/04; A61K 31/40; C07D 207/16; C07D 279/12; C07D 231/18; A61K 31/415; A61P 29/00; A61P 37/00

(52) U.S. Cl. 514/227.5; 564/92; 514/604; 546/328; 514/354; 544/160; 548/235; 514/237.5; 514/374; 546/338; 514/357; 548/539; 514/423; 548/536; 544/58.2; 548/374.1; 514/406

(57) ABSTRACT

Compounds are provided that act as potent antagonists of the CCR2 or CCR9 receptor. Animal testing demonstrates that these compounds are useful for treating inflammation, a hallmark disease for CCR2 and CCR9. The compounds are generally aryl sulfonamide derivatives and are useful in pharmaceutical compositions, methods for the treatment of CCR2-mediated diseases, CCR9-mediated diseases, as controls in assays for the identification of CCR2 antagonists, and as controls in assays for the identification of CCR9 antagonists.

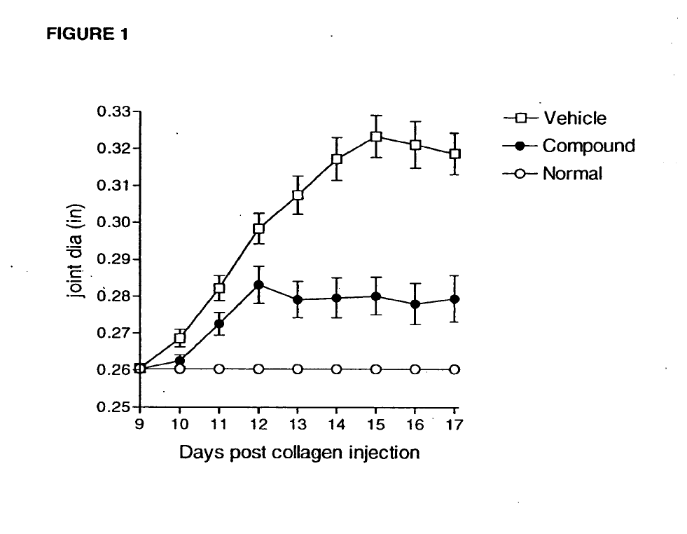

### RELATED APPLICATIONS

This application claims priority to U.S. provisional application Ser. No. 60/830,926 filed Jul. 14, 2006. The disclosure of this priority application is incorporated herein in its entirety.

### FEDERALLY SPONSORED RESEARCH OR DEVELOPMENT

The present invention described herein was supported at least in part by NIH (U19-Al056690). The government may have certain rights in the invention.

### BACKGROUND OF THE INVENTION

The present invention provides compounds, pharmaceutical compositions containing one or more of those compounds or their pharmaceutically acceptable salts, which are effective in inhibiting the binding or function of various chemokines to chemokine receptors. As antagonists or modulators of chemokine receptors, the compounds and compositions have utility in treating various immune disorder conditions and diseases.

Chemokines, also known as chemotactic cytokines, are a group of small molecular-weight proteins that are released by a wide variety of cells and have a variety of biological activities. Chemokines attract various types of cells of the immune system, such as macrophages, T cells, eosinophils, basophils and neutrophils, and cause them to migrate from the blood to various lymphoid and none-lymphoid tissues. They mediate infiltration of inflammatory cells to sites of inflammation, and are responsible for the initiation and perpetuation of many inflammation diseases (reviewed in Schall, Cytokine, 3:165-183 (1991), Schall et al., Curr. Opin. Immunol., 6:865-873 (1994)).

In addition to stimulating chemotaxis, chemokines can induce other changes in responsive cells, including changes in cell shape, granule exocytosis, integrin up-regulation, formation of bioactive lipids (e.g., leukotrienes), respiratory burst associated with leukocyte activation, cell proliferation, resistance to induction of apoptosis and angiogenesis. Thus, chemokines are early triggers of the inflammatory response, causing inflammatory mediator release, chemotaxis and extravasation to sites of infection or inflammation. They are also stimulators of a multitude of cellular processes that bear important physiological functions as well as pathological consequences.

Chemokines exert their effects by activating chemokine receptors expressed by responsive cells. Chemokine receptors are a class of G-protein coupled receptors, also known as seven-transmembrane receptors, found on the surface of a wide variety of cell types such as leukocytes, endothelial cells, smooth muscle cells and tumor cells.

Chemokines and chemokine receptors are expressed by intrinsic renal cells and infiltrating cells during renal inflammation (Segerer et al., J. Am. Soc. Nephrol., 11:152-76 (2000); Morii et al., J. Diabetes Complications, 17:11-5 (2003); Lloyd et al. J. Exp. Med., 185:1371-80 (1997); Gonzalez-Cuadrado et al. Clin. Exp. Immunol., 106:518-22 (1996); Eddy & Giachelli, Kidney Int., 47:1546-57 (1995); Diamond et al., Am. J. Physiol., 266:F926-33 (1994)). In humans, CCR2 and ligand MCP-1 are among the proteins expressed in renal fibrosis, and are correlated with the extent of macrophage infiltration into the interstitium (Yang et al., Zhonghua Yi Xue Za Zhi, 81:73-7 (2001); Stephan et al., J. Urol., 167:1497-502 (2002); Amann et al., Diabetes Care, 26:2421-5 (2003); Dai et al., Chin. Med. J. (Engl), 114:864-8 (2001)). In animal models of renal fibrosis, blockade of CCR2 or MCP-1 leads to a marked reduction in severity of renal inflammation (Kitagawa et al., Am. J. Pathol., 165:237-46 (2004); Wada et al., Am. J. Pathol., 165:237-46 (2004); Shimizu et al., J. Am. Soc. Nephrol., 14:1496-505 (2003)).

Rheumatoid arthritis is a chronic disease of the joints characterized by synovial inflammation that leads to the destruction of cartilage and bone. Although the underlying causes of the disease are unknown, it is believed that macrophages and Th-1 type T cells play a key role in the initiation and perpetuation of the chronic inflammatory process (Vervoordeldonk et al., Curr. Rheumatol. Rep., 4:208-17 (2002)).

MCP-1 is among the several chemokines, including MIP-1α and IL-8, identified in rheumatoid synovium (Villiger et al., J. Immunol., 149:722-7 (1992); Scaife et al., Rheumatology (Oxford), 43:1346-52 (2004); Shadidi et al., Scand. J. Immunol., 57:192-8 (2003); Taylor et al., Arthritis Rheum., 43:38-47 (2000); Tucci et al., Biomed. Sci. Instrum., 34:169-74 (1997)). Chemokine receptors CCR1, CCR2, CCR3 and CCR5 are up-regulated in the joints from arthritic mice (Plater-Zyberk et al., Immunol. Lett., 57:117-20 (1997). Blockade of MCP-1 activity using a CCR2 antagonist or an antibody against MCP-1 have been shown efficacious in reducing joint inflammation in experimental models of rheumatoid arthritis (Gong et al., J. Exp. Med., 186:131-7 (1997); Ogata et al., J. Pathol., 182:106-14 (1997)).

Chemokine receptor-mediated infiltration of macrophages in the fat tissues may also contribute to the complications arising from obesity, a condition resulting from excessive storage of fat in the body. Obesity predisposes the affected individuals to many disorders, such as non-insulin-dependent diabetes, hypertension, stroke, and coronary artery disease. In obesity, adipose tissues have altered metabolic and endocrine functions that lead to an increased release of fatty acids, hormones, and pro-inflammatory molecules. Adipose tissue macrophages are believed to be a key source of pro-inflammatory cytokines including TNF-alpha, iNOS and IL-6 (Weisberg et al., J. Clin. Invest., 112:1796-808 (2003)). Recruitment of macrophages to the adipose tissue is likely mediated by MCP-1 produced by adipocytes (Christiansen T, et al., Int. J. Obes. (Lond). 2005 January; 29(1):146-50; Sartipy et al., Proc. Natl. Acad. Sci. U.S.A., 100:7265-70 (2003)).

Elevated MCP-1 may induce adipocyte differentiation and insulin resistance, and contribute to pathologies associated with hyper-insulinemia and obesity. MCP-1 is over-expressed in plasma in obese mice compared to lean controls and white adipose is a major source. MCP-1 has also been shown to accelerate wound healing, and has a direct angiogenic effect on epithelial cells, and may play a direct role in the remodeling of adipose tissue in obesity. (Sartipy P, Loskutoff D J., Proc. Natl. Acad. Sci. U.S.A., 100:7265 (2003)).

MCP-1 plasma levels are substantially increased in Diet Induce Obesity (DIO) mice, and a strong correlation between plasma MCP-1 levels and body weight has been identified. Furthermore, elevation of MCP-1 induced by high fat diet causes changes in the CD11b positive monocyte population in DIO mice. (Takahashi K, et al., J. Biol. Chem., 46654 (2003)).

Furthermore, chronic inflammation in fat is thought to play a crucial role in the development of obesity-related insulin resistance (Xu H, et al., J. Clin. Invest. 2003 December; 112(12):1821-30). It has been proposed that obesity related insulin resistance is, at least in part, a chronic inflammatory disease initiated in adipose tissue. Many inflammation and macrophage specific genes are dramatically upregulated in white adipose tissue in mouse models of genetic and high fat diet-induced obesity (DIO), and this upregulation precedes a dramatic increase in circulating insulin.

Increased expression levels of monocyte CCR2 and monocyte chemoattractant protein-1 in patients with diabetes mellitus (Biochemical and Biophysical Research Communications, 344(3):780-5 (2006)) were found in a study involving diabetic patients. Serum MCP-1 concentrations and surface expression of CCR2 on monocytes in diabetic patients were significantly higher than in non-diabetics, and the serum MCP-1 levels correlated with HbA1c, triglycerides, BMI, hs-CRP. Surface expression levels of CD36 and CD68 on monocytes were significantly increased in diabetic patients and more unregulated by MCP-1 in diabetics, augmenting uptake of ox-LDL, and hence potentially foam cell transformation. Elevated serum MCP-1 and increased monocyte CCR2, CD36, CD68 expression correlated with poor blood glucose control and potentially correlate with increased vessel wall monocyte recruitment.

MCP-1 is a potential player in negative cross talk between adipose tissue and skeletal muscle (Bianco J J, et al., Endocrinology, 2458 (2006)). MCP-1 can significantly reduce insulin-stimulated glucose uptake, and is a prominent inducer of insulin resistance in human skeletal muscle cell. Adipose tissue is a major secretory and endocrine active organ producing bioactive proteins regulating energy metabolism and insulin sensitivity.

CCR2 modulates inflammatory and metabolic effects of high-fat feeding (Weisberg S P, et al., J. Clin. Invest., 115 (2006)). Genetic deficiency in CCR2 reduced food intake and attenuated the development of obesity in mice fed a high fat diet. In obese mice matched for adiposity, CCR2 deficiency reduced macrophage content and inflammatory profile of adipose tissue, increased adiponectin expression, and improved glucose homeostatis and insulin sensitivity. In lean animals, no effect of CCR2 genotype on metabolic trait was found. In high-fat diet mice, CCR2 genotype modulated feeding, the development of obesity and adipose tissue inflammation. Once established, short term antagonism was shown to attenuate macrophage accumulation in adipose tissue and insulin resistance.

Chemokine and chemokine receptors are the key regulators of immune cell trafficking. MCP-1 is a potent chemoattractant of monocytes and T cells; its expression is induced under inflammatory conditions including proinflammatory cytokine stimulations and hypoxia. The interaction between MCP-1 and CCR2 mediates migration of monocytes, macrophage as well as activated T cells and play a key role in the pathogenesis of many inflammatory diseases. Inhibition of CCR2 functions using small molecule antagonists described in this invention represents a new approach for the treatments of inflammatory disorders.

Psoriasis is a chronic inflammatory disease characterized by hyperproliferation of keratinocytes and pronounced leukocyte infiltration. It is known that keratinocytes from psoriasis lesion express abundant CCR2 ligand MCP-1, particularly when stimulated by proinflammatory cytokines such as TNF-α (Vestergaard et al., Acta. Derm. Venereal., 84(5):353-8 (2004); Gillitzer et al., J. Invest. Dermatol., 101(2):127-31 (1993); Deleuran et al., J. Dermatol. Sci., 13(3):228-36 (1996)). Since MCP-1 can attract migration of both macrophages and dendritic cells expressing CCR2 to the skin, this receptor and ligand pair is believed to be important in regulating the interaction between proliferating keratinocytes and dermal macrophage during the development of psoriasis. A small molecule antagonist may thus be useful in the treatment of psoriasis.

In addition to inflammatory diseases, chemokines and chemokine receptors have also been implicated in cancers (Broek et al., Br. J. Cancer, 88(6):855-62 (2003)). Tumor cells stimulate the formation of stroma that secretes various mediators pivotal for tumor growth, including growth factors, cytokines, and proteases. It is known that the level of MCP-1 is associated significantly with tumor-associated macrophage accumulation, and prognostic analysis reveals that high expression of MCP-1 is a significant indicator of early relapse in breast cancer (Ueno et al., Clin. Cancer Res., 6(8):3282-9 (2001)). A small molecule antagonist of a chemokine may thus be able to reduce the release of growth-stimulating cytokines by blocking accumulation of macrophages at sites of tumor formation.

T lymphocyte (T cell) infiltration into the small intestine and colon has been linked to the pathogenesis of Coeliac diseases, food allergies, rheumatoid arthritis, human inflammatory bowel diseases (IBD) which include Crohn's disease and ulcerative colitis. Blocking trafficking of relevant T cell populations to the intestine can lead to an effective approach to treat human IBD. More recently, chemokine receptor 9 (CCR9) has been noted to be expressed on gut-homing T cells in peripheral blood, elevated in patients with small bowel inflammation such as Crohn's disease and celiac disease. The only CCR9 ligand identified to date, TECK (thymus-expressed chemokine) is expressed in the small intestine and the ligand receptor pair is now thought to play a pivotal role in the development of IBD. In particular, this pair mediates the migration of disease causing T cells to the intestine. See for example, Zaballos et al., J. Immunol., 162(10):5671 5675 (1999); Kunkel et al., J. Exp. Med., 192(5):761-768 (2000); Papadakis et al., J. Immunol., 165(9):5069-5076 (2000); Papadakis et al., Gastroenterology, 121(2):246-254 (2001); Campbell et al., J. Exp. Med., 195(1):135-141 (2002); Wurbel et al., Blood, 98(9):2626-2632 (2001); and Uehara et al., J. Immunol., 168(6):2811-2819 (2002). Rivera-Nieves, et al., Gastroenterology, 2006 November; 131(5):1518-29; and Kontoyiannis et al., J. Exp. Med., Vol. 196, Number 12, Dec. 16, 2002. In addition CCR9 bearing lymphocytes have been show to mediate the pathology of filariasis (lymphatic filarial disease) and inhibition of CCR9 has been correlated with reduction of the pathology associated with such conditions. See for example Babu et al., Journal of Infectious Diseases, 191: 1018-26, 2005.

PCT Published Application WO 2003/099773 (Millennium Pharmaceuticals, Inc.) discloses compounds which can bind to CCR9 receptors of the formula

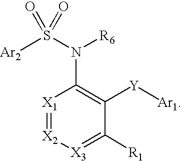

PCT Published Application WO 2005/004810 (Merck & Co., Inc.) discloses brandykinin B1 antagonists or inverse agonists of the formula

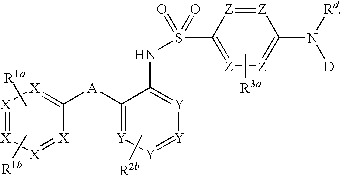

US Published Patent Application 2007/0037794 A1 (ChemoCentryx, Inc.) discloses CCR2 modulators of the formula

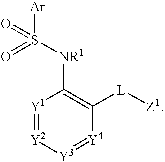

### BRIEF SUMMARY OF THE INVENTION

The present invention is directed to compounds and pharmaceutically acceptable salts thereof, compositions, and methods useful in modulating chemokine activity. The compounds and salts thereof, compositions, and methods described herein are useful in treating or preventing chemokine-mediated conditions or diseases, including certain inflammatory and immunoregulatory disorders and diseases.

The compounds of the present invention have been shown to modulate one or more of CCR1, CCR2, CCR3, CCR4, CCR5, CCR6, CCR7, CCR8, CCR9, CCR10, CXCR3, CXCR4, CXCR5, and CX3CR1. In particular, various compounds of the present invention modulate CCR2 and CCR9 as shown in the examples.

In one embodiment, the present compound may be represented by formula (I) or salts thereof:

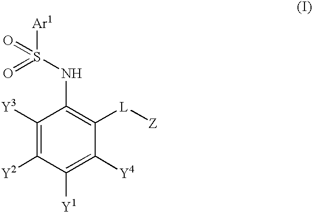

where Ar¹, Y¹, Y², Y³, Y⁴, L and Z are as defined below. Salts and N-oxides of these compounds are within the scope of the invention.

In another aspect, the present invention provides compositions useful in modulating chemokine activity. In one embodiment, a composition according to the present invention comprises a compound according to the invention and a pharmaceutically acceptable carrier or excipient.

In yet another aspect, the present invention provides a method of modulating chemokine function in a cell, comprising contacting the cell with a therapeutically effective amount of a compound or composition according to the invention.

In still another aspect, the present invention provides a method for modulating chemokine function, comprising contacting a chemokine receptor with a therapeutically effective amount of a compound or composition according to the invention.

In still another aspect, the present invention provides a method for treating a chemokine-mediated condition or disease, comprising administering to a subject a safe and effective amount of a compound or composition according to the invention.

In addition to the compounds provided herein, the present invention further provides pharmaceutical compositions containing one or more of these compounds, as well as methods for the use of these compounds in therapeutic methods, primarily to treat diseases associated with chemokine signaling activity.

### DETAILED DESCRIPTION OF THE INVENTION

General

The present invention is directed to compounds and salts thereof, compositions and methods useful in the modulation of chemokine receptor function, particularly CCR2 or CCR9 function. Modulation of chemokine receptor activity, as used herein in its various forms, is intended to encompass antagonism, agonism, partial antagonism, inverse agonism and/or partial agonism of the activity associated with a particular chemokine receptor, preferably the CCR2 or CCR9 receptor. Accordingly, the compounds of the present invention are compounds which modulate at least one function or characteristic of mammalian CCR2 or CCR9, for example, a human CCR2 or CCR9 protein. The ability of a compound to modulate the function of CCR2 or CCR9, can be demonstrated in a binding assay (e.g., ligand binding or agonist binding), a migration assay, a signaling assay (e.g., activation of a mammalian G protein, induction of rapid and transient increase in the concentration of cytosolic free calcium), and/or cellular response assay (e.g., stimulation of chemotaxis, exocytosis or inflammatory mediator release by leukocytes).

Abbreviations and Definitions

When describing the compounds, compositions, methods and processes of this invention, the following terms have the following meanings, unless otherwise indicated.

“Alkyl” by itself or as part of another substituent refers to a hydrocarbon group which may be linear, cyclic, or branched or a combination thereof having the number of carbon atoms designated (i.e., C₁₋₈ means one to eight carbon atoms). Examples of alkyl groups include methyl, ethyl, n-propyl, isopropyl, n-butyl, t-butyl, isobutyl, sec-butyl, cyclohexyl, cyclopentyl, (cyclohexyl)methyl, cyclopropylmethyl, bicyclo[2.2.1]heptane, bicyclo[2.2.2]octane, etc. Alkyl groups can be substituted or unsubstituted, unless otherwise indicated. Examples of substituted alkyl include haloalkyl, thioalkyl, aminoalkyl, and the like.

“Alkoxy” refers to —O-alkyl. Examples of an alkoxy group include methoxy, ethoxy, n-propoxy etc.

“Alkenyl” refers to an unsaturated hydrocarbon group which may be linear, cyclic or branched or a combination thereof. Alkenyl groups with 2-8 carbon atoms are preferred. The alkenyl group may contain 1, 2 or 3 carbon-carbon double bonds. Examples of alkenyl groups include ethenyl, n-propenyl, isopropenyl, n-but-2-enyl, n-hex-3-enyl, cyclohexenyl, cyclopentenyl and the like. Alkenyl groups can be substituted or unsubstituted, unless otherwise indicated.

“Alkynyl” refers to an unsaturated hydrocarbon group which may be linear, cyclic or branched or a combination thereof. Alkynyl groups with 2-8 carbon atoms are preferred. The alkynyl group may contain 1, 2 or 3 carbon-carbon triple bonds. Examples of alkynyl groups include ethynyl, n-propynyl, n-but-2-ynyl, n-hex-3-ynyl and the like. Alkynyl groups can be substituted or unsubstituted, unless otherwise indicated.

“Aryl” refers to a polyunsaturated, aromatic hydrocarbon group having a single ring (monocyclic) or multiple rings (bicyclic) which can be fused together or linked covalently. Aryl groups with 6-10 carbon atoms are preferred, where this number of carbon atoms can be designated by C₆₋₁₀, for example. Examples of aryl groups include phenyl and naphthalene-1-yl, naphthalene-2-yl, biphenyl and the like. Aryl groups can be substituted or unsubstituted, unless otherwise indicated.

“Halo” or “halogen”, by itself or as part of a substituent refers to a chlorine, bromine, iodine, or fluorine atom.

“Haloalkyl”, as a substituted alkyl group, refers to a monohaloalkyl or polyhaloalkyl group, most typically substituted with from 1-3 halogen atoms. Examples include 1-chloroethyl, 3-bromopropyl, trifluoromethyl and the like.

“Heterocyclyl” refers to a saturated or unsaturated non-aromatic group containing at least one heteroatom (typically 1 to 5 heteroatoms) selected from nitrogen, oxygen or sulfur. The heterocyclyl ring may be monocyclic or bicyclic. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Examples of heterocycle groups include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene, quinuclidine and the like. Preferred heterocyclic groups are monocyclic, though they may be fused or linked covalently to an aryl or heteroaryl ring system.

In one preferred embodiment, heterocyclic groups may be represented by formula (AA) below:

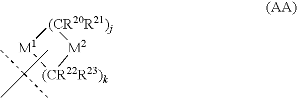

where formula (AA) is attached via a free valence on either M¹ or M²; M¹ represents O, NR²⁴, or S(O)ₗ; M² represents CR²⁵R²⁶, O, S(O)ₗ, or NR²⁴, l is 0, 1 or 2; j is 1, 2 or 3; and k is 1, 2 or 3, with the proviso that j+k is 3, 4, or 5; and R²⁰, R²¹, R²²; R²³, R²⁴, R²⁵, and R²⁶ are independently selected from the group consisting of hydrogen, halogen, unsubstituted or substituted C₁₋₈ alkyl, unsubstituted or substituted C₂₋₈ alkenyl, unsubstituted or substituted C₂₋₈ alkynyl, —C(O)R²⁷, —CO₂R²⁷, —C(O)NR²⁷R²⁸, —NR²⁷C(O)R²⁸, —S(O)₂R²⁸, —S(O)₂NR²⁸R²⁹, —NR²⁸S(O)₂R²⁹—NR²⁸R²⁹, —OR²⁸, —V¹C(O)R²⁸, —V¹CO₂R²⁸, —V¹C(O)NR²⁸R²⁹, —V¹NR²⁸C(O)R²⁹, —V¹S(O)₂R²⁸, —V¹S(O)₂NR²⁸R²⁹, —V¹NR²⁸S(O)₂R²⁹, —V¹NR²⁸R²⁹, and —V¹OR²⁸, wherein V¹ is a member selected from the group consisting of C₁₋₄ alkylene, C₂₋₄ alkenylene and C₂₋₄ alkynylene, and R²⁷, R²⁸ and R²⁹ are independently selected from the group consisting of hydrogen and C₁₋₈ alkyl, and wherein the aliphatic portions of each of the R²⁰, R²¹, R²², R²³, R²⁴, R²⁵, R²⁶, R²⁷, R²⁸ and R²⁹ substituents are optionally substituted with from one to three members selected from the group consisting of halogen, —OH, —OR³⁰, —OC(O)NHR³⁰, —OC(O)NR³⁰R³¹, —SH, —SR³⁰, —S(O)R³⁰, —S(O)₂R³⁰, —S(O)₂NH₂, —S(O)₂NHR³⁰, —S(O)₂NR³⁰R³¹, —NHS(O)₂R³⁰, —NR³⁰S(O)₂R³¹, —C(O)NH₂, —C(O)NHR³⁰, —C(O)NR³⁰R³¹, —C(O)R³⁰, NHC(O)R³⁰, —NR³⁰C(O)R³¹, —NHC(O)NH₂, —NR³⁰C(O)NH₂, —NR³⁰C(O)NHR³¹, —NHC(O)NHR³⁰, —NR³⁰C(O)NR³⁰R³¹, —NHC(O)NR³⁰R³¹, —CO₂H, —CO₂R³⁰, —NHCO₂R³⁰, —NR³⁰CO₂R³¹, —CN, —NO₂, —NH₂, —NHR³⁰, —NR³⁰R³¹, —NR³⁰S(O)NH₂ and —NR³⁰S(O)₂NHR³¹, wherein R³⁰ and R³¹ are independently an unsubstituted C₁₋₆ alkyl. Additionally, any two of R²⁰, R²¹, R²², R²³, R²⁴, R²⁵, and R²⁶ may be combined to form a bridged or spirocyclic ring system, including a fused ring system. In one embodiment the fused ring system is aryl or heteroaryl.

In one preferred embodiment, the number of R²⁰+R²¹+R²²+R²³ groups that are other than hydrogen is 0, 1 or 2. In a more preferred embodiment, R²⁰, R²¹, R²², R²³, R²⁴, R²⁵, and R²⁶ are independently selected from the group consisting of hydrogen, halogen, unsubstituted or substituted C₁₋₈ alkyl, —C(O)R²⁸, —CO₂R²⁸, —C(O)NR²⁸R²⁹, —NR²⁸C(O)R²⁹, —S(O)₂R²⁸, —S(O)₂NR²⁸R²⁹, —NS(O)₂R²⁸R²⁹, —NR²⁸R²⁹, and —OR²⁸, wherein R²⁸ and R²⁹ are independently selected from the group consisting of hydrogen, unsubstituted C₁₋₈ alkyl and wherein the aliphatic portions of each of the R²⁰, R²¹, R²², R²³, R²⁴, R²⁵ and R²⁶ substituents are optionally substituted with from one to three members selected from the group consisting of halogen, —OH, —OR³⁰, —OC(O)NHR³⁰, —OC(O)NR³⁰R³¹, —SH, —SR³⁰, —S(O)R³⁰, —S(O)₂R³⁰, —S(O)₂NH₂, —S(O)₂NHR³⁰, —S(O)₂NR³⁰R³¹, —NHS(O)₂R³⁰, —NR³⁰S(O)₂R³¹, —C(O)NH₂, —C(O)NHR³⁰, —C(O)NR³⁰R³¹, —C(O)R³⁰, —NHC(O)R³⁰, —NR³⁰C(O)R³¹, —NHC(O)NH₂, —NR³⁰C(O)NH₂, —NR³⁰C(O)NHR³¹, —NHC(O)NHR³⁰, —NR³⁰C(O)NR³⁰R³¹, —NHC(O)NR³⁰R²¹, —CO₂H, —CO₂R³⁰, —NHCO₂R³⁰, —NR³⁰CO₂R³¹, —CN, —NO₂, —NH₂, —NHR³⁰, —NR³⁰R³¹, —NR³⁰S(O)NH₂ and —NR³⁰S(O)₂NHR³¹, wherein R³⁰ and R³¹ are independently an unsubstituted C₁₋₆ alkyl.

In a more preferred embodiment, R²⁰, R²¹, R²², R²³, R²⁴, R²⁵, and R²⁶ are independently hydrogen or C₁₋₄alkyl. In another preferred embodiment, at least three of R²⁰, R²¹, R²², R²³, R²⁴, R²⁵, and R²⁶, are hydrogen.

“Heteroaryl” refers to an aromatic group containing at least one heteroatom, where the heteroaryl group may be monocyclic or bicyclic. Examples include pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, azaindole, azaindazole, benzothiazolyl, benzofuranyl, benzothienyl, indolyl, azaindolyl, azaindazolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl. Preferred heteroaryl groups are those having at least one aryl ring nitrogen atom, such as quinolinyl, quinoxalinyl, purinyl, benzimidazolyl, benzopyrazolyl, benzotriazolyl, benzothiazolyl, indolyl, quinolyl, isoquinolyl and the like. Preferred 6-ring heteroaryl systems include pyridyl, pyridazinyl, pyrazinyl, pyrimidinyl, triazinyl and the like. Preferred 5-ring heteroaryl systems include isothiazolyl, pyrazolyl, imidazolyl, thienyl, furyl, triazolyl, tetrazolyl, oxazolyl, isoxazolyl, oxadiazolyl, thiadiazolyl, pyrrolyl, thiazolyl and the like.

Heterocyclyl and heteroaryl can be attached at any available ring carbon or heteroatom. Each heterocyclyl and heteroaryl may have one or more rings. When multiple rings are present, they can be fused together or linked covalently. Each heterocyclyl and heteroaryl must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Heterocyclyl and heteroaryl groups can be substituted or unsubstituted, unless otherwise indicated. For substituted groups, the substitution may be on a carbon or heteroatom. For example, when the substitution is oxo (═O or —O⁻), the resulting group may have either a carbonyl (—C(O)—) or a N-oxide

Suitable substituents for substituted alkyl, substituted alkenyl, and substituted alkynyl include halogen, —CN, —CO₂R′, —C(O)R′, —C(O)NR′R″, oxo (═O or —O⁻), —OR′, —OC(O)R′, —OC(O)NR′R″—NO₂, —NR′C(O)R″, —NR′″C(O)NR′R″, —NR′R″, —NR′CO₂R″, —NR′S(O)₂R′″, —SR′, —S(O)R′, —S(O)₂R′, —S(O)₂NR′R″, —SiR′R″R′″, —N₃, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to (2m′+1), where m′ is the total number of carbon atoms in such radical.

Suitable substituents for substituted aryl, substituted heteroaryl and substituted heterocyclyl include halogen, —CN, —CO₂R′, —C(O)R′, —C(O)NR′R″, oxo (═O or —O⁻), —OR′, —OC(O)R′, —OC(O)NR′R″, —NO₂, —NR′C(O)R″, —NR′C(O)NR″R′″, —NR′R″, —NR′CO₂R″, —NR′S(O)₂R″, —SR′, —S(O)R′, —S(O)₂R′, —S(O)₂NR′R″, —NR′—C(NHR″)═NR′″, —SiR′R″R′″, —N₃, substituted or unsubstituted C₁₋₈ alkyl, substituted or unsubstituted C₂₋₈ alkenyl, substituted or unsubstituted C₂₋₈ alkynyl, substituted or unsubstituted C₆₋₁₀ aryl, substituted or unsubstituted 5- to 10-membered heteroaryl, and substituted or unsubstituted 3- to 10-membered heterocyclyl. The number of possible substituents range from zero to the total number of open valences on the aromatic ring system.

As used above, R′, R″ and R′″ each independently refer to a variety of groups including hydrogen, substituted or unsubstituted C₁₋₈ alkyl, substituted or unsubstituted C₂₋₈ alkenyl, substituted or unsubstituted C₂₋₈ alkynyl, substituted or unsubstituted aryl, substituted or unsubstituted heteroaryl, substituted or unsubstituted heterocyclyl, substituted or unsubstituted arylalkyl, and substituted or unsubstituted aryloxyalkyl. When R′ and R″ are attached to the same nitrogen atom, they can be combined with the nitrogen atom to form a 3-, 4-, 5-, 6-, or 7-membered ring (for example, —NR′R″ includes 1-pyrrolidinyl and 4-morpholinyl). Furthermore, R′ and R″, R″ and R′″, or R′ and R′″ may together with the atom(s) to which they are attached, form a substituted or unsubstituted 5-, 6- or 7-membered ring.

Two of the substituents on adjacent atoms of an aryl or heteroaryl ring may optionally be replaced with a substituent of the formula -T-C(O)—(CH₂)_{q}—U—, wherein T and U are independently —NR″″—, —O—, —CH₂— or a single bond, and q is an integer of from 0 to 2. Alternatively, two of the substituents on adjacent atoms of the aryl or heteroaryl ring may optionally be replaced with a substituent of the formula -A′-(CH₂)ᵣ—B′—, wherein A′ and B′ are independently —CH₂—, —O—, —NR″″—, —S—, —S(O)—, —S(O)₂—, —S(O)₂NR″″— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR″″—, —S—, —S(O)—, —S(O)₂—, or —S(O)₂NR′—. R″″ is selected from hydrogen or unsubstituted C₁₋₈ alkyl.

“Heteroatom” is meant to include oxygen (O), nitrogen (N), sulfur (S) and silicon (Si).

“Pharmaceutically acceptable” carrier, diluent, or excipient is a carrier, diluent, or excipient compatible with the other ingredients of the formulation and not deleterious to the recipient thereof.

“Pharmaceutically-acceptable salt” refers to a salt which is acceptable for administration to a patient, such as a mammal (e.g., salts having acceptable mammalian safety for a given dosage regime). Such salts can be derived from pharmaceutically-acceptable inorganic or organic bases and from pharmaceutically-acceptable inorganic or organic acids, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Salts derived from pharmaceutically-acceptable acids include acetic, ascorbic, benzenesulfonic, benzoic, camphosulfonic, citric, ethanesulfonic, fumaric, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, tartaric, p-toluenesulfonic and the like.

Also included are salts of amino acids such as arginate and the like, and salts of organic acids like glucuronic or galactunoric acids and the like (see, for example, Berge, S. M. et al, “Pharmaceutical Salts”, J. Pharmaceutical Science, 1977, 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Salt thereof” refers to a compound formed when the hydrogen of an acid is replaced by a cation, such as a metal cation or an organic cation and the like. Preferably, the salt is a pharmaceutically-acceptable salt, although this is not required for salts of intermediate compounds which are not intended for administration to a patient.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs may be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation, selection, and use of prodrugs is discussed in T. Higuchi and V. Stella, “Pro-drugs as Novel Delivery Systems,” Vol. 14 of the A.C.S. Symposium Series; “Design of Prodrugs”, ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

The compounds of the invention may be present in the form of pharmaceutically acceptable metabolites thereof. The term “metabolite” means a pharmaceutically acceptable form of a metabolic derivative of a compound of the invention (or a salt thereof). In some aspects, the metabolite may be a functional derivative of a compound that is readily convertible in vivo into an active compound. In other aspects, the metabolite may be an active compound.

“Therapeutically effective amount” refers to an amount sufficient to effect treatment when administered to a patient in need of treatment.

“Treating” or “treatment” as used herein refers to the treating or treatment of a disease or medical condition (such as a viral, bacterial or fungal infection or other infectious diseases, as well as autoimmune or inflammatory conditions) in a patient, such as a mammal (particularly a human or a companion animal) which includes ameliorating the disease or medical condition, i.e., eliminating or causing regression of the disease or medical condition in a patient; suppressing the disease or medical condition, i.e., slowing or arresting the development of the disease or medical condition in a patient; or alleviating the symptoms of the disease or medical condition in a patient.

Certain compounds of the present invention can exist in unsolvated forms as well as solvated forms, including hydrated forms. In general, both solvated forms and unsolvated forms are intended to be encompassed within the scope of the present invention. Certain compounds of the present invention may exist in multiple crystalline or amorphous forms (i.e., as polymorphs). In general, all physical forms are equivalent for the uses contemplated by the present invention and are intended to be within the scope of the present invention.

It will be apparent to one skilled in the art that certain compounds of the present invention may exist in tautomeric forms, all such tautomeric forms of the compounds being within the scope of the invention. Certain compounds of the present invention possess asymmetric carbon atoms (optical centers) or double bonds; the racemates, diastereomers, geometric isomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The compounds of the present invention may include a detectable label. A detectable label is a group that is detectable at low concentrations, usually less than micromolar, possibly less than nanomolar, and that can be readily distinguished from other molecules, due to differences in a molecular property (e. g. molecular weight, mass to charge ratio, radioactivity, redox potential, luminescence, fluorescence, electromagnetic properties, binding properties, and the like). Detectable labels may be detected by spectroscopic, photochemical, biochemical, immunochemical, electrical, magnetic, electromagnetic, optical or chemical means and the like.

A wide variety of detectable labels are within the scope of the present invention, including hapten labels (e.g. biotin, or labels used in conjunction with detectable antibodies such as horse radish peroxidase antibodies); mass tag labels (e.g. stable isotope labels); radioisotopic labels (including 3H, ¹²⁵I, ³5S, ¹⁴C, or ³²P); metal chelate labels; luminescent labels including fluorescent labels (such as fluorescein, isothiocyanate, Texas red, rhodamine, green fluorescent protein, and the like), phosphorescent labels, and chemiluminescent labels, typically having quantum yield greater than 0.1; electroactive and electron transfer labels; enzyme modulator labels including coenzymes, organometallic catalysts horse radish peroxidase, alkaline phosphatase and others commonly used in an ELISA; photosensitizer labels; magnetic bead labels including Dynabeads; colorimetric labels such as colloidal gold, silver, selenium, or other metals and metal sol labels (see U.S. Pat. No. 5,120,643, which is herein incorporated by reference in its entirety for all purposes), or colored glass or plastic (e.g., polystyrene, polypropylene, latex, etc.) bead labels; and carbon black labels. Patents teaching the use of such detectable labels include U.S. Pat. Nos. 3,817,837; 3,850,752; 3,939,350; 3,996,345; 4,277,437; 4,275,149; 4,366,241; 6,312,914; 5,990,479; 6,207,392; 6,423,551; 6,251,303; 6,306,610; 6,322,901; 6,319,426; 6,326,144; and 6,444,143, which are herein incorporated by reference in their entirety for all purposes.

Detectable labels are commercially available or may be prepared as known to one skilled in the art. Detectable labels may be covalently attached to the compounds using a reactive functional group, which can be located at any appropriate position. Methods for attaching a detectable label are known to one skilled in the art. When the reactive group is attached to an alkyl, or substituted alkyl chain tethered to an aryl nucleus, the reactive group may be located at a terminal position of an alkyl chain.

### COMPOUNDS OF THE INVENTION

The present invention provides compounds of the formula (I) and pharmaceutically acceptable salts and N-oxides thereof:

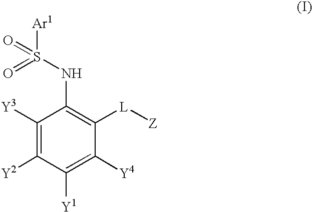

where:

L is selected from the group consisting of —O—, —S—, —S(O)—, —S(O)₂—, —CR^{a}R^{b}—, —NR^{c}—, —NR^{c}C(O)—, —C(O)NR^{c}—, —C(O)—, C=T, and a bond;

R^{c} is hydrogen, substituted or unsubstituted C₁₋₈ alkyl, substituted or unsubstituted C₂₋₆ alkenyl, substituted or unsubstituted C₂₋₆ alkynyl. —C(O)R¹, —C(O)₂R¹, or —S(O)₂R¹, and substituted or unsubstituted 3- to 10-membered heterocyclyl;

R^{a} and R^{b} are each independently hydrogen, halogen, substituted or unsubstituted C₁₋₈ alkyl, substituted or unsubstituted C₂₋₈ alkenyl, substituted or unsubstituted C₂₋₈ alkynyl, —CN, —OR¹, —NR¹R², —NHC(O)R¹, —NHSO₂R¹, —S(O)R¹, or —S(O)₂R²; or where R^{a} and R^{b}, together with the atom to which they are attached are combined to form substituted or unsubstituted C₃₋₈ cycloalkyl or substituted or unsubstituted 3- to 10-membered heterocyclic ring;

R¹ and R² are each independently selected from the group consisting of hydrogen, C₁₋₈ alkyl, C₂₋₈ alkenyl, C₂₋₈ alkynyl, C₆₋₁₀ aryl, 5- to 10-membered heteroaryl, and 3- to 10-membered heterocycle, or when attached to the same nitrogen atom, can be combined with the nitrogen atom to form a 5- or 6-membered heterocyclyl; and

where the aliphatic and aromatic portions of R¹, R², R^{a} and R^{b} can be substituted with from 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl;

T is selected from the group consisting of ═CR^{d}R^{e}, ═NOR^{d}, and ═NR^{d};

R^{d} and R^{e} are each independently selected from the group consisting of hydrogen, halogen (only for ═CR^{d}R^{e}), substituted or unsubstituted C₁₋₈ alkyl, —CN, —OR⁴¹ (only for ═CR^{d}R^{e}), —C(O)R⁴¹, —C(O)₂R⁴¹, —C(O)NR⁴¹R⁴², —SR⁴¹(only for ═CR^{d}R^{e}); —NR⁴¹R⁴² (only for ═CR^{e}R^{e}), —S(O)R⁴¹ (only for ═CR^{d}R^{e}), and —S(O)₂R⁴² (only for ═CR^{d}R^{e}); or where R^{d} and R^{e}, together with the atom to which they are attached, form substituted or unsubstituted C₃₋₈ cycloalkyl or substituted or unsubstituted 3- to 10-membered heterocyclic ring (only for ═CR^{d}R^{e});

R⁴¹ and R⁴² are each independently hydrogen, C₁₋₈ alkyl, 5- to 10-membered heteroaryl, 3 to 10 membered heterocycle, or when attached to the same nitrogen atom, can be combined with the nitrogen atom to form a 5- or 6-membered heterocyclyl, and

substituted C₁₋₈ alkyl, substituted 3- to 10-membered heterocyclic ring, substituted 5- to 10-membered heteroaryl, and the aliphatic portions of R⁴¹ and R⁴² can be substituted with from 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl.

Ar¹ is a substituted or unsubstituted C₆₋₁₀ aryl or substituted or unsubstituted 5- to 10-membered heteroaryl; each having 0 to 5 substituents selected from the group consisting of halogen, substituted or unsubstituted C₁₋₈ alkyl, substituted or unsubstituted C₂₋₈ alkenyl, substituted or unsubstituted C₂₋₈ alkynyl, —CN, —NO₂, ═O, —C(O)R³, —CO₂R³, —C(O)NR³R⁴, —OR³, —OC(O)R³, —OC(O)NR³R⁴, —NR⁵C(O)R³, —NR⁵C(O)NR³R⁴, —NR³R⁴, —NR⁵CO₂R³, —NR⁵S(O)₂R³, —SR³, —S(O)R³, —S(O)₂R³, —S(O)₂NR³R⁴, substituted or unsubstituted C₆₋₁₀ aryl, substituted or unsubstituted 5- to 10-membered heteroaryl, and substituted or unsubstituted 3- to 10-membered heterocyclyl;

suitable substituted C₁₋₈ alkyl, substituted C₂₋₈ alkenyl, or substituted C₂₋₈ alkynyl may have from 1-5 substituents independently selected from the group consisting of halogen, —OH, —CN, —NO₂, ═O, —OC(O)R³, —OR³, —C(O)R³, —C(O)NR³R⁴, —OC(O)NR³R⁴, —NR⁴C(O)R³, —NR³C(O)NR⁴R⁵, —CO₂R³, —NR³R⁴, —NR⁴CO₂R³, —SR³, —S(O)R³, —S(O)₂R³, —S(O)₂NR³R⁴, —NR³S(O)₂R⁴, unsubstituted or substituted aryl, unsubstituted or substituted heteroaryl, and unsubstituted or substituted heterocyclyl;

suitable substituted C₆₋₁₀ aryl, substituted 5- to 10-membered heteroaryl, or substituted 3- to 10-membered heterocyclyl, may have from 1-4 substituents independently selected from the group consisting of halogen, unsubstituted C₁₋₈ alkyl, unsubstituted C₁₋₈ haloalkyl, —CN, —NO₂, —OR³, ═O, —OC(O)R³, —CO₂R³, —C(O)R³, —C(O)NR³R⁴, —OC(O)NR³R⁴, —NR⁴C(O)R³, —NR³C(O)NR⁴R⁵, —NR³R⁴, —NR⁴CO₂R³, —SR³, —S(O)R³, —S(O)₂R³, —S(O)₂NR³R⁴, and —NR³S(O)₂R⁴;

where R³, R⁴, and R⁵ are each independently selected from the group consisting of hydrogen, C₁₋₈ alkyl, C₂₋₈ alkenyl, C₂₋₈ alkynyl, C₆₋₁₀ aryl, 5- to 10-membered heteroaryl, and 3- to 10-membered heterocycle, or where R³ and R⁴, together with the atom(s) to which they are attached, form an substituted or unsubstituted 5-, 6-, or 7-membered ring;

where the aliphatic and aromatic portions of R³, R⁴ and R⁵ can be substituted with from 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHR^{m}, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl.

Y¹, Y², Y³, and Y⁴ are each independently selected from the group consisting of hydrogen, halogen, substituted or unsubstituted C₁₋₄ alkyl, —CN, —C(O)R⁶, —CO₂R⁶, —OR⁶, —NO₂, —SR⁶, —S(O)R⁶, and —S(O)₂R⁶;

where substituted C₁₋₄ alkyl can have from 1 to 3 substituents independently selected from the group consisting of halogen, —OH, —OR⁶, —CN, —NO₂, ═O, —OC(O)R⁶, —CO₂R⁶, —C(O)R⁶, —C(O)NR⁶R¹³, —OC(O)NR⁶R¹³, —NR¹³C(O)R⁶, —NR⁶C(O)NR¹³R¹⁴, —NR⁶R¹³, —NR¹³CO₂R⁶, —SR⁶, —S(O)R⁶, —S(O)₂R⁶, —S(O)₂NR⁶R¹³, and —NR¹³S(O)₂R⁶;

where R⁶, R¹³, and R¹⁴ are independently selected from the group consisting of hydrogen, C₁₋₈ alkyl, C₂₋₈ alkenyl, C₂₋₈ alkynyl, C₆₋₁₀ aryl, and 5- to 10-membered heteroaryl; and

where the aliphatic and aromatic portions of R⁶, R¹³, and R¹⁴ can be substituted with from 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl.

Z is a substituted or unsubstituted C₆₋₁₀ aryl, a substituted or unsubstituted 5- to 10-membered heteroaryl, a substituted or unsubstituted 3- to 10-membered heterocyclyl or —NR¹⁷R¹⁸.

When Z is —NR¹⁷R¹⁸, R¹⁷ and R¹⁸ are each independently selected from the group consisting of hydrogen, C₁₋₈ alkyl, C₂₋₈ alkenyl, C₂₋₈ alkynyl, C₆₋₁₀ aryl, C₁₋₈ alkylaryl, C₁₋₈ alkylheteroaryl, 5- to 10-membered heteroaryl and 3- to 10-membered heterocycle, or R¹⁷ and R¹⁸, may together with the atom(s) to which they are attached, form an substituted or unsubstituted 5-, 6-, or 7-membered ring; and

where the aliphatic and aromatic portions of R¹⁷ and R¹⁸ can be substituted with 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl.

When Z is substituted or unsubstituted C₆₋₁₀ aryl, a substituted or unsubstituted 5- to 10-membered heteroaryl, a substituted or unsubstituted 3- to 10-membered heterocyclyl, it may be substituted with 0 to 4 substituents selected from the group consisting of halogen, substituted or unsubstituted C₁₋₈ alkyl, substituted or unsubstituted C₂₋₈ alkenyl, substituted or unsubstituted C₂₋₈ alkynyl, —CN, ═O, —NO₂, —OR⁷, —OC(O)R⁷, —CO₂R⁷, —C(O)R⁷, —C(O)NR⁸R⁹, —OC(O)NR⁸R⁹, —NR⁷C(O)R⁸, —NR⁷C(O)NR⁸R⁹, —NR⁸R⁹, —NR⁷CO₂R⁸, —SR⁷, —S(O)R⁷, —S(O)₂R⁷, —S(O)₂NR⁸R⁹, —NR⁷S(O)₂R⁸, substituted or unsubstituted C₆₋₁₀ aryl, substituted or unsubstituted 5- to 10-membered heteroaryl and substituted or unsubstituted 3- to 10-membered heterocyclyl;

suitable substituted C₁₋₈ alkyl, C₂₋₈ alkenyl, C₂₋₈ alkynyl may have from 1 to 5 substituents independently selected from the group consisting of halogen, —OH, OR⁷, —CN, —NO₂, ═O, —OC(O)R⁷, —CO₂R⁷, —C(O)R⁷, —C(O)NR⁷R⁸, —OC(O)NR⁷R⁸, —NR⁷C(O)R⁸, —NR⁷C(O)NR⁸R⁹, —NR⁷R⁸, ⁻NR⁷CO₂R⁸, —SR⁷, —S(O)R⁷, —S(O)₂R⁷, —S(O)₂NR⁷R⁸, —NR⁷S(O)₂R⁸, unsubstituted or substituted phenyl, unsubstituted or substituted C₅₋₆ heteroaryl, or unsubstituted or substituted C₃₋₆ heterocyclyl;

suitable substituted aryl, heteroaryl and heterocyclyl substituents may have from 1 to 5 substituents independently selected from the group consisting of halogen, —OR⁷, —CN, —NO₂, ═O, —OC(O)R⁷, —CO₂R⁷, —C(O)R⁷, —C(O)NR⁷R⁸, —OC(O)NR⁷R⁸, —NR⁷C(O)R⁸, —NR⁷C(O)NR⁸R⁹, —NR⁷R⁸, —NR⁷CO₂R⁸, —SR⁷, —S(O)R⁷, —S(O)₂R⁷, —S(O)₂NR⁷R⁸, —NR⁷S(O)₂R⁸, unsubstituted C₃₋₆ heterocyclyl, unsubstituted C₁₋₈ alkyl, and unsubstituted C₁₋₈ haloalkyl;

where R⁷, R⁸ and R⁹ are each independently selected from the group consisting of hydrogen, C₁₋₈ alkyl, C₂₋₈ alkenyl, C₂₋₈ alkynyl, C₆₋₁₀ aryl, 5 to 10 membered heteroaryl and 3- to 10-membered heterocycle, or R⁷, R⁸ and R⁹, may together with the atom(s) to which they are attached, form a substituted or unsubstituted 5-, 6-, or 7-membered ring, and

where the aliphatic and aromatic portions of R⁷, R⁸ and R⁹ can be substituted with 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂ and —NR^{m}S(O)₂NHRⁿ, where R^{m,} Rⁿ, and R^{o} are each independently unsubstituted C₁₋₆ alkyl.

In another embodiment, the present invention provides compounds of the formula (II) and pharmaceutically acceptable salts and N-oxides thereof:

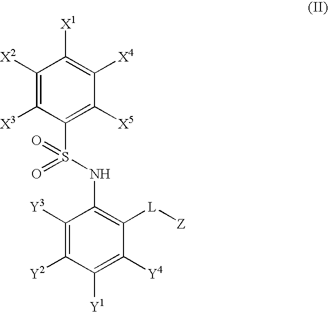

where Z, Y¹, Y², Y³, Y⁴, and L are as defined in formula (I); and

X¹, X², X³, X⁴, X⁵ are each independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, —CN, —C(O)R³, —CO₂R³, —C(O)NR³R⁴, —OR³, —OC(O)R³, —OC(O)NR³R⁴, —NO₂, —NR⁵C(O)R³, —NR⁵C(O)NR³R⁴, —NR³R⁴, —NR⁵CO₂R³, —NR⁵S(O)₂R³, —SR³, —S(O)R³, —S(O)₂R³, —S(O)₂NR³R⁴, substituted or unsubstituted C₆₋₁₀ aryl, substituted or unsubstituted 5- to 10-membered heteroaryl, and substituted or unsubstituted 3- to 10-membered heterocyclyl;

where R³, R⁴, and R⁵ are each independently selected from the group consisting of hydrogen, C₁₋₈ alkyl, C₂₋₈ alkenyl, C₂₋₈ alkynyl, C₆₋₁₀ aryl, 5- to 10-membered heteroaryl, and 3- to 10-membered heterocycle, or where R³ and R⁴, together with the atom(s) to which they are attached, form an substituted or unsubstituted 5-, 6-, or 7-membered ring.

In another embodiment, the present invention provides compounds of the formula (III) and pharmaceutically acceptable salts and N-oxides thereof,

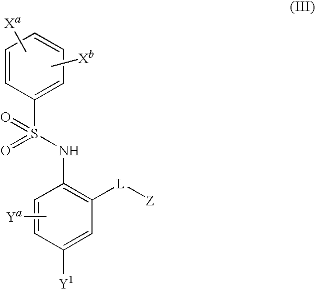

where X^{a} and X^{b} are each independently as defined for X¹ in formula (II); Y¹ and Y^{a} are each independently as defined for Y¹ in formula (I); and Z and L are as defined for formula (I).

In another embodiment, the present invention provides compounds of the formula (IV) and pharmaceutically acceptable salts and N-oxides thereof,

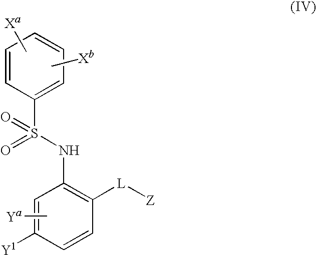

where X^{a} and X^{b} are each independently as defined for X¹ in formula (II); Y¹ and Y^{a} are each independently as defined for Y¹ in formula (I); and Z and L are as defined for formula (I).

In another embodiment, the present invention provides compounds of the formula (V) and pharmaceutically acceptable salts and N-oxides thereof,

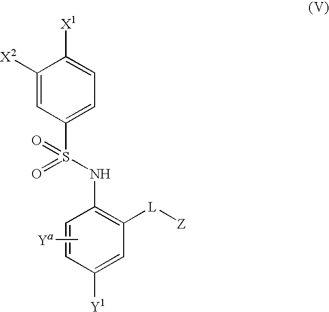

where X¹ and X² are each independently as defined in formula (II); Y¹ and Y^{a} are each independently as defined for Y¹ in formula (I); and Z and L are as defined for formula (I).

In another embodiment, the present invention provides compounds of the formula (VI) and pharmaceutically acceptable salts and N-oxides thereof,

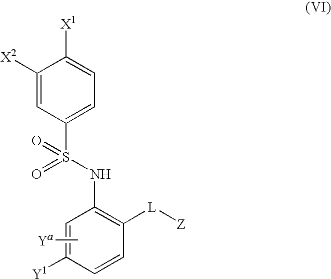

where X¹ and X² are each independently as defined in formula (II); Y¹ and Y^{a} are each independently as defined for Y¹ in formula (I); and Z and L are as defined for formula (I).

In another embodiment, the present invention provides compounds of the formula (VII) and pharmaceutically acceptable salts and N-oxides thereof,

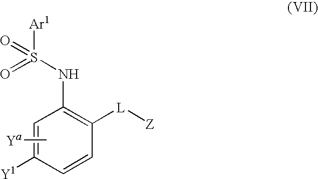

where Ar¹, Y¹ and Y^{a} are each independently as defined for Y¹ in formula (I); and Z and L are as defined for formula (I). Preferably L is —C(O)—, —C(O)NR^{c}—, —NR^{c}C(O)—, or a bond.

In another embodiment, the present invention provides compounds of the formula (VIII) and pharmaceutically acceptable salts and N-oxides thereof,

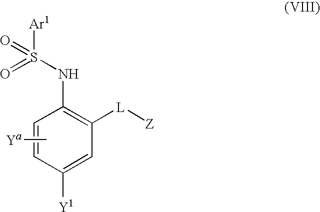

where Ar¹, Y¹ and Y^{a} are each independently as defined for Y¹ in formula (I); and Z and L are as defined for formula (I). Preferably L is —C(O)—, —C(O)NR^{c}—, —NR^{c}C(O)—, or a bond.

In other embodiments, the present invention provides compounds of the formula (XX-CXXXVI) and pharmaceutically acceptable salts and N-oxides thereof:

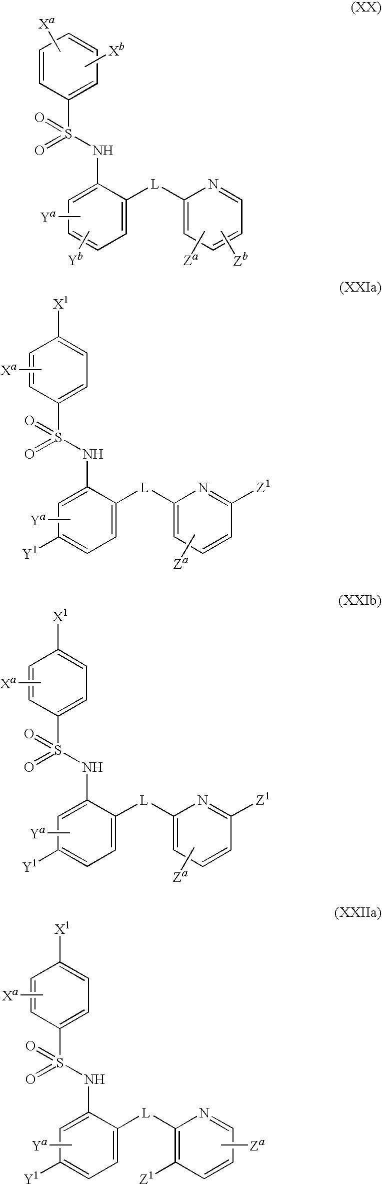

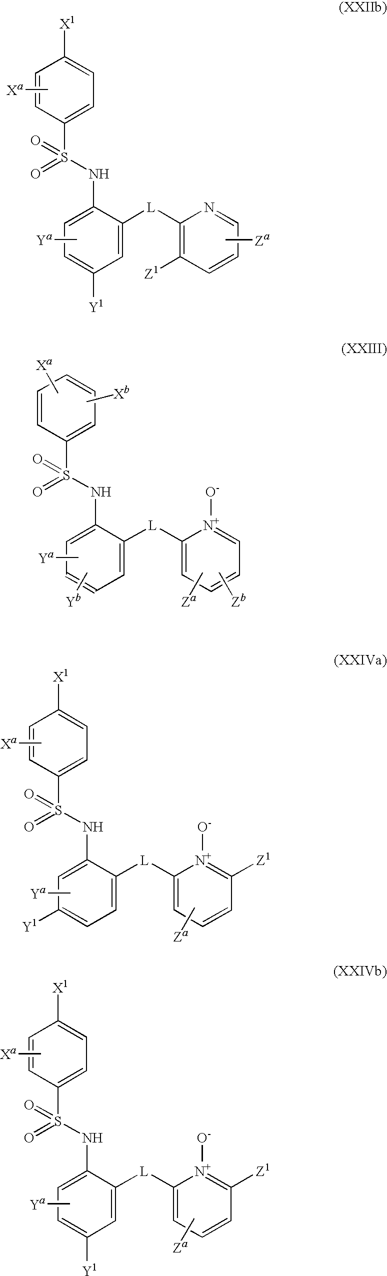

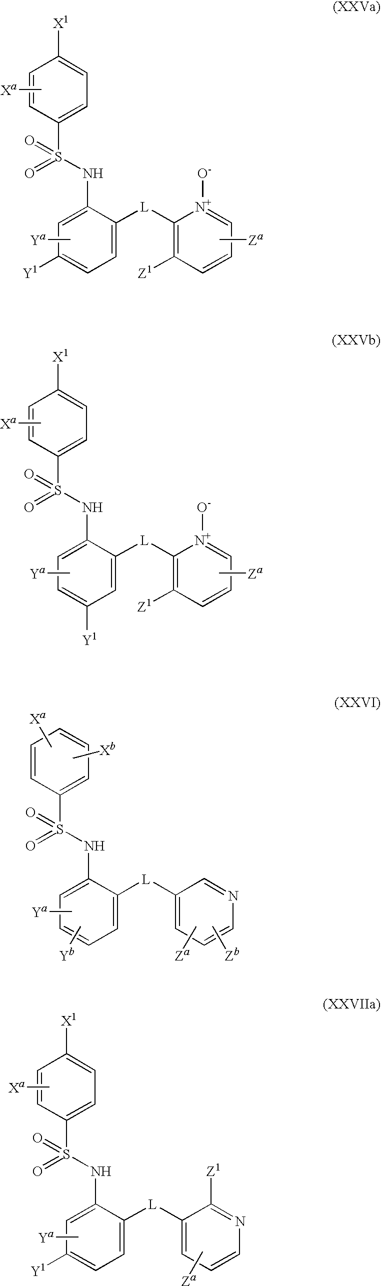

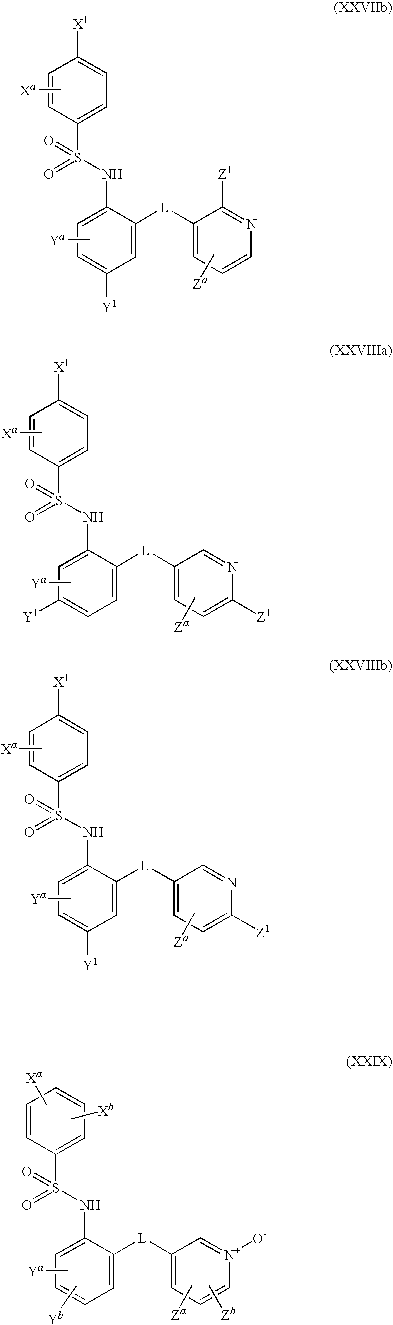

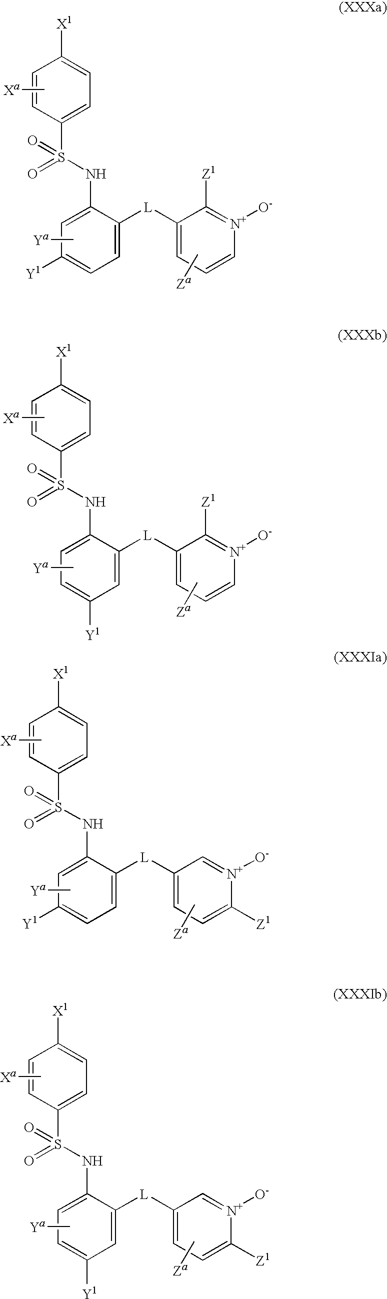

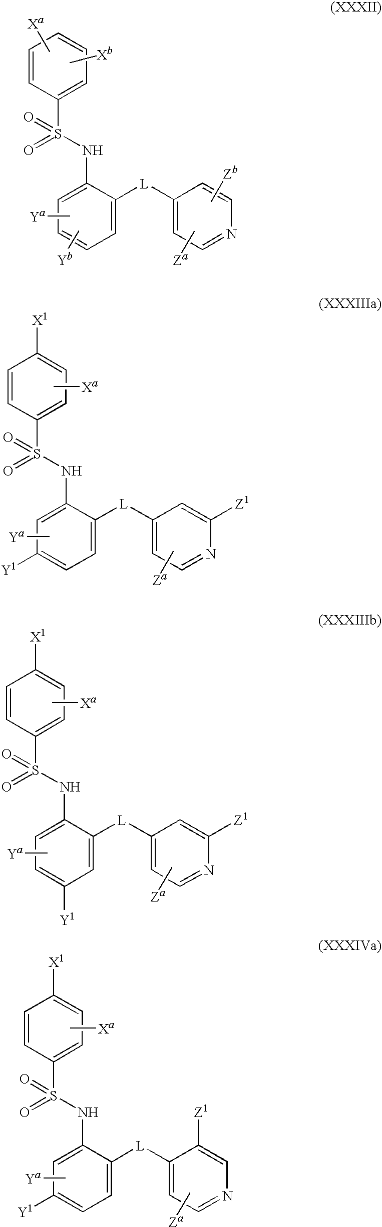

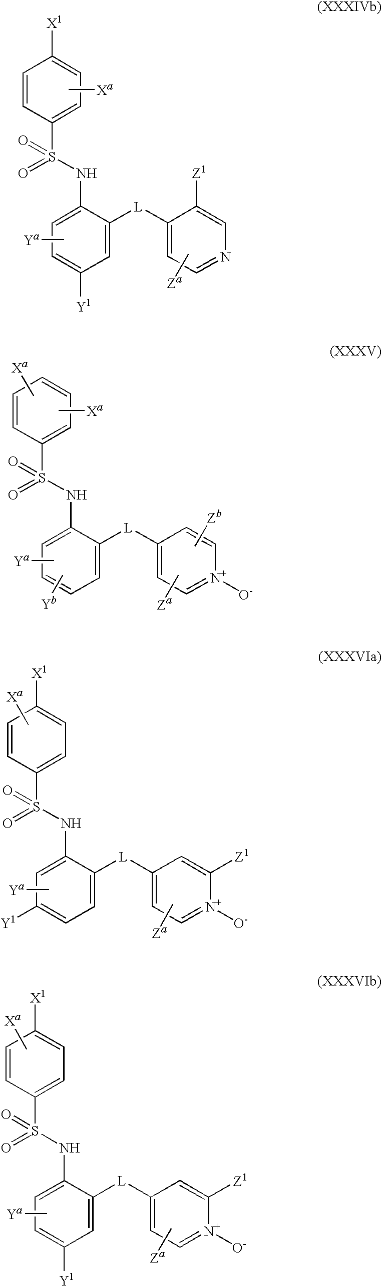

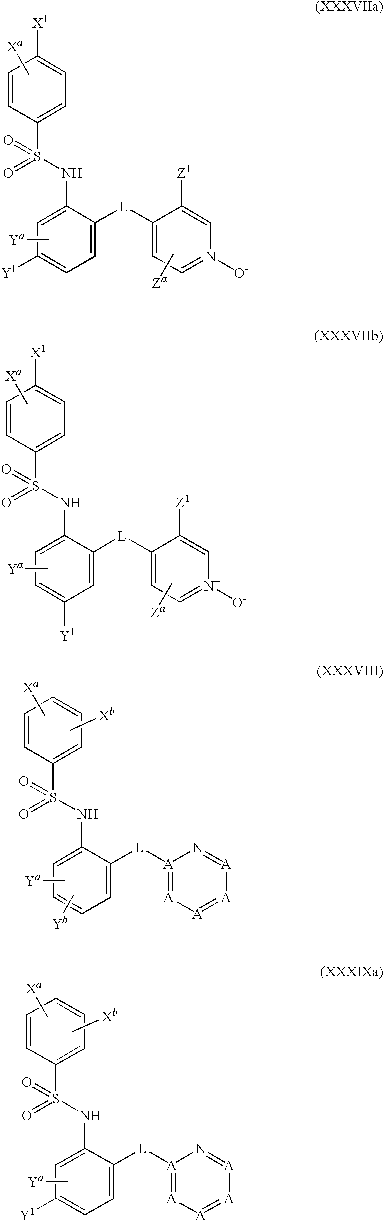

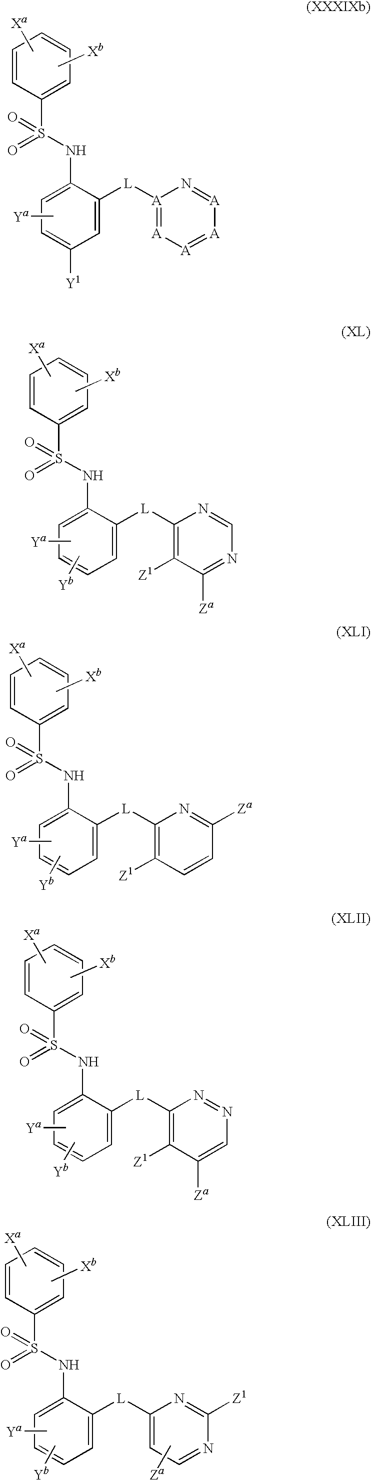

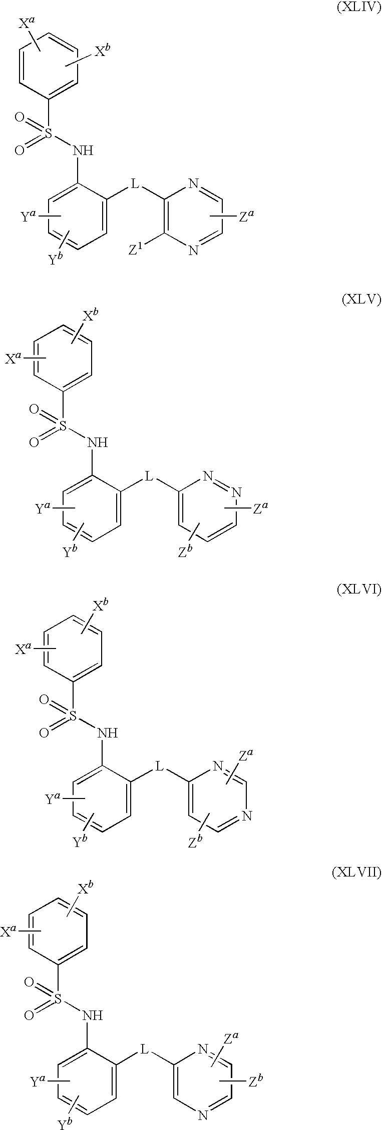

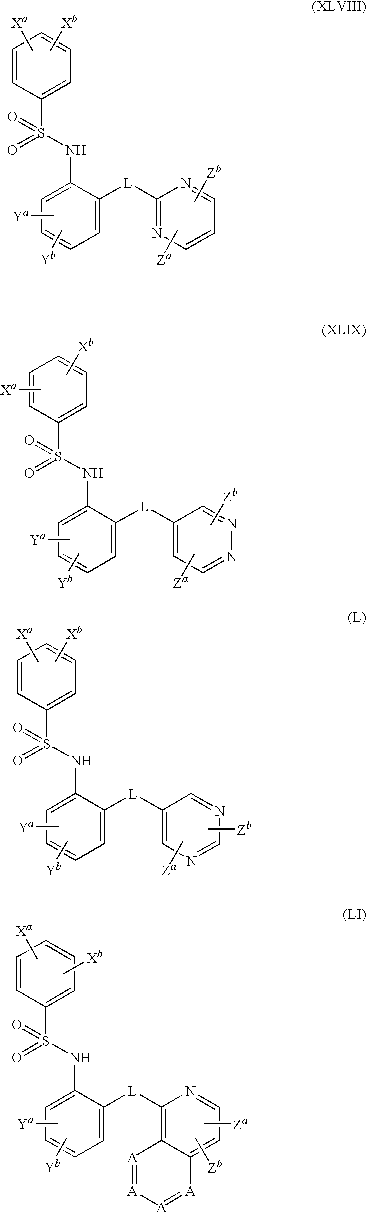

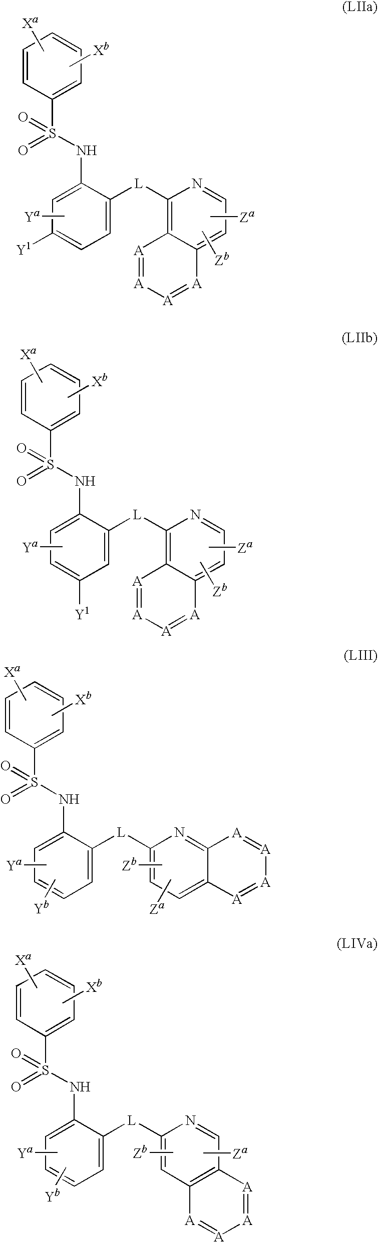

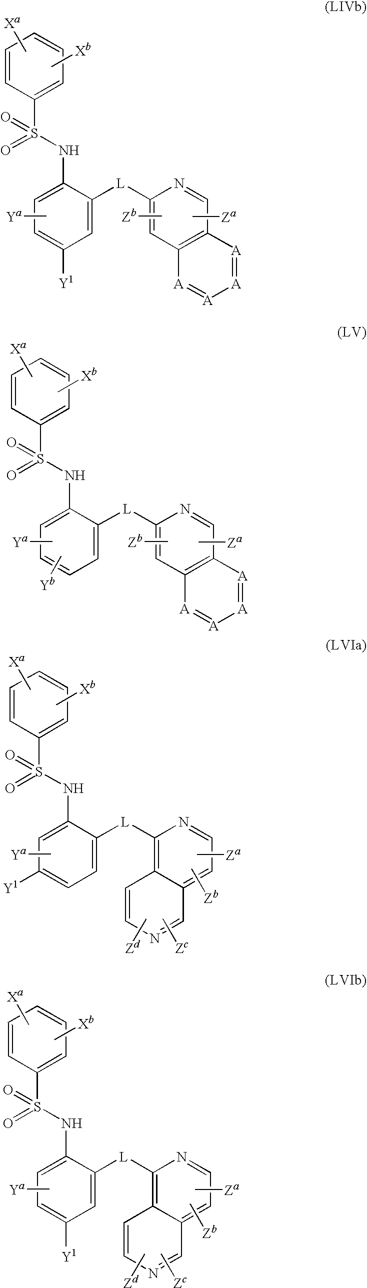

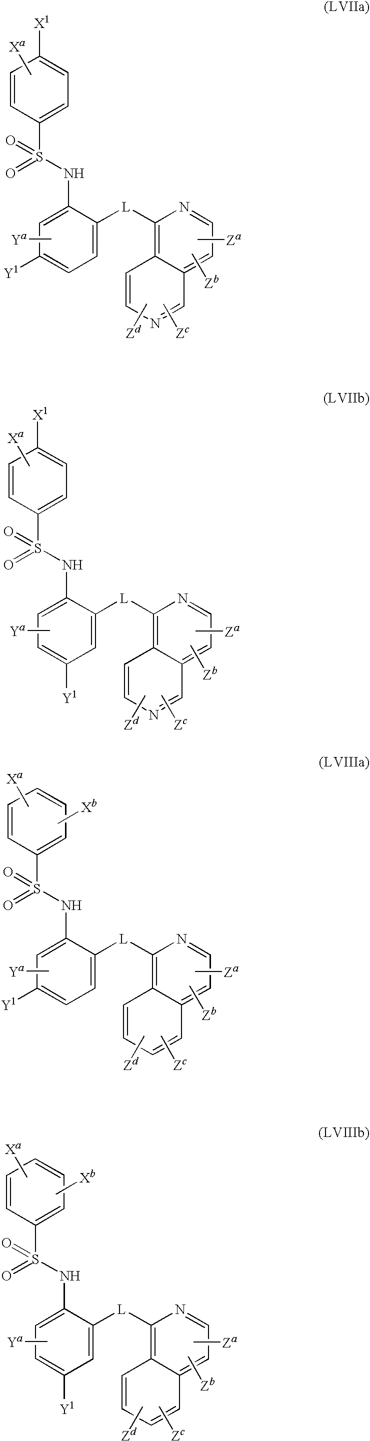

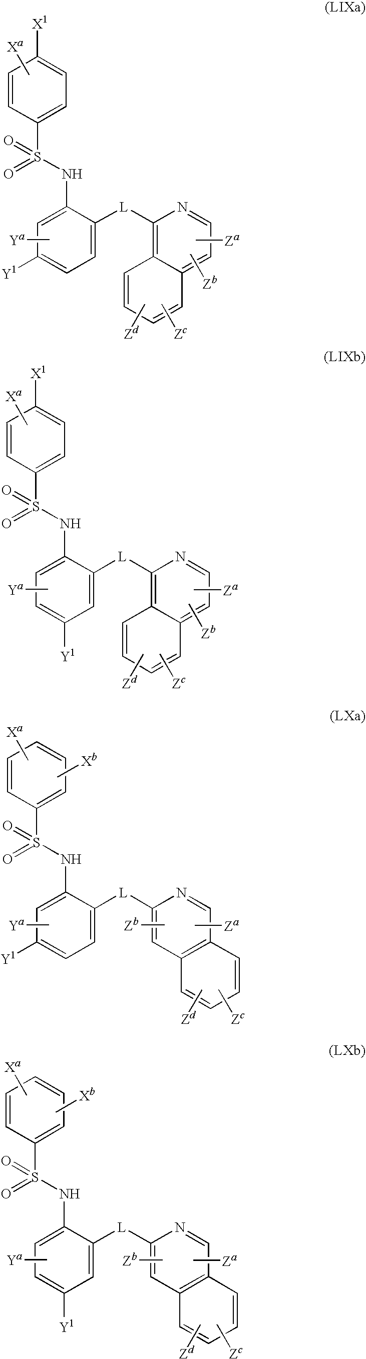

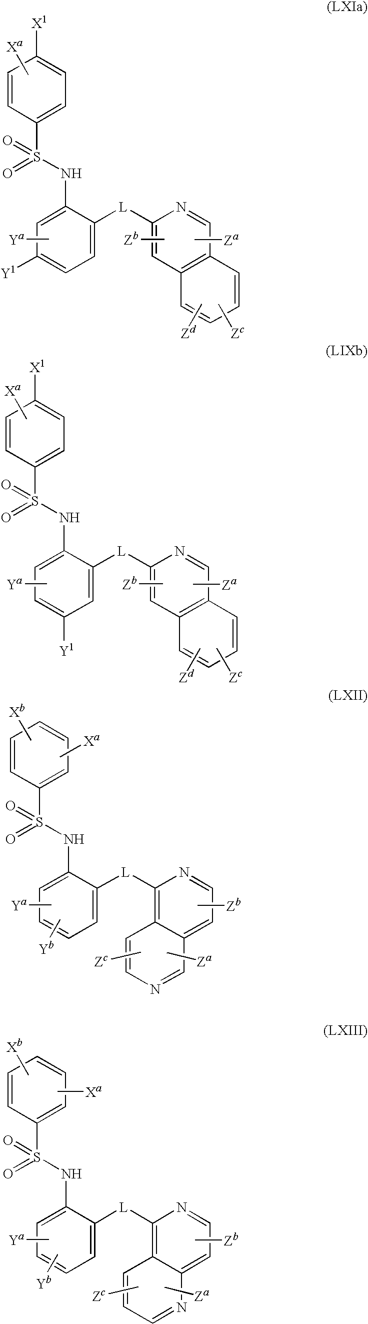

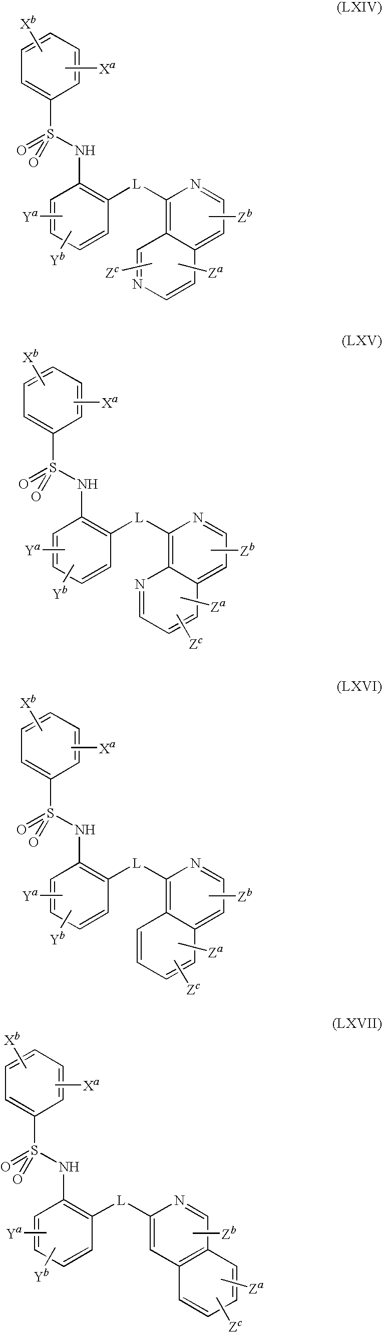

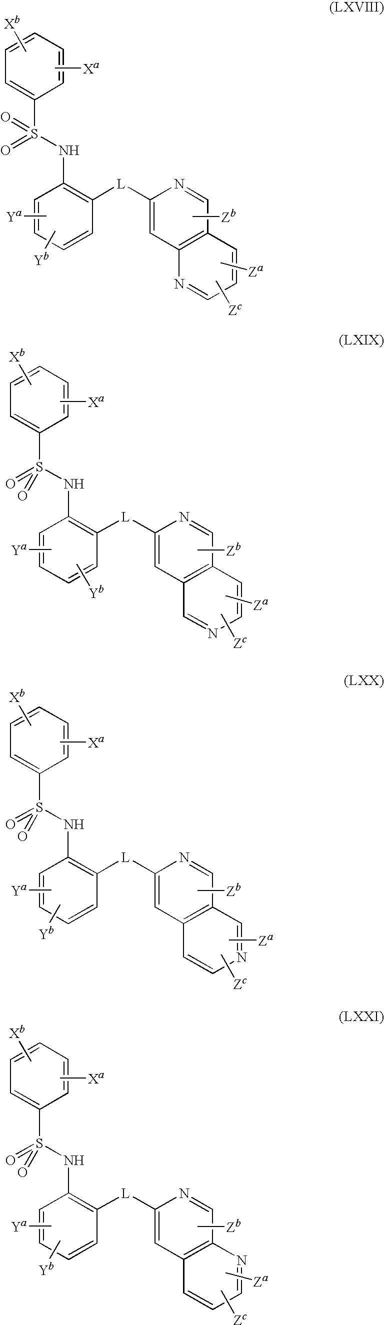

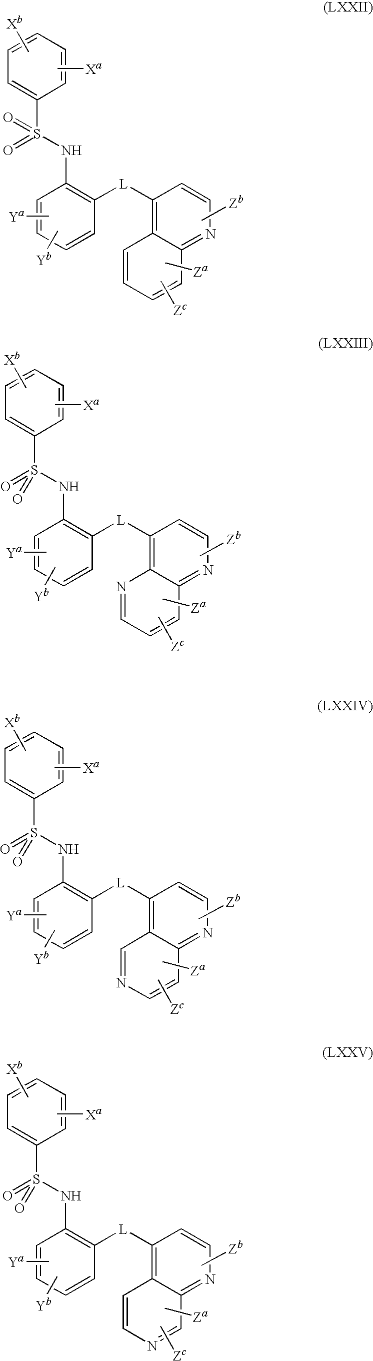

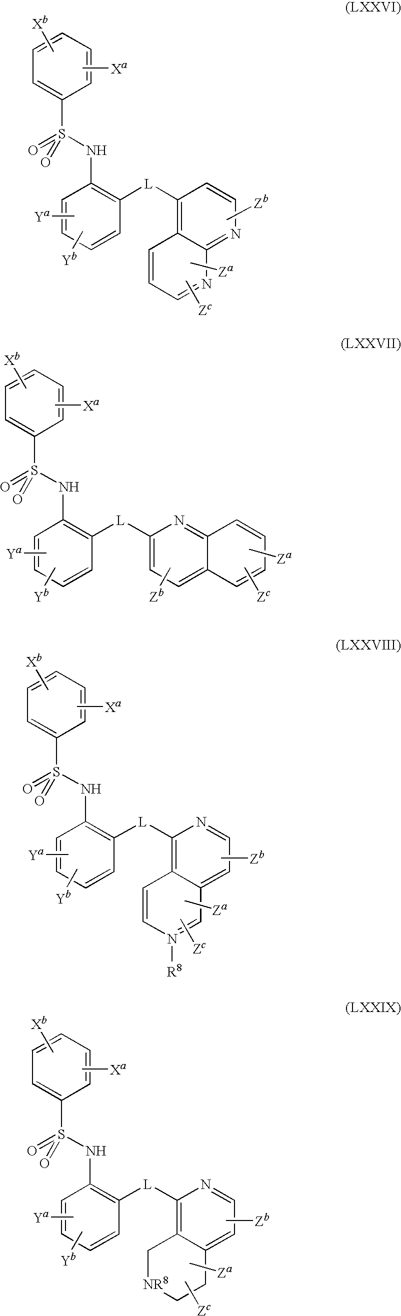

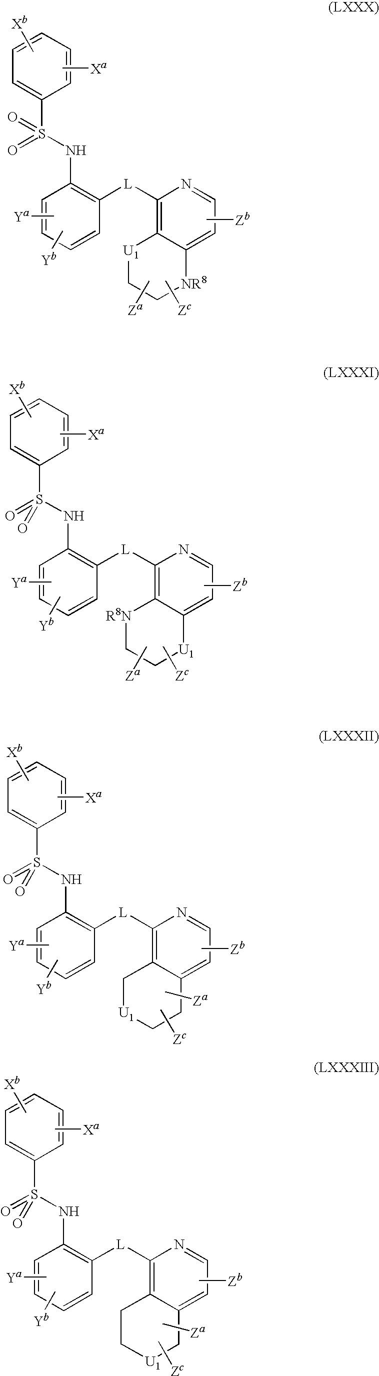

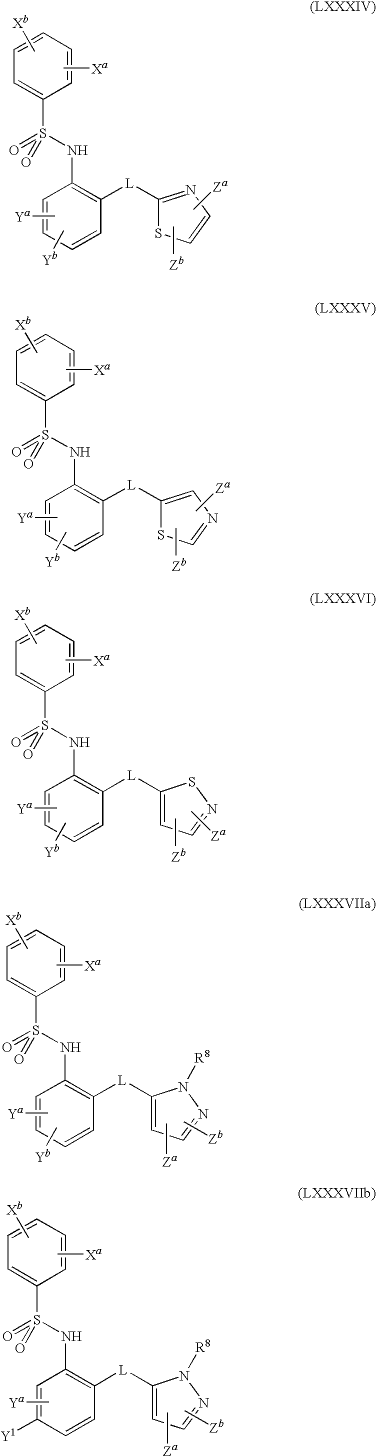

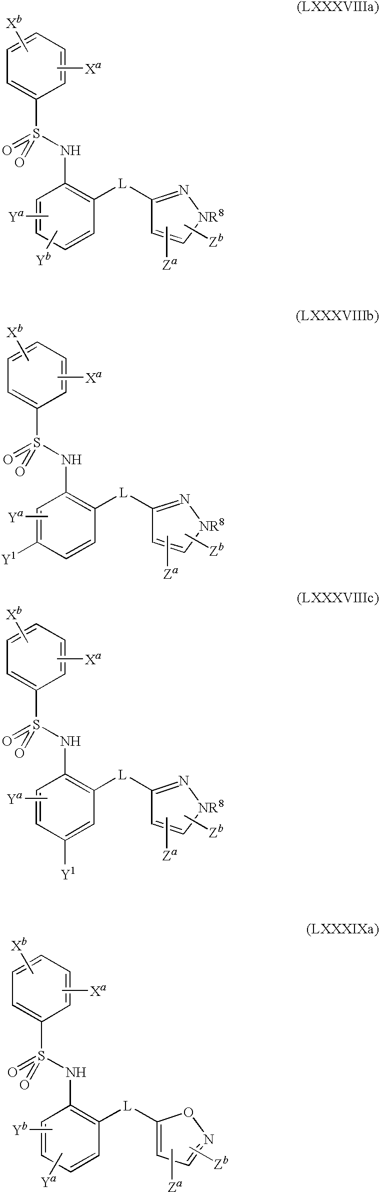

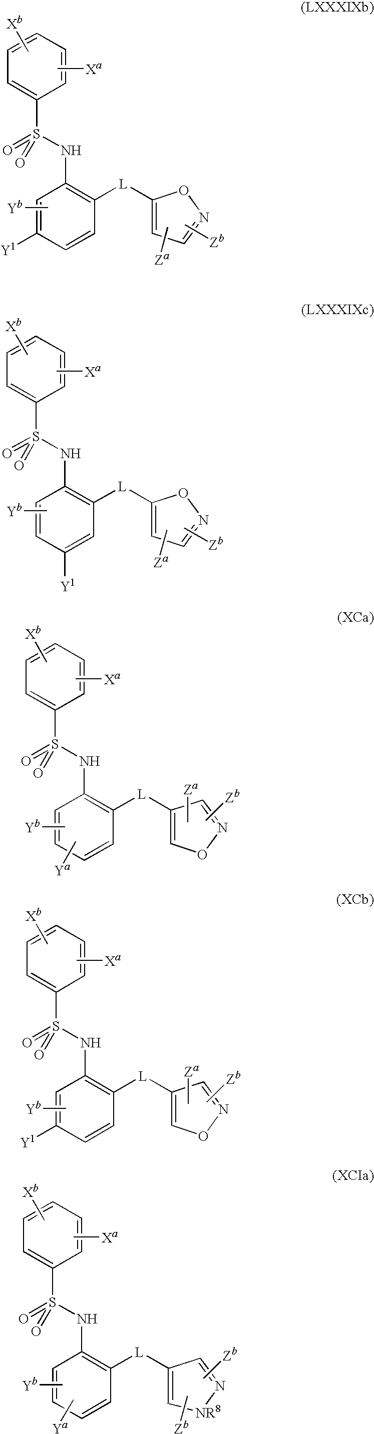

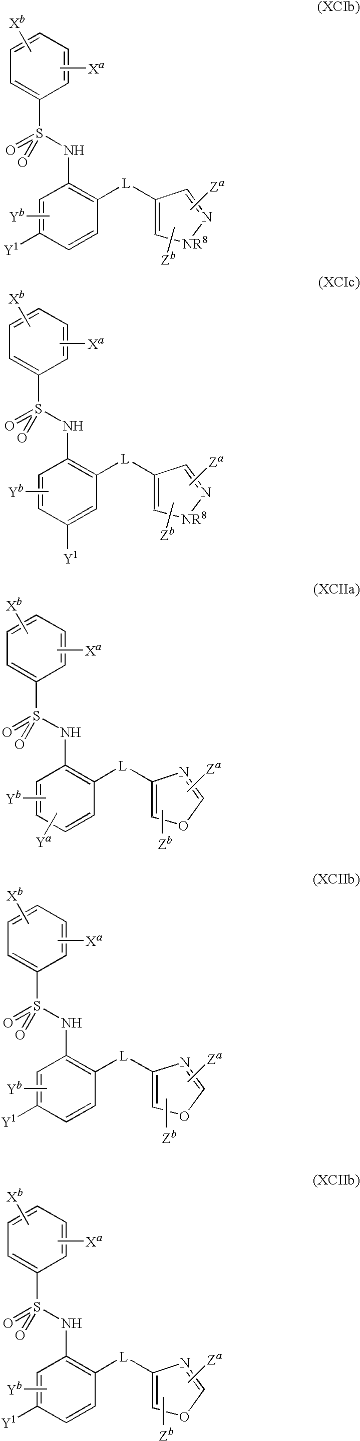

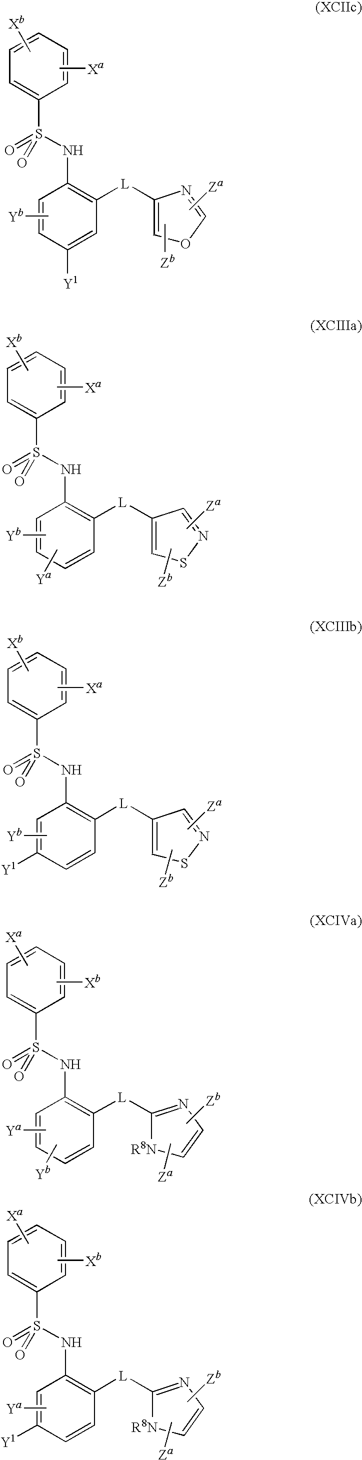

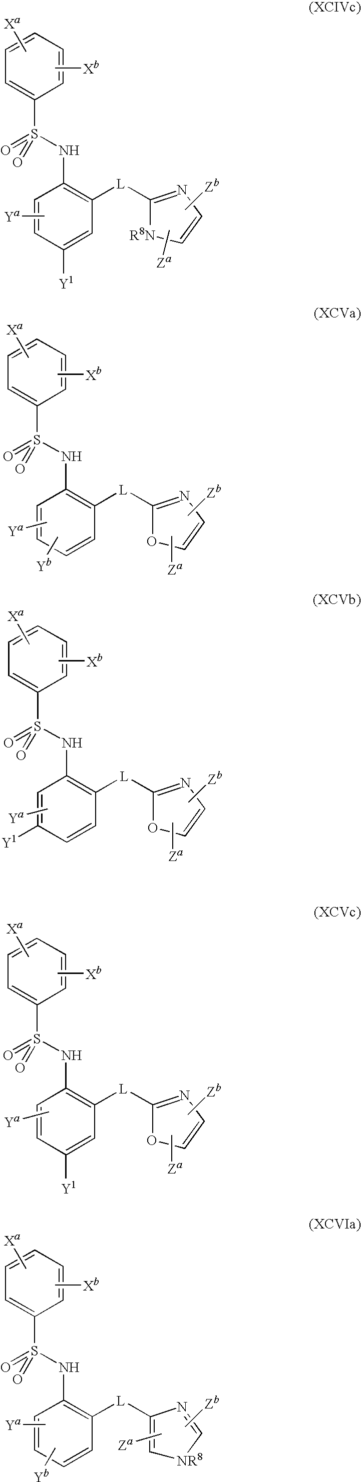

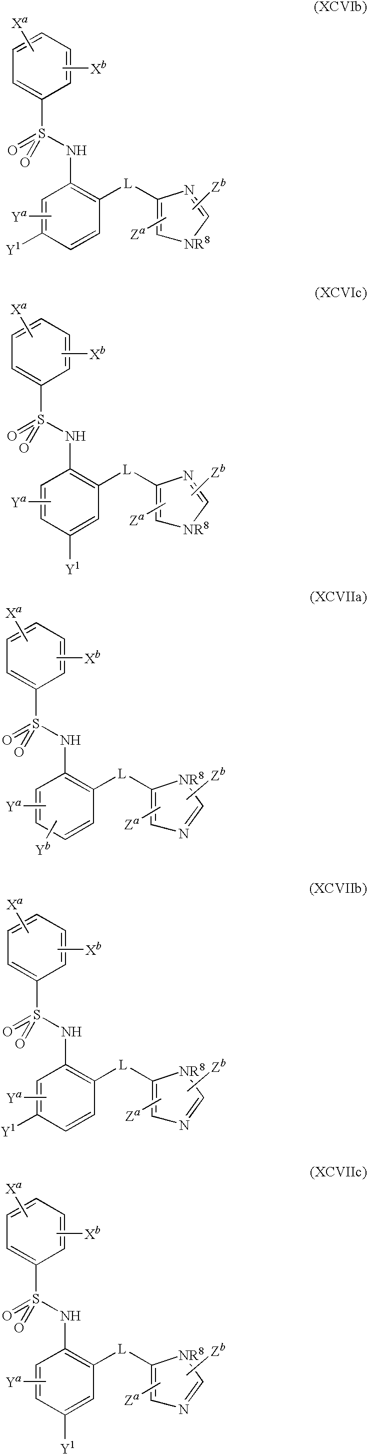

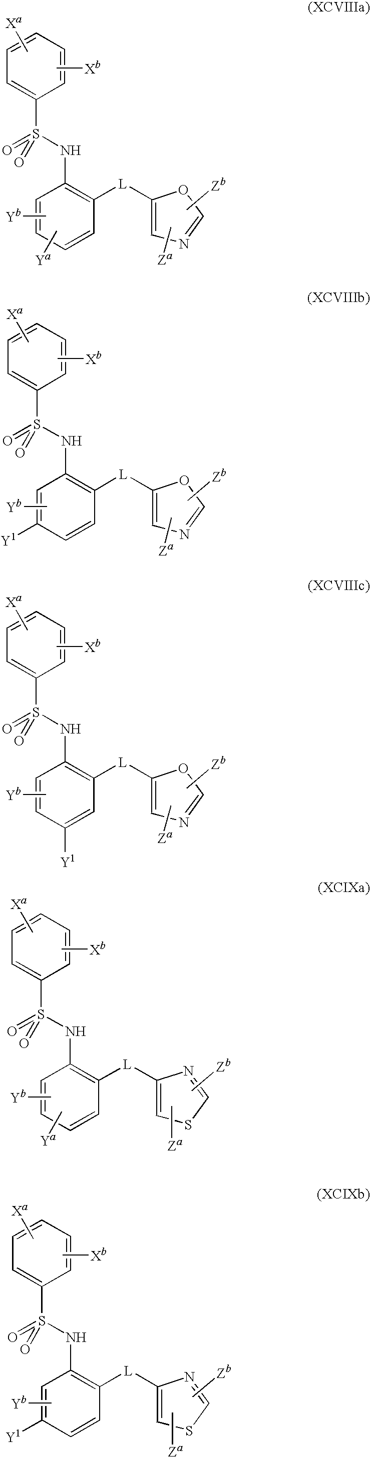

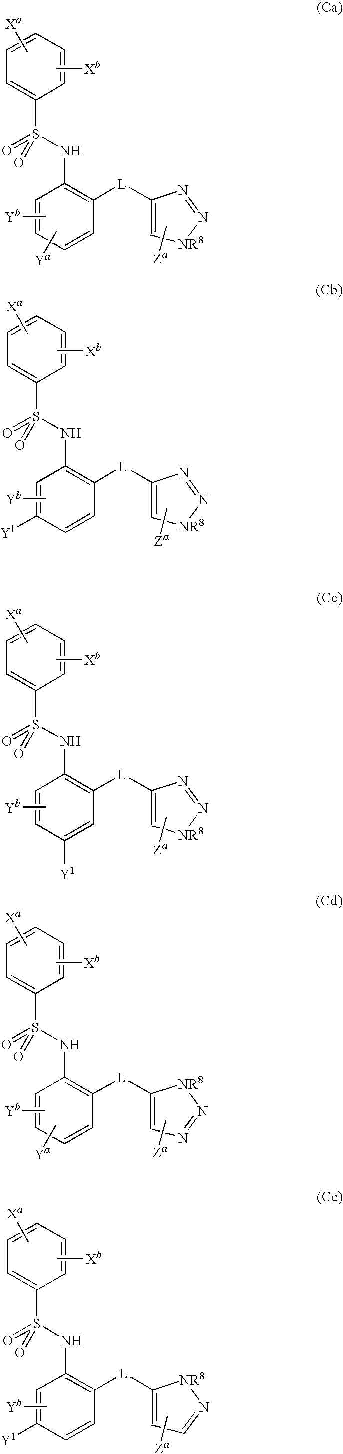

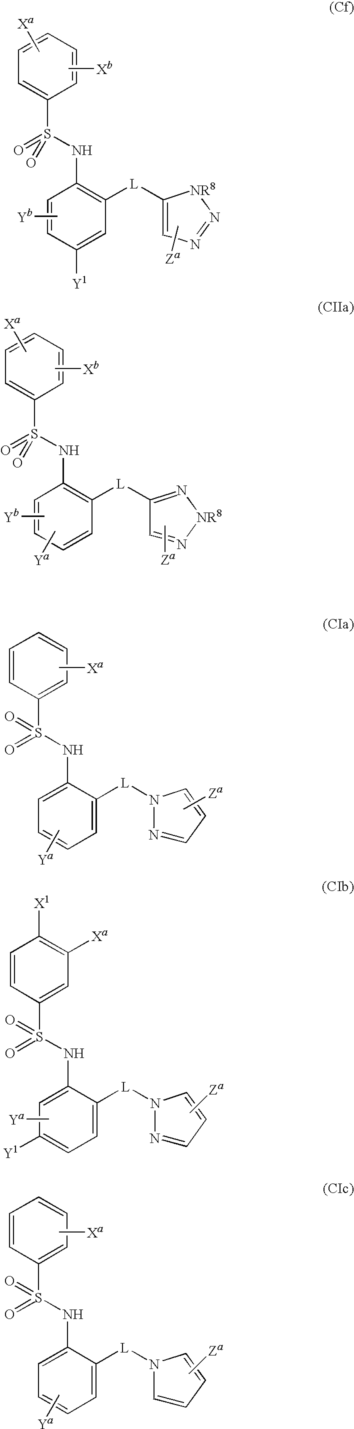

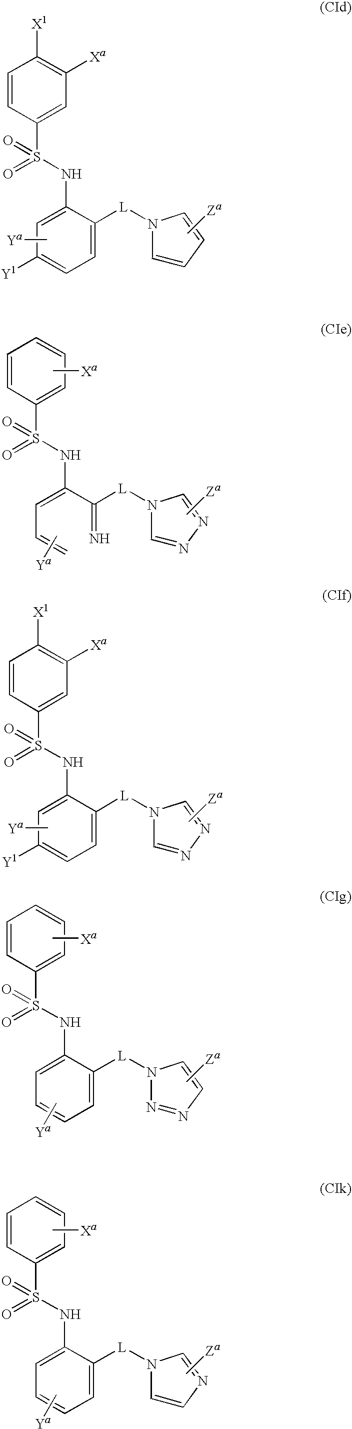

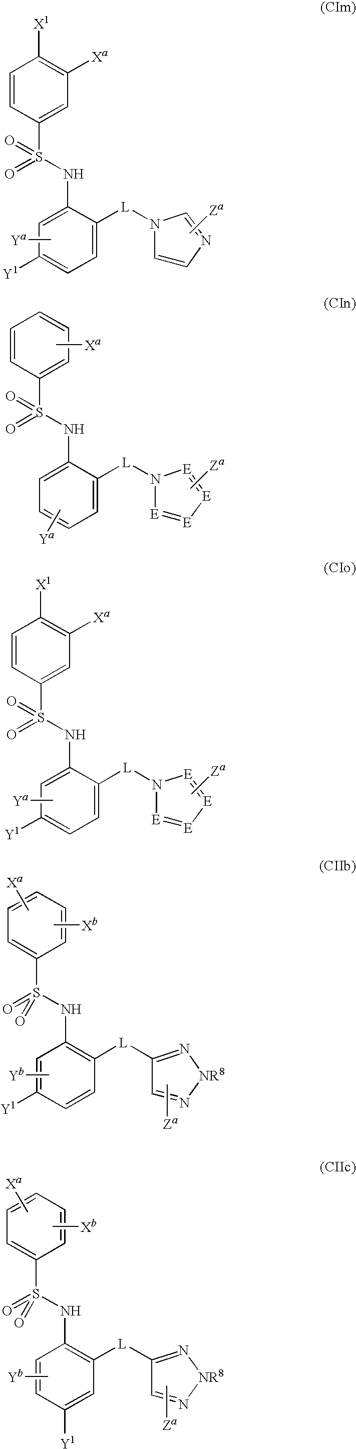

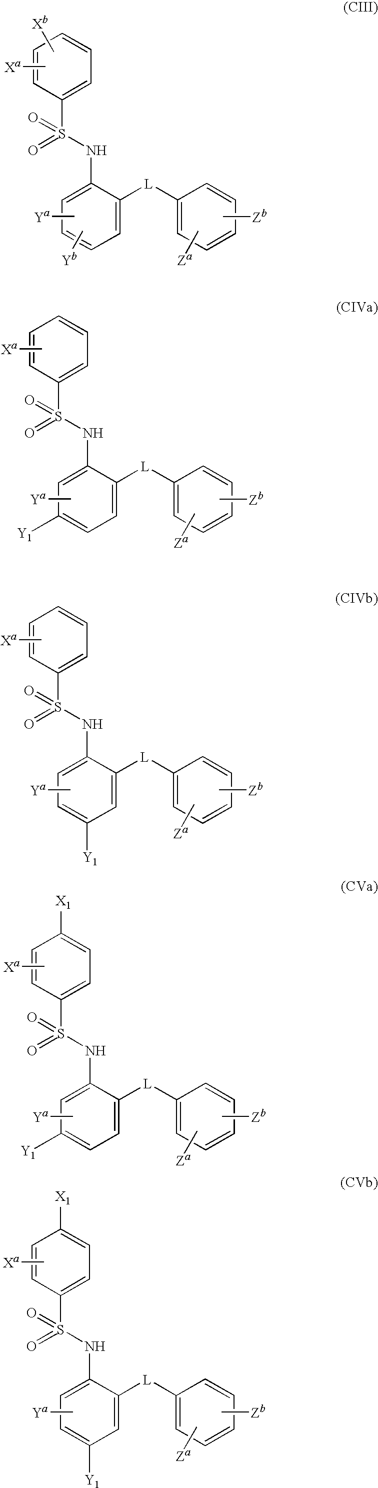

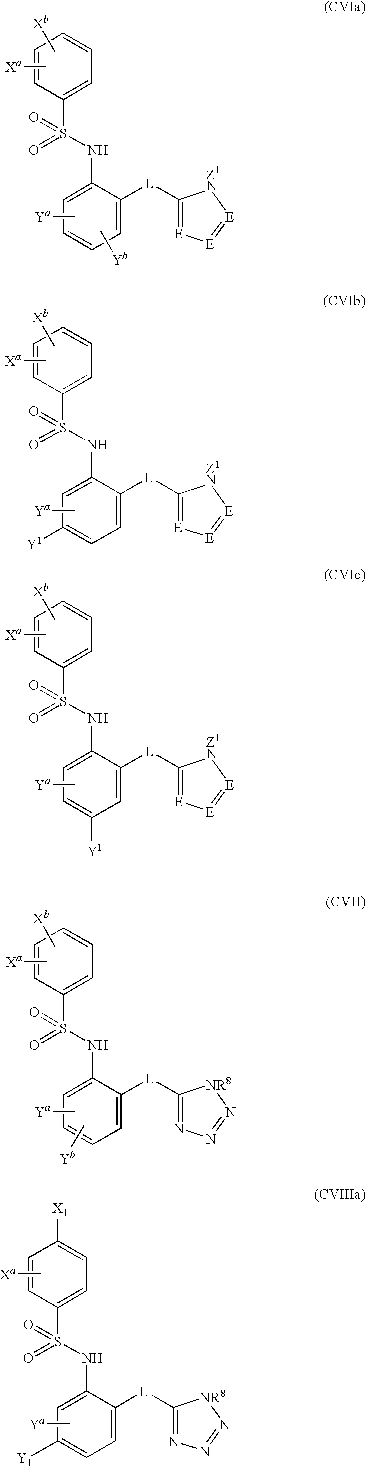

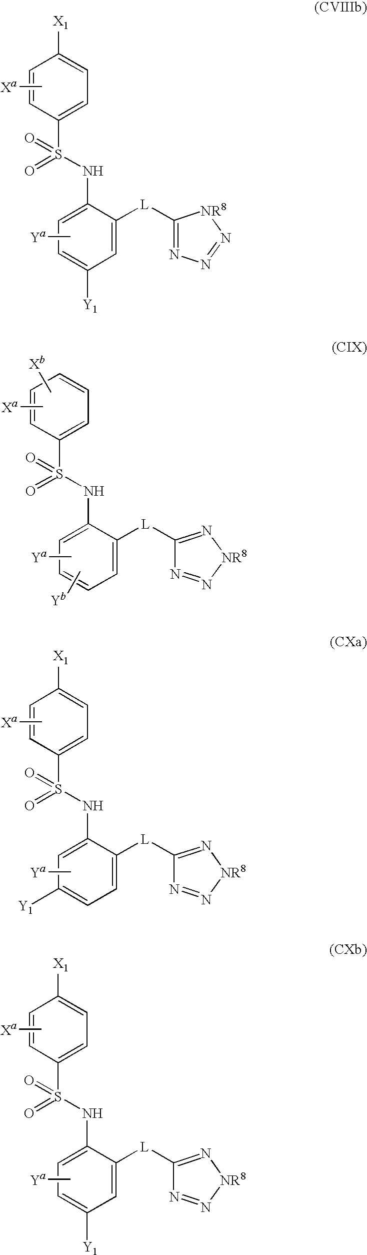

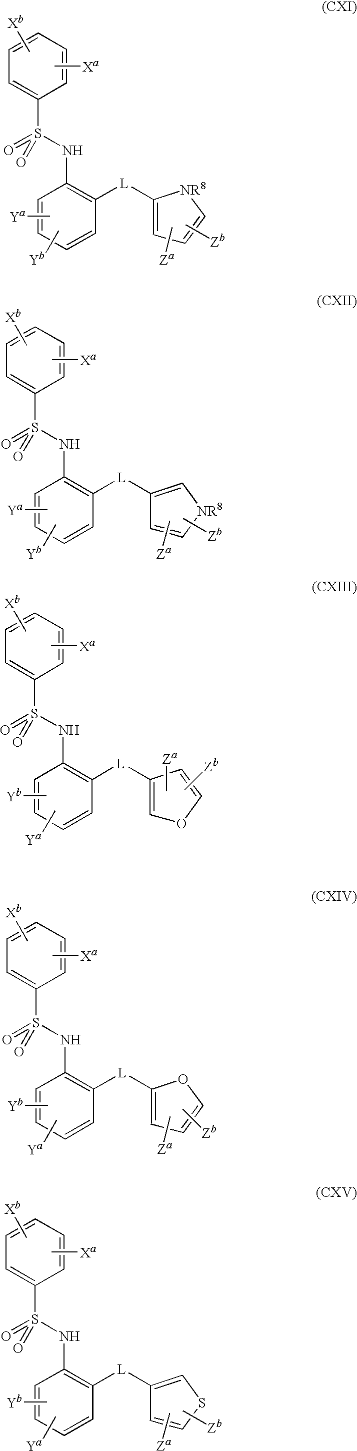

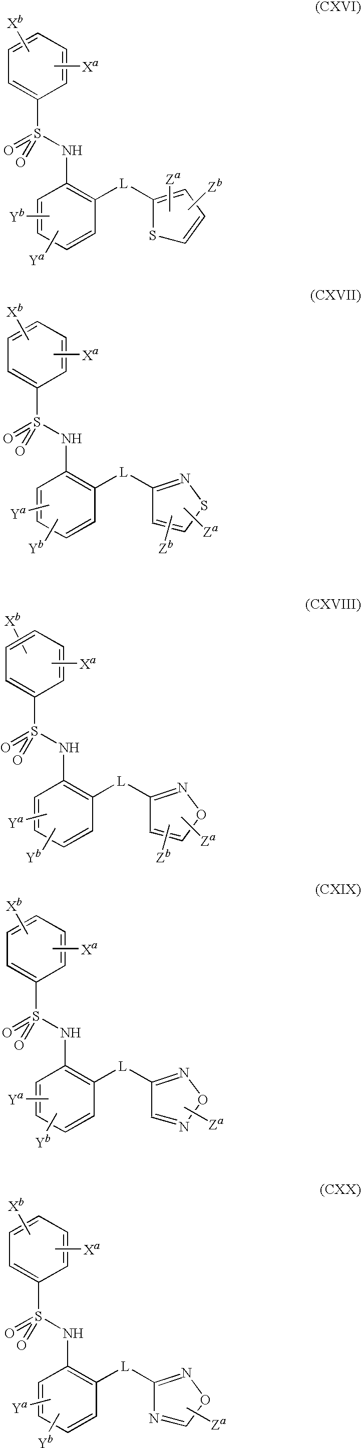

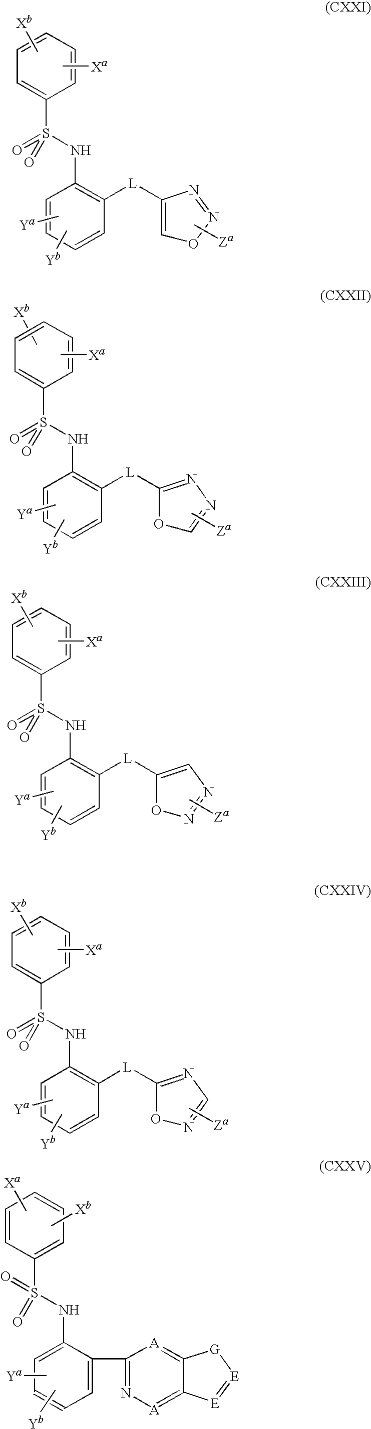

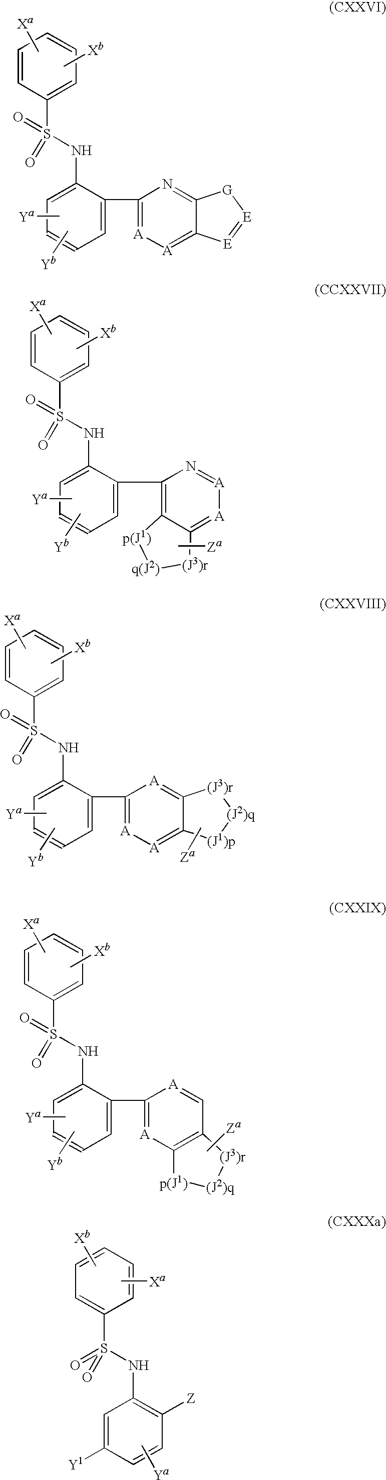

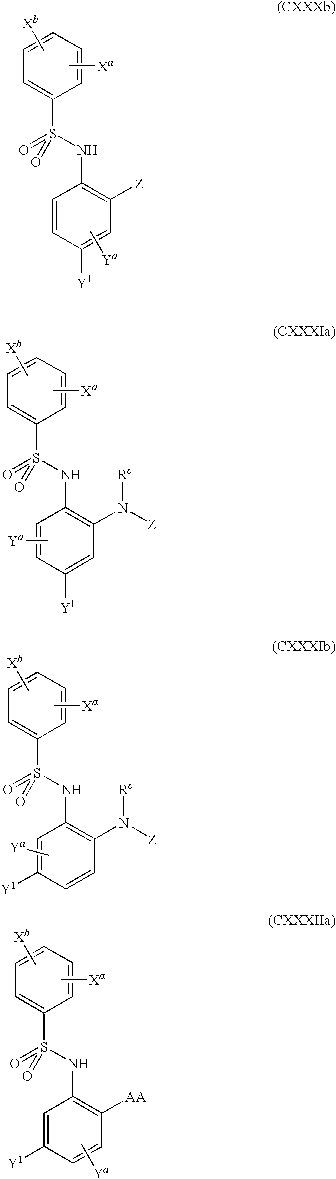

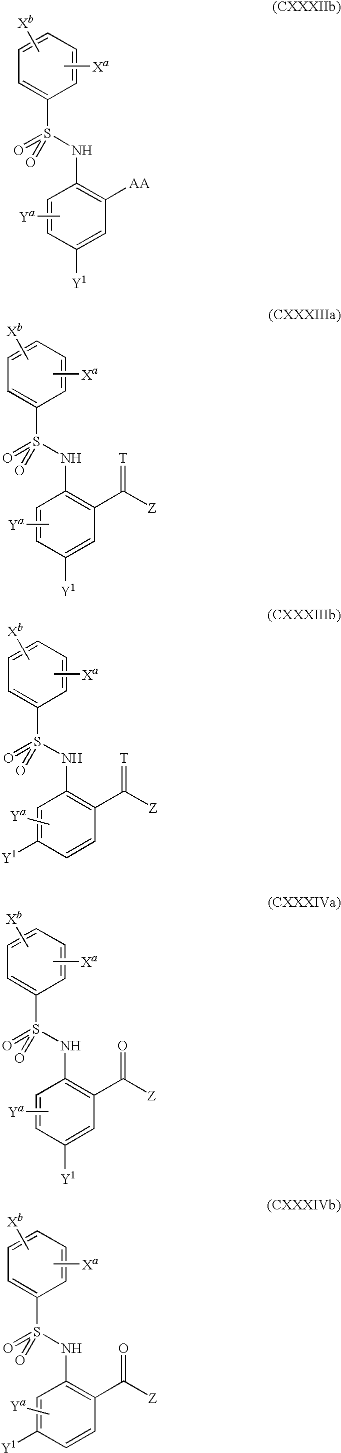

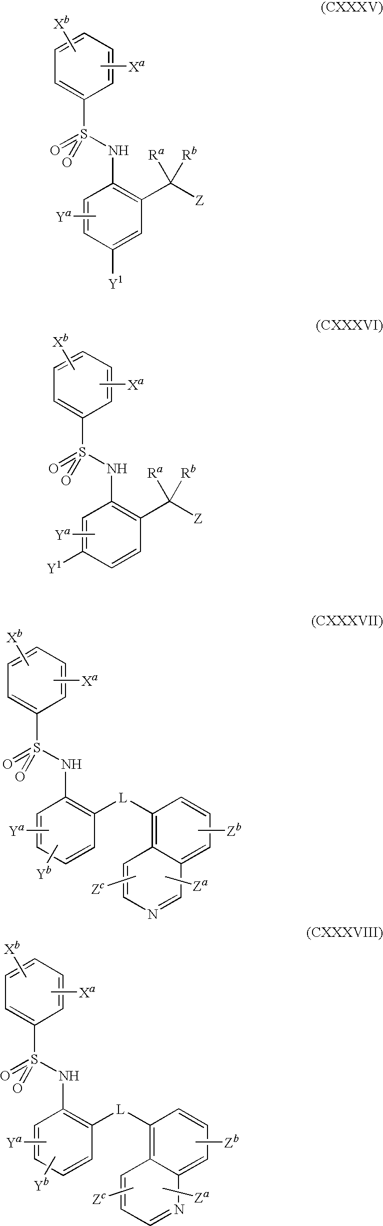

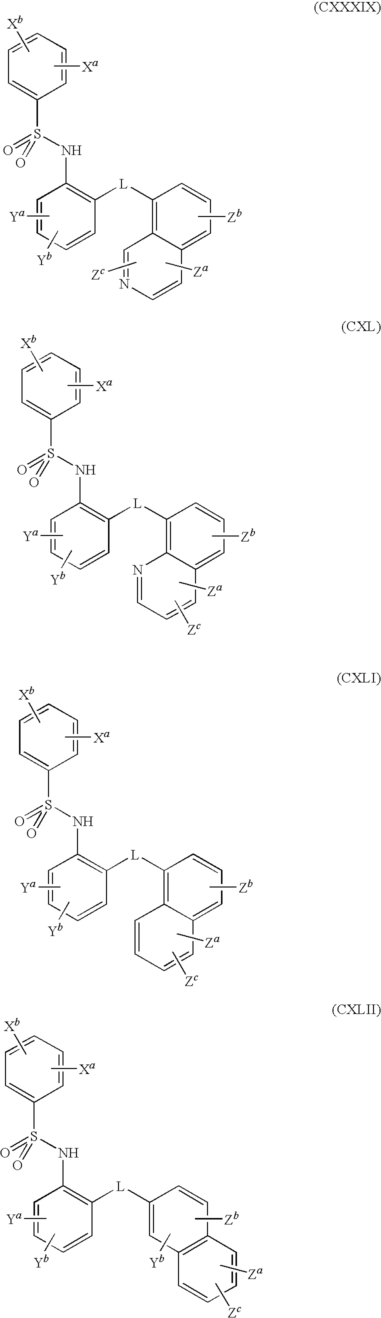

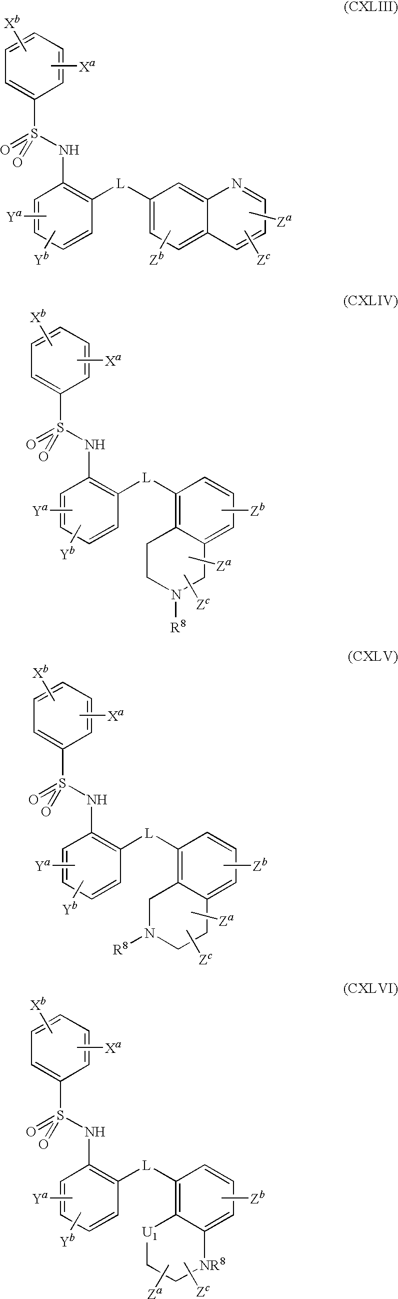

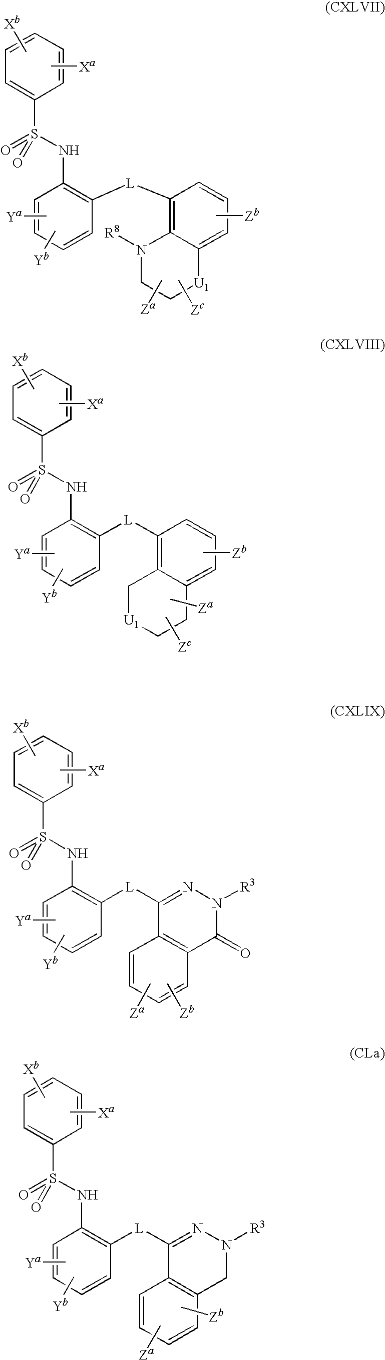

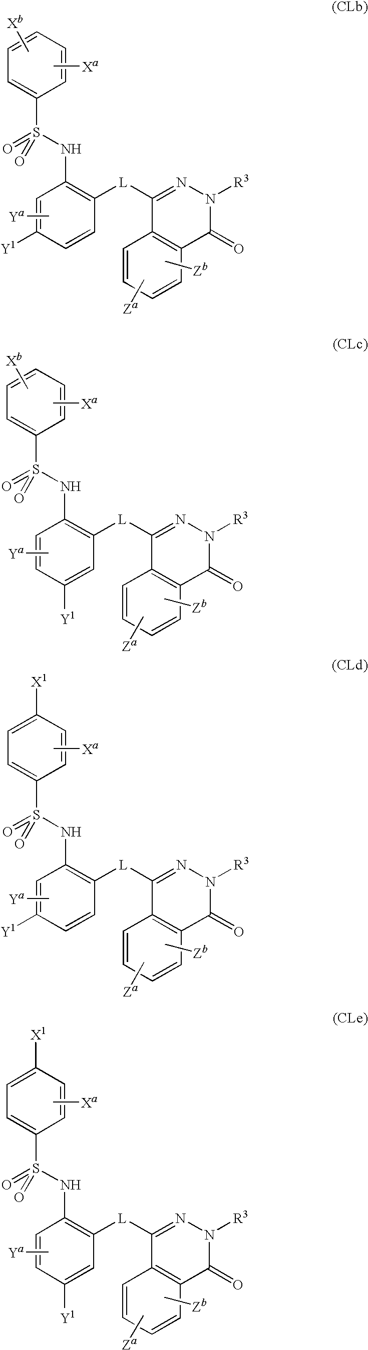

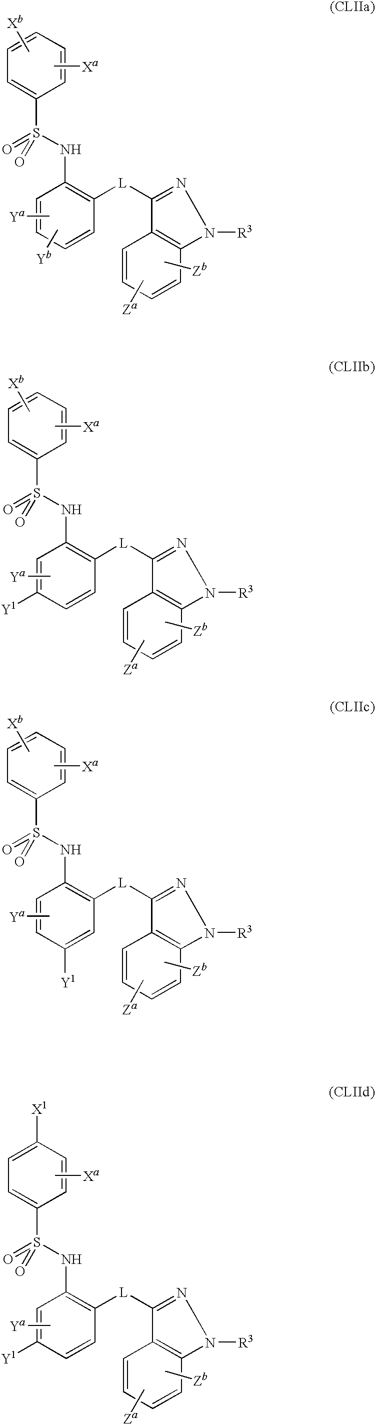

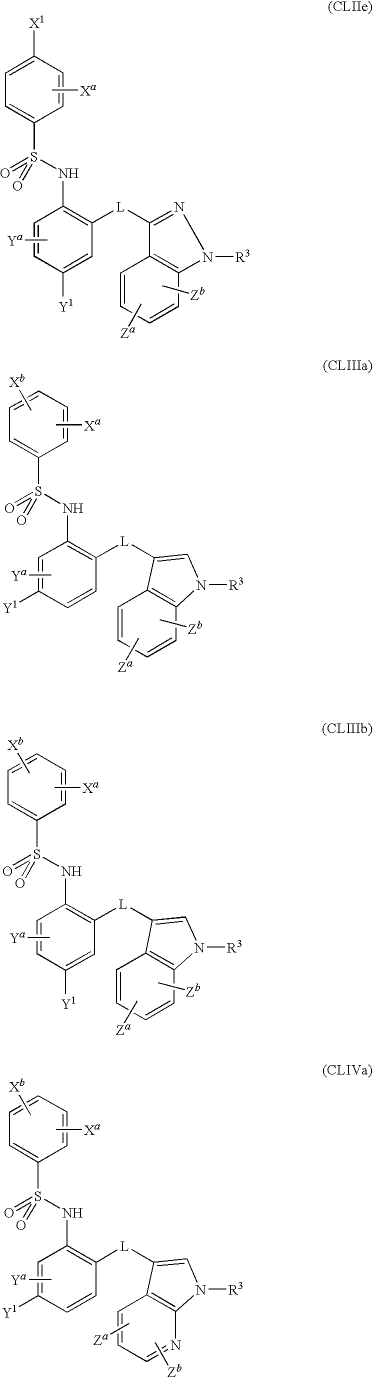

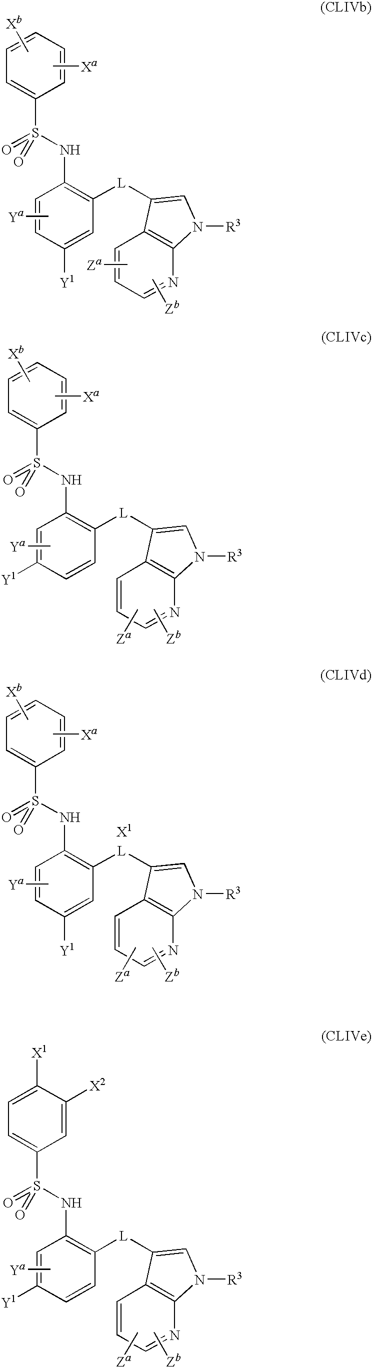

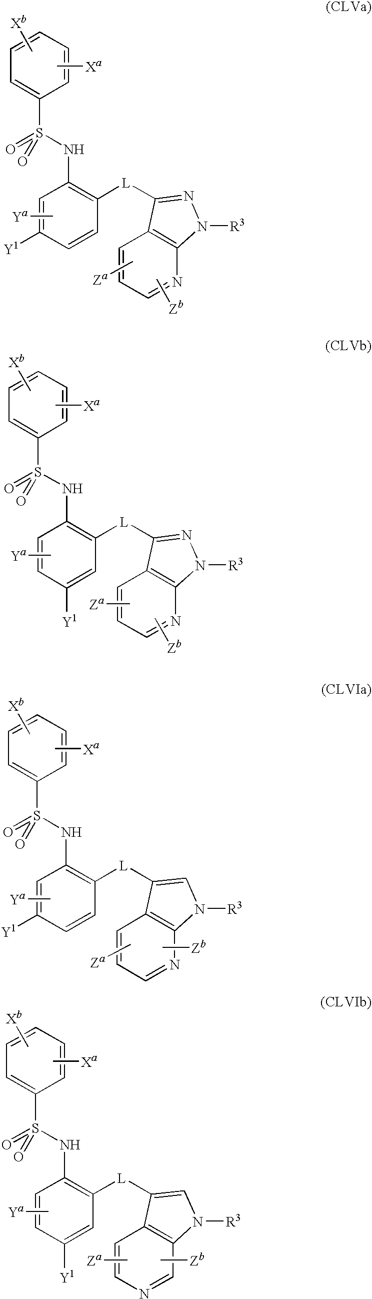

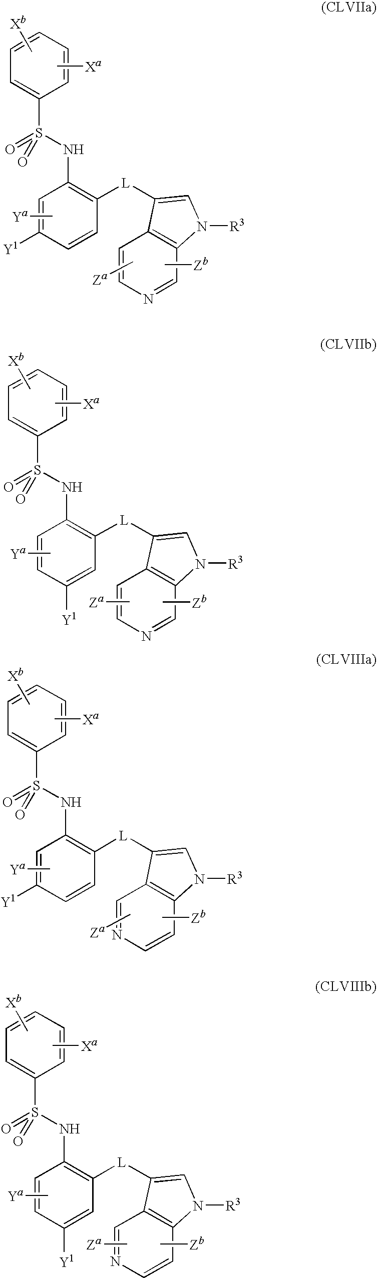

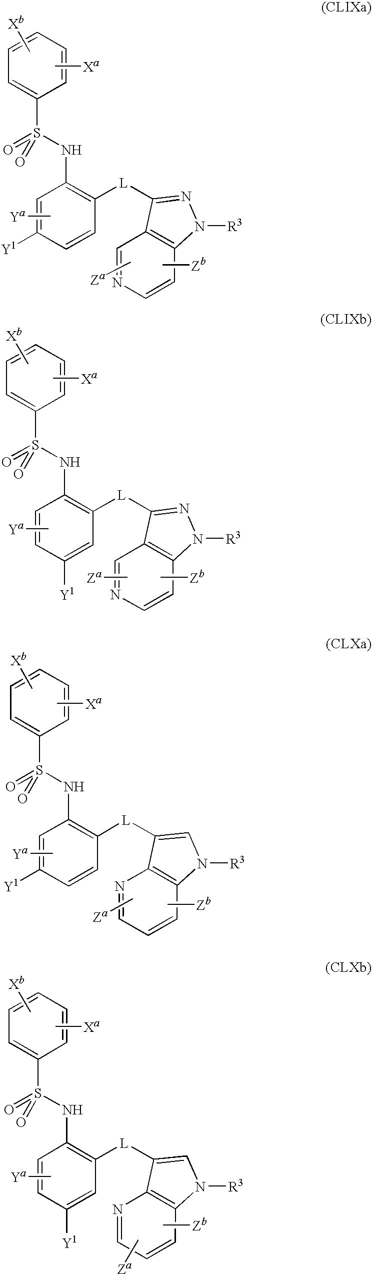

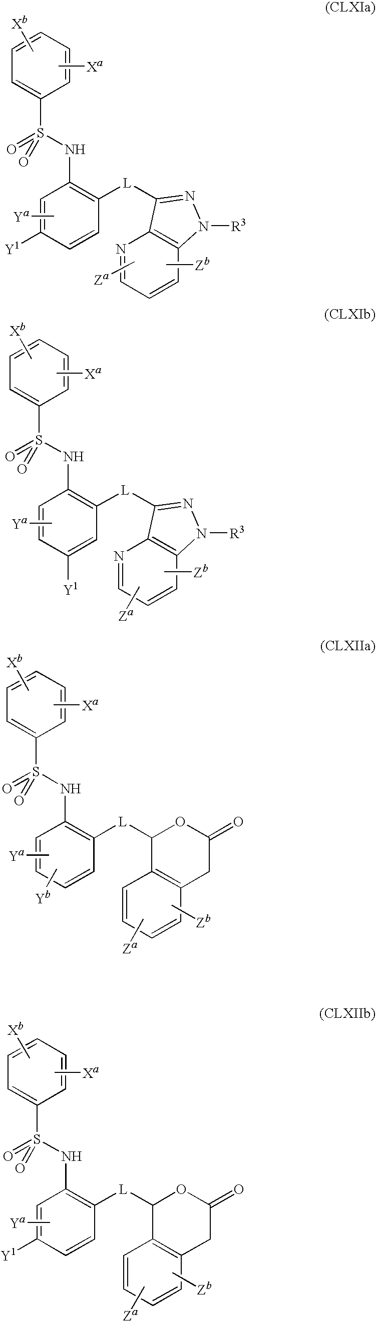

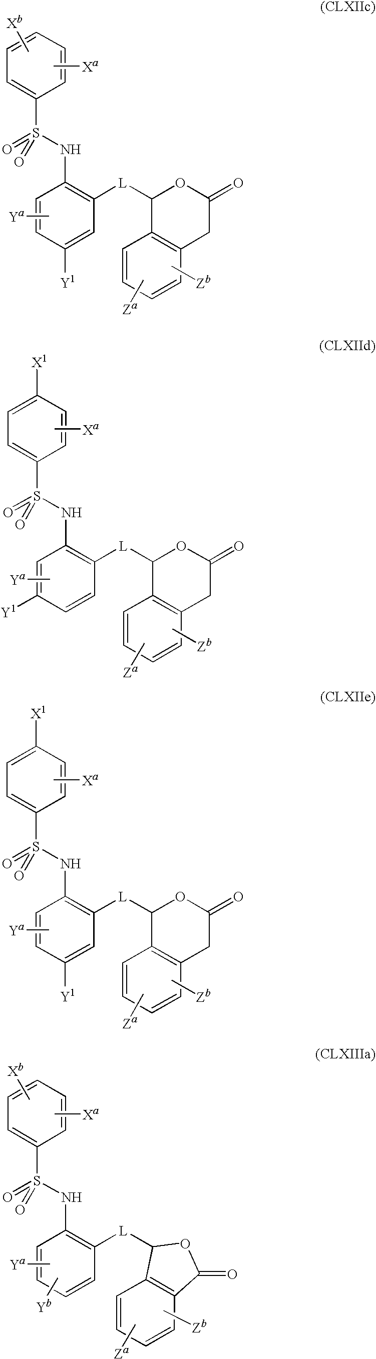

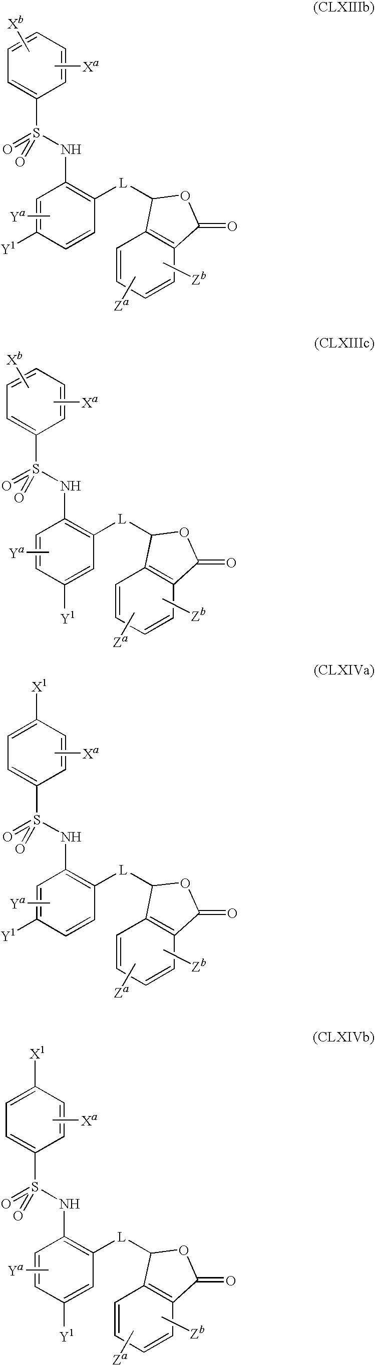

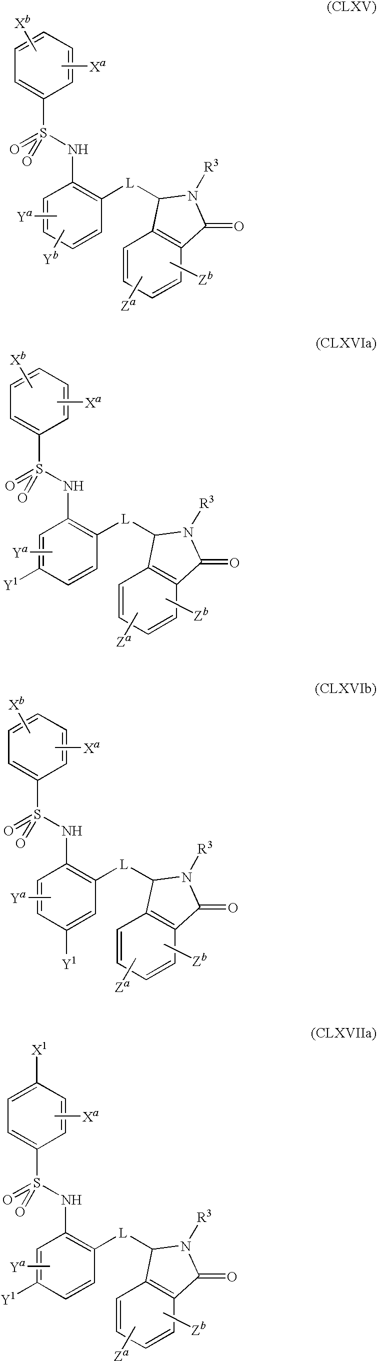

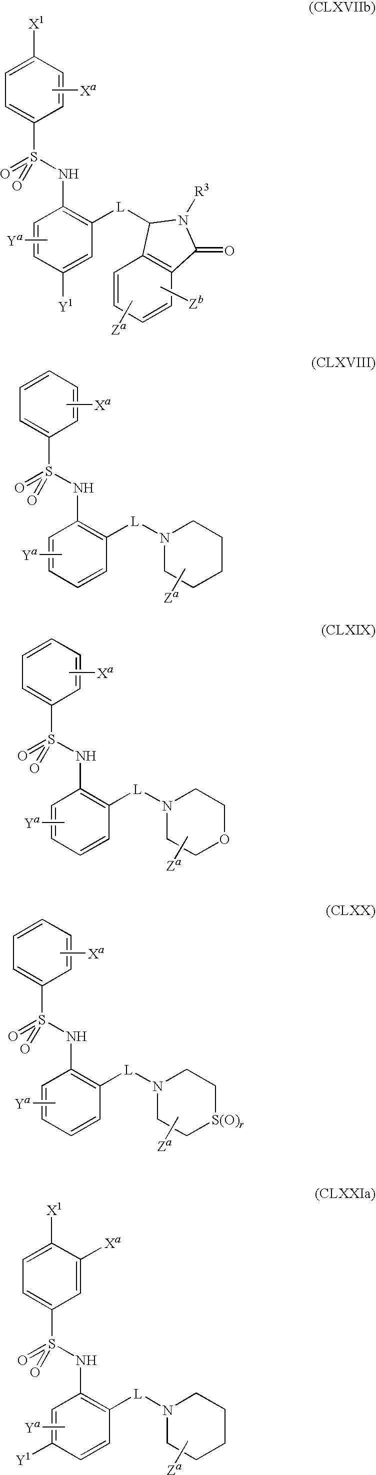

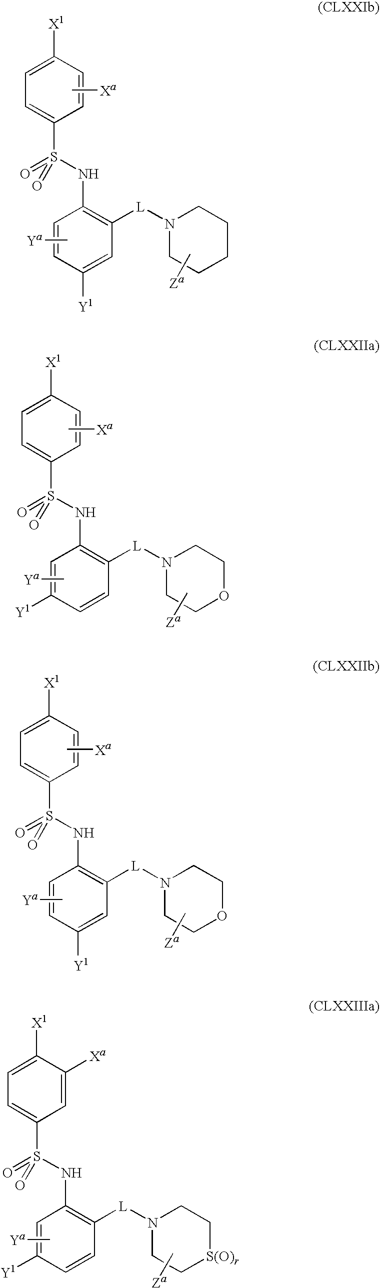

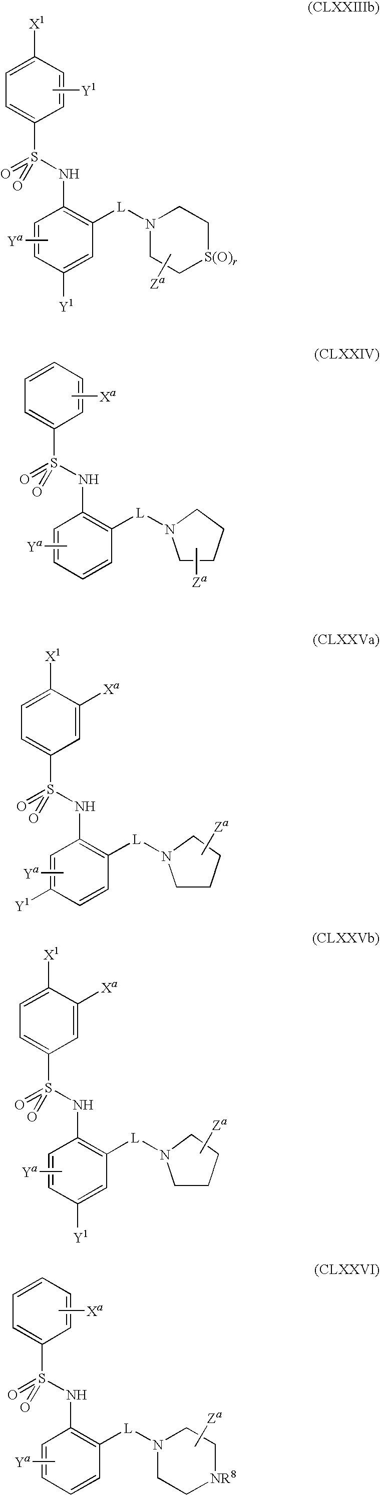

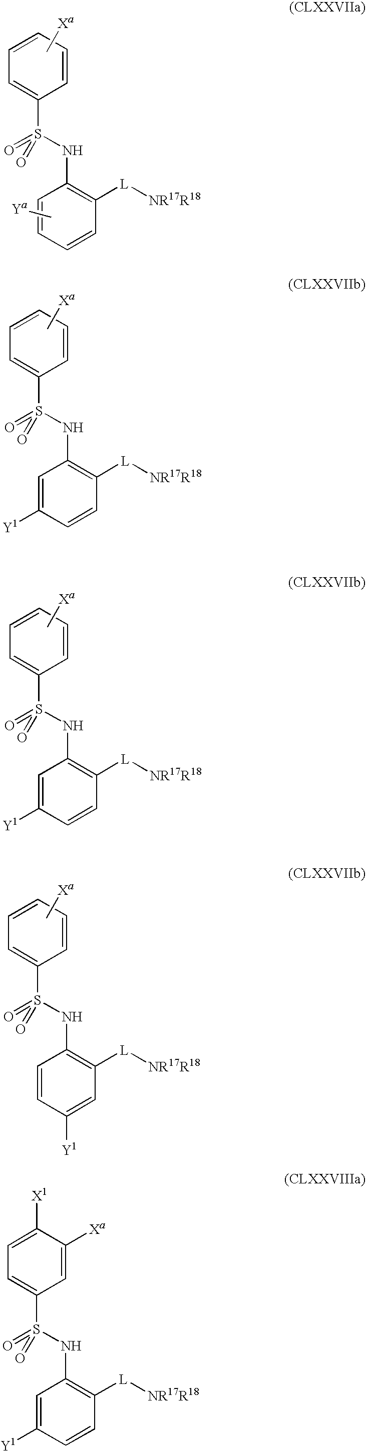

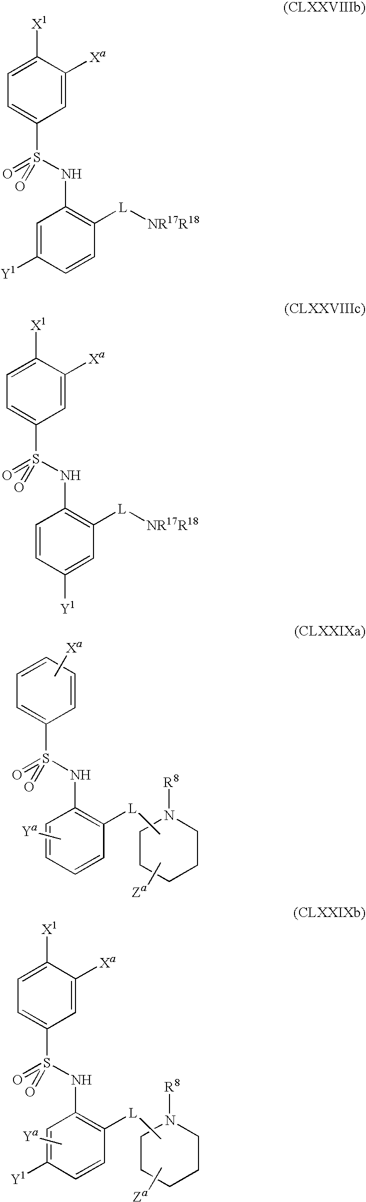

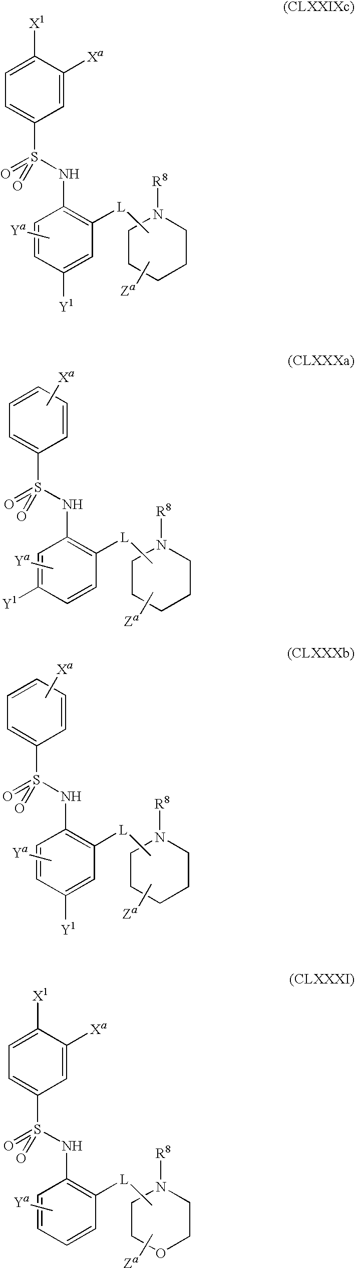

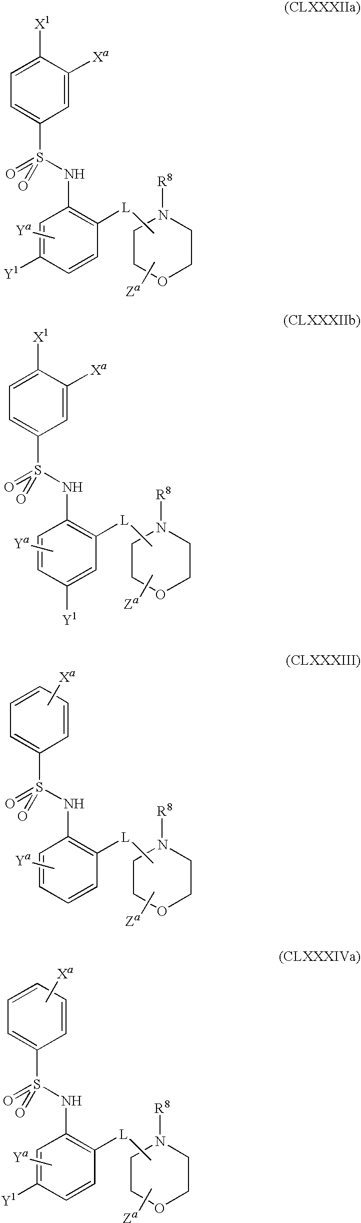

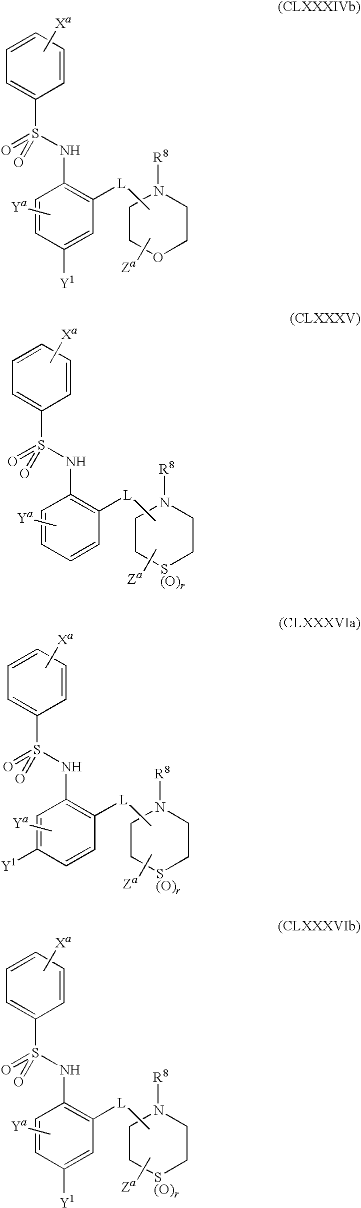

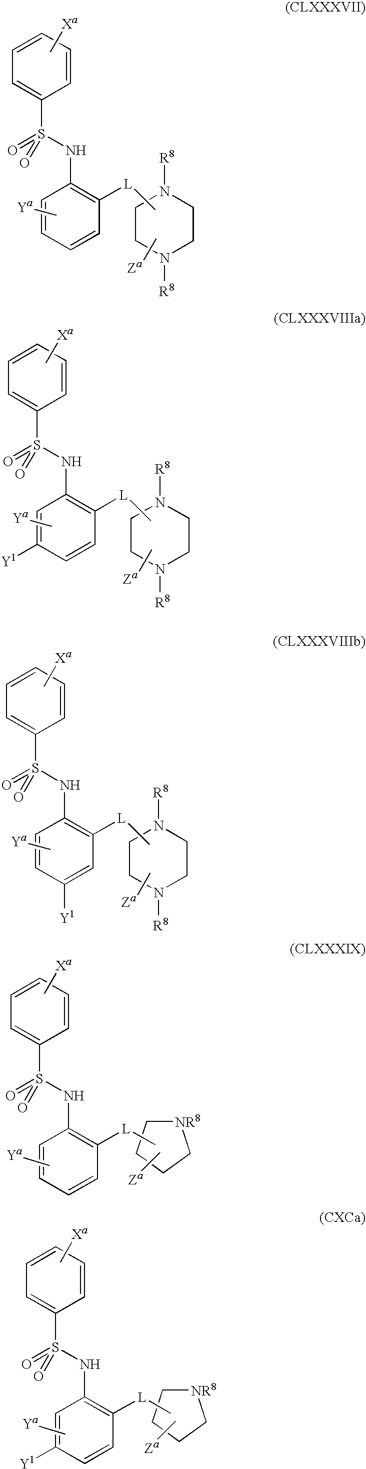

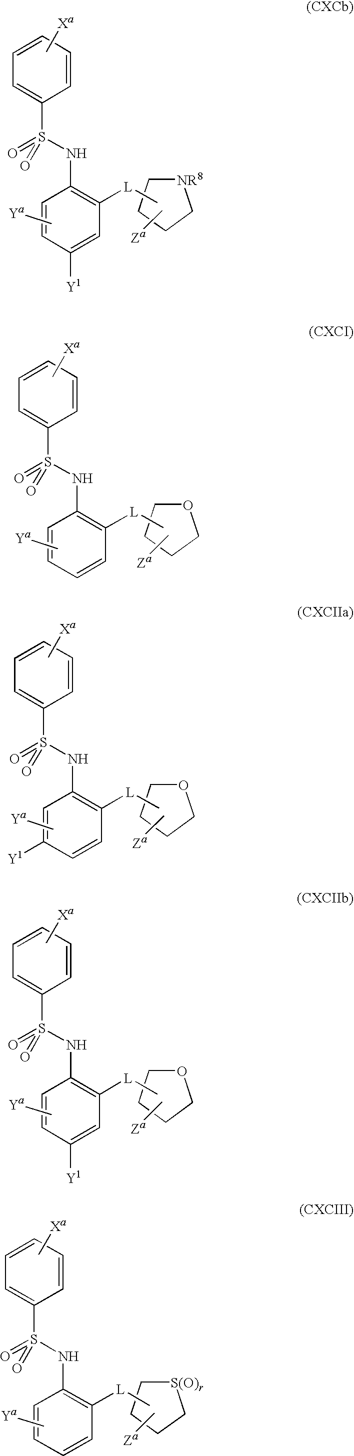

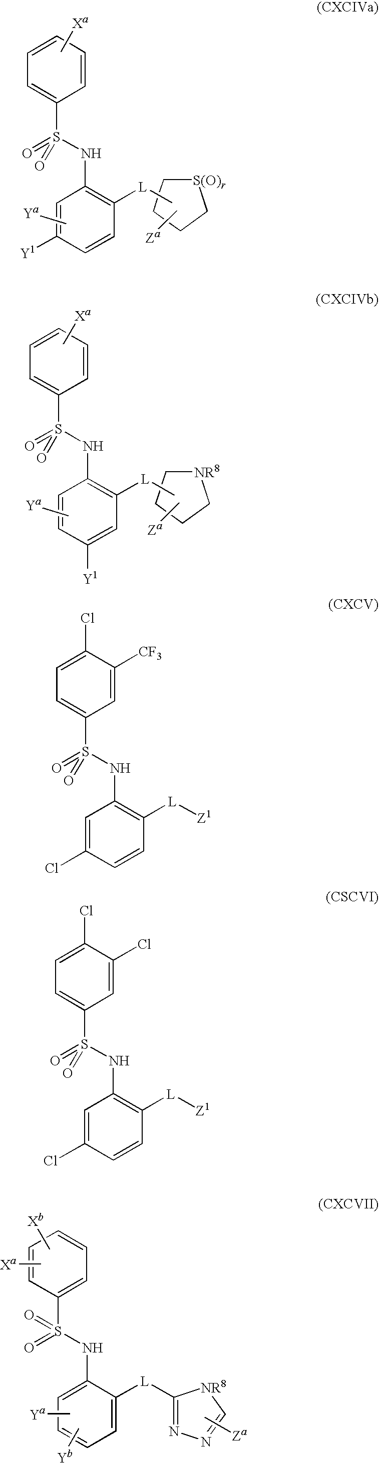

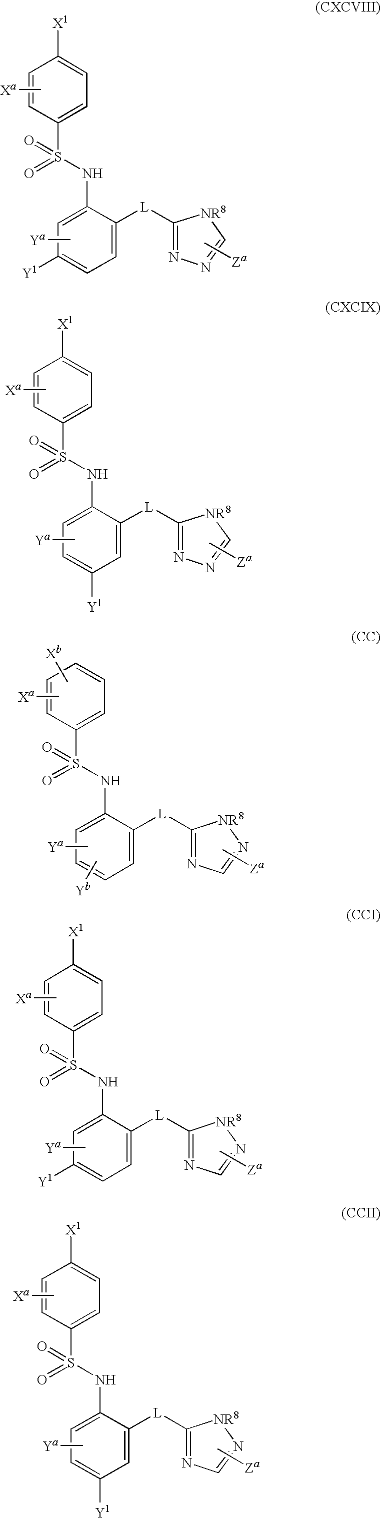

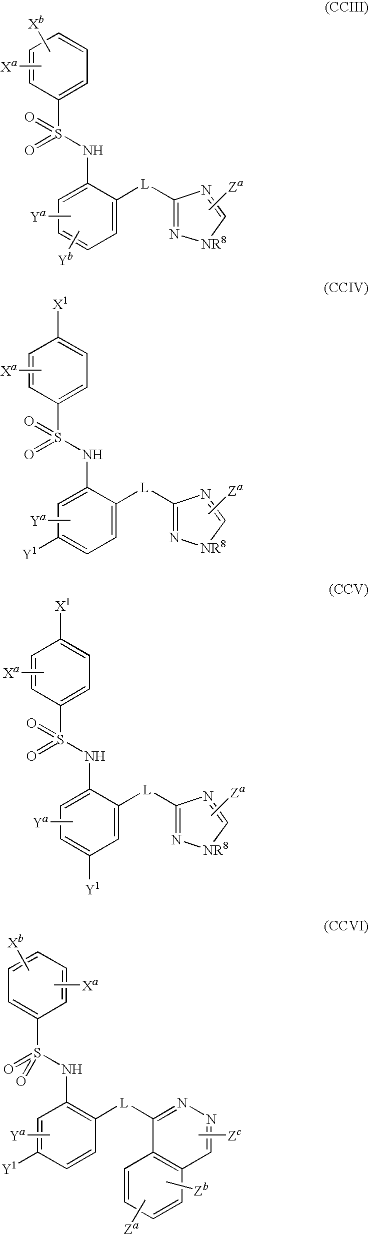

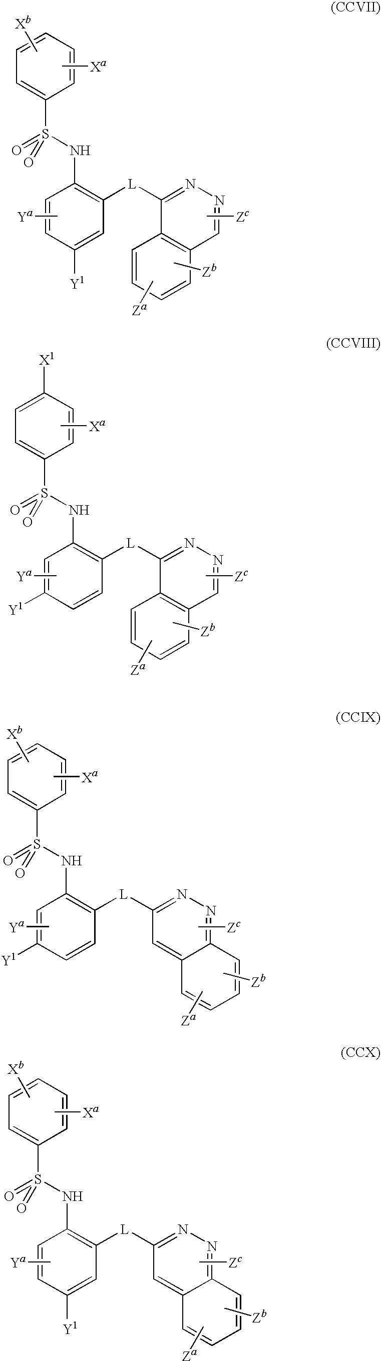

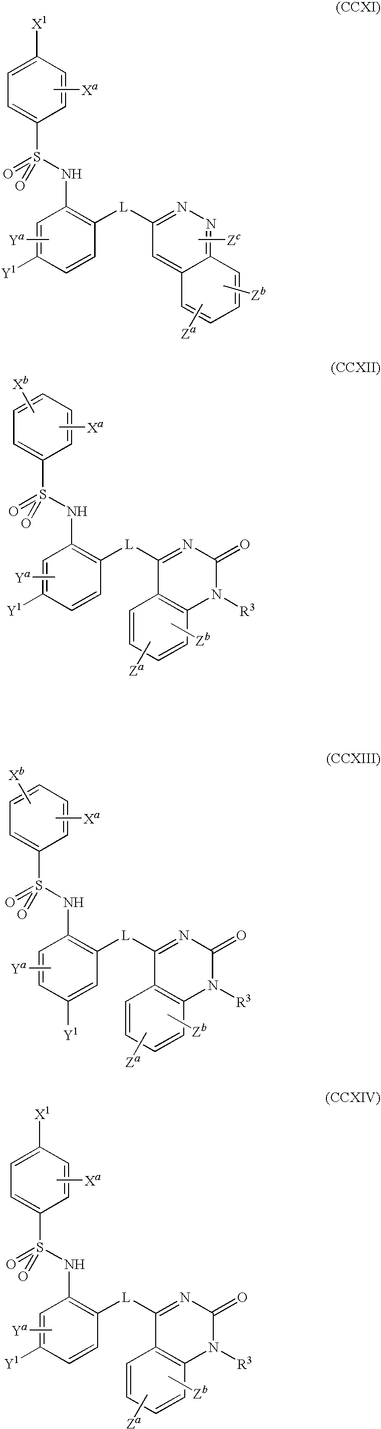

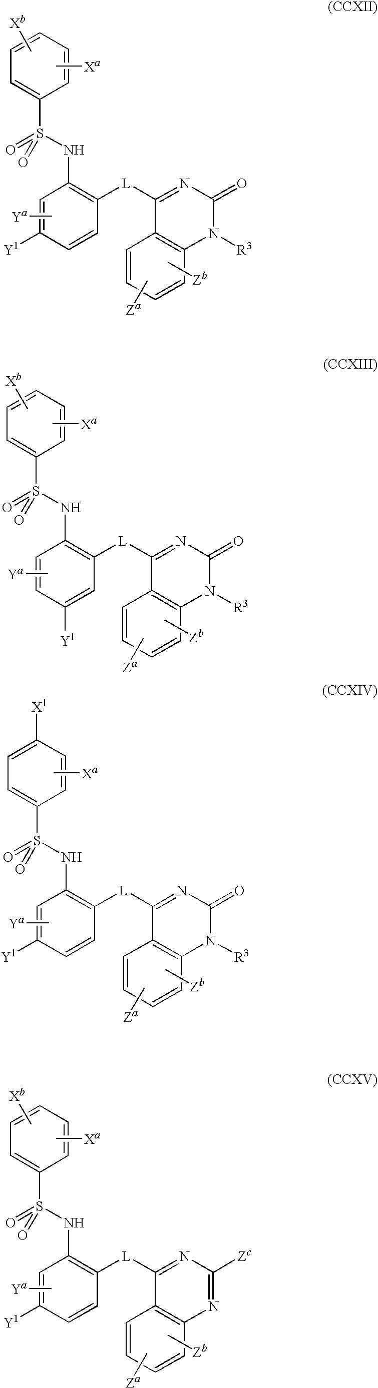

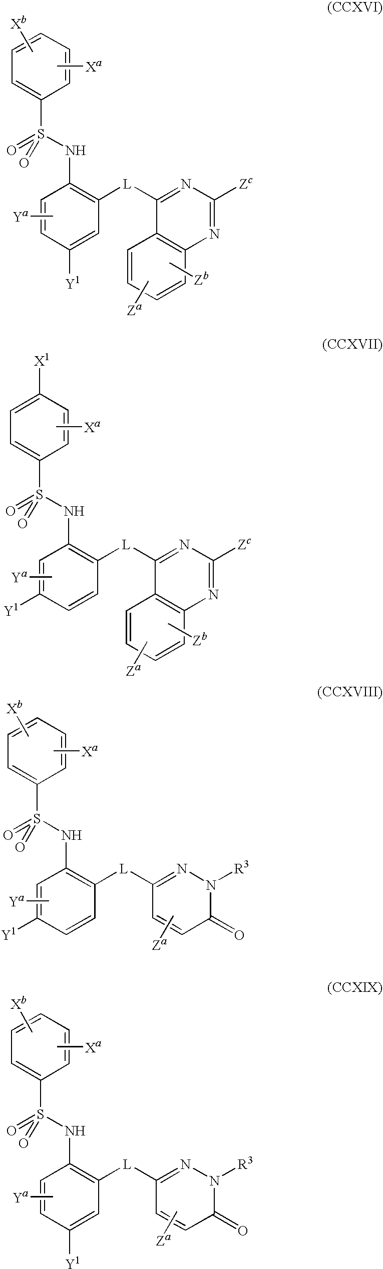

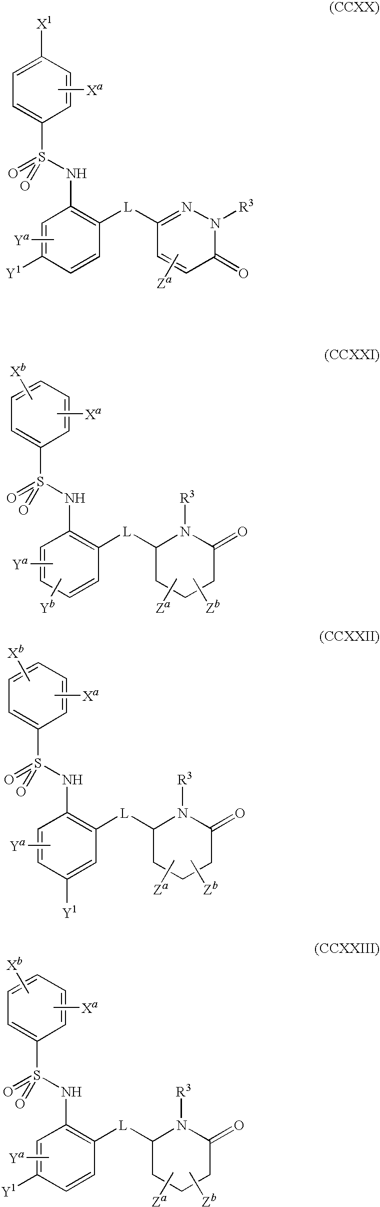

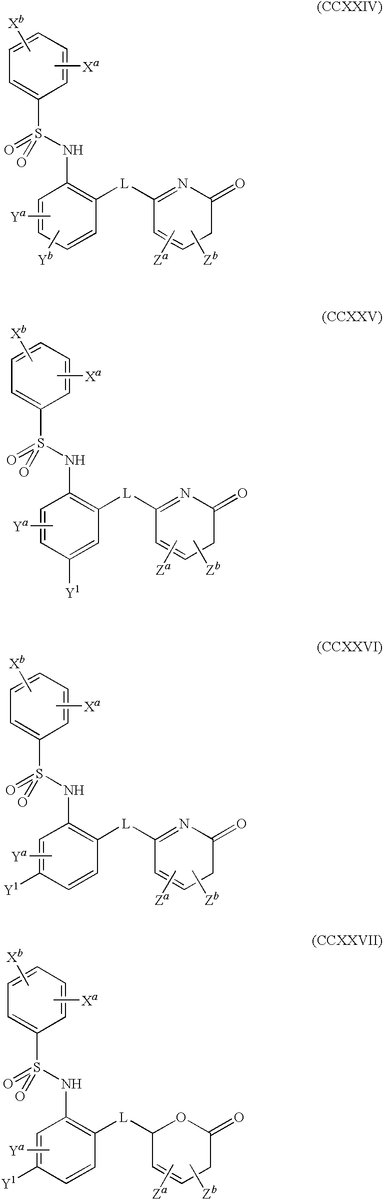

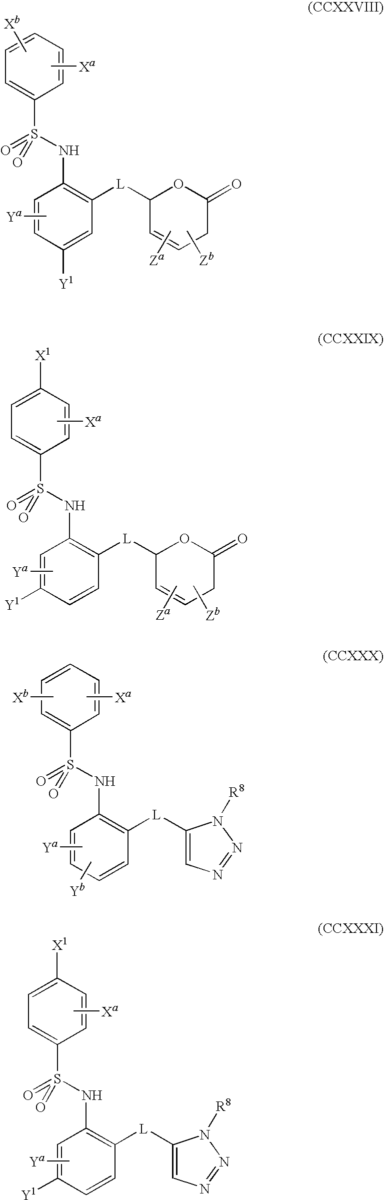

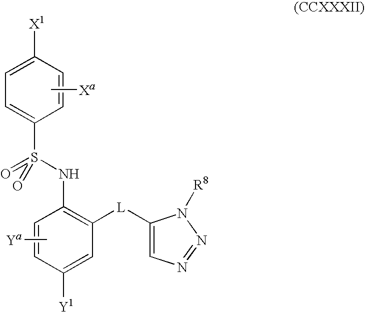

The following descriptions and embodiments only refer to those formulae (I-CCXXXII) that are applicable (i.e., those formulae with the applicable substituents). In the following embodiments, when one substituent is specified, the remaining substituents remain as defined during their first appearance, unless otherwise specified. For example, if X¹ is defined, then X², X³, X⁴, and X⁵ remain as defined in formula (II).

In each of the formula (XX-CCXXXII), L is selected from the group consisting of —O—, —S—, —S(O)—, S(O)₂—, —CR^{a}R^{b}—, —NR^{c}—, —NR^{c}C(O)—, —C(O)—, —C(O) NR^{c}—, C=T, and a bond;

R^{a}, R^{b}, R^{c}, and T are defined as in formula (I);

r is 0, 1 or 2;

R¹⁷ and R¹⁸ are as described above in formula (I);

R³ and R⁸ are each independently a substituent as described in paragraph [0051];

X¹, X², X^{a} and X^{b} are each independently as defined for X¹ in formula (II);

Y¹, Y^{a}, and Y^{b} are each independently as defined for Y¹ in formula (I); and

Z¹, Z^{a}, Z^{b} and Z^{c} are each independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, —CN, ═O, —NO₂, —OR¹⁰, —OC(O)R¹⁰, —CO₂R¹⁰, —C(O)R¹⁰, —C(O)NR¹¹R¹², —OC(O)NR¹¹R¹², —NR¹⁰C(O)R¹¹, —NR¹⁰C(O)NR¹¹R¹², —NR¹¹R¹², —NR¹⁰CO₂R¹¹, —SR¹⁰, —S(O)R¹⁰, —S(O)₂R¹⁰, —S(O)₂NR¹¹R¹², —NR¹⁰S(O)₂R¹¹, substituted or unsubstituted C₆₋₁₀ aryl, substituted or unsubstituted 5- to 10-membered heteroaryl and substituted or unsubstituted 3- to 10-membered heterocyclyl; or alternatively where two of Z¹, Z^{a}, Z^{b} and Z^{c}, together with the atoms which they substitute, form a carbocyclic or heterocyclic ring such that Z is a bi- or tri-cyclic ring;

where suitable substituted C₁₋₈ alkyl, C₂₋₈ alkenyl, C₂₋₈ alkynyl may have from 1 to 3 substituents independently selected from the group consisting of halogen, —OR¹⁰, —CN, —NO₂, ═O, —OC(O)R¹⁰, —CO₂R¹⁰, —C(O)R¹⁰, —C(O)NR¹⁰R¹¹, —OC(O)NR¹⁰R¹¹, —NR¹⁰C(O)R¹¹, —NR¹⁰C(O)NR¹¹R¹², —NR¹⁰R¹¹, —NR¹⁰CO₂R¹¹, —SR¹⁰, —S(O)R¹⁰, —S(O)₂R¹⁰, —S(O)₂NR¹⁰R¹¹, —NR¹⁰S(O)₂R¹¹, unsubstituted or substituted phenyl, unsubstituted or substituted C₅₋₆ heteroaryl, and unsubstituted or substituted C₃₋₆ heterocyclyl;

where suitable substituted aryl, heteroaryl and heterocyclyl substituents may have from 1 to 3 substituents independently selected from the group consisting of halogen, —OR¹⁰, —CN, —NO₂, ═O, —OC(O)R¹⁰, —OC(O)R¹⁰, —CO₂R¹⁰, —C(O)R¹⁰, —C(O)NR¹⁰R¹¹, —OC(O)NR¹⁰R¹¹, —NR²⁰C(O)R¹¹, —NR¹⁰C(O)NR¹¹R¹², —NR¹⁰R¹¹, —NR¹⁰CO₂R¹¹, —SR¹⁰, —S(O)R¹⁰, —S(O)₂R¹⁰, —S(O)₂NR¹⁰R¹¹, —NR¹⁰S(O)₂R¹¹, unsubstituted 4- to 7-membered ring heterocyclyl, unsubstituted C₁₋₈ alkyl and unsubstituted C₁₋₈ haloalkyl;

R¹⁰, R¹¹ and R¹² are each hydrogen, C₁₋₈ alkyl, C₂₋₈ alkenyl, C₂₋₈ alkynyl, aryl, or heteroaryl; or R¹⁰ and R¹¹, or R¹¹ and R¹², or R¹⁰ and R¹², together with the atom(s) to which they are attached, form a substituted or unsubstituted 5-, 6-, or 7-membered ring; and

the aliphatic and aromatic portions of R¹⁰, R¹¹ and R¹² are optionally further substituted with from 1 to 3 substituents selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂, and —NR^{m}S(O)₂NHRⁿ, where R^{m,} Rⁿ, and R^{o} are each independently unsubstituted C₁₋₆ alkyl.

J¹, J² and J³ are each independently selected from the group consisting of CR²⁵R²⁶, O, S(O)ₗ, and NR²⁴, where l is 0, 1 or 2 with the proviso that p+q+r is 3, 4 or 5; and with the proviso that the resulting ring system does not contain a hydrazine functionality, a peroxide functionality nor a hydroxylamine derived functionality, and where R²⁴, R²⁵ and R²⁶ are as defined for formula (AA) in [0045] in the section describing abbreviations. In one preferred embodiment, R²⁴, R²⁵ and R²⁶ are independently hydrogen or unsubstituted C₁₋₆ alkyl.

Each A is independently CZ¹, N or N⁺—O⁻, where Z¹ is an Z substituent, as defined for formula (I), independently selected from the group consisting of: hydrogen, halogen, substituted or unsubstituted C₁₋₈ alkyl, substituted or unsubstituted C₂₋₈ alkenyl, substituted or unsubstituted C₂₋₈ alkynyl, —CN, ═O, —NO₂, —OR⁷, —OC(O)R⁷, —CO₂R⁷, —C(O)R⁷, —C(O)NR⁸R⁹, —OC(O)NR⁸R⁹, —NR⁷C(O)R⁸, —NR⁷C(O)NR⁸R⁹, —NR⁸R⁹, —NR⁷CO₂R⁸, —SR⁷, —S(O)R⁷, —S(O)₂R⁷, —S(O)₂NR⁸R⁹, —NR⁷S(O)₂R⁸, substituted or unsubstituted C₆₋₁₀ aryl, substituted or unsubstituted 5- to 10-membered heteroaryl and substituted or unsubstituted 3- to 10-membered heterocyclyl; or where two or more A are CZ¹, then the Z¹ substituents together can form a carbocyclic or heterocyclic ring;

where R⁷, R⁸, R⁹, substituted C₁₋₈ alkyl, substituted C₂₋₈ alkenyl, substituted C₂₋₈ alkynyl, substituted C₆₋₁₀ aryl, substituted 5- to 10-membered heteroaryl, and substituted 3- to 10-membered heterocyclyl are as defined for formula (III).

Each E is independently CZ¹, or N, where Z¹ is defined as in [00125].

Each G is independently O, S, or NZ¹, where Z¹ is defined as in [00125].

Each U₁ is independently selected from the group consisting of CH₂, O, NR⁸, where R⁸ is as defined in formula (I).

AA refers to formula (AA) as defined in paragraphs [0045-0048].

Compounds that Modulate CCR2 or CCR9 Activity

The present invention provides compounds that modulate at least one of CCR2 or CCR9 activity. Chemokine receptors are integral membrane proteins which interact with an extracellular ligand, such as a chemokine, and mediate a cellular response to the ligand, e.g., chemotaxis, increased intracellular calcium ion concentration, etc. Therefore, modulation of a chemokine receptor function, e.g., interference with a chemokine receptor ligand interaction, will modulate a chemokine receptor mediated response, and treat or prevent a chemokine receptor mediated condition or disease. Modulation of a chemokine receptor function includes both inducement and inhibition of the function. The type of modulation accomplished will depend on the characteristics of the compound, i.e., antagonist or full, partial or inverse agonist.

Without intending to be bound by any particular theory, it is believed that the compounds provided herein interfere with the interaction between a chemokine receptor and one or more cognate ligands. In particular, it is believed that the compounds interfere with the interaction between CCR2 and a CCR2 ligand, such as MCP-1. Compounds contemplated by the invention include, but are not limited to, the exemplary compounds provided herein and salts thereof.

For example, compounds of this invention act as potent CCR2 antagonists, and this antagonistic activity has been further confirmed in animal testing for inflammation, one of the hallmark disease states for CCR2. Accordingly, the compounds provided herein are useful in pharmaceutical compositions, methods for the treatment of CCR2-mediated diseases, and as controls in assays for the identification of competitive CCR2 antagonists.

The compounds of the invention are thought to interfere with inappropriate T-cell trafficking by specifically modulating or inhibiting a chemokine receptor function. Without intending to be bound by any particular theory, it is believed that the compounds provided herein interfere with the interaction between a chemokine receptor and one or more cognate ligands. In particular, it is believed that the compounds interfere with the interaction between CCR9 and a CCR9 ligand, such as TECK. Compounds contemplated by the invention include, but are not limited to, the exemplary compounds provided herein and salts thereof.

For example, compounds of this invention act as potent CCR9 antagonists, and this antagonistic activity has been further confirmed in animal testing for inflammation, one of the hallmark disease states for CCR9. Accordingly, the compounds provided herein are useful in pharmaceutical compositions, methods for the treatment of CCR9-mediated diseases, and as controls in assays for the identification of competitive CCR9 antagonists.

### Known Compounds

The following meta-substituted benzene-sulfonamides in Table 1 are known, but not as CCR2 modulators (these compounds are explicitly excluded from modulators of formula (I-CCXXXII)):

TABLE 1
Meta-substituted benzene-sulfonamides

4-chloro-N-[5-chloro-2-(1H-1,2,4-triazol-1-yl)phenyl]-3-(trifluoromethyl)-
benzenesulfonamide
N-(4,5-dimethoxy-2-(1,2,4-oxadiazol-5-yl)phenyl)-4-fluoro-3-
(trifluoromethyl)benzenesulfonamide
N-(4,5-dimethoxy-2-(oxazol-2-yl)phenyl)-3-
(trifluoromethyl)benzenesulfonamide
N-(4-hydroxy-2-(oxazol-2-yl)phenyl)-3-(trifluoromethyl)benzenesulfonamide
N-(4-(dimethylamino)-2-(oxazol-2-yl)phenyl)-3-
(trifluoromethyl)benzenesulfonamide
N-(4-(methylamino)-2-(oxazol-2-yl)phenyl)-3-
(trifluoromethyl)benzenesulfonamide
N-(2-(oxazol-2-yl)phenyl)-3-(trifluoromethyl)benzenesulfonamide
N-(4-Methoxy-2-oxazol-2-yl-phenyl)-3-trifluoromethyl-benzenesulfonamide
N-[4,5-dimethoxy-2-(3-methyl-[1,2,4]oxadiazol-5-yl)-phenyl-3-trifluoromethyl-
benzenesulfonamide
N-(4-ethoxy-2-(oxazol-2-yl]phenyl)-3-(trifluoromethyl)benzenesulfonamide
N-(4-(2-methoxyethoxy)-2-(oxazol-2-yl)phenyl)-3-
(trifluoromethyl)benzenesulfonamide
N-(3-(oxazol-2-yl)-4-(3-(trifluoromethyl)phenylsulfonamido)phenyl)acetamide
N-(6-(oxazol-2-yl)benzo[d][1,3]dioxol-5-yl)-3-
(trifluoromethyl)benzenesulfonamide
N-(2-(1,3,4-oxadiazol-2-yl)phenyl)-3-(trifluoromethyl)benzenesulfonamide
N-(4-methoxy-2-(oxazol-2-yl)phenyl)-3-(trifluoromethyl)benzenesulfonamide
N-(2-Oxazol-2-yl-phenyl)-3-trifluoromethyl-benzenesulfonamide
N-(5-chloro-2-(oxazol-2-yl)phenyl)-3-(trifluoromethyl)benzenesulfonamide
N-(2-furan-2-yl-phenyl)-3-trifluoromethyl-benzenesulfonamide
N-(4-Methyl-2-oxazol-2-yl-phenyl)-3-trifluoromethyl-benzenesulfonamide
N-(2-(oxazol-5-yl)phenyl)-3-(trifluoromethyl)benzenesulfonamide
N-(2-(5-methyloxazol-2-yl)phenyl)-3-(trifluoromethyl)benzenesulfonamide
N-(2-(oxazol-2-yl)-5-(trifluoromethyl)phenyl)-3-
(trifluoromethyl)benzenesulfonamide
N-(4,5-difluoro-2-(oxazol-2-yl)phenyl)-3-(trifluoromethyl)benzenesulfonamide
N-[2-(3-methyl-[1,2,4]oxadiazol-5-yl)-phenyl]-3-trifluoromethyl-
benzenesulfonamide
N-[2-(3-methoxy-[1,2,4]oxadiazol-5-yl)-phenyl]-3-trifluoromethyl-
benzenesulfonamide
N-(4-hydroxy-2-(3-methyl-1,2,4-oxadiazol-5-yl)phenyl)-3-
(trifluoromethyl)benzenesulfonamide
N-(4,5-dimethoxy-2-(3-methyl-1,2,4-oxadiazol-5-yl)phenyl)-3-
(trifluoromethyl)benzenesulfonamide
N-(5-methoxy-2-(3-methyl-1,2,4-oxadiazol-5-yl)phenyl)-3-
(trifluoromethyl)benzenesulfonamide
N-(4-methoxy-2-(3-methyl-1,2,4-oxadiazol-5-yl)phenyl)-3-
(trifluoromethyl)benzenesulfonamide
N-(4-hydroxy-2-(3-methyl-1,2,4-oxadiazol-5-yl)phenyl)-3-
(trifluoromethyl)benzenesulfonamide
N-(5-methoxy-2-(oxazol-2-yl)phenyl)-3-(trifluoromethyl)benzenesulfonamide
N-(5-hydroxy-2-(oxazol-2-yl)phenyl)-3-(trifluoromethyl)benzenesulfonamide
N-(6-(1,2,4-oxadiazol-5-yl)benzo[d][1,3]dioxol-5-yl)-3-
(trifluoromethyl)benzenesulfonamide
N-(4,5-dihydroxy-2-(1,2,4-oxadiazol-5-yl)phenyl)-3-
(trifluoromethyl)benzenesulfonamide
N-(4,5-dimethoxy-2-(1,2,4-oxadiazol-5-yl)phenyl)-3-
(trifluoromethyl)benzenesulfonamide
N-(6-(3-methyl-1,2,4-oxadiazol-5-yl)benzo[d][1,3]dioxol-5-yl)-3-
(trifluoromethyl)benzenesulfonamide
N-(6-(furan-2-yl)benzo[d][1,3]dioxol-5-yl)-3-
(trifluoromethyl)benzenesulfonamide
N-(2-(furan-2-yl)-4,5-dimethoxyphenyl)-3-
(trifluoromethyl)benzenesulfonamide
N-(2-(furan-2-yl)-4-methoxyphenyl)-3-(trifluoromethyl)benzenesulfonamide
N-(2-(furan-2-yl)-5-methoxyphenyl)-3-(trifluoromethyl)benzenesulfonamide
N-(2-(furan-2-yl)-5-hydroxyphenyl)-3-(trifluoromethyl)benzenesulfonamide
N-(2-(furan-2-yl)-4-methoxyphenyl)-3-(trifluoromethyl)benzenesulfonamide
N-(5-methoxy-2-(oxazol-2-yl)phenyl)-3-(trifluoromethyl)benzenesulfonamide
N-(2-(furan-2-yl)-4-hydroxyphenyl)-3-(trifluoromethyl)benzenesulfonamide
N-(4-hydroxy-2-(oxazol-2-yl)phenyl)-3-(trifluoromethyl)benzenesulfonamide
N-(4,5-dimethoxy-2-(oxazol-2-yl)phenyl)-3-
(trifluoromethyl)benzenesulfonamide
3,4-dichloro-N-[2-(1H-1,2,4-triazol-1-yl)-5-(trifluoromethyl)phenyl]-
benzenesulfonamide
3-chloro-4-fluoro-N-(4-methoxy-2-(oxazol-2-yl)phenyl)benzenesulfonamide
3-chloro-4-fluoro-N-(5-methoxy-2-(oxazol-2-yl)phenyl)benzenesulfonamide
3-chloro-4-fluoro-N-(5-hydroxy-2-(oxazol-2-yl)phenyl)benzenesulfonamide
3-chloro-4-fluoro-N-(4-hydroxy-2-(oxazol-2-yl)phenyl)benzenesulfonamide
3-chloro-4-fluoro-N-(6-(oxazol-2-yl)benzo[d][1,3]dioxol-5-
yl)benzenesulfonamide
3-chloro-4-fluoro-N-(4-methoxy-2-(oxazol-2-yl)phenyl)benzenesulfonamide
3-chloro-N-(4,5-dimethoxy-2-(1,2,4-oxadiazol-5-yl)phenyl)-4-
fluorobenzenesulfonamide
N-(6-(1,2,4-oxadiazol-5-yl)benzo[d][1,3]dioxol-5-yl)-3-chloro-4-
fluorobenzenesulfonamide
3-chloro-N-(4,5-dihydroxy-2-(1,2,4-oxadiazol-5-yl)phenyl)-4-
fluorobenzenesulfonamide
3-chloro-N-(4,5-dimethoxy-2-(3-methyl-1,2,4-oxadiazol-5-yl)phenyl)-4-
fluorobenzenesulfonamide
3-chloro-N-(4,5-dihydroxy-2-(3-methyl-1,2,4-oxadiazol-5-yl)phenyl)-4-
fluorobenzenesulfonamide
3-chloro-N-(2-(oxazol-2-yl)phenyl)benzenesulfonamide
3-chloro-N-(5-methoxy-2-(oxazol-2-yl)phenyl)benzenesulfonamide
3-chloro-N-(5-hydroxy-2-(oxazol-2-yl)phenyl)benzenesulfonamide
3-chloro-N-(4-hydroxy-2-(oxazol-2-yl)phenyl)benzenesulfonamide
3-chloro-N-(6-(oxazol-2-yl)benzo[d][1,3]dioxol-5-yl)benzenesulfonamide
3-chloro-N-(4,5-dimethoxy-2-(1,2,4-oxadiazol-5-
yl)phenyl)benzenesulfonamide
3-chloro-N-(4,5-dimethoxy-2-(3-methyl-1,2,4-oxadiazol-5-
yl)phenyl)benzenesulfonamide
N-(6-(1,2,4-oxadiazol-5-yl)benzo[d][1,3]dioxol-5-yl)-3-
chlorobenzenesulfonamide
3-chloro-N-(4,5-dihydroxy-2-(1,2,4-oxadiazol-5-
yl)phenyl)benzenesulfonamide
3-chloro-N-(4,5-dihydroxy-2-(3-methyl-1,2,4-oxadiazol-5-
yl)phenyl)benzenesulfonamide
2-chloro-5-[[[2-(4-morpholinyl)-5-(trifluoromethyl)phenyl]amino]sulfonyl]-
benzoic acid
3-fluoro-N-(4-methoxy-2-(oxazol-2-yl)phenyl)benzenesulfonamide
3,4-difluoro-N-(4-methoxy-2-(oxazol-2-yl)phenyl)benzenesulfonamide
3-fluoro-N-(5-hydroxy-2-(oxazol-2-yl)phenyl)benzenesulfonamide
3-fluoro-N-(4-hydroxy-2-(oxazol-2-yl)phenyl)benzenesulfonamide
3-fluoro-N-(4-hydroxy-2-(oxazol-2-yl)phenyl)-4-methylbenzenesulfonamide
N-(4-hydroxy-2-(oxazol-2-yl)phenyl)-3-isopropylbenzenesulfonamide
3-isopropyl-N-(4-methoxy-2-(oxazol-2-yl)phenyl)benzenesulfonamide
3-isopropyl-N-(6-(oxazol-2-yl)benzo[d][1,3]dioxol-5-yl)benzenesulfonamide
N-(6-(1,2,4-oxadiazol-5-yl)benzo[d][1,3]dioxol-5-yl)-3-
isopropylbenzenesulfonamide
N-(4,5-dimethoxy-2-(3-methyl-1,2,4-oxadiazol-5-yl)phenyl)-3-
isopropylbenzenesulfonamide
N-(4,5-dihydroxy-2-(3-methyl-1,2,4-oxadiazol-5-yl)phenyl)-3-
isopropylbenzenesulfonamide
4-bromo-3-methyl-N-[2-(4-morpholinyl)-5-(trifluoromethyl)phenyl]-
benzenesulfonamide
4-bromo-N-[5-chloro-2-(1H-1,2,4-triazol-1-yl)phenyl]-3-methyl-
benzenesulfonamide
3-[[(4-fluoro-3-methylphenyl)sulfonyl]amino]-N-(2-phenylethyl)-4-(1,5,6,8-
tetrahydro-8-oxo-1,5-methano-2H-pyrido[1,2-a][1,5]diazocin-3(4H)-yl)-
benzamide
3-[[(4-fluoro-3-methylphenyl)sulfonyl]amino]-N-(phenylmethyl)-4-(1,5,6,8-
tetrahydro-8-oxo-1,5-methano-2H-pyrido[1,2-a][1,5]diazocin-3(4H)-yl)-
benzamide
4-fluoro-N-(4-methoxy-2-(oxazol-2-yl)phenyl)-3-methylbenzenesulfonamide
3-methyl-N-(6-(oxazol-2-yl)benzo[d][1,3]dioxol-5-yl)benzenesulfonamide
N-(4-hydroxy-2-(oxazol-2-yl)phenyl)-3-methylbenzenesulfonamide
N-(4,5-dimethoxy-2-(3-methyl-1,2,4-oxadiazol-5-yl)phenyl)-3-
methylbenzenesulfonamide
N-(4-hydroxy-2-(oxazol-2-yl)phenyl)-3,4-dimethylbenzenesulfonamide
N-(4-methoxy-2-(oxazol-2-yl)phenyl)-3,4-dimethylbenzenesulfonamide
N-[5-chloro-2-(1H-1,2,4-triazol-1-yl)phenyl]-3,4-dimethyl-
benzenesulfonamide
3,4-dimethyl-N-[2-(4-morpholinyl)-5-(trifluoromethyl)phenyl]-
benzenesulfonamide
4-chloro-N-(4-methoxy-2-(oxazol-2-yl]phenyl)-3-nitrobenzenesulfonamide
N-(4-methoxy-2-(oxazol-2-yl)phenyl)-3-nitrobenzenesulfonamide
N--{2-[5-(benzo[1,3]dioxol-5-ylamino)-[1,3,4] oxadiazol-2-yl]-phenyl}-3-
trifluoromethoxy-benzenesulfonamide
N-{2-[5-(benzo[1,3]dioxol-5-ylamino)-[1,3,4]oxadiazol-2-yl]-phenyl}-3-
methoxy-benzenesulfonamide
N-{2-[5-(benzo[1,3]dioxol-5-ylamino)-[1,3,4]oxadiazol-2-yl]-phenyl}-3,4-
dimethoxy-benzenesulfonamide
3-tert-butyl-N-(4-hydroxy-2-(oxazol-2-yl)phenyl)benzenesulfonamide
N-(4-chloro-2-nicotinoylphenyl)naphthalene-2-sulfonamide
2,3-Dihydro-benzofuran-6-sulfonic acid (2-benzoyl-4-chloro-phenyl)-amide
N-(4-chloro-2-isonicotinoylphenyl)-1,2,3,4-tetrahydroisoquinoline-7-
sulfonamide
N-(2-benzoyl-4-chlorophenyl)-4-methyl-3-(4-morpholinylcarbonyl)-
benzenesulfonamide
N-(2-benzoyl-4-chlorophenyl)-3,4-dichlorobenzenesulfonamide
N-[4-(2-benzoyl-4-chloro-phenylsulfamoyl)-2-chloro-phenyl]-acetamide
N-(2-benzoyl-4-chloro-phenyl)-3-cyano-benzenesulfonamide
N-(4-chloro-2-isonicotinoylphenyl)-3-cyanobenzenesulfonamide
4-(2-(4-bromo-3-fluorophenylsulfonamido)-5-chlorobenzoyl)pyridine 1-oxide
4-bromo-N-(4-chloro-2-(6-methylnicotinoyl)phenyl)-3-
fluorobenzenesulfonamide
4-bromo-N-(4-chloro-2-isonicotinoylphenyl)-3-fluorobenzenesulfonamide
4-bromo-N-(4-chloro-2-nicotinoylphenyl)-3-fluorobenzenesulfonamide
5-(2-(4-bromo-3-fluorophenylsulfonamido)-5-chlorobenzoyl)-2-methylpyridine
1-oxide
4-(5-chloro-2-(4-(cis-2,6-dimethylmorpholino)-3-
fluorophenylsulfonamido)benzoyl)pyridine 1-oxide
3-(5-chloro-2-(4-(cis-2,6-dimethylmorpholino)-3-
fluorophenylsulfonamido)benzoyl)pyridine 1-oxide
5-(5-chloro-2-(4-(cis-2,6-dimethylmorpholino)-3-
fluorophenylsulfonamido)benzoyl)-2-methylpyridine 1-oxide
N-(4-chloro-2-(6-methylnicotinoyl)phenyl)-4-(cis-2,6-dimethylmorpholino)-3-
fluorobenzenesulfonamide
N-(4-chloro-2-isonicotinoylphenyl)-4-(cis-2,6-dimethylmorpholino)-3-
fluorobenzenesulfonamide
N-(4-chloro-2-nicotinoylphenyl)-4-(cis-2,6-dimethylmorpholino)-3-
fluorobenzenesulfonamide
N-(2-benzoyl-4-chloro-phenyl)-3-fluoro-benzenesulfonamide
4-(5-chloro-2-(3-fluoro-4-morpholinophenylsulfonamido)benzoyl)pyridine 1-
oxide
5-(5-chloro-2-(3-fluoro-4-morpholinophenylsulfonamido)benzoyl)-2-
methylpyridine 1-oxide
N-(4-chloro-2-(6-methylnicotinoyl)phenyl)-3-fluoro-4-
morpholinobenzenesulfonamide
N-(4-chloro-2-isonicotinoylphenyl)-3-fluoro-4-
morpholinobenzenesulfonamide
N-(2-benzoyl-4-chlorophenyl)-3-methoxybenzenesulfonamide

### Preferred Embodiments

The following preferred embodiments of formulae (I, II and XX-CLXVII) are applicable to [00146-00727]:

Preferred R⁹, R¹⁷ and R¹⁸ groups

In one embodiment of formula (XX-CCXXXII), R⁹ is selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, —S(O)R³, —S(O)₂R³; —C(O)R³, and —C(O)₂R³.

In one embodiment of formula (I-CCXXXII) R¹⁷ and R¹⁸ are each independently selected from the group consisting of hydrogen, substituted or unsubstituted C₁₋₈ alkyl, substituted or unsubstituted C₂₋₈ alkenyl, substituted or unsubstituted C₂₋₈ alkynyl, substituted or unsubstituted 3- to 10-membered heterocyclyl, substituted or unsubstituted C₆₋₁₀ aryl, and substituted or unsubstituted 5- to 10-membered heteroaryl; or together with the nitrogen which they substitute, form a substituted or unsubstituted 4-, 5-, 6-, or 7-membered heterocyclyl.

In one embodiment of formula (I-CCXXXII), at least one of R¹⁷ and R¹⁸ is other than hydrogen.

In one embodiment of formula (I-CCXXXII), both of R¹⁷ and R¹⁸ are other than hydrogen.

Preferred L Groups

In one embodiment of formula (I-CCXXXII), L is a bond.

In one embodiment of formula (I-CCXXXII), L is —C(O)—.

In one embodiment of formula (I-CCXXXII), L is —S—.

In one embodiment of formula (I-CCXXXII), L is —O—.

In one embodiment of formula (I-CCXXXII), L is —S(O)—.

In one embodiment of formula (I-CCXXXII), L is —S(O)₂—.

In one embodiment of formula (I-CCXXXII), L is 13 CR^{a}R^{b}—.

In one embodiment of any of formulae (I-CCXXXII) where L is —CR^{a}R^{b}—, R^{a} is hydrogen, halogen, —OR¹ (where R¹ is as defined in formula (I) and preferably is hydrogen or C₁₋₄ alkyl), substituted or unsubstituted C₁₋₄ alkyl, or substituted or unsubstituted C₁₋₄ alkenyl.

In one embodiment of any of formulae (I-CCXXXII) where L is —CR^{a}R^{b}—, R^{a} is hydrogen, halogen, —OR¹ (where R¹ is as defined in formula (I) and preferably is hydrogen or C₁₋₄ alkyl), substituted or unsubstituted

C₂₋₄ alkyl, or substituted or unsubstituted C₂₋₄ alkenyl.

In one embodiment of any of formulae (I-CCXXXII) where L is —CR^{a}R^{b}—, R^{b} is hydrogen, halogen, or —OR¹ (where R¹ is as defined in formula (I) and preferably is hydrogen or C₁₋₄ alkyl).

In one embodiment of any of formulae (I-CCXXXII) where L is —CR^{a}R^{b}—, one of R^{a} and R^{b} is other than hydrogen.

In one embodiment of any of formulae (I-CCXXXII) where L is —CR^{a}R^{b}—, R^{a} and R^{b} are both halogen, and more preferably, are both fluorine.

In one embodiment of any of formulae (I-CCXXXII) where L is —CR^{a}R^{b}—, R^{a} is hydrogen and R^{b} is —OR¹ (where R¹ is as defined in formula (I) and is preferably hydrogen or C₁₋₄ alkyl).

In one embodiment of any of formulae (I-CCXXXII) where L is —CR^{a}R^{b}—, R^{a} and R^{b} are both —OR¹ (where R¹ is as defined in formula (I)) and where both R¹ groups are combined together with the atoms to which they are attached to form a 5-7 membered heterocyclic acetal ring system.

In one embodiment of any of formulae (I-CCXXXII) where L is —CR^{a}R^{b}—, R^{a} is C₁₋₄ alkyl or C₂₋₄ alkenyl and R^{b} is —OR¹ (where R¹ is as defined in formula (I) and is preferably hydrogen or C₁₋₄ alkyl).

In one embodiment of formula (I-CCXXXII), L is —NR^{c}—.

In one embodiment of formula (I-CCXXXII), L is —NR^{c}—, R^{c} is selected from the group consisting of hydrogen, C(O)R¹, S(O)₂R¹, C(O)₂R¹, substituted or unsubstituted C₃₋₈ alkyl, substituted or unsubstituted 3- to 10-membered heterocyclyl, substituted or unsubstituted C₂₋₈ alkenyl, and substituted or unsubstituted C₂₋₈ alkynyl.

In one embodiment, in each of the formulae (I-CCXXXII) where L is —NR^{c}—, R^{c} is hydrogen or —C(O)Me.

In one embodiment, in each of the formulae (I-CCXXXII) where L is —NR^{c}—, R^{c} is hydrogen or —C(O)Me.

In one preferred embodiment of any of formulae (I-CCXXXII) where L is —NR^{c}—, R^{c} is hydrogen, —S(O)₂R¹ or —C(O)R¹.

In one embodiment of formula (I-CCXXXII), L is —NR^{c}C(O)—.

In one embodiment of formula (I-CCXXXII), L is —C(O)NR^{c}—.

In one embodiment of formula (I-CCXXXII) where L is —NR^{c}C(O)— or —C(O)NR^{c}—, R^{c} is hydrogen.

In one embodiment of formula (I-CCXXXII), L is C═T.

In one embodiment of formula (I-CCXXXII) where L is C═T, T is ═CR^{d}R^{e}.

In other preferred embodiments for each of the formulae (I-CCXXXII) where L is C═T, T is ═CR^{d}R^{e}, and R^{d} and R^{e} are each independently selected from the group consisting of hydrogen, —C(O)R⁴¹ and —C(O)₂R⁴¹. More preferably, R^{d} and R^{e} are each independently selected from the group consisting of hydrogen, —C(O)Me and —C(O)₂Me.

In one embodiment of formula (I-CCXXXII) where L is C═T, T is ═NOR^{d}.

In other preferred embodiments for each of the formulae (I-CCXXXII) where L is C═T, T is ═NOR^{d} and R^{d} is a substituted or unsubstituted C₁₋₈ alkyl, more preferably methyl.

In one embodiment of formula (I-CCXXXII) where L is C═T, T is ═NOH.

In one embodiment of formula (I-CCXXXII) where L is C═T, T is ═NR^{d}.

In one embodiment of formula (I-CCXXXII) where L is C═T, R^{d} is hydrogen or unsubstituted C₁₋₆ alkyl.

In one embodiments for each of the formulae (I-CCXXXII) where L is C═T, T is ═CR^{d}R^{e} or ═NOR^{d}.

In other embodiments for each of the formulae (I-CCXXXII) where L is C═T, T is ═CR^{d}R^{e} or ═NOR^{d} and R^{d} and R^{e} are each independently selected from the group consisting of hydrogen, substituted or unsubstituted C₁₋₈ alkyl, —C(O)R⁴¹ and —C(O)₂R⁴¹.

Preferred Ar¹ Groups and X Substituents

In one embodiment of formula (I and VII), Ar¹ is a substituted or unsubstituted C₆₋₁₀ aryl. Preferably, Ar¹ is a substituted or unsubstituted phenyl.

In one embodiment of formulae (I and VII), Ar¹ is a substituted or unsubstituted 5- to 10-membered heteroaryl selected from pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl.

In one embodiment of formulae (I and VII), Ar¹ is substituted or unsubstituted 5- to 10-membered heteroaryl and comprises from 0 to 2 sulfur atoms, 0 to 2 oxygen atoms and 0 to 5 nitrogen atoms.

In one embodiment of formula (I and VII), heterocycle groups as substituents on Ar¹ can include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, or tetrahydrothiophene.

In one embodiment of formula (I and VII), 6-membered heteroaryl systems as substituents on Ar¹ include pyridyl; pyridazinyl, pyrazinyl, pyrimidinyl and triazinyl.

In one embodiment of formula (I and VII), 5-ring heteroaryl systems as substituents on Ar¹ include isothiazolyl, pyrazolyl, imidazolyl, triazolyl, tetrazolyl, oxazolyl, isoxazolyl, oxadiazolyl, thiadiazolyl, pyrrolyl and thiazolyl.

In one embodiment of formula (I and VII), Ar¹ is a substituted or unsubstituted 5- or 6-membered heteroaryl or substituted or unsubstituted phenyl, each optionally having 1 to 5 substituents as defined in formula (I).

In one embodiment of formula (I and II), Ar¹ is substituted or unsubstituted phenyl, having 1 to 5 substituents (X¹, X², X³, X⁴, X⁵) as defined in formula (II).

In one embodiment of formula (I and VII), Ar¹ is a substituted or unsubstituted phenyl and Z is substituted or unsubstituted 5- to 10-membered monocyclic or bicyclic heteroaryl, phenyl or naphthalenyl, each with 0 to 4 substituents.

In one embodiment of formula (I and VII), Ar¹ is a substituted or unsubstituted phenyl and Z is substituted or unsubstituted phenyl, or a substituted or unsubstituted 5- or 6-membered heteroaryl.

In one embodiment of formula (I and VII), Ar¹ is substituted or unsubstituted phenyl and Z is a substituted or unsubstituted fused 5,6-ring and 6,6-ring heteroaryl.

In one embodiment of formula (I and VII), Ar¹ is substituted or unsubstituted phenyl and Z is 3- to 10-membered heterocyclyl or —NR¹⁷R¹⁸.

In one embodiment of formula (I), Lisa bond, Ar¹ is substituted or unsubstituted phenyl and Z is 5- to 10-membered monocyclic or bicyclic heteroaryl ring system, where one ring heteroatom is located alpha (ortho) to the biaryl bond.

In one embodiment of formula (I and VII), L is a bond, Ar¹ is substituted or unsubstituted phenyl and Z is 5- to 10-membered monocyclic or bicyclic heteroaryl ring system containing one or more ring nitrogen atoms, and where one or more ring nitrogen atoms is located alpha (ortho) to the biaryl bond.

In one embodiment of formulae (I and VII), Ar¹ is selected from the group consisting of:

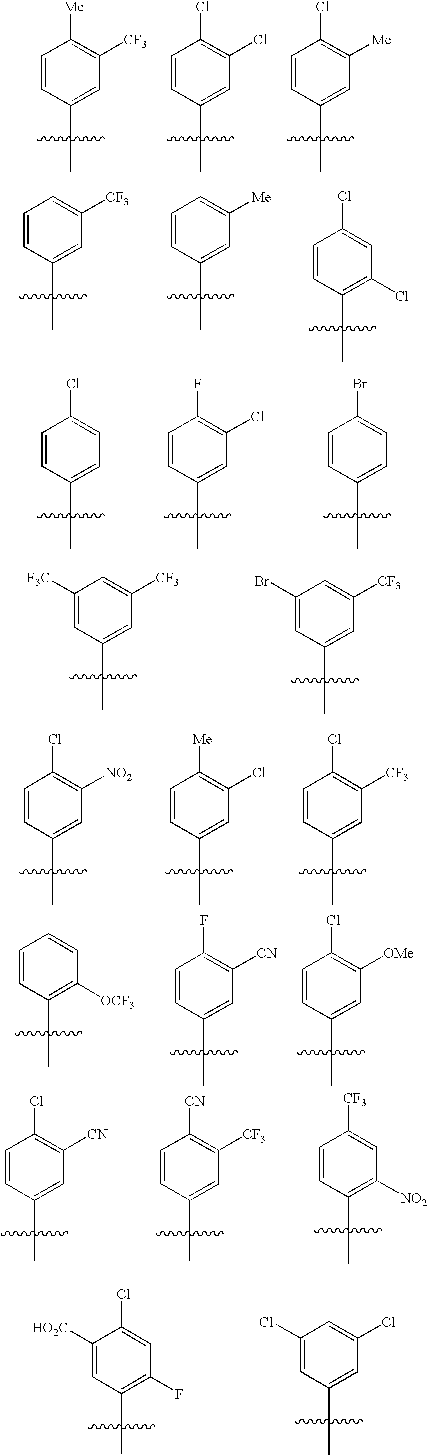

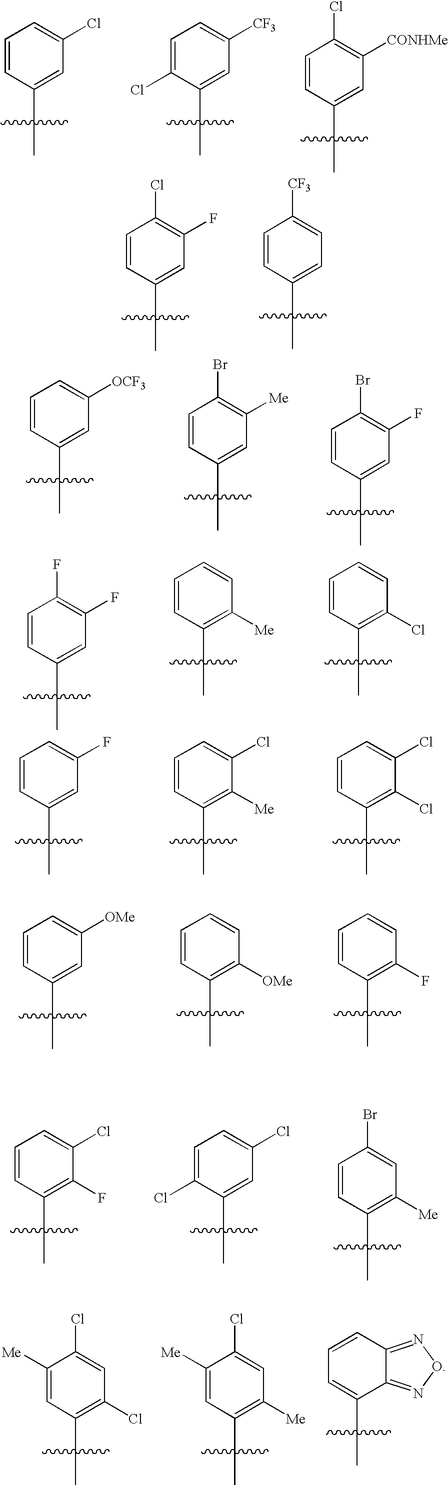

In one embodiment of any of formulae (I-CCXXXII), when Ar¹ is monosubstituted para to the sulfonyl group, the substituent is other than NR⁵C(O)R³, —NR⁵C(O)NR³R⁴, —NR⁵CO₂R³, or —NR⁵S(O)₂R³.

In one embodiment of formula (II), at least one of X¹, X², X³, X⁴, and X⁵ is other than hydrogen.

In one embodiment of formula (II), X¹ is other than hydrogen and at least 2 of X², X³, X⁴, and X⁵ are hydrogen. Preferably, at least 3 of X², X³, X⁴, and X⁵ are hydrogen; more preferably, X², X³, X⁴, and X⁵ are hydrogen.

In one embodiment of formula (II), X¹ is other than hydrogen and at least 2 of X², X³, X⁴, and X⁵ are hydrogen. Preferably, at least 3 of X², X³, X⁴, and X⁵ are hydrogen; more preferably, X², X³, X⁴, and X⁵ are hydrogen; also more preferably X², X³, and X⁵ are hydrogen.

In one embodiment of formula (II), X¹ is other than hydrogen and at least 3 of X², X³, X⁴, and X⁵ are hydrogen. Preferably, X³, X⁴, and X⁵ are hydrogen.

In one embodiment of any of formulae (III, IV, and XX-CCXXXII), X¹ is other than NR⁵C(O)R³, —NR⁵C(O)NR³R⁴, —NR⁵CO₂R³, or —NR⁵S(O)₂R³.

In one embodiment of any of formulae (III, IV, and XX-CCXXXII), X¹, X², X^{a}, and X^{b} are selected from the group consisting of halogen, —NO₂, —CN, substituted or unsubstituted C₁₋₈ alkyl, —OR³, —CO₂R³.

In one embodiment of any of formulae (III, IV and XX-CCXXXII), X¹, X², X^{a}, and X^{b} are selected from the group consisting of —Cl, —F, —Br, —NO₂, —CN, —OCH₃, —OCF₃, —CH₃, —CF₃, —CONHCH₃, and —CO₂H.

In one embodiment of any of formulae (II-VI, and XX-CCXXXII), any one of X¹, X², X³, X⁴, X⁵, X^{a}, and X^{b} may be a heterocyclic group represented by formula (AA) below, where formula (AA) is attached via a free valence on either M¹ or M², and where formula (AA) and the substituents therein are defined in [0045]-[0048].

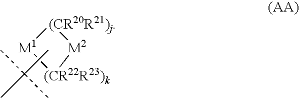

In one embodiment of each of the formulae (I-CCXXXII), when a heterocyclic group represented by formula (AA) is present, R²⁰, R²¹, R²², R²³, R²⁴, R²⁵, and R²⁶ are independently hydrogen or C₁₋₄alkyl. In another preferred embodiment, at least three of R²⁰, R²¹, R²², R²³, R²⁴, R²⁵, and R²⁶ are hydrogen, j is 1 or 2, k is 1 or 2 with the proviso that j+k is 3 or 4. In another preferred embodiments, at least five of R²⁰, R²¹, R²², R²³, R²⁴, R²⁵, and R²⁶ are hydrogen, j is 1 or 2, k is 1 or 2 with the proviso that j+k is 3 or 4.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, —NR⁵C(O)R³, —NR⁵C(O)₂R³, —NR⁵C(O)NR³R⁴, —NR⁵S(O)₂R³, —S(O)₂NR³R⁴, substituted or unsubstituted C₁₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, —S(O)₂NR³R⁴, substituted or unsubstituted C₁₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, NR⁵C(O)R³, —NR⁵C(O)₂R³, —NR⁵C(O)NR³R⁴, —NR⁵S(O)₂R³, —S(O)₂NR³R⁴, substituted C₁₋₈ alkyl, unsubstituted C₂₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, substituted C₁₋₈ alkyl, unsubstituted C₂₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, —S(O)₂NR³R⁴, substituted or unsubstituted C₁₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In one embodiment, in each of formulae (II-VI, and XX-CCXXXII), X¹, X², X³, X⁴, X⁵, X^{a}, and X^{b} are each independently selected from the group consisting of hydrogen, halogen, —CN, —NO₂, —OH, —OR³, —C(O)R³, —CO₂R³, —O(CO)R³, —OC(O)NR³R⁴, —SR³, —S(O)R³, —S(O)₂R³, —NR³R⁴, substituted or unsubstituted C₁₋₈ alkyl, substituted or unsubstituted C₂₋₈ alkenyl, substituted or unsubstituted C₂₋₈ alkynyl, substituted or unsubstituted phenyl, substituted or unsubstituted 5- or 6-membered heteroaryl, and substituted or unsubstituted 4- to 7-membered heterocyclyl.

In another embodiment of formulae (II, V, VI and XX-CCXXXII), X¹, X², X³, X⁴ and X⁵ are each independently selected from the group consisting of halogen, substituted or unsubstituted C₁₋₈ alkyl, —OR³, —NO₂, —CN, —CO₂R³, and —CO₂H.

In another embodiment of formulae (II, V, VI and XX-CCXXXII), X¹, X², X³, X⁴ and X⁵ are each independently selected from the group consisting of —Cl, —F, —Br —CF₃, —OCF₃, —OCH₃—CH₃, —NO₂, —CN, —CO₂H, and —CONHMe.

In another embodiment of formula (II), X¹, X², X³, X⁴ and X⁵ are each independently selected from the group consisting of hydrogen, halogen, —CN, —NO₂, —OR³, —C(O)R³, —SO₂R³, —NR³R⁴, unsubstituted or substituted C₁₋₈ alkyl, unsubstituted or substituted phenyl, unsubstituted or substituted 5- or 6-membered heteroaryl, and unsubstituted or substituted 5- or 6-membered heterocyclyl; with the proviso that at least two of X¹, X², X³, X⁴ and X⁵ are other than hydrogen; or with the proviso that at least one of X¹, X², X³, X⁴ and X⁵ is other than hydrogen.

In another embodiment of formula (II), X¹, X², X³, X⁴ and X⁵ are each independently selected from the group consisting of: hydrogen, halogen, —CN, —NO₂, —OR³, —C(O)R³, —SO₂R³, —NR³R⁴, unsubstituted or substituted C₁₋₈ alkyl, unsubstituted or substituted phenyl, unsubstituted or substituted 5- or 6-membered heteroaryl, and unsubstituted or substituted 5- or 6-membered heterocyclyl; with the proviso that at least two of X¹, X², and X⁴ are other than hydrogen; or with the proviso at least one of X¹, X², and X⁴ is other than hydrogen.

In a further embodiment of formula (II), X¹, X², X³, X⁴ and X⁵ are each independently selected from the group consisting of: hydrogen, halogen, —CN, —NO₂, —OR³, —C(O)R³, —SO₂R³, and —NR³R⁴; with the proviso that at least three of X¹, X², X³, X⁴ and X⁵ are other than hydrogen; or with the proviso that at least two of X¹, X², X³, X⁴ and X⁵ is other than hydrogen; or with the proviso that at least one of X¹, X², X³, X⁴ and X⁵ is other than hydrogen.

In a further embodiment of formula (II), X¹, X², X³, X⁴ and X⁵ are each independently selected from the group consisting of: hydrogen, halogen, unsubstituted or substituted C₁₋₈ alkyl, unsubstituted or substituted phenyl, unsubstituted or substituted 5- or 6-membered heteroaryl, and unsubstituted or substituted 5- or 6-membered heterocyclyl; with the proviso that at least three of X¹, X², X³, X⁴ and X⁵ are other than hydrogen; or with the proviso that at least two of X¹, X², X³, X⁴ and X⁵ is other than hydrogen; or with the proviso that at least one of X¹, X², X³, X⁴ and X⁵ is other than hydrogen.

In one embodiment of any of formulae (I-CCXXXII), X¹, X^{a}, and X^{b} are each independently selected from the group consisting of hydrogen, halogen, —NO₂, —OR³, —C(O)R³, —S(O)₂R³, —NR³R⁴, substituted or unsubstituted C₁₋₈ alkyl, substituted or unsubstituted phenyl, substituted or unsubstituted 5- or 6-membered heteroaryl, and substituted or unsubstituted 5- or 6-membered heterocyclyl. In one preferred embodiment, X^{a} or X² is hydrogen. In another, X^{a} or X² is halogen, —CN, —CH₃ or —CF₃.

In one embodiment of any of formulae (I-CCXXXII), X¹, X^{a}, and X^{b} are each independently selected from the group consisting of hydrogen, halogen, —NO₂, —OR³, —C(O)R³, —S(O)₂R³, —NR³R⁴, substituted C₁₋₈ alkyl, unsubstituted C₂₋₈ alkyl, substituted or unsubstituted phenyl, substituted or unsubstituted 5- or 6-membered heteroaryl, and substituted or unsubstituted 5- or 6-membered heterocyclyl. In one preferred embodiment, X^{a} or X² is hydrogen. In another, X^{a} or X² is halogen, —CN, —CH₃ or —CF₃.

In one embodiment of each of the formulae (II-VI, and XX-CCXXXII), at least one of X¹, X^{a}, and X^{b} is a substituted or unsubstituted 5- or 6-membered heterocyclic ring, and the heterocycle is selected from the group consisting of pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of each of the formulae (II-VI, and XX-CCXXXII), at least one of X¹, X^{a}, and X^{b} is a substituted or unsubstituted 5- or 6-membered heteroaryl ring selected from the group consisting of pyridyl, pyridazinyl, pyrazinyl, pyrimidinyl, triazinyl, isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl.

In one embodiment of each of the formulae (II-VI, and XX-CCXXXII), at least one of X^{a}, X^{b}, X¹, X², X³, X⁴, X⁵ or at least one X is substituted or unsubstituted heterocyclic group selected from the group consisting of pyrrolidinyl, piperidinyl, piperazinyl, morpholinyl, 1,3-dioxalanyl, thiomorpholinyl, thiomorpholinyl-S,S-dioxide, piperazinyl and pyranyl.

In one embodiment of each of the formulae (II-VI, and XX-CCXXXII), at least one of X^{a}, X^{b}, X¹, X², X³, X⁴, X⁵ or at least one X is a substituted C₁₋₈ alkyl, where suitable substituents are as defined for formula (II). Preferably, the substituent is a substituted or unsubstituted heterocyclic group of the formula (AA) as defined in paragraphs [0045]-[0048]. More preferably, the substituent is selected from the group including pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of each of the formulae (II-VI, and XX-CCXXXII), at least one of X^{a}, X^{b}, X¹, X², X³, X⁴, X⁵ or at least one X is a substituted C₁₋₈ alkyl, where suitable substituents are as defined for formula (II). In one preferred embodiment, the substituted C₁₋₈ alkyl is substituted with a 5- or 6-membered heteroaryl selected from the group consisting of pyridyl, pyridazinyl, pyrazinyl, pyrimidinyl, triazinyl, isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl. More preferably, the substituted C₁₋₈ alkyl is substituted with oxazolyl.

In one embodiment of each of the formulae (I-CCXXXII), a suitable substituent for substituted C₁₋₈ alkyl (as X, X¹, X², X³, X⁵, X^{a}, X^{b}, or X) can be selected from the group consisting of —CN, —OR¹, —C(O)R¹, —CO₂R¹, —O(CO)R¹, —SO₂R¹ and halogen.

In one embodiment of each of the formulae (II-VI, and XX-CCXXXII), at least one of X^{a}, X^{b}, X¹, X², X³, X⁴, X⁵ or at least one X is halogen, particularly chlorine.

In one embodiment of each of the formulae (II-VI, and XX-CCXXXII), at least one of X^{a}, X^{b}, X¹, X², X³, X⁴, X⁵ or at least one X is an unsubstituted C₁₋₈ alkyl.

In one embodiment of each of the formulae (II-VI, and XX-CCXXXII), at least one of X^{a}, X^{b}, X¹, X², X³, X⁴, X⁵ or at least one X is an unsubstituted C₂₋₈ alkyl.

In one embodiment of each of the formulae (II-VI, and XX-CCXXXII), at least one of X^{a}, X^{b}, X¹, X², X³, X⁴, X⁵ or at least one X is t-butyl.

In one embodiment of each of the formulae (II-VI, and XX-CCXXXII), at least one of X^{a}, X^{b}, X¹, X², X³, X⁴, X⁵ or at least one X is oxazolyl.

In one embodiment of each of the formulae (II-VI, and XX-CCXXXII), at least one of X^{a}, X^{b}, X¹, X², X³, X⁴, X⁵ or at least one X is trifluoromethoxy.

In one embodiment of each of the formulae (II-VI, and XX-CCXXXII), at least one of X^{a}, X^{b}, X¹, X², X³, X⁴, X⁵ or at least one X is —SO₂R³. In one particular embodiment, R³ is methyl.

In one embodiment of each of the formulae (II-VI, and XX-CCXXXII), at least one of X^{a}, X^{b}, X¹, X², X³, X⁴, X⁵ or at least one X is —OR³. In one particular embodiment, R³ is methyl.

In one embodiment of each of the formulae (II-VI, and XX-CCXXXII), at least one of X^{a}, X^{b}, X¹, X², X³, X⁴, X⁵ or at least one X is —SR³. In one particular embodiment, R³ is methyl.

In one embodiment of each of the formulae (II-VI, and XX-CCXXXII), at least one of X^{a}, X^{b}, X¹, X², X³, X⁴, X⁵ or at least one X is unsubstituted C₁₋₆ alkyl (in particular methyl) or C₁₋₆ haloalkyl (in particular —CF₃).

In one embodiment of each of the formulae (II-VI, and XX-CCXXXII), at least one of X^{a}, X^{b}, X¹, X², X³, X⁴, X⁵ or at least one X is substituted C₁₋₆ alkyl (preferably not C₁₋₆ haloalkyl).

In one embodiment of each of the formulae (II-VI, and XX-CCXXXII), at least one of X^{a}, X^{b}, X¹, X², X³, X⁴, X⁵ or at least one X is isopropyl.

In one embodiment of each of the formulae (II-VI, and XX-CCXXXII), at least one of X^{a}, X^{b}, X¹, X², X³, X⁴, X⁵ or at least one X is a cyano.

In one embodiment of each of the formulae (II-VI, and XX-CCXXXII), at least one of X^{a}, X^{b}, X¹, X², X³, X⁴, X⁵ or at least one X is a cyano, halogen or trifluoromethyl group.

In one embodiment of each of the formulae (II-VI, and XX-CCXXXII), at least one of X^{a}, X^{b}, X¹, X², X³, X⁴, X⁵ or at least one X is —C(Me)₂CH₂OH.

In one embodiment of each of the formulae (II-VI, and XX-CCXXXII), at least one of X^{a}, X^{b}, X¹ X², X³, X⁴, X⁵ or at least one X is —C(O)Me.

In one embodiment of each of the formulae (II-VI, and XX-CCXXXII), at least one of X^{a}, X^{b}, X¹, X², X³, X⁴, X⁵ or at least one X is —(CH₂)₂CO₂Me.

In one embodiment of each of the formulae (II-VI, and XX-CCXXXII), at least one of X^{a}, X^{b}, X¹, X², X³, X⁴, X⁵ or at least one X is isoamyl.

In one embodiment of each of the formulae (II-VI, and XX-CCXXXII), at least one of X^{a}, X^{b}, X¹, X², X³, X⁴, X⁵ or at least one X is 1,3-dioxalanyl.

In one embodiment of each of the formulae (II-VI, and XX-CCXXXII), at least one of X^{a}, X^{b}, X¹, X², X³, X⁴, X⁵ or at least one X is furyl.

In one embodiment of each of the formulae (II-VI, and XX-CCXXXII), at least one of X^{a}, X^{b}, X¹, X², X³, X⁴, X⁵ or at least one X is pyrazolyl.

In one embodiment of each of the formulae (II-VI, and XX-CCXXXII), at least one of X^{a}, X^{b}, X¹, X², X³, X⁴, X⁵ or at least one X is thienyl.

In one embodiment of paragraphs [00226] to [00253], the remaining substituents are hydrogen. In another embodiment, the remaining substituents are selected from the group consisting of hydrogen, halogen, cyano, or trifluoromethyl.

In one embodiment of formula (II-VI, and XX-CCXXXII), at least one of X¹, X², X³, X⁴, X⁵, X^{a} and X^{b} is other than hydrogen.

In one embodiment of formula (II-VI, and XX-CCXXXII), at least two of X¹, X², X³, X⁴, X⁵, X^{a} and X^{b} is other than hydrogen.

In one embodiment of formula (II-VI, and XX-CCXXXII), at least three of X¹, X², X³, X⁴, X⁵, X^{a} and X^{b} is other than hydrogen.

In one embodiment of formula (II, V, VI and XX-CCXXXII), X¹ is other than hydrogen.

In one embodiment of formula (II, V, VI), X¹ and X² are other than hydrogen.

In one embodiment of formula (II, V, VI and XX-CCXXXII), X¹ is other than —NR⁵C(O)R³, —NR⁵C(O)₂R³, —NR⁵C(O)NR³R⁴, —NR⁵S(O)₂R³.

In one embodiment of formula (II, V, VI and XX-CCXXXII), X¹ is chlorine.

In one embodiment of formula (II, V, VI), X¹ is chlorine and X² is —CF₃.

In one embodiment of formula (II, V, VI), X¹ is chlorine and X² is selected from the group consisting of —CF₃, —Cl, —NO₂.

In one embodiment of formula (II, V, VI), X¹ is chlorine and X² is selected from the group consisting of —F, —Cl, —Br, —CN, —CF₃, —NO₂, —CH₃.

In one embodiment of formula (II, V, VI), X¹ and X² are each independently selected from the group consisting of halogen, —CN, —CF₃, —NO₂, C₁₋₈ alkyl, unsubstituted or substituted heteroaryl, and unsubstituted or substituted heterocyclyl.

In one embodiment of formulae (II, V, VI), X¹ is methyl and X² is selected from the group consisting of halogen, —CN, —CF₃.

In one embodiment of formulae (II, V, VI), X¹ is methyl and X² is selected from the group consisting of halogen, —CN, —NO₂, —OCF₃, —OCH₃, —CH₃, —CF₃, —CO₂H, —CONHCH₃, or hydrogen.

In one embodiment of formula (II, V, VI), X² is other than hydrogen.

In one embodiment of formula (II, V, VI), X² is selected from the group consisting of —CN, —C(O)R³, —CO₂R³, —C(O)NR³R⁴, —NO₂, —S(O)R³, —S(O)₂R³, —S(O)₂NR³R⁴.

In one embodiment of formula (III, IV, and XX-CCXXXII), X^{a} is chlorine and X^{b} is —CF₃.

In one embodiment of formula (III, IV, and XX-CCXXXII), X^{a} is chlorine and X^{b} is selected from the group consisting of —CF₃, —Cl, —NO₂.

In one embodiment of formula (III, IV, and XX-CCXXXII), X^{a} is chlorine and X^{b} is selected from the group consisting of —F, —Br, —CN.

In another embodiment of formulae (III, IV, and XX-CCXXXII), X^{a} and X^{b} are both other than hydrogen.

In one embodiment of formulae (III, IV, and XX-CCXXXII), at least one of X^{a} and X^{b} is selected from the group consisting of halogen, —CN, —NO₂, —OR³, —C(O)R³, —SO₂R³, —NR³R⁴, unsubstituted or substituted C₁₋₈ alkyl, unsubstituted or substituted phenyl, unsubstituted or substituted 5- or 6-membered heteroaryl, and unsubstituted or substituted 5- or 6-membered heterocyclyl.

In another embodiment of formulae (III, IV, and XX-CCXXXII), X^{a} and X^{b} are both selected from the group consisting of halogen, —CN, —NO₂, —OR³, —C(O)R³, —SO₂R³, —NR³R⁴, unsubstituted or substituted C₁₋₈ alkyl, unsubstituted or substituted phenyl, unsubstituted or substituted 5- or 6-membered heteroaryl, and unsubstituted or substituted 5- or 6-membered heterocyclyl.

In one embodiment of each of the formulae (II-VI, and XX-CCXXXII), at least one of X, X¹, X², X³, X⁴, X⁵, X^{a}, and X^{b} are independently selected from the group consisting of:

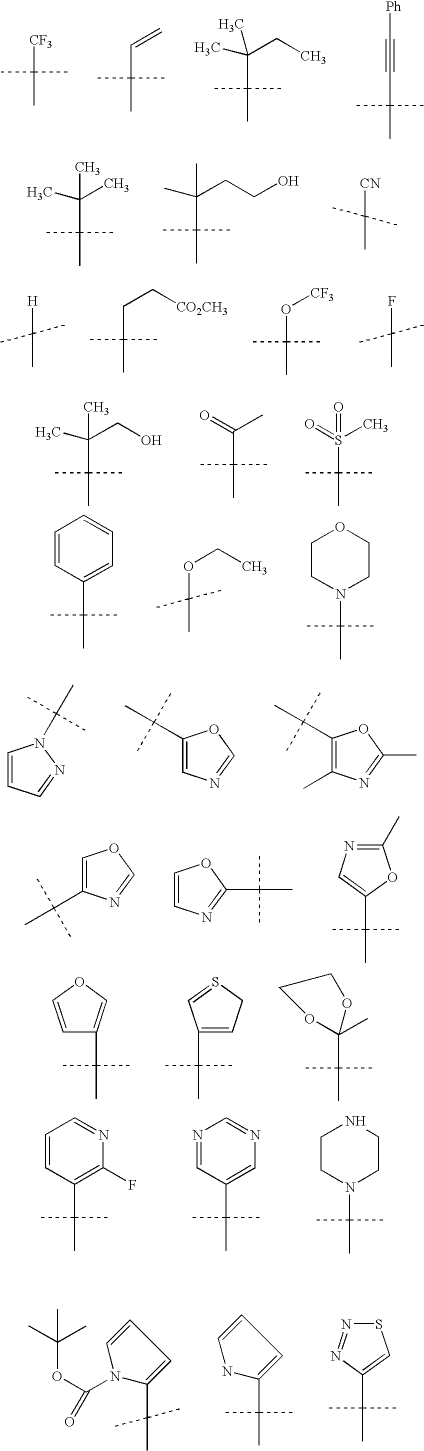

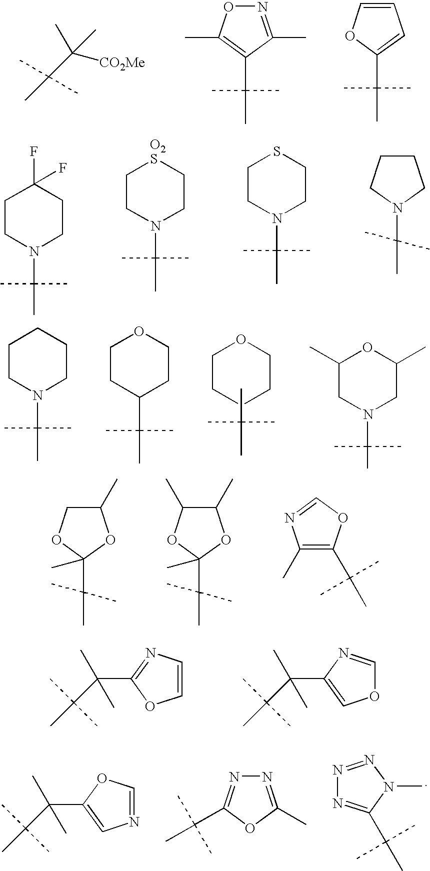

In one embodiment of each of the formulae (II-VI, and XX-CCXXXII), at least one of X, X¹, X², X³, X⁴, X⁵, X^{a}, X^{b} are independently selected from the group consisting of:

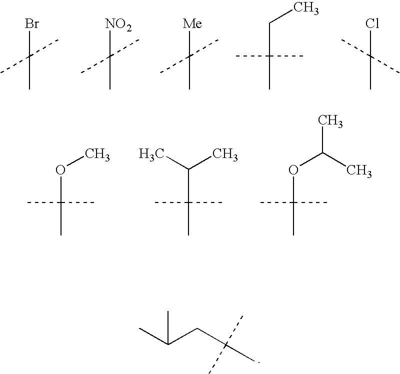

In one embodiment of each of the formulae (II-VI, and XX-CCXXXII), X, X¹, X^{a}, and X^{b} are independently selected from the group consisting hydrogen, —OMe, —OEt, ethyl, methyl, iso-propyl, isoamyl, or —CF₃ with the proviso that at least one substituent is other than hydrogen.

In one embodiment of each of the formulae (II-VI, and XX-CCXXXII), X¹, X^{a}, and X^{b} are independently selected from the group consisting of hydrogen, —S(O)₂R³, —NR⁵C(O)NR³R⁴, —NR⁵S(O)₂R³, —S(O)₂NR³R⁴, substituted C₁₋₈ alkyl (but not C₁₋₈ haloalkyl), substituted C₂₋₈ alkenyl, substituted C₂₋₈ alkynyl, substituted or unsubstituted 5- or 6-membered heteroaryl, or substituted or unsubstituted 4- to 7-membered heterocyclyl, with the proviso that at least one substituent is other than hydrogen.

In one embodiment of each of the formulae (II-VI, and XX-CCXXXII), X¹, X^{a}, and X^{b} are independently selected from the group consisting of hydrogen, —S(O)₂R³, —S(O)₂NR³R⁴, substituted C₁₋₈ alkyl (but not C₁₋₈ haloalkyl), substituted C₂₋₈ alkenyl, substituted C₂₋₈ alkynyl, substituted or unsubstituted 5- or 6-membered heteroaryl, or substituted or unsubstituted 4- to 7-membered heterocyclyl, with the proviso that at least one substituent is other than hydrogen.

In one embodiment of each of the formulae (II-VI, and XX-CCXXXII), X¹, X^{a}, and X^{b} are independently selected such that at least one substituent is unsubstituted C₁₋₈ alkyl.

In one embodiment of each of the formulae (II-VI, and XX-CCXXXII), X¹, X^{a}, and X^{b} are independently selected such that at least one substituent is —CF₃, —CN, —Cl or —Br.

In one embodiment of each of the formulae (II-VI, and XX-CCXXXII), X¹, X^{a}, and X^{b} are independently selected from the group consisting of hydrogen, —NO₂, —OR³,13 C(O)R³, —S(O)₂R³, —NR³R⁴, substituted unsubstituted C₁₋₈ alkyl, substituted or unsubstituted phenyl, substituted or unsubstituted 5- or 6-membered heteroaryl, or substituted or unsubstituted 5- or 6-membered heterocyclyl. Preferably, X^{a} is hydrogen. In another preferred embodiment, X^{a} is fluorine, chlorine, —CN, —CF₃.

In one embodiment of each of the formulae (II-VI, and XX-CCXXXII), X^{a} is hydrogen, and X^{b} is other than hydrogen.

In one embodiment, in each of formulae (II-VI, and XX-CCXXXII), at least one of X¹, X^{a}, and X^{b} is a substituted or unsubstituted C₁₋₈ alkyl as defined for formula (I). In certain preferred embodiments, X^{a} or X² is hydrogen, and X¹ and X^{b} are substituted C₁₋₈ alkyl. In certain other preferred embodiments, X^{a} or X² is hydrogen, and X¹ and X^{b} are unsubstituted C₁₋₈ alkyl. In certain other preferred embodiments, X^{a} or X² is hydrogen, and X¹ and X^{b} are unsubstituted C₂₋₈ alkyl.

In one embodiment, in each of formulae (II-VI, and XX-CCXXXII), at least one of X¹, X^{a}, and X^{b} is a halogen atom, —CF₃, —CN, substituted or unsubstituted C₁₋₈ alkyl, substituted or unsubstituted 5- or 6-membered heteroaryl, or substituted or unsubstituted 4- to 7-membered heterocyclyl and when an additional X¹, X^{a}, and X^{b} group is present, it is a halogen atom or —CF₃.

In one embodiment of any of formulae (II-VI, and XX-CCXXXII), at least one of X¹, X^{a}, and X^{b} is a substituted or unsubstituted 5- or 6-membered heteroaryl ring selected from the group consisting of pyridyl, pyridazinyl, pyrazinyl, pyrimidinyl, triazinyl, isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl; and wherein additional X¹, X², X³, X⁴, X⁵, X^{a}, and X^{b}, when present, are defined as for formula (II).

In one embodiment of any of formulae (II-VI, and XX-CCXXXII), at least one of X¹, X^{a}, and X^{b} is a substituted or unsubstituted oxazolyl.

In one embodiment of any of formulae (II-VI, and XX-CCXXXII), at least one of X¹, X^{a}, and X^{b} is a substituted or unsubstituted morpholinyl.

In one embodiment of any of formulae (II-VI, and XX-CCXXXII), at least one of X¹, X^{a}, and X^{b} is a substituted or unsubstituted 5- or 6-membered heterocyclic ring, and the heterocycle is selected from the group consisting of pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene; and wherein additional X¹, X², X³, X⁴, X⁵, X^{a} and X^{b}, when present, are defined as for X¹ in formula (II).

In one embodiment of any of formulae (II-VI, and XX-CCXXXII), at least one of X¹, X^{a}, and X^{b} is a substituted or unsubstituted 5- or 6-membered heterocyclic ring, and the heterocycle is selected from the group consisting of pyrrolidine, piperidine, imidazolidine, pyrazolidine, dioxolane, piperidine, 1,4-dioxane, morpholine, thiomorpholine, thiomorpholine-S-oxide, thiomorpholine-S,S-dioxide, piperazine, pyran, 3-pyrroline, thiopyran, pyrone, tetrahydrofuran, and tetrahydrothiophene; and wherein additional X¹, X², X³, X⁴, X⁵, X^{a}, and X^{b}, when present, are defined as for X¹ in formula (II).

In a preferred embodiment of any of formulae (II-VI, and XX-CCXXXII), one of X¹ or X^{b} is selected from the above group and X^{a} or X² is selected from the group consisting of —F, —Cl, —CN, —CH₃ and —CF₃. In another preferred embodiment, one of X¹ or X^{b} is selected from the above group and X^{a} or X² is hydrogen.

In embodiments for each of the formulae (II-VI, and XX-CCXXXII), where X¹ or X^{b} is either a substituted C₁₋₈ alkyl, a substituted 5- or 6-membered heteroaryl, or a substituted 4- to 7-membered heterocycle, the aromatic or aliphatic portions of X¹ or X^{b} may have from 1 to 3 substituents independently selected from the group consisting of halogen, —CN, ═O, —OC(O)R³, —OR³, —C(O)R³, —C(O)NR³R⁴, —NR⁵C(O)R³, —CO₂R³, —NR³R⁴, —S(O)₂R³, substituted or unsubstituted phenyl, substituted or unsubstituted 5- or 6-membered heteroaryl, and substituted or unsubstituted 4- to 7-membered heterocycle.

In embodiments for each of the formulae (II-VI, and XX-CCXXXII), where X¹ or X^{b} is either a substituted C₁₋₈ alkyl, a substituted 5- or 6-membered heteroaryl, or a substituted 4- to 7-membered heterocycle, the aromatic or aliphatic portions of X¹ or X^{b} may have from 1 to 3 substituents independently selected from the group consisting of halogen, —CN, ═O, —OC(O)R³, —OR³, —C(O)R³, —C(O)NR³R⁴, —CO₂R³, —NR³R⁴, —S(O)₂R³, substituted or unsubstituted phenyl, substituted or unsubstituted 5- or 6-membered heteroaryl, and substituted or unsubstituted 4- to 7-membered heterocycle.

In one embodiment, in each of formulae (II-VI, and XX-CCXXXII), X¹ and X^{b} are independently selected from the group consisting of —CN, halogen, —NO₂, —OR³, —C(O)R³, —S(O)₂R³, —NR³R⁴, substituted or unsubstituted C₁₋₈ alkyl, substituted or unsubstituted phenyl, substituted or unsubstituted 5- or 6-membered heteroaryl, and substituted or unsubstituted 5- or 6-membered heterocyclyl. In one preferred embodiment, X^{a} or X² is hydrogen. In another embodiment, X^{a} or X² is other than hydrogen, and in a further embodiment, X^{a} or X² is halogen, cyano or C₁₋₈ haloalkyl.

In one embodiment, in each of formulae (II-VI, and XX-CCXXXII), X¹ and X^{b} are independently selected from the group consisting of:

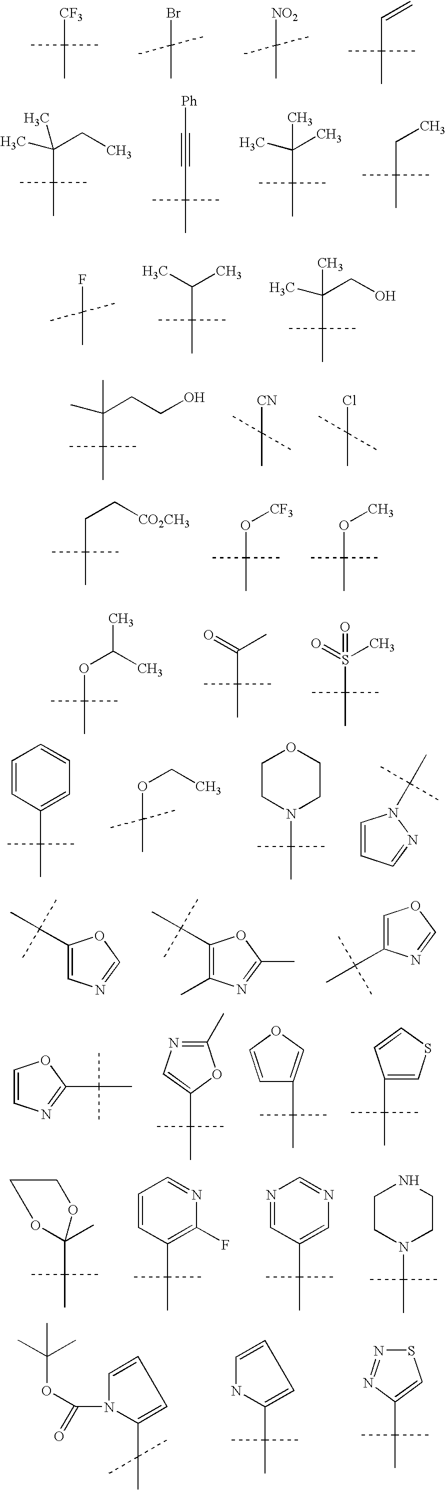

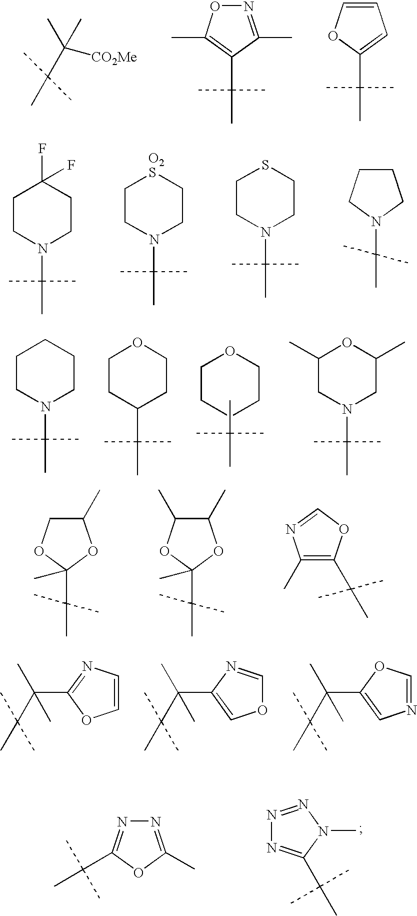

X² and X^{a} are selected from either hydrogen or fluorine; and X³, X⁴ and X⁵ are hydrogen.

In one embodiment of any of formulae (II-VI, and XX-CCXXXII), each of X^{a}, X², X³, X⁴ and X⁵ is hydrogen, and X¹ or X^{b} is other than hydrogen.

In one embodiment, in each of formulae (II-VI, and XX-CCXXXII), each of X^{a}, X², X³, X⁴ and X⁵ is hydrogen and X¹ or X^{b} is hydrogen and the other is C₁₋₈ alkyl, optionally having from 1 to 3 substituents independently selected from the group consisting of halogen, —CN, ═O, —OC(O)R³, —OR³, —C(O)R³, —C(O)NR³R⁴, —NR⁴C(O)R³, —CO₂R³, —NR³R⁴, —SR³, —S(O)R³, —S(O)₂R³, —NR³S(O)₂R⁴, substituted or unsubstituted phenyl, and substituted or unsubstituted 5- or 6-membered heteroaryl, and substituted or unsubstituted 4- to 7-membered heterocycle. In other embodiments, one of X^{a} and X² is selected from the group consisting of halogen, cyano or C₁₋₈ haloalkyl.

In one embodiment, in each of formulae (II-VI, and XX-CCXXXII), each of X^{a}, X², X³, X⁴ and X⁵ is hydrogen and X¹ or X^{b} is hydrogen and the other is C₁₋₈ alkyl, optionally having from 1 to 3 substituents independently selected from the group consisting of halogen, —CN, ═O, —OC(O)R³, —OR³, —C(O)R³, —C(O)NR³R⁴, —CO₂R³, —NR³R⁴, —SR³, —S(O)R³, —S(O)₂R³, substituted or unsubstituted phenyl, and substituted or unsubstituted 5- or 6-membered heteroaryl, and substituted or unsubstituted 4- to 7-membered heterocycle. In other embodiments, one of X^{a} and X² is selected from the group consisting of halogen, cyano or C₁₋₈ haloalkyl.

In one embodiment, in each of formulae (II-VI, and XX-CCXXXII), each of X^{a}, X², X³, X⁴ and X⁵ is hydrogen and X¹ or X^{b} is C₆₋₁₀ aryl or a heteroaryl, optionally having from 1 to 3 substituents independently selected from the group consisting of halogen, —CN, —OH, —OR³, ═O, —OC(O)R³, —CO₂R³, —C(O)R³, —C(O)NR³R⁴, —NR⁵C(O)R³, —NR³R⁴, —SR³, —S(O)R³, —S(O)₂R³, —NR⁵S(O)₂R³, and substituted or unsubstituted C₁₋₈ alkyl. In other embodiments, one of X^{a} and X¹ is selected from the group consisting of halogen, cyano, nitro C₁₋₈ alkyl and C₁₋₈ haloalkyl.

In one embodiment, in each of formulae (II-VI, and XX-CCXXXII), each of X^{a}, X², X³, X⁴ and X⁵ is hydrogen and X¹ or X^{b} is C₆₋₁₀ aryl or a heteroaryl, optionally having from 1 to 3 substituents independently selected from the group consisting of halogen, —CN, —OH, —OR³, ═O, —OC(O)R³, —CO₂R³, —C(O)R³, —C(O)NR³R⁴, —NR³R⁴, —SR³, —S(O)R³, —S(O)₂R³ and substituted or unsubstituted C₁₋₈ alkyl. In other embodiments, one of X^{a} and X¹ is selected from the group consisting of halogen, cyano, nitro C₁₋₈ alkyl and C₁₋₈ haloalkyl.

In one embodiment, in each of formulae (II-VI, and XX-CCXXXII), each of X^{a}, X², X³, X⁴ and X⁵ is hydrogen and X¹ or X^{b} is a 5- or 6-membered heterocyclyl, optionally having 1 to 2 substituents independently selected from the group consisting of substituted or unsubstituted C₁₋₈ alkyl, —OR³, —C(O)R³, —C(O)NR³R⁴, —NR³R⁴, and —S(O)₂R³. In other embodiments, one of X^{a} and X² is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is substituted phenyl or substituted 5- or 6-membered heteroaryl, optionally having from 1 to 3 substituents independently selected from the group consisting of halogen, —OR³, —NO₂, ═O—C(O)R³, —C(O)NR³R⁴, —NR⁵C(O)R³, —NR³R⁴, —SR³, —NR⁵S(O)₂R³, —S(O)₂R³, and substituted or unsubstituted C₁₋₈ alkyl. In other embodiments, one of X^{a} and X² is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is substituted phenyl or substituted 5- or 6-membered heteroaryl, optionally having from 1 to 3 substituents independently selected from the group consisting of halogen, —OR³, —NO₂, ═O—C(O)R³, —C(O)NR³R⁴, —NR³R⁴, —SR³, —S(O)₂R³, and substituted or unsubstituted C₁₋₈ alkyl. In other embodiments, one of X^{a} and X² is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is a 4- to 7-membered heterocyclyl, optionally having from 1 to 3 substituents independently selected from the group consisting of substituted or unsubstituted C₁₋₈ alkyl, —OR³, —OC(O)R³, —CO₂R³, —C(O)R³, —CONR³R⁴, —NR³R⁴, —NR⁵C(O)R³, —S(O)₂R³, —SR³ and —NR⁵S(O)₂R³. In embodiments, one of X^{a} and X¹ is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is a 4- to 7-membered heterocyclyl, optionally having from 1 to 3 substituents independently selected from the group consisting of substituted or unsubstituted C₁₋₈ alkyl, —OR³, —OC(O)R³, —CO₂R³, —C(O)R³, —CONR³R⁴, —NR³R⁴, —S(O)₂R³, and —SR³. In other embodiments, one of X^{a} and X¹ is selected from the group consisting of halogen, cyano, nitro, C₁₋₈ haloalkyl and C₁₋₈ haloalkyl.

In one embodiment of any of formulae (II-VI, and XX-CCXXXII), each of X^{a}, X², X³, X⁴ and X⁵ is hydrogen, and X¹ or X^{b} is other than hydrogen.

In one embodiment, in each of formulae (II-VI, and XX-CCXXXII), each of X^{a}, X², X³, X⁴ and X⁵ is hydrogen, and X¹ or X^{b} is C₁₋₈ alkyl, optionally having from 1 to 3 substituents independently selected from the group consisting of halogen, —OH, —CN, ═O, —OC(O)R³, —OR³, —C(O)R³, —C(O)NR³R⁴, —NR⁵C(O)R³, —CO₂R³, —NR³R⁴, —S(O)₂R³, substituted or unsubstituted phenyl, substituted or unsubstituted 5- or 6-membered heteroaryl, and substituted or unsubstituted 4- to 7-membered heterocycle. In other embodiments, X^{a} or X² is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is C₁₋₈ alkyl, optionally having from 1 to 3 substituents independently selected from the group consisting of halogen, —OH, —CN, ═O, —OC(O)R³, —OR³, —C(O)R³, —C(O)NR³R⁴, —CO₂R³, —NR³R⁴, —S(O)₂R³, substituted or unsubstituted phenyl substituted or unsubstituted 5- or 6-membered heteroaryl, and substituted or unsubstituted 4- to 7-membered heterocycle. In other embodiments, X^{a} or X² is selected from the group consisting of halogen, cyano, nitro, C₁₋₈ haloalkyl and C₁₋₈ haloalkyl.

In one embodiment of any of formulae (II-VI, and XX-CCXXXII), each of X^{a}, X², X³, X⁴ and X⁵ is hydrogen, and X¹ or X^{b} is C₁₋₈ alkyl, having 1 to 3 substituents independently selected from the group consisting of halogen, —CN, ═O, —OC(O)R³, —OR³, —C(O)R³, —C(O)NR³R⁴, —NR⁴C(O)R³, —CO₂R³, —NR³R⁴, —SR³, —S(O)R³, —S(O)₂R³, —NR³S(O)₂R⁴, substituted or unsubstituted phenyl, and substituted or unsubstituted 5- or 6-membered heteroaryl, and substituted or unsubstituted 4- to 7-membered heterocycle. In certain other preferred embodiments, X^{a}, X², X³, X⁴ and X⁵ is hydrogen, and X¹ and X^{b} are unsubstituted C₁₋₈ alkyl. In yet other preferred embodiments, X^{a}, X², X³, X⁴ and X⁵ is hydrogen, and X¹ and X^{b} is selected from the group consisting of halogen, cyano, nitro, C₁₋₈ haloalkyl and C₁₋₈ haloalkyl.

In one embodiment of any of formulae (II-VI, and XX-CCXXXII), each of X^{a}, X², X³, X⁴ and X⁵ is hydrogen, and X¹ or X^{b} is C₁₋₈ alkyl, having 1 to 3 substituents independently selected from the group consisting of halogen, —CN, ═O, —OC(O)R³, —OR³, —C(O)R³, —C(O)NR³R⁴, —CO₂R³, —NR³R⁴, —SR³, —S(O)R³, —S(O)₂R³, substituted or unsubstituted phenyl, and substituted or unsubstituted 5- or 6-membered heteroaryl, and substituted or unsubstituted 4- to 7-membered heterocycle. In certain other preferred embodiments, X^{a}, X², X³, X⁴ and X⁵ is hydrogen, and X¹ and X^{b} are unsubstituted C₁₋₈ alkyl. In yet other preferred embodiments, X^{a}, X², X³, X⁴ and X⁵ is hydrogen, and X¹ and X^{b} is selected from the group consisting of halogen, cyano, nitro, C₁₋₈ haloalkyl and C₁₋₈ haloalkyl.

In one embodiment, in each of formulae (II-VI, and XX-CCXXXII), each of X^{a}, X², X³, X⁴ and X⁵ is hydrogen, and X¹ or X^{b} is selected from the group consisting of —CN, —CF₃, halogen, —OR³, —S(O)₂R³, —C(O)R³, a substituted or unsubstitued C₁₋₈ alkyl, a substituted or unsubstituted 5- or 6-membered heteroaryl, and a substituted or unsubstituted 4- to 7-membered heterocycle.

In one embodiment, in each of formulae (II-VI, and XX-CCXXXII), each of X^{a}, X², X³, X⁴ and X⁵ is hydrogen, and X¹ or X^{b} is selected from the group consisting of —CN, halogen, —OR³, —SO₂R³, —C(O)R³, substituted or unsubstituted C₁₋₈ alkyl, substituted or unsubstituted 5- or 6-membered heteroaryl, and substituted or unsubstituted 4- to 7-membered heterocycle. In other embodiments, X^{a} and X² is selected from the group consisting of halogen, cyano and C₁₋₈ haloalkyl. In yet other preferred embodiments, X^{a}, and X² are selected from the group consisting of halogen, cyano, nitro, C₁₋₈ haloalkyl and C₁₋₈ haloalkyl.

In one embodiment of any of formulae (II-VI, and XX-CCXXXII), each of X^{a}, X², X³, X⁴ and X⁵ is hydrogen, and X¹ or X^{b} is unsubstituted C₁₋₈ alkyl.

In one embodiment of any of formulae (II-VI, and XX-CCXXXII), each of X^{a}, X², X³, X⁴ and X⁵ is hydrogen, and X¹ or X^{b} is a t-butyl group.

In another embodiment, in each of formulae (II-VI, and XX-CCXXXII), each of X^{a}, X², X³, X⁴ and X⁵ is hydrogen, and X¹ or X^{b} is selected from the group consisting of halogen, —CN, —CF₃, unsubstituted or substituted C₁₋₆ alkyl, substituted or unsubstituted 5- or 6-membered heteroaryl, and substituted or unsubstituted 4- to 7-membered heterocyclyl. In another embodiment, one of X^{a} and X² is other than hydrogen.

In one embodiment of any of formulae (II-VI, and XX-CCXXXII), each of X^{a}, X², X³, X⁴ and X⁵ is hydrogen, and X¹ or X^{b} is C₆₋₁₀ aryl or a heteroaryl, having from 0 to 3 substituents independently selected from the group consisting of halogen, —CN, —OH, —OR³, ═O, —OC(O)R³, —CO₂R³, —C(O)R³, —C(O)NR³R⁴, —NR³R⁴, —SR³, —S(O)R³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment of any of formulae (II-VI, and XX-CCXXXII), each of X^{a}, X², X³, X⁴ and X⁵ is hydrogen, and X¹ or X^{b} is C₆₋₁₀ aryl or a heteroaryl, having from 0 to 3 substituents independently selected from the group consisting of halogen, —CN, —OH, —OR³, ═O, —OC(O)R³, —CO₂R³, —C(O)R³, —C(O)NR³R⁴, —NR⁵C(O)R³, —NR³R⁴, —SR³, —S(O)R³, —NR⁵S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment of any of formulae (II-VI, and XX-CCXXXII), each of X^{a}, X², X³, X⁴ and X⁵ is hydrogen, and X¹ or X^{b} is substituted phenyl or substituted 5- or 6-membered heteroaryl, having from 0 to 3 substituents independently selected from the group consisting of halogen, —OR³, —NO₂,═O—C(O)R³, —C(O)NR³R⁴, —NR⁵C(O)R³, —NR³R⁴, —SR³, —NR⁵S(O)₂R³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment of any of formulae (II-VI, and XX-CCXXXII), each of X^{a}, X², X³, X⁴ and X⁵ is hydrogen, and X¹ or X^{b} is substituted phenyl or substituted 5- or 6-membered heteroaryl, having from 0 to 3 substituents independently selected from the group consisting of halogen, —OR³, —NO₂, ═O—C(O)R³, —C(O)NR³R⁴, —NR³R⁴, —SR³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment of any of formulae (II-VI, and XX-CCXXXII), each of X^{a}, X², X³, X⁴ and X⁵ is hydrogen, and X¹ or X^{b} is a 4- to 7-membered heterocyclyl, having 0 to 3 substituents independently selected from the group consisting of substituted or unsubstituted C₁₋₈ alkyl, —OR³, —OC(O)R³, —CO₂R³, —C(O)R³, —C(O)NR³R⁴, —NR³R⁴, —NR⁵C(O)R³, —S(O)₂R³, —SR³ and —NR⁵S(O)₂R³. In other embodiments, X^{a} or X² is halogen, cyano, nitro, C₁₋₈ haloalkyl or C₁₋₈ haloalkyl.

In one embodiment of any of formulae (II-VI, and XX-CCXXXII), each of X^{a}, X², X³, X⁴ and X⁵ is hydrogen, and X¹ or X^{b} is a 4- to 7-membered heterocyclyl, having 0 to 3 substituents independently selected from the group consisting of substituted or unsubstituted C₁₋₈ alkyl, —OR³, —OC(O)R³, —CO₂R³, —C(O)R³, —C(O)NR³R⁴, —NR³R⁴, —S(O)₂R³, and —SR³. In other embodiments, X^{a} or X² is halogen, cyano, nitro, C₁₋₈ haloalkyl or C₁₋₈ haloalkyl.

In one embodiment of any of formulae (II, III and XX-CLXVII), each of X^{a}, X², X³, X⁴ and X⁵ is hydrogen, and X¹ or X^{b} is a 5- or 6-membered heterocyclyl, having 0 to 2 substituents independently selected from the group consisting of substituted or unsubstituted C₁₋₈ alkyl, —OR³, —C(O)R³, —C(O)NR³R⁴, —NR³R⁴, and —S(O)₂R³. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is C₆₋₁₀ aryl or a heteroaryl, optionally having from 1 to 3 substituents independently selected from the group consisting of halogen, —CN, —OH, —OR³, ═O, —NO₂, —OC(O)R³, —CO₂R³, —C(O)R³, —C(O)NR³R⁴, —NR⁵C(O)R³, —NR³R⁴, —SR³, —S(O)R³, —S(O)₂R³, —NR⁵S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is C₆₋₁₀ aryl or a heteroaryl, optionally having from 1 to 3 substituents independently selected from the group consisting of halogen, —CN, —OH, —OR³, ═O, —NO₂, —OC(O)R³, —CO₂R³, —C(O)R³, —C(O)NR³R⁴, —NR³R⁴, —SR³, —S(O)R³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is phenyl or 5- or 6-membered heteroaryl, optionally having from 1 to 3 substituents independently selected from the group consisting of halogen, —OR³, ═O, —NO₂, —C(O)R³, —C(O)NR³R⁴, —NR⁵C(O)R³, —NR³R⁴, —SR³ , —NR⁵S(O)₂R³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is phenyl or 5- or 6-membered heteroaryl, optionally having from 1 to 3 substituents independently selected from the group consisting of halogen, —OR³, ═O, —NO₂, —C(O)R³, —C(O)NR³R⁴, —NR³R⁴, —SR³, —S(O)₂R³, and substituted or unsubstituted C₁₋₈ alkyl. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is a 4- to 7-membered heterocyclyl, optionally having from 1 to 3 substituents independently selected from the group consisting of substituted or unsubstituted C₁₋₈ alkyl, —OR³, —OH, —NR⁵C(O)R³, —OC(O)R³, —CO₂R³, —C(O)R³, —C(O)NR³R⁴, —NR³R⁴, —S(O)₂R³, —SR³ and —NR⁵S(O)₂R³. In other embodiments, X^{a} or X² is halogen, cyano, nitro, C₁₋₈ haloalkyl or C₁₋₈ haloalkyl.

In one embodiment, in each of formulae (II-VI, and XX-CCXXXII), each of X^{a}, X², X³, X⁴ and X⁵ is hydrogen, and X¹ or X^{b} is a 5- or 6-membered heterocyclyl, optionally having 1 to 2 substituents independently selected from the group consisting of substituted or unsubstituted C₁₋₈ alkyl, —OR³, —C(O)R³, —C(O)NR³R⁴, —NR³R⁴, and —S(O)₂R³. In other embodiments, X^{a} or X² is halogen, cyano, nitro, C₁₋₈ haloalkyl or C₁₋₈ haloalkyl.

In one embodiment where L is CR^{a}R^{b} or NR^{c}, the substituents Z, Y¹, Y², Y³, Y⁴, R^{a}, and R^{b} are as defined in formula (I); X¹, X², X³, X⁴, and X⁵ are as defined in formula (II), with the proviso that X¹ is not —NR⁵C(O)R³, —NR⁵C(O)NR³R⁴, —NR⁵CO₂R³, —NR⁵S(O)₂R³.

In another embodiment where L is CR^{a}R^{b} or NR^{c}, the substituents Z, Y¹, Y², Y³, Y⁴, R^{a}, and R^{b} are as defined in formula (I); X¹, X², X³, X⁴, and X⁵ are as defined in formula (II), with the proviso that X¹ is not butyrolactam, valerolactam, imidazolidinone, hydantoin, phthalimide, and pyridone.

Preferred Y Substituents

In one embodiment, in each of formulae (I-CCXXXII), Y² or Y^{a} is hydrogen, and Y¹ is selected from the group consisting of —Cl, —Br, —F, and —OCH₃.

In one embodiment, in each of formulae (I-CCXXXII), Y¹ is hydrogen, and Y² or Y^{a} is selected from the group consisting of —Cl, —Br, —F, —CH₃, —CF₃, and —CN.

In one embodiment, in each of formulae (I-CCXXXII), Y² or Y^{a} is —Cl and Y¹ is —CH₃.

In one embodiment, in each of formulae (I-CCXXXII), Y¹ and Y² or Y^{a} are each —F.

In one embodiment, in each of formulae (I-CCXXXII), each of Y^{a}, Y¹, Y² is selected from the group consisting of —Cl, —Br, —F, —OCH₃, —CH₃,

—CF₃, and —CN.

In one embodiment, in each of formulae (I-CCXXXII), each of Y^{a}, Y¹, Y² is selected from the group consisting of halogen, —CN, —OR⁶, and substituted or unsubstituted C₁₋₄ alkyl.

In one embodiment, in each of formulae (I-CCXXXII), each of Y^{b}, Y², Y³, and Y³ is hydrogen, and Y¹ or Y^{a} is other than hydrogen.

In one embodiment of any of formulae (I-CCXXXII), Y³ is hydrogen, Y¹ is other than hydrogen, and one of Y² and Y⁴ is hydrogen and the other is other than hydrogen. In a preferred embodiment, Y¹, Y², Y³, Y⁴, Y^{a} and Y^{b} are selected from the group consisting of hydrogen, halogen, —CF₃, C₁₋₈ alkyl, —C(O)R¹, —SO₂R¹, and —C(O)NR¹R², where at least one of Y¹, Y², Y³, Y⁴, Y^{a} and Y^{b} is other than hydrogen.

In one embodiment of any of formulae (I-CCXXXII), at least one of Y¹, Y², Y³, Y⁴, Y^{a} and Y^{b} is halogen, —CN, —NO₂, —OR⁶, —C(O)R⁶, —SR⁶, —CF₃, —S(O)R⁶, —S(O)₂R⁶ or substituted or unsubstituted C₁₋₄ alkyl.

In one embodiment of any of formulae (I-CCXXXII), at least one of Y¹, Y², Y³, Y⁴, Y^{a} and Y^{b} is halogen, —CN, —NO₂, —C(O)R⁶, —SR⁶, —CF₃, —S(O)R⁶, —S(O)₂R⁶ or substituted or unsubstituted C₁₋₄ alkyl.

In one embodiment of any of formulae (I-CCXXXII), Y¹ or Y^{b} is hydrogen and Y^{a}, Y², Y³, and Y⁴ is other than hydrogen.

In one embodiment of any of formulae (I-CCXXXII), Y¹, Y^{a} and Y^{b} represent from 1 to 3 substituents independently selected from the group consisting of hydrogen, halogen, —CN, —NO₂, —OR⁶, —C(O)R⁶, —SR⁶, —CF₃, —S(O)R⁶, and —S(O)₂R⁶ and substituted or unsubstituted C₁₋₆ alkyl.

In one embodiment of any of formulae (I-CCXXXII), Y¹, Y^{a} and Y^{b} represent from 1 to 3 substituents independently selected from the group consisting of hydrogen, halogen, —CN, —NO₂, —C(O)R⁶, —SR⁶, —CF₃, —S(O)R⁶, and —S(O)₂R⁶ and substituted or unsubstituted C₁₋₆ alkyl.

In one embodiment of any of formulae (I-CCXXXII), Y¹, Y^{a} and Y^{b} represent from 1 to 3 substituents independently selected from the group consisting of hydrogen, halogen, —CN, —NO₂, —OCH₃, —CH₃, —CF₃, and —S(O)₂Me.

In one embodiment of any of formulae (I-CCXXXII), one of Y¹, Y^{a} and Y^{b} is halogen and one of the others is selected from the group consisting of hydrogen, halogen, —CN, —NO₂, —OH, —OR⁶, —C(O)R⁶, —CO₂R⁶, —SR⁶, —S(O)R⁶, —S(O)₂R⁶ and substituted or unsubstituted C₁₋₄ alkyl.

In one embodiment of any of formulae (I-CCXXXII), one of Y¹, Y^{a} and Y^{b} is halogen and one of the others is selected from the group consisting of hydrogen, halogen, —CN, —NO₂, —C(O)R⁶, —CO₂R⁶, —SR⁶, —S(O)R⁶, —S(O)₂R⁶ and substituted or unsubstituted C₁₋₄ alkyl.

In one embodiment, in each of formulae (I-CCXXXII), one of Y¹, Y^{a} and Y^{b} is substituted alkyl, having from 1 to 3 substituents independently selected from the group consisting of halogen, —OH, —OR^{m}, —CN, —NO₂, ═O, —OC(O)R^{m}, —CO₂R^{m}, —C(O)R^{m}, —C(O)NHRⁿ, —C(O)NH₂, —C(O)NR^{m}Rⁿ, —NR^{m}C(O)Rⁿ, —NHC(O)Rⁿ, —NR^{m}Rⁿ, —NHR^{m}, —NH₂, —SR^{m}, —S(O)R^{m}, —S(O)₂R^{m}, —NR^{m}S(O)₂Rⁿ, and —NHS(O)₂R^{m}, where R^{m} and Rⁿ are each independently unsubstituted C₁₋₆ alkyl.

In one embodiment of any of formulae (I-CCXXXII), Y¹, Y^{a} and Y^{b} are each independently selected from the group consisting of hydrogen, halogen, —CN, —NO₂, —OH, —OR⁶, —C(O)R⁶, —CO₂R⁶, —SR⁶, —S(O)R⁶, —S(O)₂R⁶, and substituted or unsubstituted C₁₋₄ alkyl, with the proviso that Y¹ and Y^{a} or Y^{b} and Y^{a} cannot both be hydrogen simultaneously.

In one embodiment of any of formulae (I-CCXXXII), Y¹, Y^{a} and Y^{b} are each independently selected from the group consisting of hydrogen, halogen, —CN, —NO₂, —C(O)R⁶, —CO₂R⁶, —SR⁶, —S(O)R⁶, —S(O)₂R⁶, and substituted or unsubstituted C₁₋₄ alkyl, with the proviso that Y¹ and Y^{a} or Y^{b} and Y^{a} cannot both be hydrogen simultaneously.

In one embodiment of any of formulae (I-CCXXXII), Y¹ or Y^{b}, and Y^{a} are each independently hydrogen or halogen, with the proviso that one or both are halogen.

In one embodiment of any of formulae (I-CCXXXII), Y¹ or Y^{b} is hydrogen and Y^{a} is chloro, fluoro or bromo; in another embodiment, Y^{a} is hydrogen and Y¹ or Y^{b} is chloro, fluoro or bromo; in another embodiment Y¹ or Y^{b} and Y^{a} are both chloro, fluoro, or bromo (particularly fluoro).

In one embodiment, in each of formulae (I-CCXXXII), Y¹, Y^{a}, and Y^{b} are each independently selected from the group consisting of hydrogen, halogen, —CN, —NO₂, —OR⁶, —C(O)R⁶, —SR⁶, —S(O)R⁶, —S(O)₂R⁶ and substituted or unsubstituted C₁₋₆ alkyl.

In one embodiment, in each of formulae (I-CCXXXII), Y¹, Y^{a}, and Y^{b} are each independently selected from the group consisting of hydrogen, halogen, —CN, —NO₂, —C(O)R⁶, —SR⁶, —S(O)R⁶, —S(O)₂R⁶ and substituted or unsubstituted C₁₋₆ alkyl.

In one embodiment, in each of formulae (I-CCXXXII), Y¹, Y^{a}, and Y^{b} are each independently selected from the group consisting of hydrogen, halogen, —CN, —NO₂, —CF₃, and —SO₂Me.

In one embodiment, in each of formulae (I-CCXXXII), one of Y¹, Y^{a}, and Y^{b} is halogen and the other is selected from the group consisting of hydrogen, halogen, —CN, —NO₂, —OH, —OR⁶, —C(O)R⁶, —CO₂R⁶, —SR⁶, —S(O)R⁶, —S(O)₂R⁶ and substituted or unsubstituted C₁₋₄ alkyl.

In one embodiment, in each of formulae (I-CCXXXII), one of Y¹, Y^{a}, and Y^{b} is halogen and the other is selected from the group consisting of hydrogen, halogen, —CN, —NO₂, —C(O)R⁶, —CO₂R⁶, —SR⁶, —S(O)R⁶, —S(O)₂R⁶ and substituted or unsubstituted C₁₋₄ alkyl.

In one embodiment, in each of formulae (I-CCXXXII), one of Y¹, Y^{a}, and Y^{b} is substituted alkyl, having from 1 to 3 substituents independently selected from the group consisting of halogen, —OH, —OR^{m}, —CN, —NO₂, ═O, —OC(O)R^{m}, —CO₂R^{m}, —C(O)R^{m}, —C(O)NHRⁿ, —C(O)NH₂, —C(O)NR^{m}Rⁿ, —NR^{m}C(O)Rⁿ, —NHC(O)Rⁿ, —NR^{m}Rⁿ, —NHR^{m}, —NH₂, —SR^{m}, —S(O)R^{m}, —S(O)₂R^{m}, —NR^{m}S(O)₂Rⁿ, and —NHS(O)₂R^{m}, where R^{m} and Rⁿ are each independently unsubstituted C₁₋₆ alkyl.

In one embodiment of any of formulae (I-CCXXXII), Y¹, Y^{a}, and Y^{b} are each independently selected from the group consisting of hydrogen, halogen, —CN, —NO₂, —OH, —OR⁶, —C(O)R⁶, —CO₂R⁶, —SR⁶, —S(O)R⁶, —S(O)₂R⁶, and substituted or unsubstituted C₁₋₄ alkyl, with the proviso that Y¹ and Y^{a}, or Y^{a} and Y^{b} cannot both be hydrogen simultaneously.

In one embodiment of any of formulae (I-CCXXXII), Y¹, Y^{a}, and Y^{b} are each independently selected from the group consisting of hydrogen, halogen, —CN, —NO₂, —C(O)R⁶, —CO₂R⁶, —SR⁶, —S(O)R⁶, —S(O)₂R⁶, and substituted or unsubstituted C₁₋₄ alkyl, with the proviso that Y¹ and Y^{a}, or Y^{a} and Y^{b} cannot both be hydrogen simultaneously.

In one embodiment, in each of formulae (I-CCXXXII), Y¹, Y^{a}, and Y^{b} are each independently hydrogen or halogen, with the proviso that one or both of Y¹ and Y^{a}, or Y^{a} and Y^{b} are halogen.

In one embodiment, in each of formulae (I-CCXXXII), Y¹, Y^{a}, and Y^{b} are each independently hydrogen, halogen or C₁₋₈ alkyl (preferably methyl), with the proviso that one or both of Y¹ and Y^{a}, or Y^{a} and Y^{b} are C₁₋₈ alkyl (preferably methyl).

In one embodiment of any of formulae (I-CCXXXII), Y¹ or Y^{b} is hydrogen and Y^{a} is chloro, fluoro or bromo; Y^{a} is hydrogen and Y¹ or Y^{b} is chloro, fluoro or bromo; or Y¹, Y^{a}, Y^{b} are each independently chloro, fluoro, or bromo (particularly fluoro).

In one embodiment, in each of formulae (I-CCXXXII), Y¹ or Y^{b} is hydrogen and Y^{a} is chloro, fluoro or bromo; or Y^{a} is hydrogen and Y¹ or Y^{b} is chloro, fluoro or bromo; or Y¹, Y^{b} and Y^{a} are each independently selected from the group consisting of chloro, fluoro, and bromo (and in one particular embodiment, both Y¹ and Y^{a} are fluoro or both Y^{b} and Y^{a} are fluoro).

In one embodiment of formulae (II), at least one of Y¹ to Y⁴ is other than hydrogen. Preferably one or two of Y¹ to Y⁴ are other than hydrogen. More preferably, Y³ is hydrogen.

In one embodiment of any of formulae (II), at least one of Y¹ to Y⁴ is halogen, —CN, —NO₂, —OR⁶, —C(O)R⁶, —SR⁶, —CF₃, —S(O)R⁶, —S(O)₂R⁶ or substituted or unsubstituted C₁₋₄ alkyl.

In one embodiment of any of formulae (II), at least one of Y¹ to Y⁴ is halogen, —CN, —NO₂, —C(O)R⁶, —SR⁶, —CF₃, —S(O)R⁶, —S(O)₂R⁶ or substituted or unsubstituted C₁₋₄ alkyl.

In one embodiment of formula (II), Y³ is hydrogen and Y¹ is chlorine or fluorine, and when Y¹ is chlorine, both Y² and Y⁴ are hydrogen or when Y¹ is fluorine, either Y² or Y⁴ is also fluorine or halogen.

In one embodiment of formula (II), Y³ is hydrogen and Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen.

In one embodiment of formulae (II), Y² is chlorine and Y¹, Y³, and Y⁴ are hydrogen.

In one embodiment of formulae (II), Y² is halogen and Y³ is hydrogen, Y¹and Y⁴ are each independently selected from the group consisting of halogen and hydrogen.

Preferred Z Groups and Substituents

In one embodiment of any of formulae (I-CCXXXII), Z is a substituted or unsubstituted aryl.

In one embodiment of any of formulae (I-CCXXXII), Z is a substituted or unsubstituted phenyl, having 0 to 5 substituents as defined in formula (I).

In one preferred embodiment of formulae (I-CCXXXII), Z is a substituted or unsubstituted phenyl.

In one embodiment of any of formulae (I-CCXXXII), Z is a substituted or unsubstituted naphthalyl, having 0 to 5 substituents as defined in formula (I).

In one embodiment of any of formulae (I-CCXXXII), Z is a substituted or unsubstituted 5- to 10-membered heteroaryl, having 0 to 5 substituents as defined in formula (I).

In one embodiment of any of formulae (I-CCXXXII), Z is substituted or unsubstituted 5- to 10-membered monocylic or bicyclic heteroaryl with 0 to 4 substituents.

In one embodiment of any of formulae (I-CCXXXII), Z is substituted or unsubstituted 3- to 10-membered heterocyclyl with 0 to 4 substituents.

In one embodiment of any of formulae (I-CCXXXII), Z is —NR¹⁷R¹⁸.

In one embodiment of any of the formulae (I-CCXXXII) where L is a bond, Z is 5- to 10-membered monocyclic or bicyclic heteroaryl ring system, where one ring heteroatom is located alpha (ortho) to the biaryl bond.

In one embodiment of any of the formulae (I-CCXXXII) where L is a bond, Z is 5- to 10-membered monocyclic or bicyclic heteroaryl ring system containing one or more ring nitrogen atoms, and where one or more ring nitrogen atoms is located alpha (ortho) to the biaryl bond.

In one embodiment, in any of the formulae (I-CCXXXII), Z is a substituted or unsubstituted 5- to 10-membered heteroaryl selected from the group consisting of pyridyl, pyrrolyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, pyrrolopyridinyl, imidazopyridinyl, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl, thienyl, 4-azaindolyl, 5-azaindolyl, 6-azaindolyl, 7-azaindolyl, 4-azaindazolyl, 5-azaindazolyl, 6-azaindazolyl, 7-azaindazolyl.

In one embodiment, in any of the formulae (I-CCXXXII), Z is substituted or unsubstituted 6-membered heteroaryl with 1 to 2 nitrogen atoms and with 0 to 2 substituents independently selected from the group consisting of halogen, unsubstituted or substituted C₁₋₆ alkyl, unsubstituted or substituted C₁₋₆ alkenyl, unsubstituted or substituted C₁₋₆ alkynyl, ═O, —CN, —NO₂ —OR¹⁰, —C(O)R¹⁰, —C(O)NR¹⁰R¹¹, —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, —OC(O)R¹⁰, —CO₂R¹⁰, —OC(O)NR¹¹R¹², —NR¹⁰C(O)NR¹¹R¹², —NR¹⁰CO₂R¹¹, unsubstituted or substituted 5- or 6-membered heteroaryl and unsubstituted or substituted 3- to 7-membered heterocyclyl. In this embodiment, Z can be any chemically allowed regioisomer of pyridyl, pyrimidinyl, pyridazinyl, pyrazinyl and the like, and their respective N-oxides.

In one embodiment in any of the formulae (I-CCXXXII), Z is a substituted or unsubstituted 6-membered heteroaryl with 1 to 2 nitrogen atoms and with 0 to 2 substituents independently selected from the group consisting of halogen, substituted C₁₋₆ alkyl (but not C₁₋₆ haloalkyl), unsubstituted or substituted C₁₋₆ alkenyl, unsubstituted or substituted C₁₋₆ alkynyl, ═O, —CN, —C(O)R¹⁰, —C(O)NR¹⁰R¹¹, —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, —OC(O)R¹⁰, —CO₂R¹⁰ (but not —CO₂H), —OC(O)NR¹¹R¹², —NR¹⁰C(O)NR¹¹R¹², —NR¹⁰CO₂R¹¹, unsubstituted or substituted 5- or 6-membered heteroaryl and a unsubstituted or substituted 3- to 7-membered heterocyclyl. Preferred substituents include chlorine, ═O, —CN, —SCH₃, —SO₂CH₃. In this embodiment, Z can be any chemically allowed regioisomer of pyridyl, pyrimidinyl, pyridazinyl, pyrazinyl and the like, and their respective N-oxides.

In one embodiment, in any of the formulae (I-CCXXXII), Z is substituted or unsubstituted 6-membered heteroaryl with 1 to 2 nitrogen atoms and optionally with 1 or 2 substituents independently selected from the group consisting of halogen, unsubstituted or substituted C₁₋₆ alkyl, unsubstituted or substituted C₁₋₆ alkenyl, unsubstituted or substituted C₁₋₆ alkynyl, ═O, —CN, —NO₂, —OR¹⁰, —C(O)R¹⁰, —C(O)NR¹⁰R¹¹, —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, —OC(O)R¹⁰, —CO₂R¹⁰, —OC(O)NR¹¹R¹², —NR¹⁰C(O)NR¹¹R¹², —NR¹⁰CO₂R¹¹, 5- or 6-membered heteroaryl and a 3- to 7-membered heterocyclyl. In this embodiment, Z can be any chemically allowed regioisomer of pyridyl, pyrimidinyl, pyridazinyl, pyrazinyl and the like, and their respective N-oxides.

In one embodiment in any of the formulae (I-CCXXXII), Z is substituted or unsubstituted 6-membered heteroaryl with 1 to 2 nitrogen atoms and with 0 to 2 substituents independently selected from the group consisting of unsubstituted C₁₋₆ alkyl, ═O, C₁₋₆ haloalkyl, —COOH, —NO₂, or —OR¹⁰. In this embodiment, Z can be any chemically allowed regioisomer of pyridyl, pyrimidinyl, pyridazinyl, pyrazinyl and the like, and their respective N-oxides.

In one embodiment in any of the formulae (I-CCXXXII), Z is substituted or unsubstituted 6-membered heteroaryl with 1 to 2 nitrogen atoms and with 0 to 2 substituents independently selected from the group consisting of —CH₃, ═O, —CF₃, —OCH₃. In this embodiment, Z can be any chemically allowed regioisomer of pyridyl, pyrimidinyl, pyridazinyl, pyrazinyl and the like, and their respective N-oxides.

In one embodiment of formula (I-CCXXXII), Z is a substituted or unsubstituted fused 5,6-ring and 6,6-ring heteroaryl selected from the group which includes isoquinolinyl, quinolizinyl, pyrrolizinyl, quinoxalinyl, quinazolinyl, cinnolinyl, phthalazinyl, benzotriazinyl, purinyl, benzimidazolyl, benzopyrazolyl, isoquinolinyl, quinolizinyl, benzotriazolyl, benzisoxazolyl, isobenzofuryl, isoindolyl, indolizinyl, thienopyridinyl, thienopyrimidinyl, pyrazolopyrimidinyl, pyrazolopyridinyl, imidazopyridinyl, pyridinopyridinyl, pyridinopyrimidinyl, pyridinopyridizinyl, pyridinopyrazinyl, pyrazolopyridinyl, triazolopyridinyl, pyrrolopyrazinyl, pyrrolopyridinyl, imidazotriazinyl, imidazopyrimidinyl, naphthyridinyl, benzothiazolyl, benzofuranyl, benzothienyl, indolyl, azaindolyl, benzopyrrolyl, benzisoxazolyl, benzisothiazolyl, quinolyl, isoquinolyl, indazolyl, pteridinyl, azaindolyl, benzopyrrolyl, benzisoxazolyl, benzisothiazolyl, and the like.

In one embodiment of each of the formulae (I-CCXXXII), Z is a substituted or unsubstituted 5- to 10-membered heteroaryl with 1 to 4 nitrogen atoms and with 0 to 4 substituents independently selected from the group consisting of halogen, unsubstituted or substituted C₁₋₆ alkyl, unsubstituted or substituted C₂₋₆ alkenyl, unsubstituted or substituted C₂₋₆ alkynyl, ═O, —CN, —NO₂—OR¹⁰, —C(O)R¹⁰, —C(O)NR¹⁰R¹¹, —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, —OC(O)R¹⁰, —CO₂R¹⁰, —OC(O)NR¹¹R¹², —NR¹⁰C(O)NR¹¹R¹², —NR¹⁰CO₂R¹¹, unsubstituted or substituted 5- or 6-membered heteroaryl and a unsubstituted or substituted 3- to 7-membered heterocyclyl.

In one embodiment of any of formulae (I-CCXXXII), Z is a substituted or unsubstituted 6-membered heteroaryl selected from the group consisting of pyridyl, pyridazinyl, pyrazinyl, pyrimidinyl, and triazinyl.

In one embodiment of any of formulae (I-CCXXXII), Z is a substituted or unsubstituted 5-membered heteroaryl selected from the group consisting of isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl.

In one embodiment in formulae (I-CCXXXII), suitable 6-membered ring heteroaryl systems as substituents on Z include pyridyl, pyridazinyl, pyrazinyl, pyrimidinyl and triazinyl.

In one embodiment in formulae (I-CCXXXII), suitable 5-membered ring heteroaryl systems as substituents on Z include isothiazolyl, pyrazolyl, imidazolyl, triazolyl, tetrazolyl, oxazolyl, isoxazolyl, oxadiazolyl, thiadiazolyl, pyrrolyl and thiazolyl.

In one embodiment of any of formulae. (I-CCXXXII), Z is any substituted or unsubstituted chemically allowed regioisomers of pyridyl, pyrimidinyl, pyridazinyl, pyrazinyl and the like and their respective N-oxides.

In one embodiment of any of formulae (I-CCXXXII), Z is a substituted or unsubstituted 2-pyridyl.

In one embodiment of any of formulae (I-CCXXXII), Z is a substituted or unsubstituted 2-pyridyl-N-oxide.

In one embodiment of any of formulae (I-CCXXXII), Z is a substituted or unsubstituted 3-pyridyl.

In one embodiment of any of formulae (I-CCXXXII), Z is a substituted or unsubstituted 3-pyridyl-N-oxide.

In one embodiment of any of formulae (I-CCXXXII), Z is a substituted or unsubstituted 4-pyridyl.

In one embodiment of any of formulae (I-CCXXXII), Z is a substituted or unsubstituted 4-pyridyl-N-oxide.

In one embodiment of any of formulae (I-CCXXXII), Z is a substituted or unsubstituted pyrrolopyridinyl.

In one embodiment of any of formulae (I-CCXXXII), Z is a substituted or unsubstituted 1 H-pyrrolo[2,3-b]pyridinyl.

In one preferred embodiment of any of formulae (I-CCXXXII), Z is a substituted or unsubstituted 2-pyridyl, 3-pyridyl or 4-pyridyl ring.

In one preferred embodiment of each of formulae (I-CCXXXII), Z is a substituted or unsubstituted 3-pyridazinyl, 4-pyridazinyl, 5-pyridazinyl, 6-pyridazinyl, 2-pyrazinyl, 3-pyrazinyl, 5-pyrazinyl, 6-pyrazinyl, 2-pyrimidinyl, 4-pyrimidinyl, 5-pyrimidinyl or 6-pyrimidinyl ring.

In one embodiment of any of formulae (I-CCXXXII), Z is pyridinyl with from 0 to 3 substituents; pyrimidinyl with from 0 to 3 substituents; pyrazinyl with from 0 to 3 substituents; or pyridazinyl with from 0 to 3 substituents (especially, where one ring nitrogen has a ═O substituent).

In one embodiment of any of formulae (I-CCXXXII), Z has from 0 to 3 substituents; (and in one particular embodiment, one ring nitrogen has a ═O substituent).

In one embodiment of any of formulae (I-CCXXXII), Z is pyrazolyl with from 0 to 3 substituents; or imidazolyl with from 0 to 3 substituents.

In one preferred embodiment of each of formulae (I-CCXXXII), Z is a substituted or unsubstituted N-pyrazolyl, 3-pyrazolyl, 4-pyrazolyl, 5-pyrazolyl, 2-imidazolyl, 4-imidazolyl or 5-imidazolyl.

In one embodiment of any of formulae (I-CCXXXII), Z is a substituted or unsubstituted pyrazolyl, 4-(1,2,3-triazolyl), or 2-(1,2,4-triazolyl).

In one embodiment of any of formulae (I-CCXXXII), Z is tetrazolyl with 0 or 1 substituents.

In one embodiment of any of formulae (I-CCXXXII), Z is a substituted or unsubstituted imidazolyl.

In one embodiment of any of formulae (I-CCXXXII), Z is imidazolyl with 0 or 1 substituents.

In one embodiment of any of formulae (I-CCXXXII), Z is a substituted or unsubstituted thiazolyl.

In one embodiment of any of formulae (I-CCXXXII), Z is a substituted or unsubstituted triazolyl.

In one embodiment of any of formulae (I-CCXXXII), Z is a substituted or unsubstituted 4-(1,2,3-triazolyl).

In one embodiment of any of formulae (I-CCXXXII), Z is a substituted or unsubstituted 2-(1,2,4-triazolyl).

In one embodiment of formula (I-CCXXXII), Z is phthalazinonyl with 0 or 3 substituents.

In one embodiment of formula (I-CCXXXII), Z is dihydroisoquinolinonyl with 0 or 3 substituents.

In one embodiment of formula (I-CCXXXII), Z is indazolyl with 0 or 3 substituents.

In one embodiment of formula (I-CCXXXII), Z is isochromanonyl with 0 or 3 substituents.

In one embodiment of formula (I-CCXXXII), Z is isobenzofuranonyl with 0 or 3 substituents.

In one embodiment of formula (I-CCXXXII), Z is pyridazinyl with 0 or 3 substituents.

In one embodiment of formula (I-CCXXXII), Z is pyrimidinyl with 0 or 3 substituents.

In one embodiment of formula (I-CCXXXII), Z is pyrazinyl with 0 or 3 substituents.

In one embodiment of any of formulae (I-CCXXXII), Z is a substituted or unsubstituted C₆₋₁₀ aryl or substituted or unsubstituted 5- to 10-membered heteroaryl selected from pyridyl, pyridazinyl, pyrazinyl, pyrimidinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiazolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl or thienyl.

In one embodiment of any of formulae (I-CCXXXII), Z is a substituted or unsubstituted C₆₋₁₀ aryl selected from phenyl or naphthalenyl or is a substituted or unsubstituted 5- to 10-membered heteroaryl selected from the group which includes isoquinolinyl, quinolizinyl, pyrrolizinyl, quinoxalinyl, quinazolinyl, cinnolinyl, phthalazinyl, benzotriazinyl, purinyl, benzimidazolyl, benzopyrazolyl, isoquinolinyl, quinolizinyl, benzotriazolyl, benzisoxazolyl, isobenzofuryl, isoindolyl, indolizinyl, thienopyridinyl, thienopyrimidinyl, pyrazolopyridinyl, pyrazolopyrimidinyl, imidazopyridinyl, pyridinopyridinyl, pyridinopyrimidinyl, pyridinopyridizinyl, pyridinopyrazinyl, pyrrolopyrazinyl, imidazotriazinyl, imidazopyrimidinyl, triazolopyridinyl, naphthyridinyl, benzothiazolyl, benzofuranyl, benzothienyl, indolyl, azaindolyl, benzopyrrolyl, benzisoxazolyl, benzisothiazolyl, quinolyl, isoquinolyl, isothiazolyl, pyrazolyl, indazolyl, pteridinyl, imidazolyl, triazolyl, tetrazolyl, oxazolyl, isoxazolyl, oxadiazolyl, thiadiazolyl, pyrrolyl, thiazolyl, furyl, thienyl, azaindolyl, benzopyrrolyl, benzisoxazolyl, benzisothiazolyl, and the like.

In one embodiment of any of formulae (I-CCXXXII), Z is monocyclic.

In one embodiment of any of formulae (I-CCXXXII), Z is a fused bicyclic.

In one embodiment in any of the formulae (I-CCXXXII), at least one substituent on the group Z is cyano.

In one embodiment in any of the formulae (I-CCXXXII), at least one substituent on the group Z is —S(O)₂R⁷. In one particular embodiment, R⁷ is methyl.

In one embodiment in any of the formulae (I-CCXXXII), at least one substituent on the group Z is halogen, particularly chlorine.

In one embodiment in any of the formulae (I-CCXXXII), at least one substituent on the group Z is —OR⁷. In one particular embodiment, R⁷ is methyl.

In one embodiment in any of the formulae (I-CCXXXII), at least one substituent on the group Z is —SR⁷. In one particular embodiment, R⁷ is methyl.

In one embodiment in any of the formulae (I-CCXXXII), at least one substituent on the group Z is unsubstituted C₁₋₆ alkyl (in particular methyl) or C₁₋₆ haloalkyl (in particular —CF₃).

In one embodiment in any of the formulae (I-CCXXXII), at least one substituent on the group Z is substituted C₁₋₆ alkyl (preferably not C₁₋₆ haloalkyl).

In one embodiment of any of the formulae (I-CCXXXII), at least one substituent on the group Z is a heterocyclyl selected from the group including pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment in any of the formulae (I-CCXXXII), no substituents, except hydrogen, exist on the group Z.

In one embodiment of any of formulae (I-CCXXXII), Z is a substituted or unsubstituted 3- to 10-membered heterocycle, having 0 to 3 substituents as defined in formula (I).

In one embodiment of any of the formulae (I-CCXXXII), Z is 5- to 10-membered monocyclic or bicyclic heterocyclyl ring system, where one ring heteroatom is located alpha (ortho) to L.

In one embodiment of any of formulae (I-CCXXXII), Z is substituted or unsubstituted 5- to 10-membered monocytic or bicyclic heterocyclyl with 0 to 4 substituents.

In one embodiment of any of the formulae (I-CCXXXII), Z is 5- to 10-membered monocyclic or bicyclic heterocyclyl ring system containing one or more ring nitrogen atoms, and where one or more ring nitrogen atoms is located alpha (ortho) to L.

In one embodiment of each of the formulae (I-CCXXXII), Z is a substituted or unsubstituted 5- to 10-membered heterocyclyl with 1 to 4 nitrogen atoms and with 0 to 4 substituents independently selected from the group consisting of halogen, unsubstituted or substituted C₁₋₆ alkyl, unsubstituted or substituted C₂₋₆ alkenyl, unsubstituted or substituted C₂₋₆ alkynyl, ═O, —CN, —NO₂, —OR⁷, —C(O)R⁷, —C(O)NR⁸R⁹, —NR⁷C(O)R⁸, —NR⁸R⁹, SR⁷, —S(O)R⁷, —S(O)₂R⁷, —S(O)₂NR⁸R⁹, —NR⁷S(O)₂R⁸, —OC(O)R⁷, —CO₂R⁷, —OC(O)NR⁸R⁹, —NR⁷C(O)NR⁸R⁹, —NR⁷CO₂R⁸, unsubstituted or substituted 5- or 6-membered heteroaryl and a unsubstituted or substituted 3- to 7-membered heterocyclyl.

In one embodiment of each of the formulae (I-CCXXXII), Z is a substituted or unsubstituted 5- to 10-membered heterocyclyl with 1 to 4 oxygen atoms and with 0 to 4 substituents independently selected from the group consisting of halogen, unsubstituted or substituted C₁₋₆ alkyl, unsubstituted or substituted C₂₋₆ alkenyl, unsubstituted or substituted C₂₋₆ alkynyl, ═O, —CN, —NO₂, —OR⁷, —C(O)R⁷, —C(O)NR⁸R⁹, —NR⁷C(O)R⁸, —NR⁸R⁹, —SR⁷, —S(O)R⁷, —S(O)₂R⁷, —S(O)₂NR⁸R⁹, —NR⁷S(O)₂R⁸, —OC(O)R⁷, —CO₂R⁷, —OC(O)NR⁸R⁹, —NR⁷C(O)NR⁸R⁹, —NR⁷CO₂R⁸, unsubstituted or substituted 5- or 6-membered heteroaryl and a unsubstituted or substituted 3- to 7-membered heterocyclyl.

In one embodiment of each of the formulae (I-CCXXXII), Z is a substituted or unsubstituted 5- to 10-membered heterocyclyl with 1 to 4 sulfur atoms and with 0 to 4 substituents independently selected from the group consisting of halogen, unsubstituted or substituted C₁₋₆ alkyl, unsubstituted or substituted C₂₋₆ alkenyl, unsubstituted or substituted C₂₋₆ alkynyl, ═O, —CN, —NO₂, —OR⁷, —C(O)R⁷, —C(O)NR⁸R⁹, —NR⁷C(O)R⁸, —NR⁸R⁹, —SR⁷, —S(O)R⁷, —S(O)₂R⁷, —S(O)₂NR⁸R⁹, —NR⁷S(O)₂R⁸, —OC(O)R⁷, —CO₂R⁷, —OC(O)NR⁸R⁹, —NR⁷C(O)NR⁸R⁹, —NR⁷CO₂R⁸, unsubstituted or substituted 5- or 6-membered heteroaryl and a unsubstituted or substituted 3- to 7-membered heterocyclyl.

In one embodiment of any of formulae (I-CCXXXII), Z is a substituted or unsubstituted 3- to 10-membered heterocycle selected from the group consisting of hydropyrimidine, pyrrolidine, piperidine, imidazolidine, pyrazolidine, butyrolactam, valerolactam, imidazolidinone, hydantoin, dioxolane, phthalimide; 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of any of formulae (I-CCXXXII), Z is a substituted or unsubstituted 3- to 10-membered heterocycle selected from the group consisting of pyrimidine, pyrrolidine, piperidine, imidazolidine, pyrazolidine, dioxolane, 1,4-dioxane, morpholine, thiomorpholine, thiomorpholine-S-oxide, thiomorpholine-S,S-dioxide, piperazine, pyran, 3-pyrroline, thiopyran, pyrone, tetrahydrofuran, and tetrahydrothiophene.

In one embodiment of any of formulae (I-CCXXXII), Z is a substituted or unsubstituted heterocyclic ring system selected from pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran or tetrahydrothiophene. Preferably, Z is selected from substituted and unsubstituted piperidine, substituted and unsubstituted piperazine, and substituted and unsubstituted morpholine.

In one embodiment of any of formulae (I-CCXXXII), Z is a substituted or unsubstituted heterocyclic ring system selected from pyrrolidine, piperidine, imidazolidine, pyrazolidine, dioxolane, piperidine, 1,4-dioxane, morpholine, thiomorpholine, thiomorpholine-S-oxide, thiomorpholine-S,S-dioxide, piperazine, pyran, 3-pyrroline, thiopyran, pyrone, tetrahydrofuran or tetrahydrothiophene.

In one embodiment of any of formulae (I-CCXXXII), Z is pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, tetrahydrothiophene.

In one embodiment of any of formulae (I-CCXXXII), Z is pyrrolidine, piperidine, imidazolidine, pyrazolidine, dioxolane, piperidine, 1,4-dioxane, morpholine, thiomorpholine, thiomorpholine-S-oxide, thiomorpholine-S,S-dioxide, piperazine, pyran, 3-pyrroline, thiopyran, pyrone, tetrahydrofuran, tetrahydrothiophene.

In one embodiment of any of formulae (I-CCXXXII), Z is a heterocyclic group represented by formula (AA) below, where formula (AA) is attached via a free valence on either M¹ or M², and where formula AA and the substituents therein are defined in [0045].

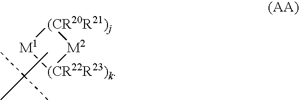

In one embodiment of any of formulae (I-CCXXXII), heterocycle groups as substituents on Z include pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of any of formulae (I-CCXXXII), heterocycle groups as substituents on Z include pyrrolidine, piperidine, imidazolidine, pyrazolidine, dioxolane, piperidine, 1,4-dioxane, morpholine, thiomorpholine, thiomorpholine-S-oxide, thiomorpholine-S,S-dioxide, piperazine, pyran, 3-pyrroline, thiopyran, pyrone, tetrahydrofuran, and tetrahydrothiophene.

In one embodiment of any of formulae (I-CCXXXII), Z is a substituted or unsubstituted heterocycle selected from the group consisting of tetrahydrobenzotriazinyl, dihydrobenzotriazinyl, tetrahydrocinnolinyl, dihydrocinnolinyl, tetrahydroquinoxalinyl, dihydroquinoxalinyl, tetrahydroquinazolinyl, dihydroquinazolinyl, tetrahydroisoquinolinyl, dihydroisoquinolinyle, tetrahydrophthalazinyl, dihydrophthalazinyl, indolinyl, isoindolinyl, isoindolyl, dihydroindazolyl, indazolyl, dihydrobenzotriazolyl, benzotriazolyl, dihydrobenzofuranyl, dihydroisobenzofuranyl, dihydrobenzoisoxazolyl, dihydrobenzooxazolyl, dihydrobenzothiophenyl, dihydrobenzoisothiazolyl, dihydrobenzothiazolyl, chromanyl, chromenyl, thiochromanyl, thiochromenyl, isochromanyl, isochromenyl, isothiochromanyl, and isothiochromenyl.

In one embodiment of any of formulae (I-CCXXXII), Z is a substituted or unsubstituted heterocycle selected from the group consisting of 1,2,3,4-tetrahydrobenzo[d]1,2,3]triazinyl, 3,4-dihydrobenzo[d][1,2,3]triazinyl, 1,4-dihydrobenzo[d][1,2,3]triazinyl, 1,2-dihydrobenzo[d][1,2,3]triazinyl, 1,2,3,4-tetrahydrobenzo[e][1,2,4]triazinyl, 1,2-dihydrobenzo[e][1,2,4]triazinyl, 1,4-dihydrobenzo[e][1,2,4]triazinyl, 3,4-dihydrobenzo[e][1,2,4]triazinyl, 1,2,3,4-tetrahydrobenzo[e][1,2,3,4]tetrazinyl, 1,2-dihydrobenzo[e][1,2,3,4]tetrazinyle, 1,4-dihydrobenzo[e][1,2,3,4]tetrazinyl, 1,2-dihydrobenzo[e][1,2,3,4]tetrazinyl, 1,2,3,4-tetrahydrocinnolinyl, 3,4-dihydrocinnolinyl, 1,4-dihydrocinnolinyl, 1,2-dihydrocinnolinyl, 1,2,3,4-tetrahydroquinoxalinyl, 1,2-dihydroquinoxalinyl, 1,4-dihydroquinoxalinyl, 1,2,3,4-tetrahydroquinolinyl, 3,4-dihydroquinolinyl, 1,2-dihydroquinolinyl, 1,4-dihydroquinolinyl, 1,2,3,4-tetrahydroquinazolinyl, 3,4-dihydroquinazolinyl, 1,4-dihydroquinazolinyl, 1,2-dihydroquinazolinyl, 1,2,3,4-tetrahydroisoquinolinyl, 3,4-dihydroisoquinolinyl, 1,4-dihydroisoquinolinyl, 1,2-dihydroisoquinolinyl, 1,2,3,4-tetrahydrophthalazinyl, 1,2-dihydrophthalazinyl, and 1,4-dihydrophthalazinyl.

In one embodiment of any of formulae (I-CCXXXII), Z is a substituted or unsubstituted heterocycle selected from the group consisting of indolinyl, 3H-indolyl, 1H-indolyl, isoindolinyl, 1H-isoindolyl, 2,3-dihydro-1H-indazolyl, 3H-indazolyl, 1H-indazolyl, 2,3-dihydro-1H-benzo[d][1,2,3]triazolyl, and 1H-benzo[d][1,2,3]triazolyl.

In one embodiment of any of formulae (I-CCXXXII), Z is a substituted or unsubstituted heterocycle selected from the group consisting of 2,3-dihydrobenzofuranyl, 1,3-dihydroisobenzofuranyl, 2,3-dihydrobenzo[d]isoxazolyl, 1,3-dihydrobenzo[c]disoxazolyl, 2,3-dihydrobenzo[c]oxazolyl, 2,3-dihydrobenzo[b]thiophenyl, 1,3-dihydrobenzo[c]thiophenyl, 2,3-dihydrobenzo[d]isothiazolyl, 1,3-dihydrobenzo[c]isothiazolyl, and 2,3-dihydrobenzo[d]thiazolyl.

In one embodiment of any of formulae (I-CCXXXII), Z is a substituted or unsubstituted heterocycle selected from the group consisting of chromanyl, 4H-chromenyl, 2H-chromenyl, thiochromanyl, 4H-thiochromenyl, 2H-thiochromenyl, isochromanyl, 1H-isochromenyl, isothiochromanyl, and 1H-isothiochromenyl.

In one embodiment of any of formulae (I-CCXXXII), heterocycle groups as substituents on Z include pyrrolidine, piperidine, imidazolidine, pyrazolidine, butyrolactam, valerolactam, imidazolidinone, hydantoin, dioxolane. phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of any of the formulae (I-CCXXXII) where L is a bond, Z is 3- to 10-membered monocyclic or bicyclic heterocyclyl ring system, where one ring heteroatom is located alpha (ortho) to bond L.

In one embodiment of any of the formulae (I-CCXXXII) where L is a bond, Z is 3- to 10-membered monocyclic or bicyclic heterocyclyl ring system containing one or more ring nitrogen atoms, and where one or more ring nitrogen atoms is located alpha (ortho) to the bond L.

In one embodiment of the formulae (I-CCXXXII), Z has one or more substituents selected from the group consisting of:

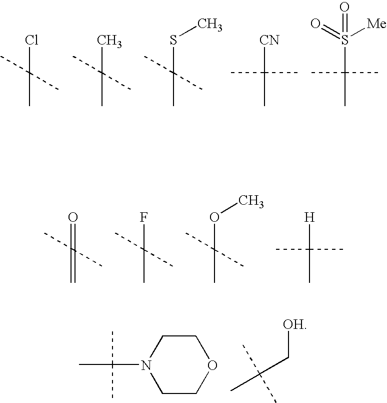

In one embodiment of any of formulae (I-CCXXXII), Z is the following residue:

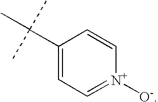

In another embodiment of any of formulae (I-CCXXXII), Z is selected from one of the following residues:

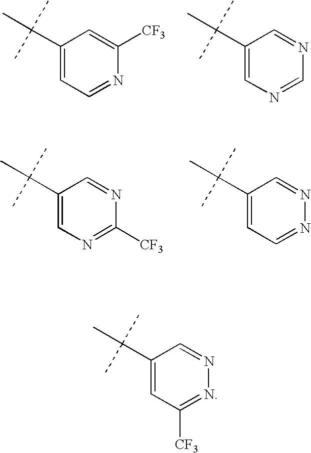

In another embodiment of any of formulae (I-CCXXXII), Z is selected from one of the following residues:

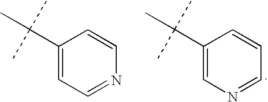

In another embodiment of any of formulae (I-CCXXXII), Z is selected from one of the following residues:

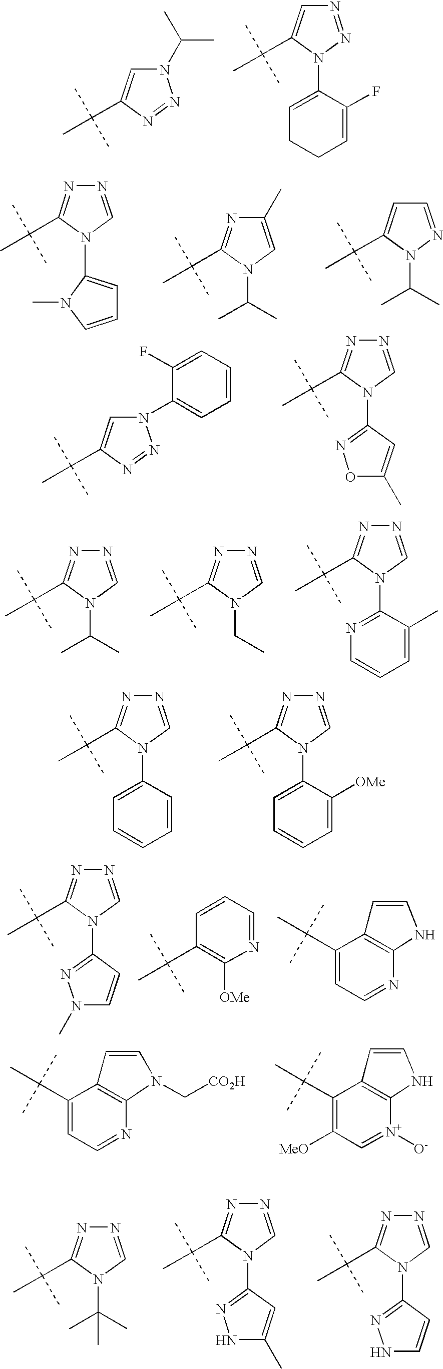

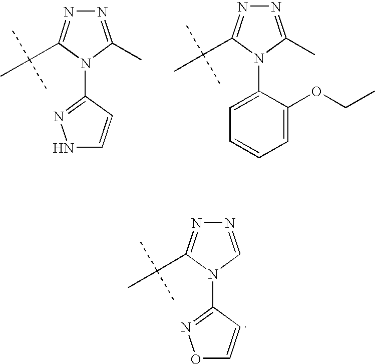

In another embodiment of any of formulae (I-CCXXXII), L is C═O, and Z is selected from one of the following residues:

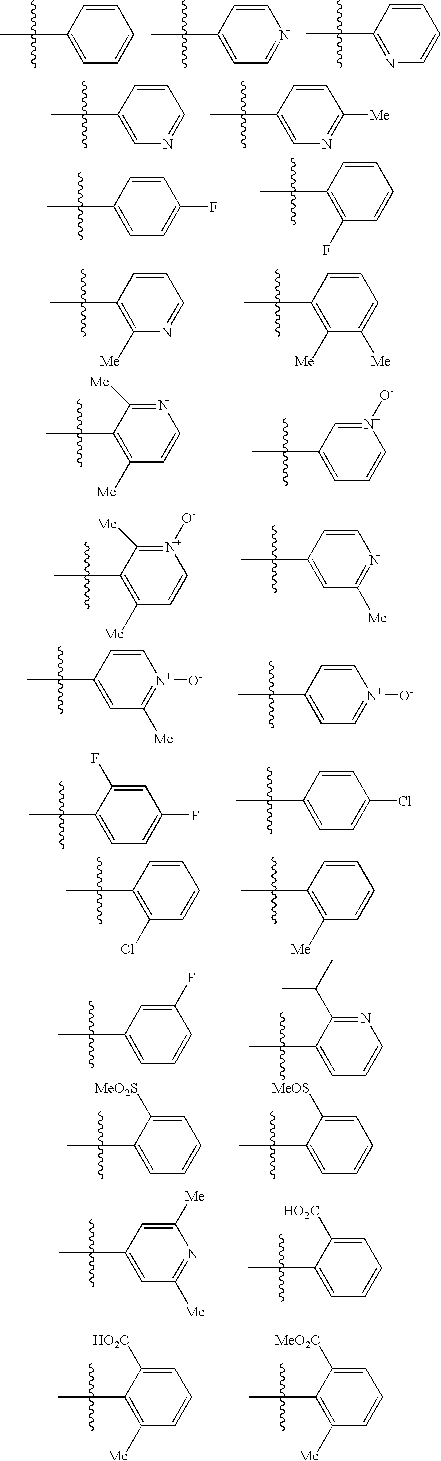

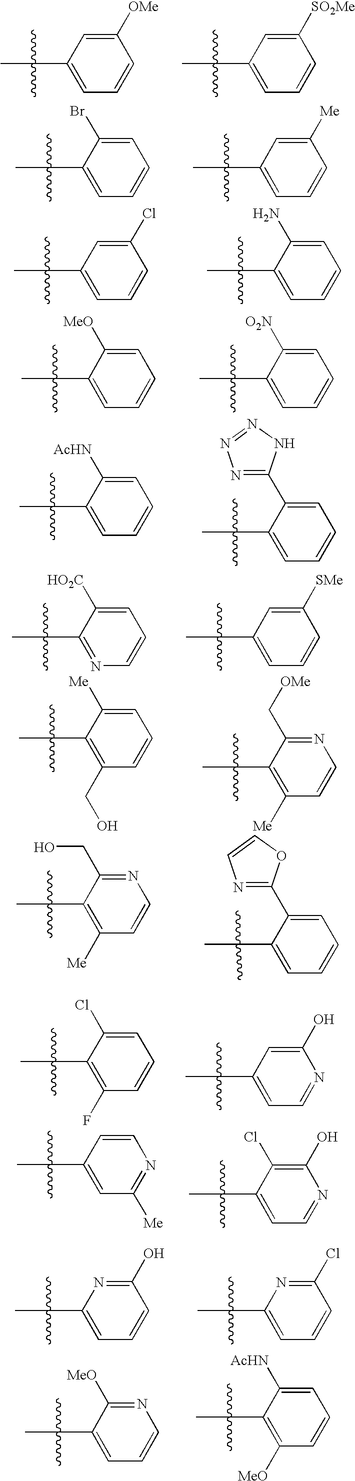

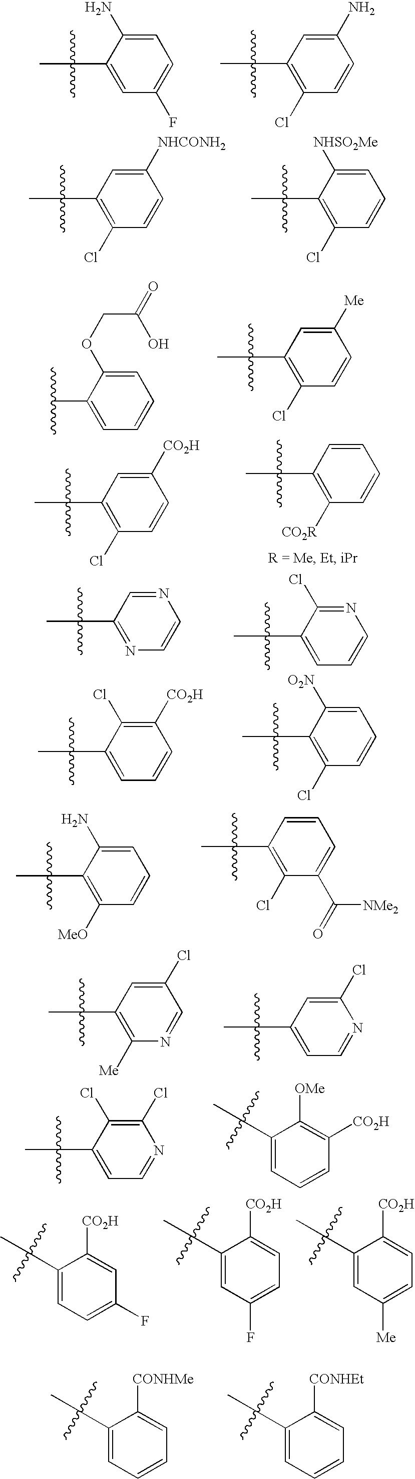

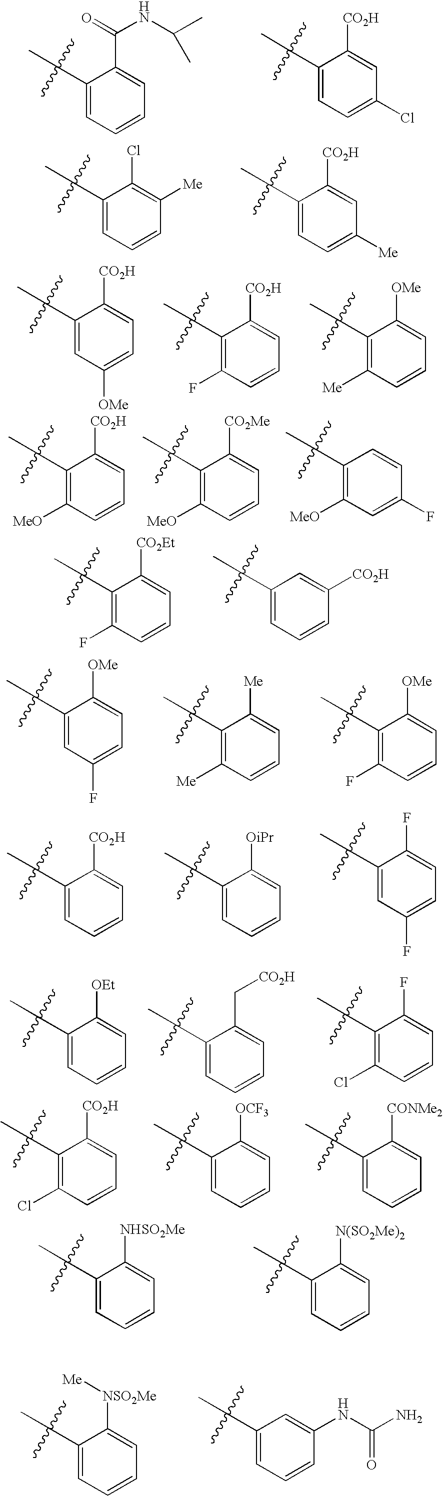

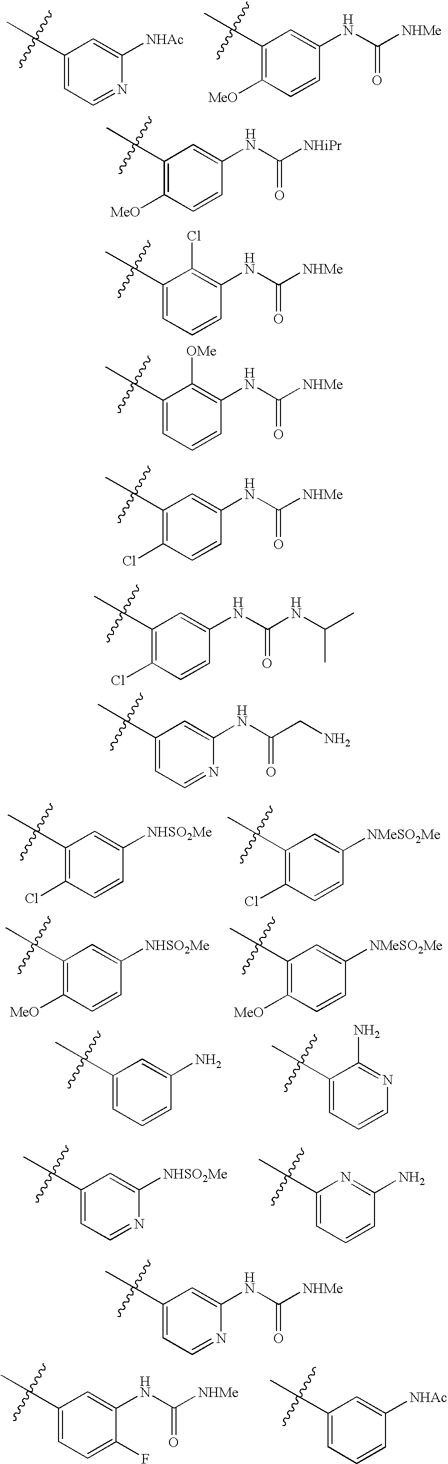

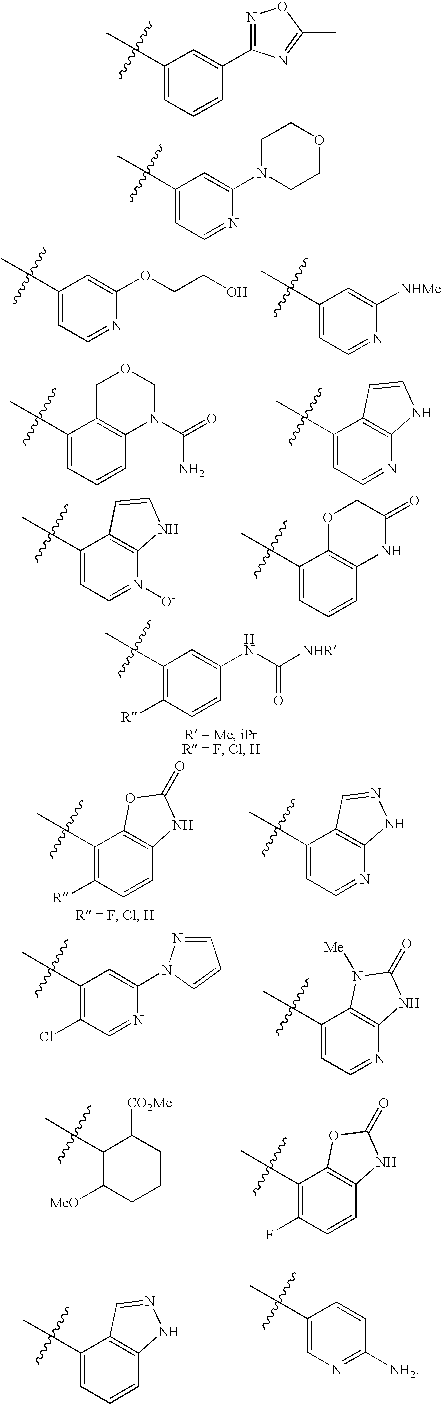

In another embodiment of any of formulae (I-CCXXXII), L is C═O. or a bond, and Z is selected from one of the following residues:

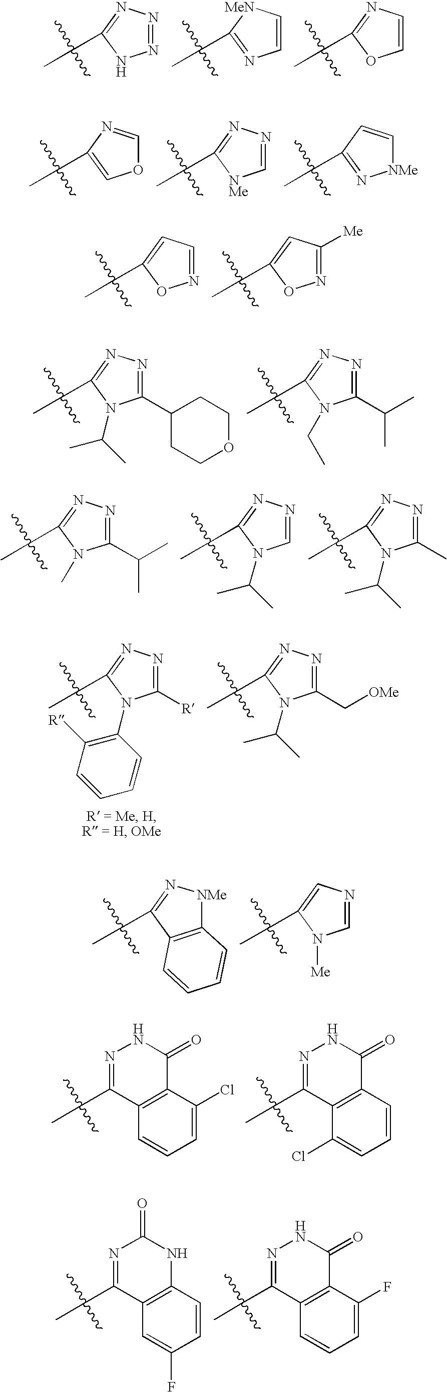

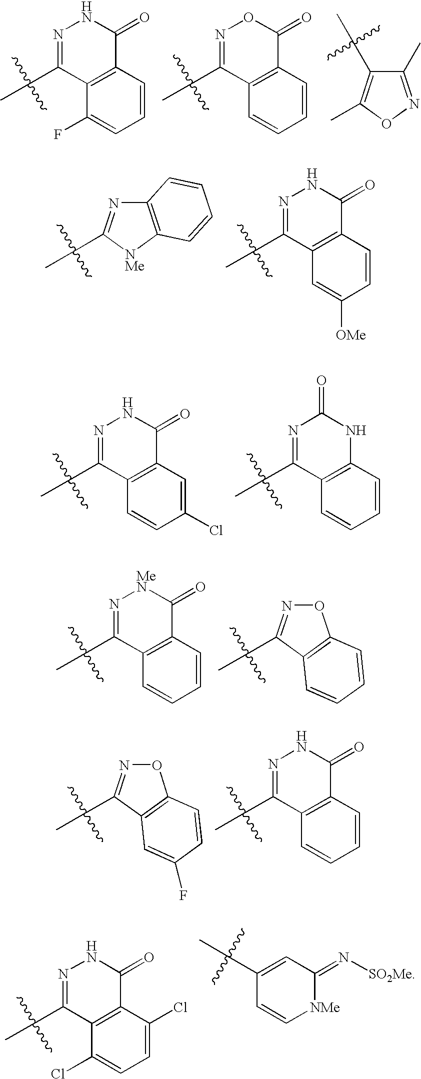

In another embodiment of any of formulae (I-CCXXXII), L is C═O, and Z is selected from one of the following residues:

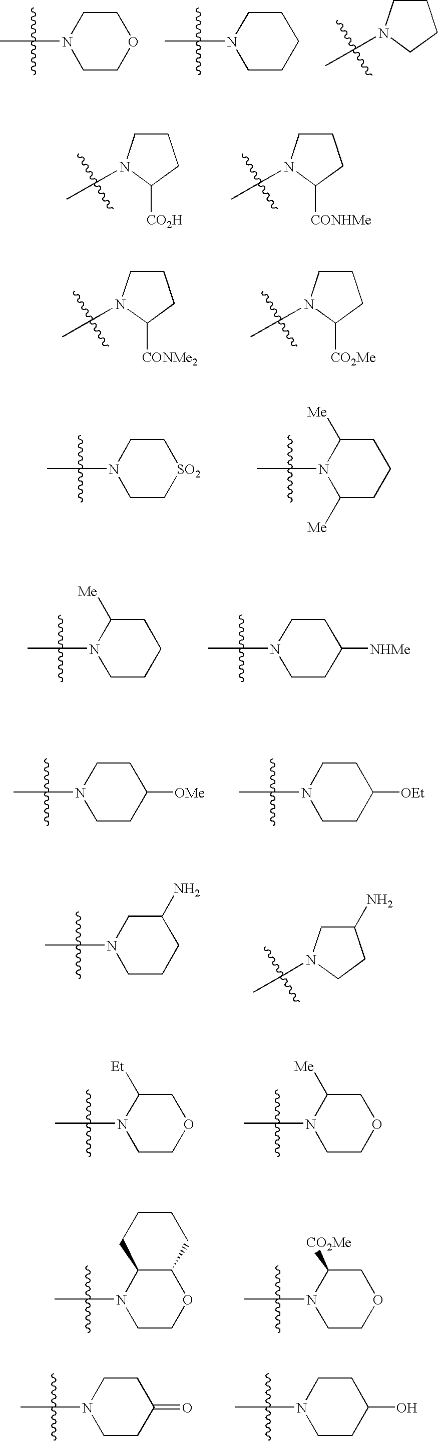

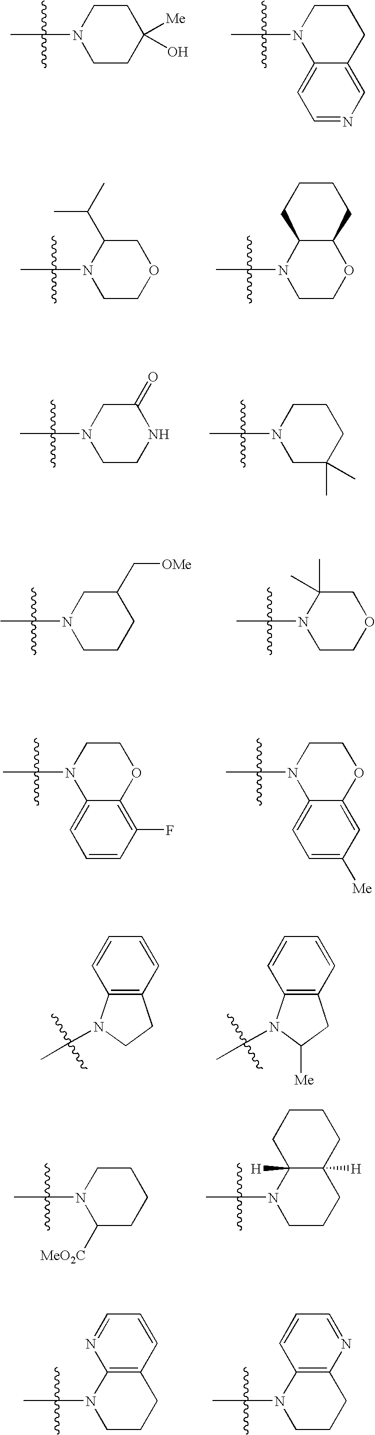

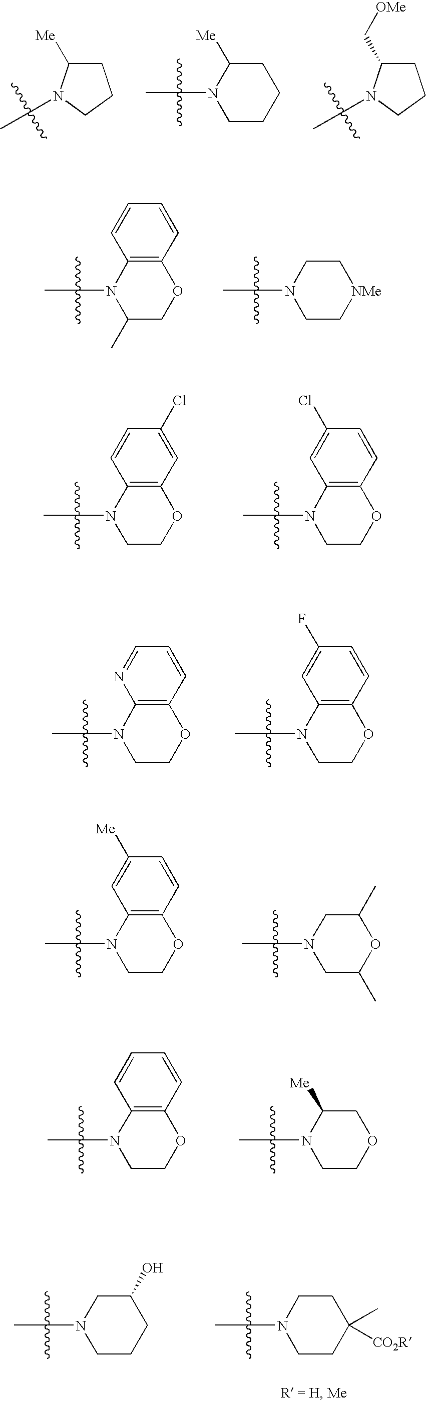

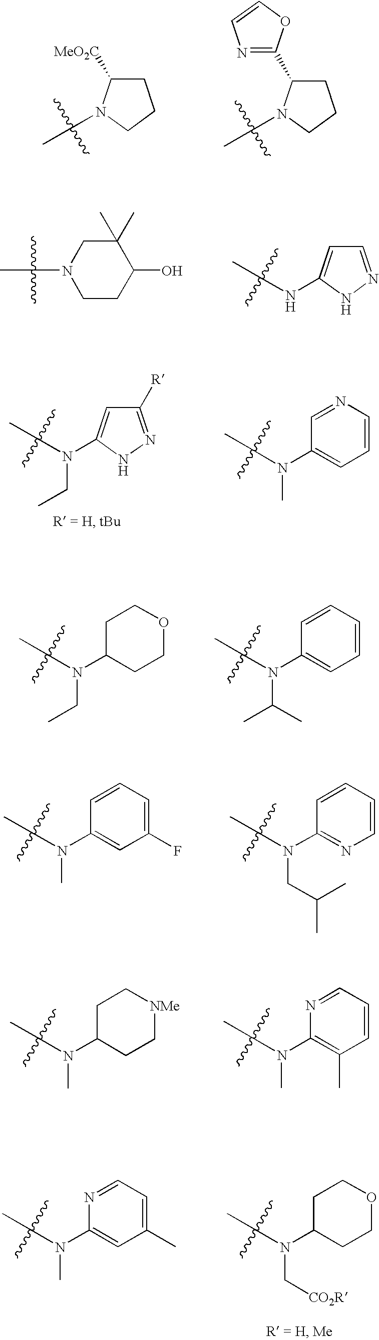

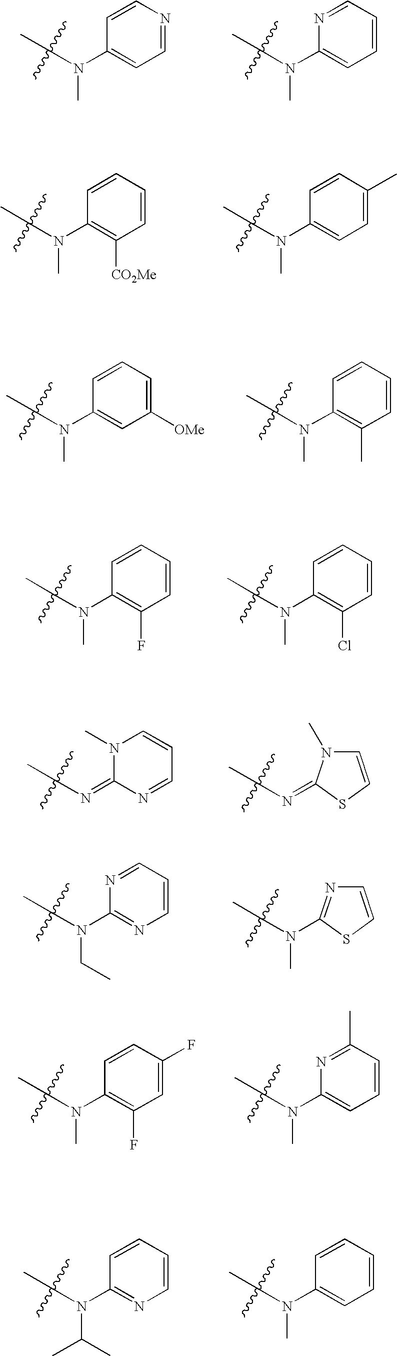

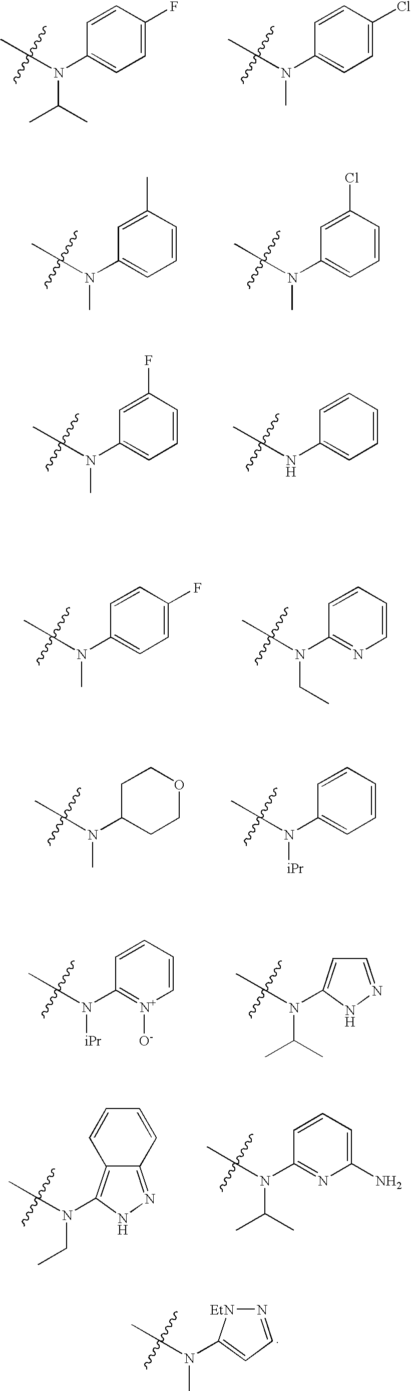

In another embodiment of any of formulae (I-CCXXXII), Z is selected from one of the following residues:

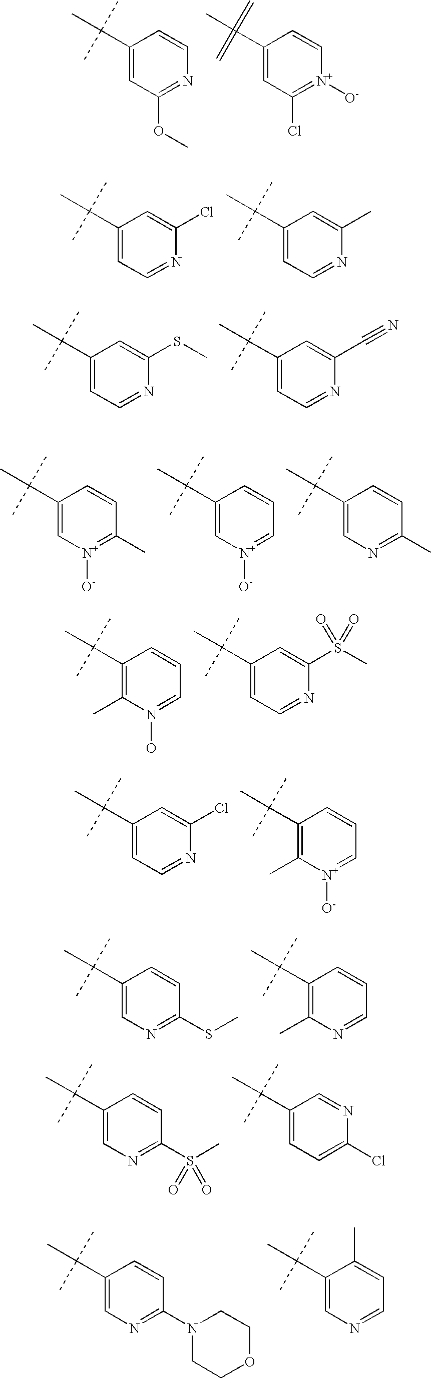

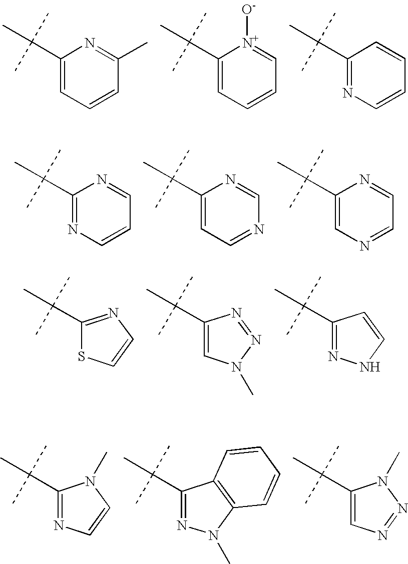

In other embodiments for each of the formulae (I-CCXXXII). Z is selected from the group consisting of:

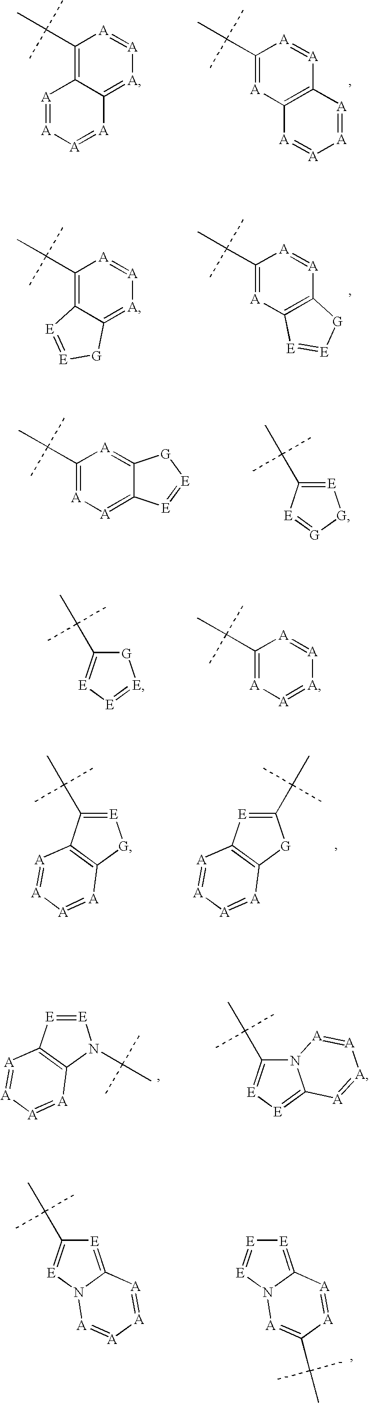

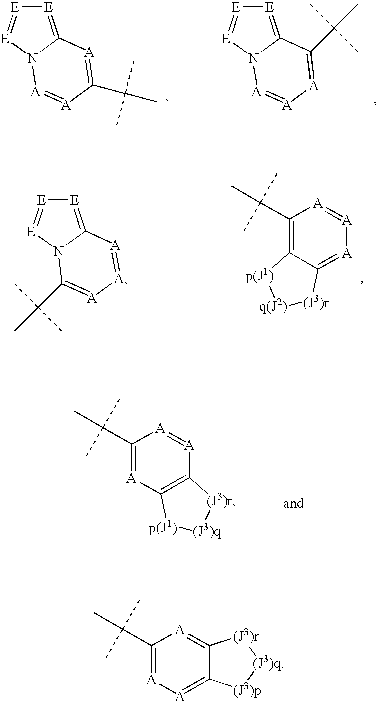

In other embodiments for each of the formulae (I-CCXXXII), Z is selected from the group consisting of:

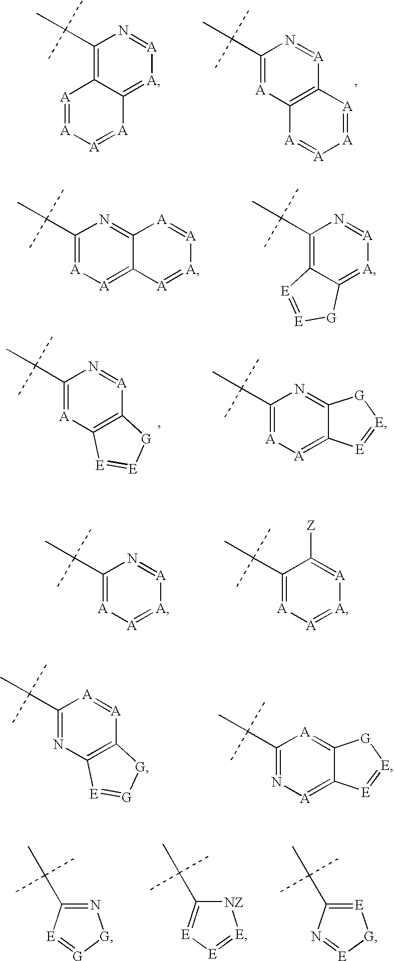

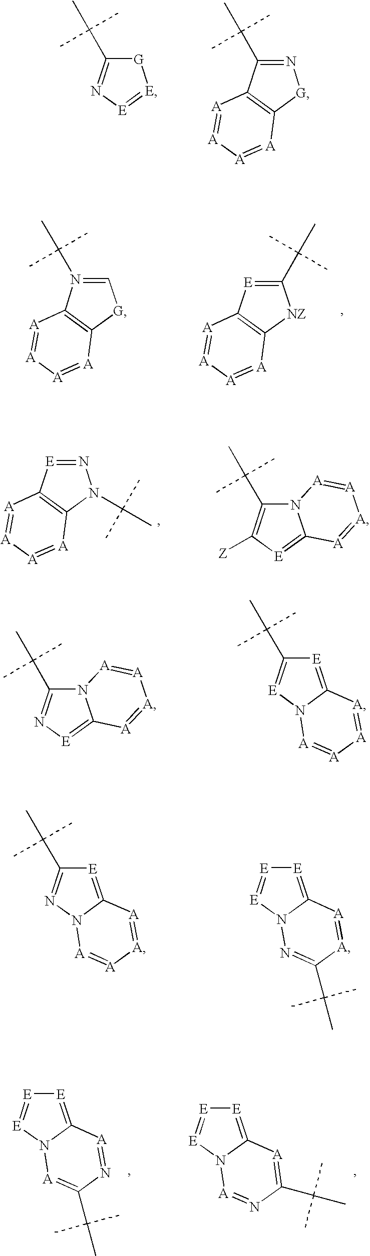

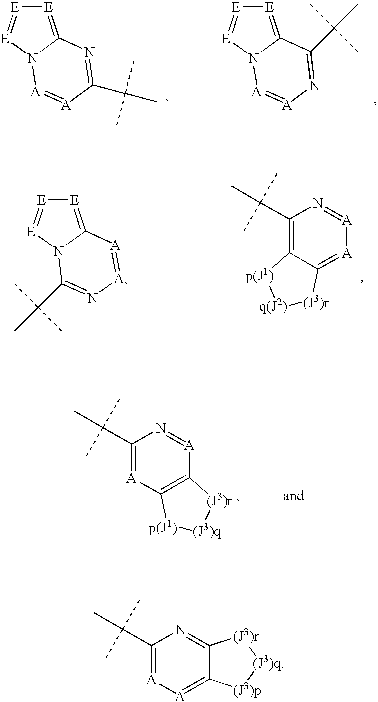

In one embodiment of any of formulae (I-CCXXXII), Z has one or more substituents selected from the group consisting of:

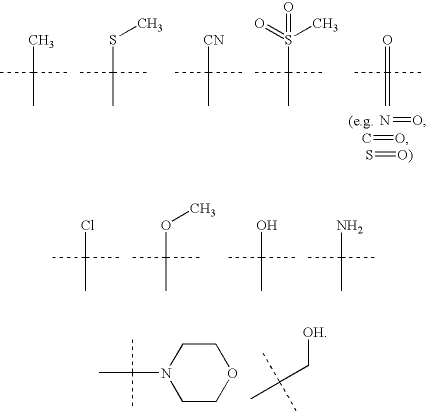

with the proviso that at least one substituent is other than hydrogen. In another embodiment, all substituents on Z are hydrogen.

In one embodiment for any of formulae (XX-CCXXXII), Z¹, Z^{a} and Z^{b} are all simultaneously hydrogen.

In one embodiment, in each of the formula (XX-CCXXXII); Z¹, Z^{a}, Z^{b} or Z^{c} are independently selected from the group consisting of hydrogen, halogen —OR¹⁰, —CN, —NO₂, ═O, —OC(O)R¹⁰, —CO₂R¹⁰, —C(O)R¹⁰, —CONR¹¹R¹², —NR¹⁰C(O)R¹², —NR¹¹R¹², —SR¹⁰, —S(O)R¹⁰, —S(O)₂R¹⁰, —NR¹⁰S(O)₂R¹¹, substituted or unsubstituted C₁₋₈ alkyl, substituted or unsubstituted 4- to 7-membered heterocyclyl, substituted or unsubstituted 5 or 6 ring heteroaryl, substituted or unsubstituted phenyl, substituted or unsubstituted C₁₋₈ alkenyl and substituted or unsubstituted C₁₋₁₃ alkynyl;

suitable substituted C₁₋₈ alkyl, C₂₋₈ alkenyl and C₂₋₈ alkynyl may have from 1 to 3 substituents independently selected from the group consisting of halogen, —OR¹⁰, —CN, —NO₂, ═O, —OC(O)R¹⁰, —CO₂R¹⁰, —C(O)R¹⁰, —CONR¹⁰R¹¹, —OC(O)NR¹⁰R¹¹, —NR¹⁰C(O)R¹¹, —NR¹⁰C(O)NR¹¹R¹², —NR¹⁰R¹¹, —NR¹⁰CO₂R¹¹, —SR¹⁰, —SOR¹⁰, —SO₂R¹⁰, —SO₂NR¹⁰R¹¹, —NR¹⁰SO₂R¹¹, unsubstituted or substituted phenyl, unsubstituted or substituted C₅₋₆ heteroaryl, and unsubstituted or substituted C₃₋₆ heterocyclyl;

suitable substituted aryl, heteroaryl and heterocycle substituents may have from 1 to 3 substituents independently selected from the group consisting of halogen, —OR¹⁰, —CN, —NO₂, ═O, —OC(O)R¹⁰, —OC(O)R¹⁰, —CO₂R¹⁰, —C(O)R¹⁰, —CONR¹⁰R¹¹, —OC(O)NR¹⁰R¹¹, —NR¹⁰C(O)R¹¹, —NR¹⁰C(O)NR¹¹R¹², —NR¹⁰R¹¹, —NR¹⁰CO₂R¹¹, —SOR¹⁰, —SO₂R¹⁰, —SO₂NR¹⁰R¹¹, —NR¹⁰SO₂R¹¹ and unsubstituted 4- to 7-membered heterocyclyl, unsubstituted C₁₋₈ alkyl and unsubstituted C₁₋₈ haloalkyl; with the proviso that where the substituent Z¹, Z^{a}, Z^{b} and Z^{c} is a heterocycle, suitable substituents on this heterocycle preferably do not include another heterocycle;

R¹⁰, R¹¹ and R¹² are each independently hydrogen, C₁₋₈ alkyl, C₂₋₈ alkenyl, C₂₋₈ alkynyl, aryl, or heteroaryl, or where R¹⁰ and R¹¹, or R¹¹ and R¹², or R¹⁰ and R¹², together with the atom(s) to which they are attached, form an substituted or unsubstituted 5-, 6-, or 7-membered ring; and

the aromatic and aliphatic portions of R¹⁰, R¹¹ and R¹² are optionally further substituted with from one to three members selected from the group consisting of halogen, —OH, —OR^{m}, —OC(O)NHR^{m}, —OC(O)NR^{m}Rⁿ, —SH, —SR^{m}, —S(O)R^{m}, —S(O)₂R^{m}, —S(O)₂NH₂, —S(O)₂NHR^{m}, —S(O)₂NR^{m}Rⁿ, —NHS(O)₂R^{m}, —NR^{m}S(O)₂Rⁿ, —C(O)NH₂, —C(O)NHR^{m}, —C(O)N(R^{m})₂, —C(O)R^{m}, —NHC(O)R^{m}, —NR^{m}C(O)Rⁿ, —NHC(O)NH₂, —NR^{m}C(O)NH₂, —NR^{m}C(O)NHRⁿ, —NHC(O)NHR^{m}, —NR^{o}C(O)NR^{m}Rⁿ, —NHC(O)N(R^{m})₂, —CO₂H, —CO₂R^{m}, —NHCO₂R^{m}, —NR^{m}CO₂Rⁿ, —CN, —NO₂, —NH₂, —NHRⁿ, —NR^{m}Rⁿ, —NR^{m}S(O)NH₂, and —NR^{m}S(O)₂NHRⁿ, where R^{m}Rⁿ, and R^{o} are each independently unsubstituted C₁₋₆ alkyl.

In one embodiment, any of formulae (XX-CCXXXII), Z¹, Z^{a}, Z^{b} and Z^{c} are independently selected from the group consisting of hydrogen, halogen, substituted or unsubstituted C₁₋₈ alkyl, —CN, ═O, —NO₂, —OR¹⁰, —OC(O)R¹⁰, —CO₂R¹⁰, —C(O)NR¹¹R¹², —NR¹⁰C(O)R¹¹, —NR¹⁰C(O)NR¹¹R¹², —NR¹¹R¹², —SR¹⁰, —S(O)R¹⁰, —S(O)₂R¹⁰, —S(O)₂NR¹¹R¹², and —NR¹⁰S(O)₂R¹¹.

In one embodiment, any of formulae (XX-CCXXXII), Z¹, Z^{a}, Z^{b} and Z^{c} are independently selected from the group consisting of hydrogen, substituted or unsubstituted C₆₋₁₀ aryl, substituted or unsubstituted 5- to 10-membered heteroaryl and substituted or unsubstituted 3- to 10-membered heterocyclyl.

In one embodiment, any of formulae (XX-CCXXXII), Z¹, Z^{a}, Z^{b} and Z^{c} are independently selected from the group consisting of —F, —Cl, —Br, —NO₂, —CH₃, —CH₂CH₃, —CH(CH₃)₂, —CH₂OH, —CH₂CO₂H, —OH, —OCH₃, —OCH₂CH₃, —OCH(CH₃)₂, —OCH₂CH₂OCH₃, —OCF₃, ═O, —NH₂, —NHCH₃, —NHCOCH₃, —NHCONH₂, —NHCONHCH₃, —NHCONHCH(CH₃)₂, —NHCOCH₂NH₂, —NHSO₂CH₃, —NCH₃SO₂CH₃, —N(SO₂CH₃)₂, —CO₂H, —CO₂CH₃, —CO₂CH₃, —CO₂CH(CH₃)₂, —CON(CH₃)₂, —CONHCH₃, —CONHCH₂CH₃, —CONHCH(CH₃)₂, —SCH₃, —SOCH₃, —SO₂CH₃, morpholinyl, tetrazole, oxazole, 5-methyl-1,2,4-oxadiazolyl, and pyrazolyl.

In one embodiment, any of formulae (XX-CCXXXII), Z¹, Z^{a}, Z^{b} and Z^{c} are independently selected from the group consisting of hydrogen, —F, —Cl, —OCH₃, —CH₃, —CH₂CH₃, and CH(CH₃)₂.

In one embodiment, any of formulae (XX-CCXXXII), Z¹, Z^{a}, Z^{b} and Z^{c} are independently selected from the group consisting of unsubstituted C₁₋₈ alkyl.

In one embodiment, any of formulae (XX-CCXXXII), Z¹, Z^{a}, Z^{b} and Z^{c} are independently selected from the group consisting of substituted C₁₋₈ alkyl.

In one embodiment, any of formulae (XX-CCXXXII), Z¹, Z^{a}, Z^{b} and Z^{c} are independently selected from the group consisting of unsubstituted C₁₋₆ alkyl (not —Me), ═O, C₁₋₆ haloalkyl (not —CF₃), —COOH, —NO₂, or —OR¹⁰ (not —OMe). In this embodiment, Z can be any chemically allowed regioisomer of pyridyl, pyrimidinyl, pyridazinyl, pyrazinyl and the like, and their respective N-oxides.

In one embodiment, any of formulae (XX-CCXXXII), Z¹, Z^{a}, Z^{b} and Z^{c} are independently selected from the group consisting of —CH₃, ═O, —CF₃, —OCH₃. In this embodiment, Z can be any chemically allowed regioisomer of pyridyl, pyrimidinyl, pyridazinyl, pyrazinyl and the like, and their respective N-oxides.

In one embodiment, any of formulae (XX-CCXXXII), Z¹, Z^{a}, Z^{b} and Z^{c} are independently selected from the group consisting of halogen, substituted C₁₋₆ alkyl (but not C₁₋₆ haloalkyl), unsubstituted or substituted C₁₋₆ alkenyl, unsubstituted or substituted C₁₋₆ alkynyl, ═O, —CN, —C(O)R¹⁰, —C(O)NR¹⁰R¹¹, —NR¹⁰C(O)R¹¹, —NR¹¹R¹², —SR¹⁰—S(O)₂R¹⁰, —S(O)₂NR¹¹R¹², —NR¹⁰S(O)₂R¹¹, —OC(O)R¹⁰, —CO₂R¹⁰ (but not —CO₂H), —OC(O)NR¹¹R¹², —NR¹⁰C(O)NR¹¹R¹², —NR¹⁰CO₂R¹¹, unsubstituted or substituted 5- or 6-membered heteroaryl and a unsubstituted or substituted 4- to 7-membered heterocyclyl. Preferred substituents include chlorine, ═O, —CN, —SCH₃, —SO₂CH₃. In this embodiment, Z can be any chemically allowed regioisomer of pyridyl, pyrimidinyl, pyridazinyl, pyrazinyl and the like, and their respective N-oxides.

In one embodiment of any of formulae (XX-CCXXXII) where L is C═T, and where the substituent Z¹, Z^{a}, Z^{b} and Z^{c} is a heterocycle, suitable substituents on this heterocycle do not include another heterocycle.

In one embodiment of any of formulae (XX-CCXXXII) where L is a bond or NR^{c}, when Z¹, Z^{a}, Z^{b} or Z^{c} are substituted C₁₋₈ alkyl, substituted C₂₋₈ alkenyl, substituted C₂₋₈ alkynyl or substituted C₁₋₈alkoxy groups, it preferably has from 1 to 3 substituents independently selected from the group consisting of halogen, —OH, —OR¹⁰, ═O, —CO₂R¹⁰, —C(O)R¹⁰, —C(O)NR¹¹R¹², —NR¹⁰C(O)R¹¹, —NR¹¹R¹², —SR¹⁰, —S(O)R¹⁰, —S(O)₂R¹⁰, —NR¹⁰S(O)₂R¹¹, substituted or unsubstituted phenyl, substituted or unsubstituted 5- or 6-membered heteroaryl, and substituted or unsubstituted 4- to 7-membered heterocyclyl. More preferably, it has from 1 to 3 substituents independently selected from the group consisting of halogen, —OH, —OR¹⁰, ═O, —C(O)R¹⁰, —CO₂R¹⁰, —C(O)NR¹¹R¹², —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, and 4- to 7-membered heterocyclyl.

In one embodiment, in each of the formula (XX-CCXXXII), when one of Z¹, Z^{a}, Z^{b}, Z^{c}, and Z^{d} is a substituted C₁₋₈ alkyl, substituted C₂₋₈ alkenyl, substituted C₂₋₈ alkynyl, it preferably has from 1 to 3 substituents independently selected from the group consisting of halogen, —OR¹⁰, ═O, —CO₂R¹⁰, —C(O)R¹⁰, —C(O)NR¹¹R¹², —NR₁₀C(O)R¹¹, —NR¹¹R¹², —SR¹⁰, —S(O)R¹⁰, —S(O)₂R¹⁰, —NR¹⁰S(O)₂R¹¹, substituted or unsubstituted phenyl, substituted or unsubstituted 5- or 6-membered heteroaryl, and substituted or unsubstituted 4- to 7-membered heterocyclyl. More preferably, it has from 1 to 3 substituents independently selected from the group consisting of halogen, —OR¹⁰, ═O, —C(O)R¹⁰, —CO₂R¹⁰, —CONR¹¹R¹², —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, and 4- to 7-membered heterocyclyl.

In one embodiment of any of formulae (XX-CCXXXII), when Z¹, Z^{a}, Z^{b} or Z^{c} is substituted heterocyclyl, it preferably has from 1 to 2 substituents independently selected from the group consisting of substituted or unsubstituted C₁₋₈ alkyl, —OR¹⁰, —OH, —C(O)R¹⁰, —C(O)NR¹¹R¹², —NR¹¹R¹², and —S(O)₂R¹⁰.

In one embodiment of any of formulae (XX-CCXXXII), when Z¹, Z^{a}, Z^{b}, Z^{c}, and Z^{d} is substituted heterocyclyl, it preferably has from 1 to 2 substituents independently selected from the group consisting of substituted or unsubstituted C₁₋₈ alkyl, —OR¹⁰, —OH, —C(O)R¹⁰, —C(O)NR¹¹R¹², —NR¹¹R¹², and —S(O)₂R¹⁰.

In one embodiment of any of formulae (XX-CCXXXII), at least one of Z¹, Z^{a}, Z^{b} and Z^{c} is a unsubstituted or substituted heterocyclyl selected from the group including pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of any of formulae (XX-CCXXXII), Z¹, Z^{a}, Z^{b} and Z^{c} are independently selected from the group consisting of hydrogen, halogen, substituted or unsubstituted C₁₋₈ alkyl, substituted or unsubstituted C₂₋₈ alkenyl, substituted or unsubstituted C₂₋₈ alkynyl, substituted or unsubstituted C₁₋₈alkoxy, ═O, —CN, —NO₂, —OR¹⁰, —OC(O)R¹⁰, —CO₂R¹⁰, —C(O)R¹⁰, —C(O)NR¹¹R¹², —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, —OC(O)NR¹¹R¹², —NR¹⁰C(O)NR¹¹R¹², substituted or unsubstituted phenyl, substituted or unsubstituted 5- or 6-membered heteroaryl, and substituted or unsubstituted 4- to 7-membered heterocyclyl. If present, one substituent is preferably located ortho to one of the heteroatoms in the heteroaryl Z ring. Alternatively, one substituent, ═O, may be directly connected to a ring heteroatom in the heteroaryl Z ring.

In one embodiment of any of formulae (XX-CCXXXII), when Z¹, Z^{a}, Z^{b} or Z^{c} are substituted C₁₋₈ alkyl, substituted C₂₋₈ alkenyl, substituted C₂₋₈ alkynyl or substituted C₁₋₈ alkoxy groups, it preferably has from 1 to 3 substituents independently selected from the group consisting of halogen, —OH, —OR¹⁰, ═O, —CO₂R¹⁰, —C(O)R¹⁰, —C(O)NR¹¹R¹², —NR¹⁰C(O)R¹¹, —NR¹¹R¹², —SR¹⁰, —S(O)R¹⁰, —S(O)₂R¹⁰, —NR¹⁰S(O)₂R¹¹, substituted or unsubstituted phenyl, substituted or unsubstituted 5- or 6-membered heteroaryl, and substituted or unsubstituted 4- to 7-membered heterocyclyl. More preferably, it has from 1 to 3 substituents independently selected from the group consisting of halogen, ——OH, —OR¹⁰, ═O, —C(O)R¹⁰, —CO₂R¹⁰, —C(O)NR¹¹R¹², —NR¹⁰C(O)R¹¹, —NR¹¹R¹², —SR¹⁰, —S(O)R¹⁰, —S(O)₂R¹⁰, —S(O)₂NR¹¹R¹², —NR¹⁰S(O)₂R¹¹, and 4- to 7- membered heterocyclyl.

In one embodiment of each of the formulae (XX-CCXXXII) when at least one of Z¹, Z^{a}, Z^{b} and Z^{c} is a substituted C₁₋₈ alkyl, at least one substituent is a substituted or unsubstituted 4- to 7-membered heterocyclyl represented by formula (AA) as defined in [0045]-[0048]. Preferably, R²⁰, R²¹, R²², R²³, R²⁴, R²⁵, and R²⁶ are independently hydrogen or C₁₋₄alkyl. In another preferred embodiment, at least three of R²⁰, R²¹, R²², R²³, R²⁴, R²⁵, and R²⁶ are hydrogen, j is 1 or 2, k is 1 or 2 with the proviso that j+k is 3 or 4. In another preferred embodiments, at least five of R²⁰, R²¹, R²², R²³, R²⁴, R²⁵, and R²⁶ are hydrogen, j is 1 or 2, k is 1 or 2 with the proviso that j+k is 3 or 4.

In one embodiment of each of the formulae (XX-CCXXXII) when at least one of Z¹, Z^{a}, Z^{b} and Z^{c} is a substituted C₁₋₈ alkyl, at least one substituent is selected from the group including pyrrolidine, piperidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-dioxide, piperazine, pyran, pyridone, 3-pyrroline, thiopyran, pyrone, tetrahydrofuran, and tetrahydrothiophene.

In one embodiment of each of the formulae (XX-CCXXXII) when at least one of Z¹, Z^{a}, Z^{b} and Z^{c} is a substituted C₁₋₈ alkyl, at least one substituent is a substituted or unsubstituted 5- or 6-membered heteroaryl selected from the group consisting of pyridyl, pyridazinyl, pyrazinyl, pyrimidinyl, triazinyl, isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl.

In one embodiment of any of formulae (XX-CCXXXII), when Z¹, Z^{a}, Z^{b} or Z^{c} is substituted heterocyclyl, it preferably has from 1 to 2 substituents independently selected from the group consisting of substituted or unsubstituted C₁₋₈ alkyl, —OR¹⁰, —OH, —C(O)R¹⁰, —C(O)NR¹¹R¹², —NR¹¹R¹², and —S(O)₂R¹⁰.

In one embodiment of any of formulae (XX-CCXXXII), Z¹, Z^{a}, Z^{b} and Z^{c} are independently selected from the group consisting of halogen, —OH, —OR¹⁰, —CN, —NO₂, ═O, —OC(O)R¹⁰, —CO₂R¹⁰, —C(O)R¹⁰, —C(O)NR¹¹R¹², —NR¹⁰C(O)R¹², —NR¹¹R¹², —SR¹⁰, —S(O)R¹⁰, —S(O)₂R¹⁰, —NR¹⁰S(O)₂R¹¹, substituted or unsubstituted C₁₋₈ alkyl, substituted or unsubstituted 4- to 7-membered heterocyclyl, substituted or unsubstituted 5 or 6 ring heteroaryl, substituted or unsubstituted phenyl, substituted or unsubstituted C₁₋₈ alkenyl and substituted or unsubstituted C₁₋₈ alkynyl.

In one embodiment of any of formulae (XX-CCXXXII), at least one of Z¹, Z^{a}, Z^{b} and Z^{c} is a substituted or unsubstituted 5- or 6-membered heteroaryl selected from the group consisting of pyridyl, pyridazinyl, pyrazinyl, pyrimidinyl, triazinyl, isothiazolyl, pyrazolyl, imidazolyl, thienyl, furyl, triazolyl, tetrazolyl, oxazolyl, isoxazolyl, oxadiazolyl, thiadiazolyl, pyrrolyl, and thiazolyl.

In one embodiment of any of formulae (XX-CCXXXII) where L is NR^{c}, Z^{a}, and Z^{b} are independently selected from the group consisting of halogen, —OH, —OR¹⁰, —CN, —NO₂, ═O, —OC(O)R¹⁰, —CO₂R¹⁰, —C(O)R¹⁰, —C(O)NR¹¹R¹², —NR¹⁰C(O)R¹², —NR¹¹R¹², —SR¹⁰, —S(O)₂R¹⁰, —NR¹⁰S(O)₂R¹¹, substituted or unsubstituted C₁₋₈ alkyl, substituted or unsubstituted 4- to 7-membered heterocyclyl, substituted or unsubstituted 5 or 6 ring heteroaryl, substituted or unsubstituted phenyl, substituted or unsubstituted alkenyl, and substituted or unsubstituted C₁₋₈ alkynyl.

In one embodiment of the formulae (XX-CCXXXII), Z¹, Z^{a}, Z^{b} or Z^{c} are each hydrogen.

In one embodiment in any of the formulae (XX-CCXXXII), at least one substituent Z¹, Z^{a}, Z^{b} and Z^{c} is cyano.

In one embodiment in any of the formulae (XX-CCXXXII), at least one substituent Z¹, Z^{a}, Z^{b} and Z^{c} is —S(O)₂R⁷. In one particular embodiment, R⁷ is methyl.

In one embodiment in any of the formulae (XX-CCXXXII), at least one substituent Z¹, Z^{a}, Z^{b} and Z^{c} is halogen (in particular chlorine).

In one embodiment in any of the formulae (XX-CCXXXII), at least one substituent Z¹, Z^{a}, Z^{b} and Z^{c} is —OR⁷. In one particular embodiment, R⁷ is methyl.

In one embodiment in any of the formulae (XX-CCXXXII), at least one substituent Z¹, Z^{a}, Z^{b} and Z^{c} is —SR⁷. In one particular embodiment, R⁷ is methyl.

In one embodiment of any of formulae (XX-CCXXXII), any one of Z¹, Z^{a}, Z^{b}, and Z^{c} may be a heterocyclic group represented by formula (AA) below, where formula (AA) is attached via a free valence on either M¹ or M², and where formula (AA) and the substituents therein are defined in [0045].

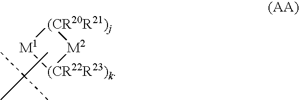

In one embodiment of the formulae (XX-CCXXXII); Z¹, Z^{a}, Z^{b} or Z^{c} are selected from one of the following residues

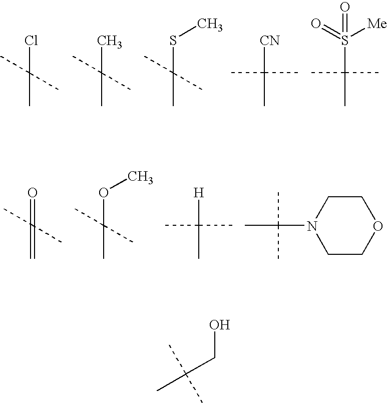

with the proviso that at least one of Z′, Z¹, Z^{a}, Z^{b} or Z^{c} are other than hydrogen.

In one embodiment of any of formulae (I-CCXXXII) where L is —CR^{a}R^{b}, Ar¹ is substituted or unsubstituted phenyl; Z is substituted or unsubstituted phenyl, or a substituted or unsubstituted 5- or 6-membered heteroaryl; and R^{a} and R^{b} are both halogen, and more preferably, are both fluorine.

In one embodiment of any of formulae (I-CCXXXII) where L is —CR^{a}R^{b}, Ar¹ is substituted or unsubstituted phenyl; Z is substituted or unsubstituted phenyl, or a substituted or unsubstituted 5- or 6-membered heteroaryl; R^{a} is hydrogen, halogen, —OR¹ (where R¹ is as defined in formula (I) and preferably is hydrogen or C₁₋₄ alkyl), substituted or unsubstituted C₁₋₄ alkyl, or substituted or unsubstituted C₁₋₄ alkenyl; and R^{b} is hydrogen, halogen, or —OR¹(where R¹ is as defined in formula (I) and preferably is hydrogen or C₁₋₄ alkyl).

In one embodiment of any of formulae (I-CCXXXIII) where L is —NR^{c}—, Ar¹ is a substituted or unsubstituted phenyl and Z is substituted or unsubstituted phenyl, or a substituted or unsubstituted 5- or 6-membered heteroaryl, or a substituted or unsubstituted 5- or 6-membered heterocyclyl, where R^{c} is hydrogen or —CO₂R¹.

In one embodiment of any of formulae (I-CCXXXII) where L is —NR^{c}—, Ar¹ is a substituted or unsubstituted phenyl and Z is substituted or unsubstituted phenyl, or a substituted or unsubstituted 5- or 6-membered heteroaryl, where R^{c} is hydrogen or —CO₂R¹.

In one embodiment of any of formulae (I-CCXXXII) where L is —NR^{c}—, Ar¹ is a substituted or unsubstituted phenyl and Z is substituted or unsubstituted phenyl, or a substituted or unsubstituted 5- or 6-membered heteroaryl, where R^{c} is hydrogen or —CO₂Me.

In one embodiment of any of formulae (I-CCXXXII) where L is —C(O)—, Ar¹ is a substituted or unsubstituted phenyl and Z is substituted or unsubstituted phenyl, a substituted or unsubstituted 5- or 6-membered heteroaryl, or a substituted or unsubstituted 3- or 10-membered heterocyclyl.

In one embodiment of any of formulae (I-CCXXXII) where L is —C(O)—, Ar¹ is a substituted or unsubstituted phenyl and Z is a substituted or unsubstituted 3- or 10-membered heterocyclyl, or —NR¹⁷R¹⁸.

In one embodiment of any of formulae (I-CCXXXII) where L is —O—, Ar¹ is a substituted or unsubstituted phenyl and Z is substituted or unsubstituted phenyl, a substituted or unsubstituted 5- or 6-membered heteroaryl, or a substituted or unsubstituted 3- or 10-membered heterocyclyl.

In another embodiment where L is a bond or C═NR^{d}, Z, Y¹, Y², Y³, and Y⁴ are as defined for formula (I); and X¹, X², X³, X⁴, and X⁵ are as defined in formula (II), with the proviso that at least one of Y¹, Y², Y³ and Y⁴ is other than hydrogen.

In another embodiment where L is a bond or C═NR^{d}, Z, Y¹, Y², Y³, and Y⁴ are as defined for formula (I); and X¹, X², X³, X⁴, and X⁵ are as defined in formula (II), with the proviso and that when three of Y¹, Y², Y³ and Y⁴ are hydrogen, the other is not —OR⁶.

In one preferred embodiment of any of formulae (I-CCXXXII) where L is NR^{c}, R^{c} is hydrogen or —CO₂R¹, Y^{a} is hydrogen, Y¹ is chlorine, Z^{a} and Z¹ are hydrogen, X^{a} is hydrogen, and X¹ is substituted or unsubstituted C₁₋₈alkyl.

In one preferred embodiment of any of formulae (I-CCXXXII) where L is NR^{c}, R^{c} is hydrogen or —CO₂R¹, Y^{a} is hydrogen, Y¹ or Y^{b} is halogen, preferably chlorine, Z^{a}, Z^{b} and Z¹ are hydrogen, X^{a} and X^{b} are hydrogen, and X¹ is substituted or unsubstituted C₁₋₈alkyl.

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine; and

Z is selected from the group consisting of:

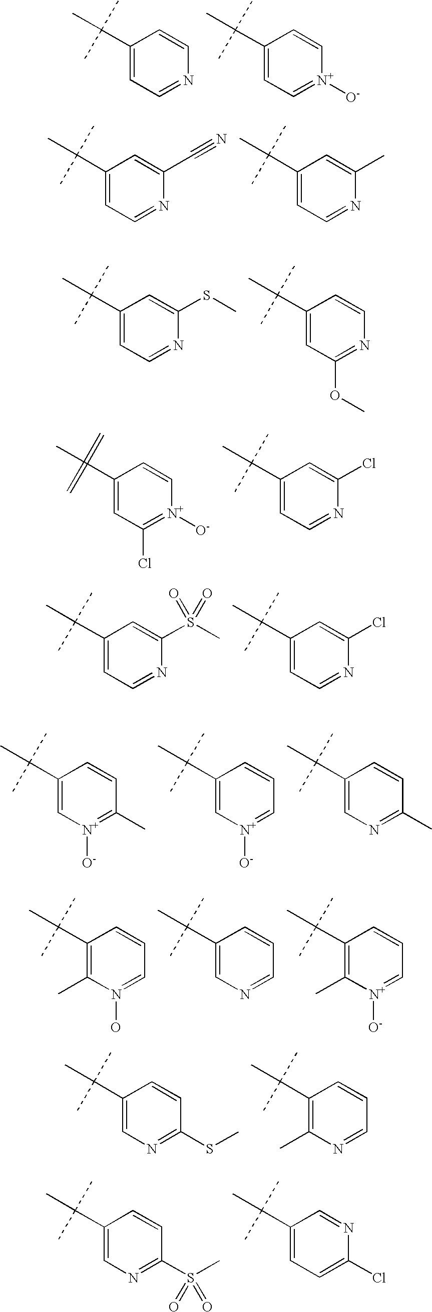

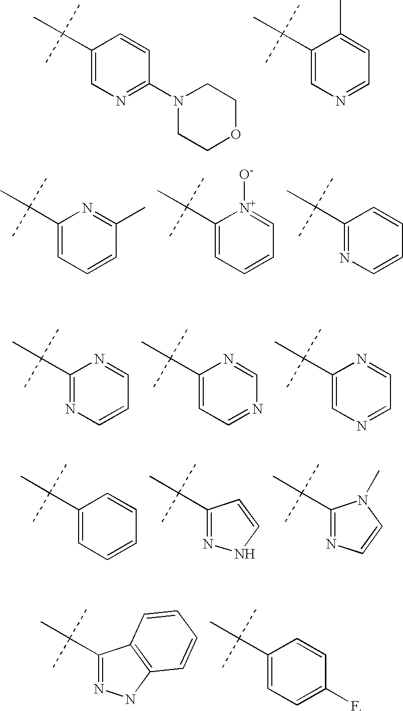

In one embodiment of any of formulae (I-CCXXXII),

Y³ is hydrogen;

Y² is halogen;

Y¹ and Y⁴ are each independently hydrogen or halogen; and

Z is selected from the group consisting of:

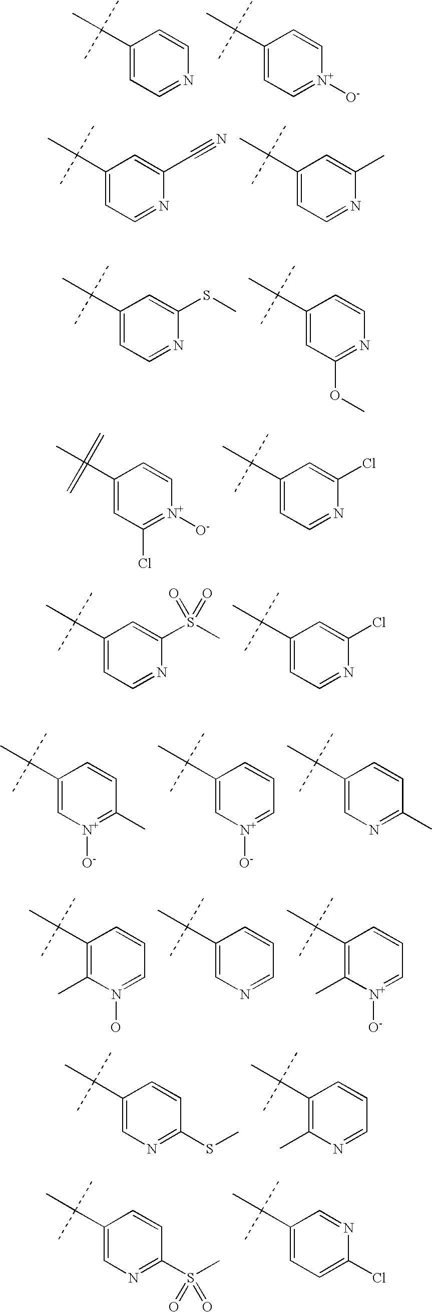

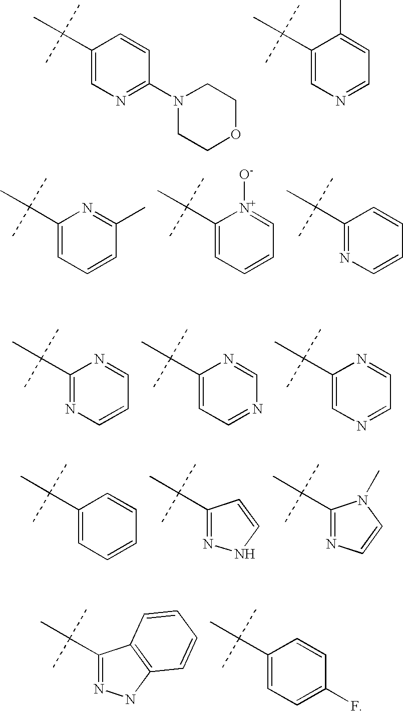

In one embodiment of any of formulae (I-CCXXXII),

Y² is halogen;

Y¹, Y³ and Y⁴ are each independently hydrogen or halogen; and

Z is selected from the group consisting of:

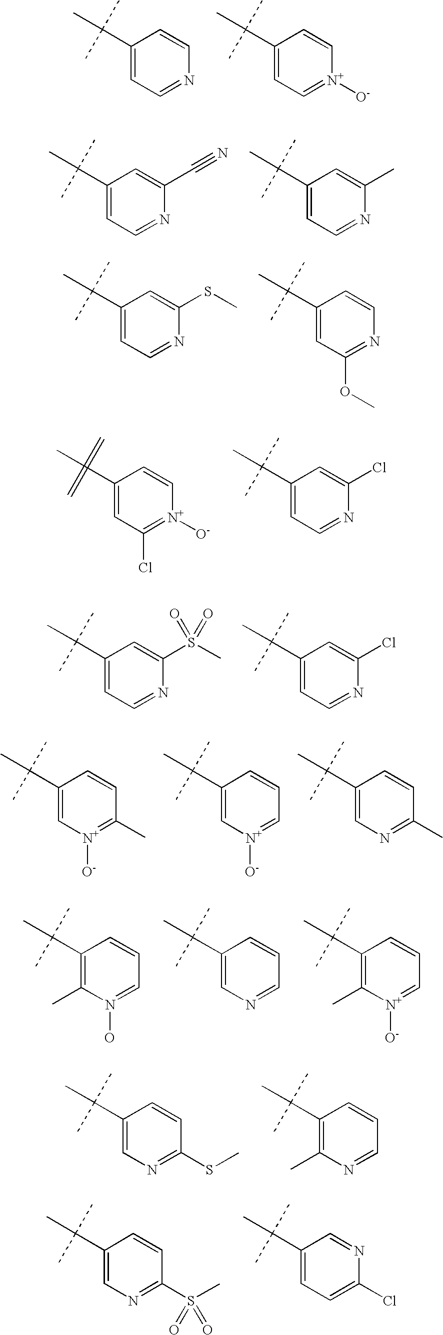

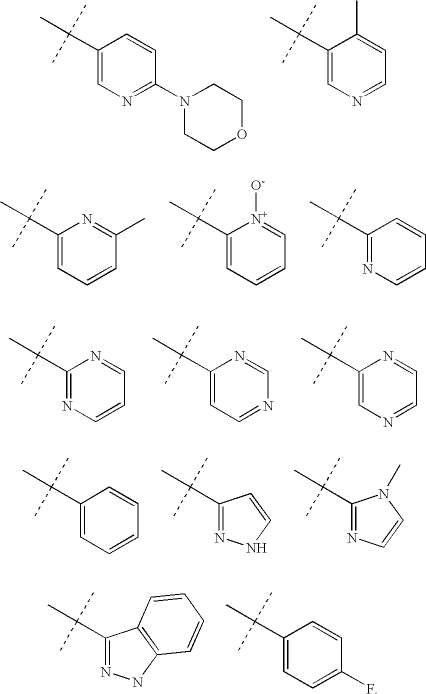

In one embodiment of any of formulae (I-CCXXXII),

Y³ is hydrogen;

Y² is halogen;

Y¹ and Y⁴ are each independently hydrogen or halogen; and

Z is selected from the group consisting of:

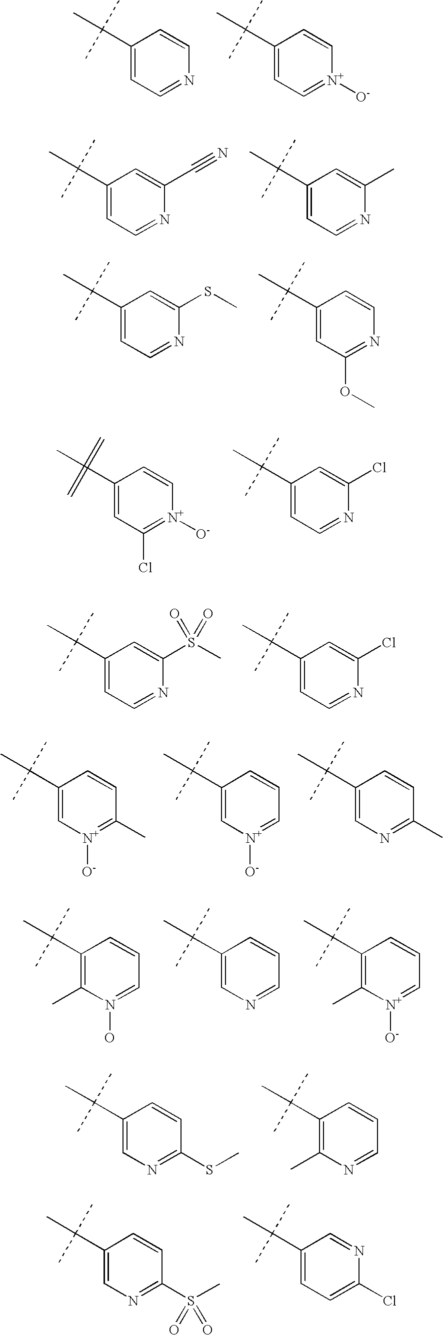

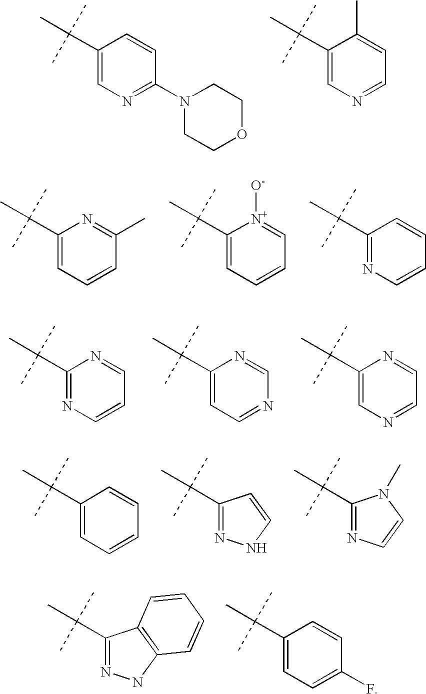

In one embodiment of any of formulae (I-CCXXXII):

Y³ is hydrogen;

Y¹ is chlorine or fluorine, with the proviso that when Y¹ is chlorine, both Y² and Y⁴ are hydrogen or alternatively, if Y¹ is fluorine, then either Y² or Y⁴ is also fluorine;

X¹ is selected from the group consisting of:

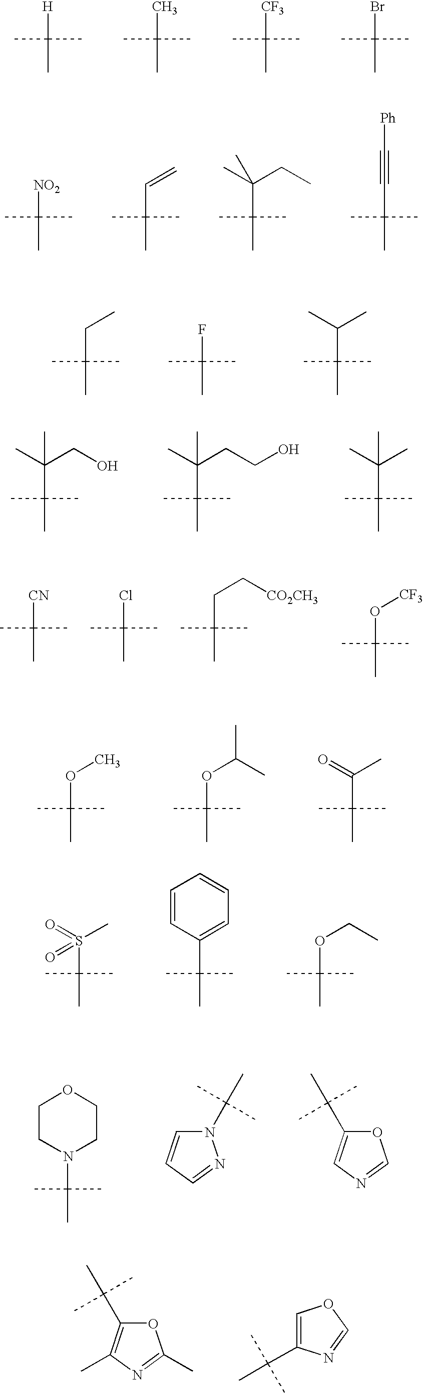

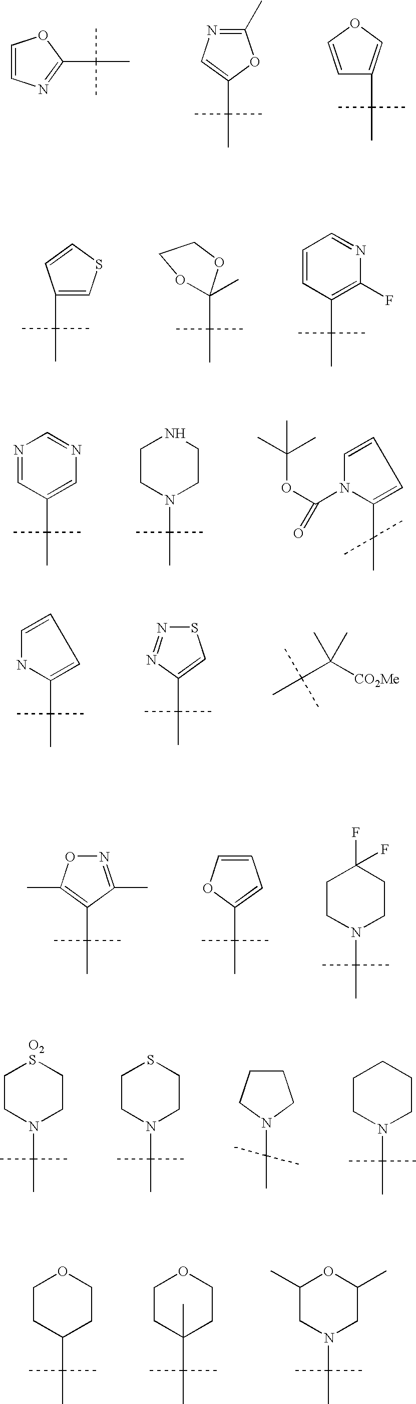

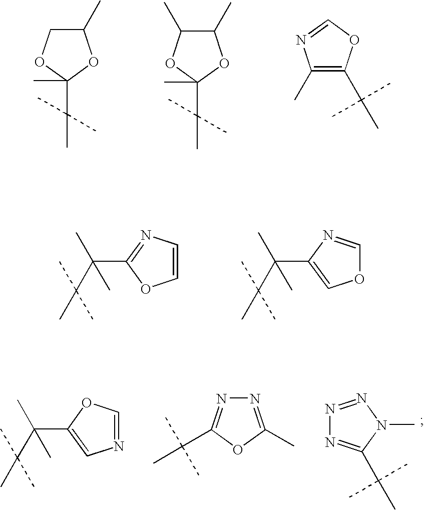

X³, X⁴, and X⁵ are hydrogen;

X² is halogen, —CN, —NO₂, —OCF₃, —OCH₃, —CH₃, —CF₃, —CO₂H, —CONHCH₃, or hydrogen;

Z is selected from the group consisting of:

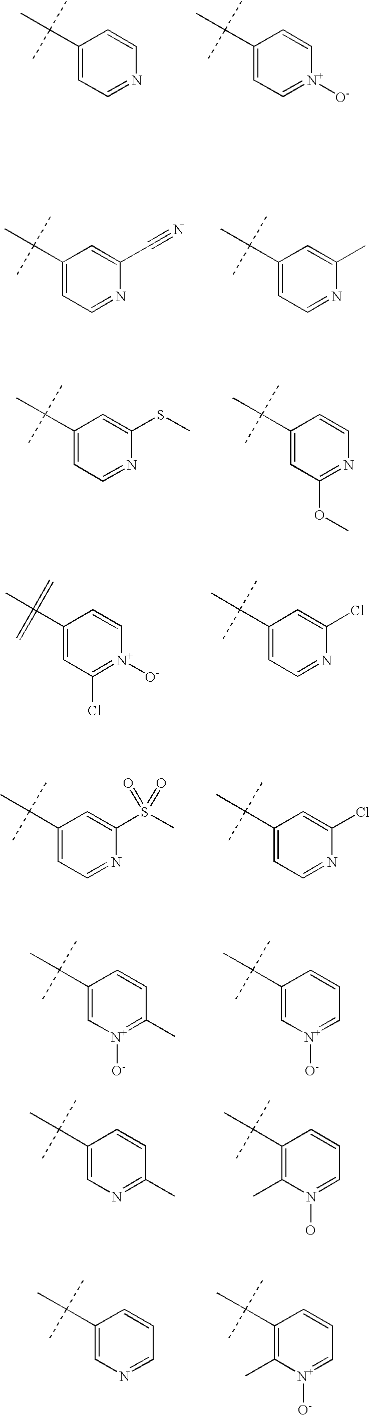

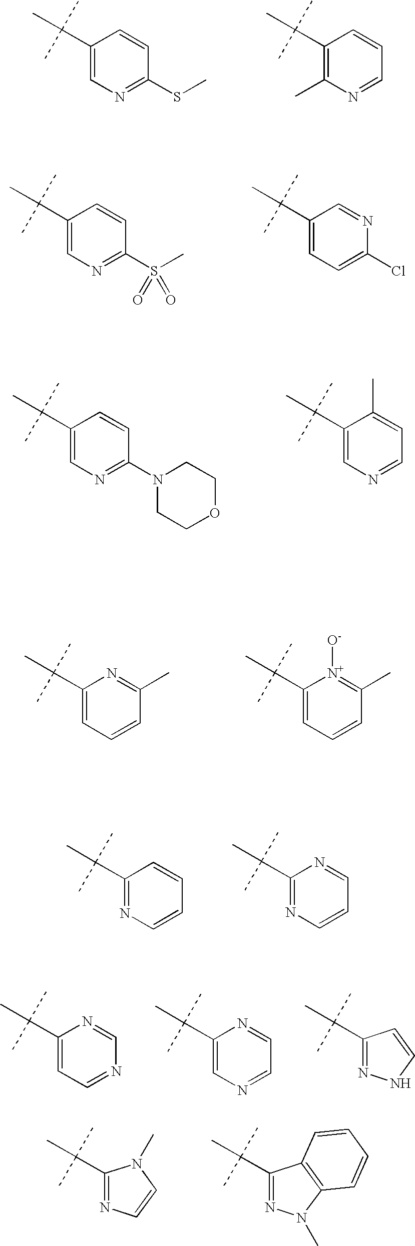

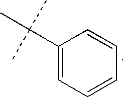

In one embodiment of any of formulae (I-CCXXXII):

Y² is halogen;

Y¹, Y³ and Y⁴ are each independently hydrogen or halogen;

X¹ is selected from the group consisting of:

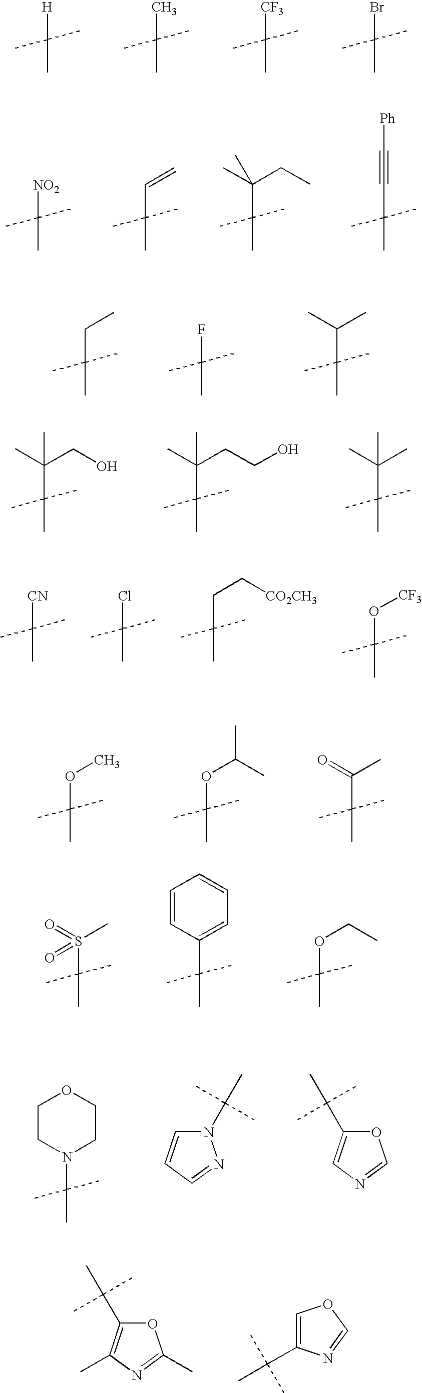

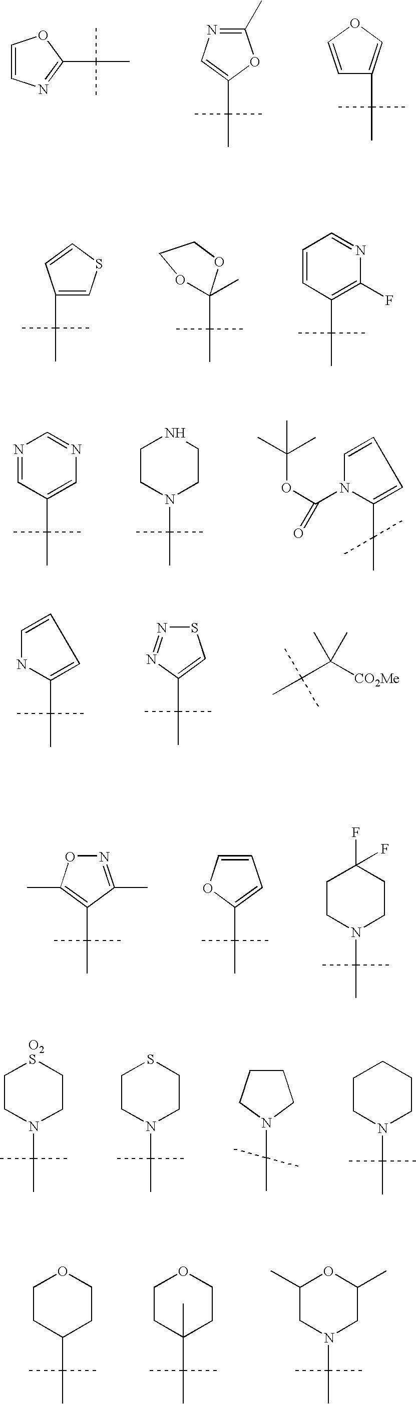

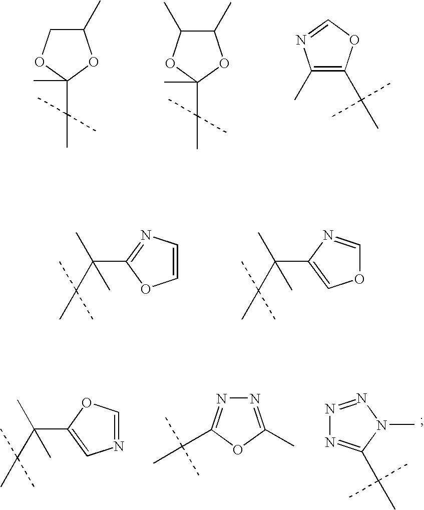

X³, X⁴, and X⁵ are hydrogen;

X² is halogen, —CN, —NO₂, —OCF₃, —OCH₃, —CH₃, —CF₃, —CO₂H, —CONHCH₃, or hydrogen;

Z is selected from the group consisting of:

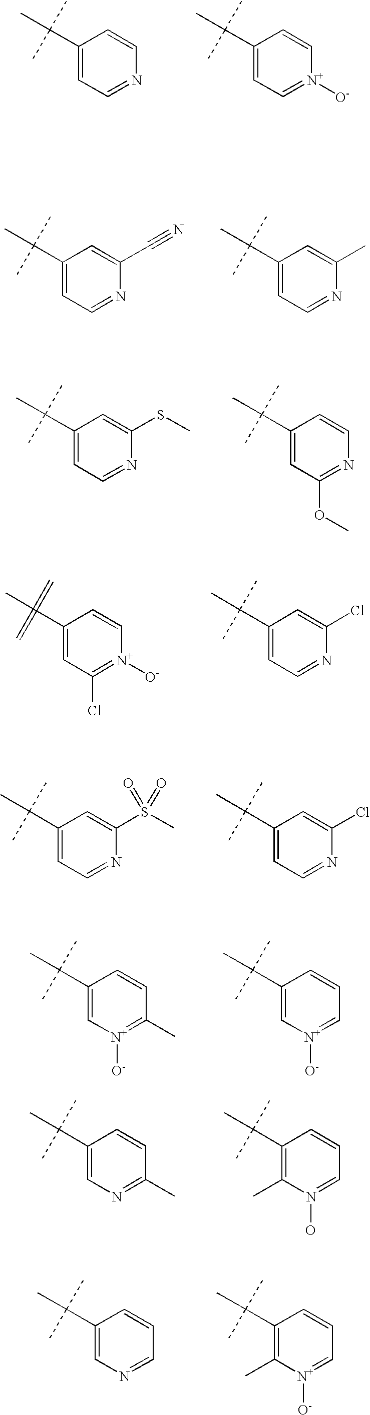

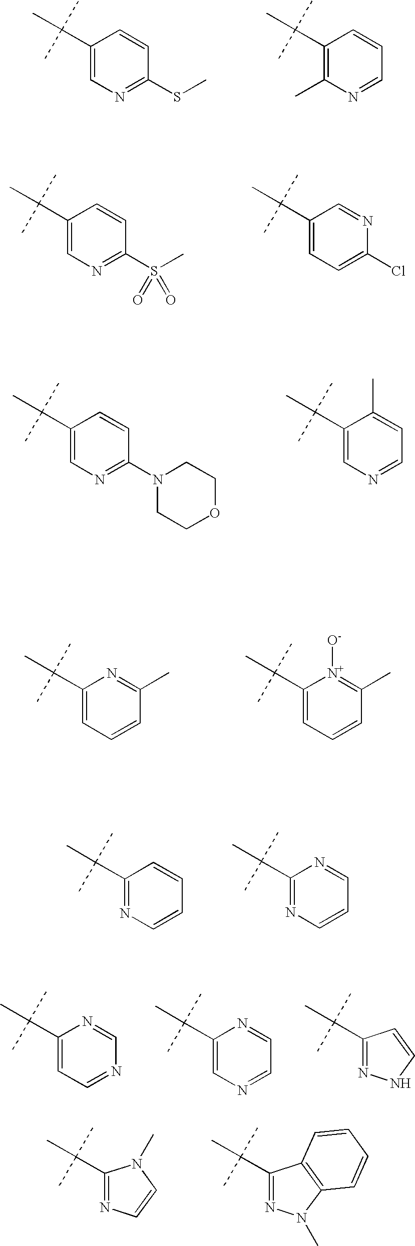

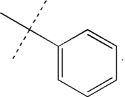

In one embodiment of formula (I-CCXXXII):

Y³ is hydrogen;

Y¹ is chlorine or fluorine, with the proviso that when Y¹ is chlorine, both Y² and Y⁴ are hydrogen or alternatively, if Y¹ is fluorine, then either Y² or Y⁴ is also fluorine;

X¹ is selected from the group consisting of:

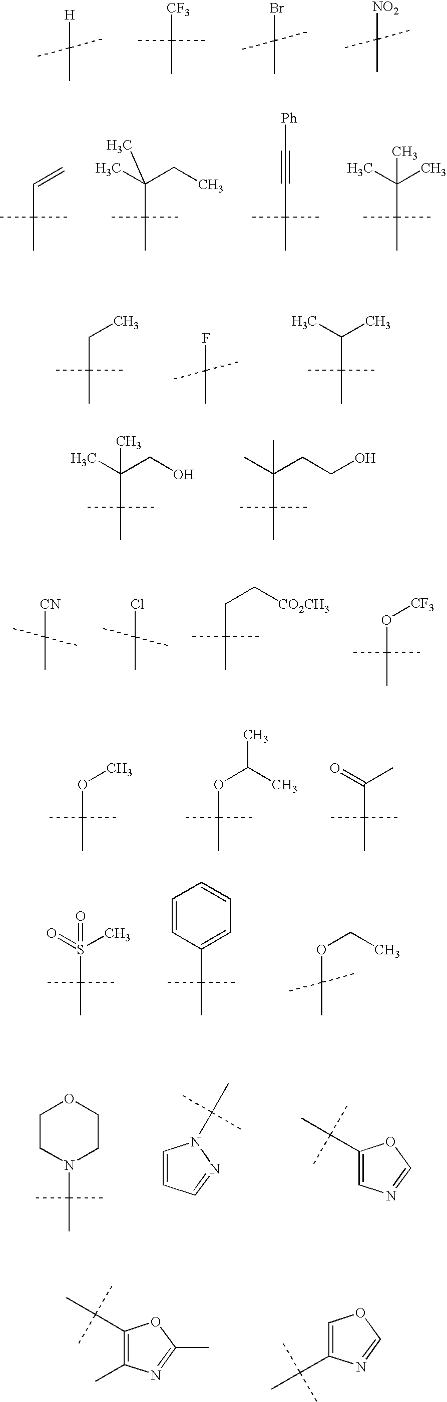

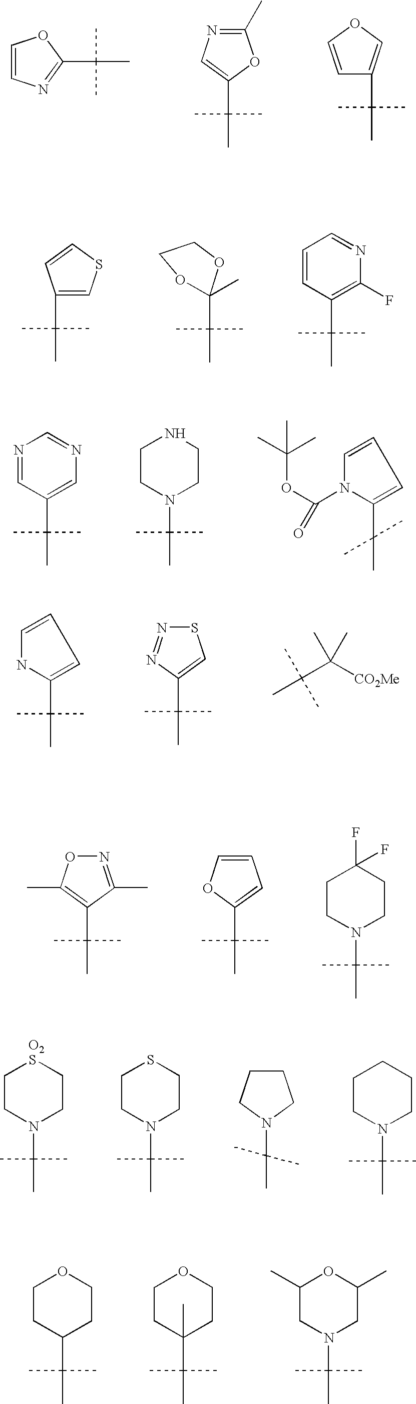

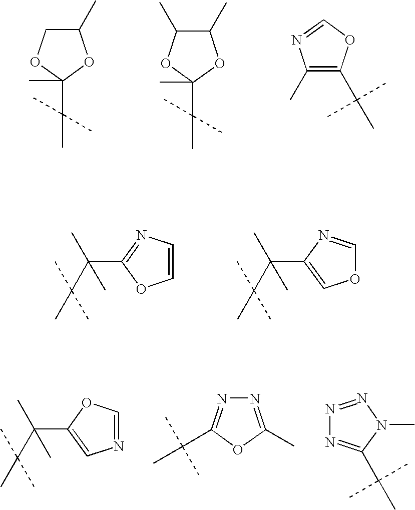

X³, X⁴, and X⁵ are hydrogen;

X² is halogen, —CN, —NO₂, —OCF₃, —OCH₃, —CH₃, —CF₃, —CO₂H, —CONHCH₃, or hydrogen;

Z is selected from the group consisting of:

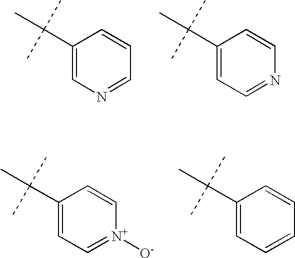

with the proviso that if X² is methyl, the X¹ is other than hydrogen.

In one embodiment of any of formulae (I-CCXXXII):

Y² is halogen;

Y³ is hydrogen;

Y¹ and Y⁴ are each independently hydrogen or halogen;

X¹ is selected from the group consisting of:

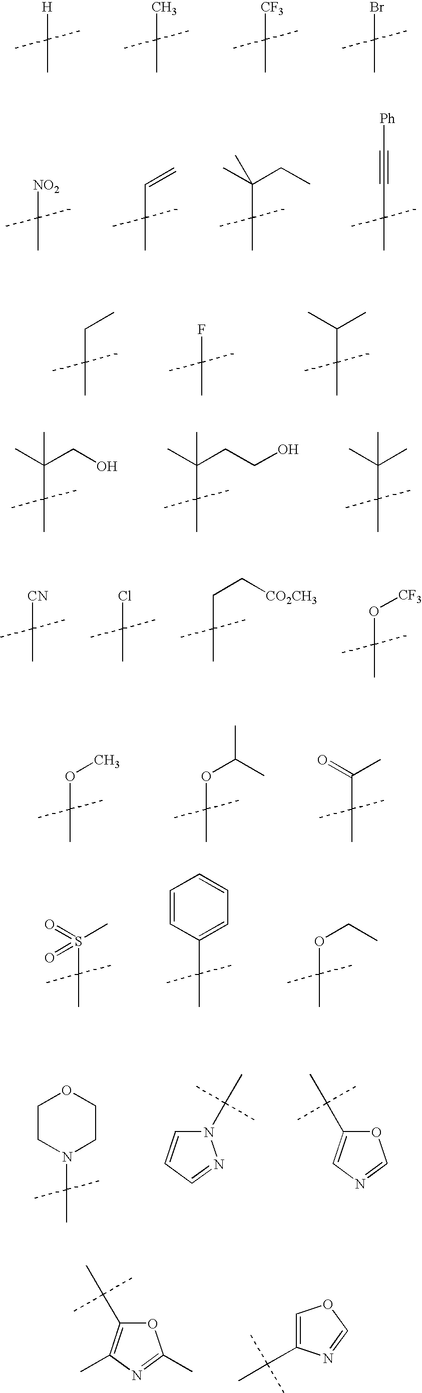

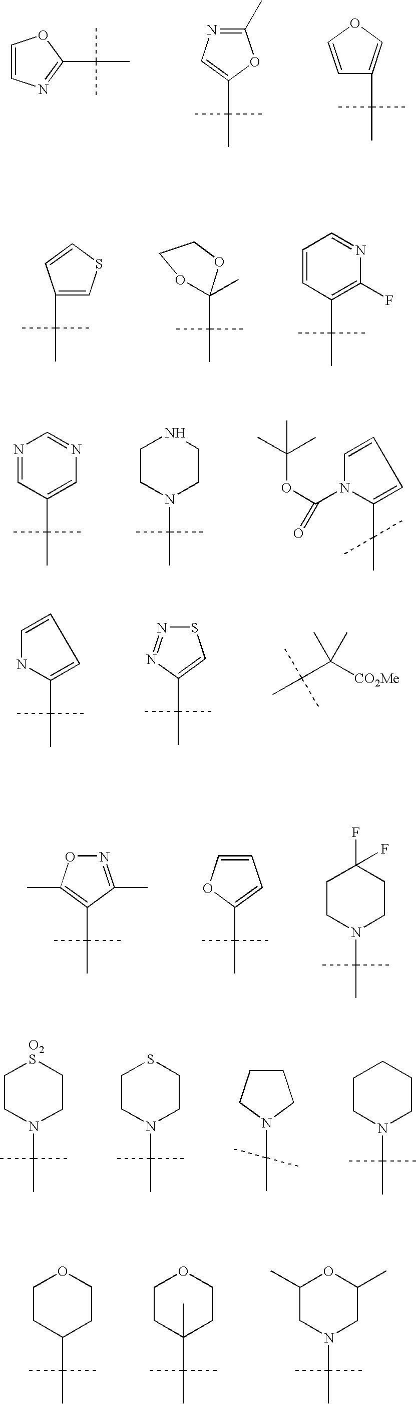

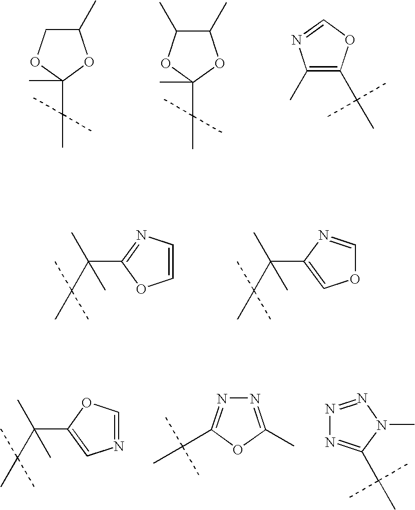

X³, X⁴, and X⁵ are hydrogen;

X² is halogen, —CN, —NO₂, —OCF₃, —OCH₃, —CH₃, —CF₃, —CO₂H, —CONHCH₃, or hydrogen;

Z is selected from the group consisting of:

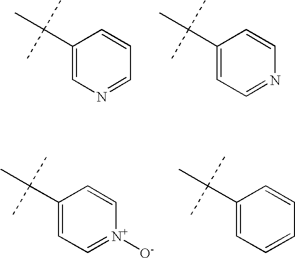

with the provisio that if X² is methyl, the X¹ is other than hydrogen.

In one embodiment of any of formulae (I-CCXXXII),

Y³ is hydrogen;

Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen; and

Z is selected from the group consisting of:

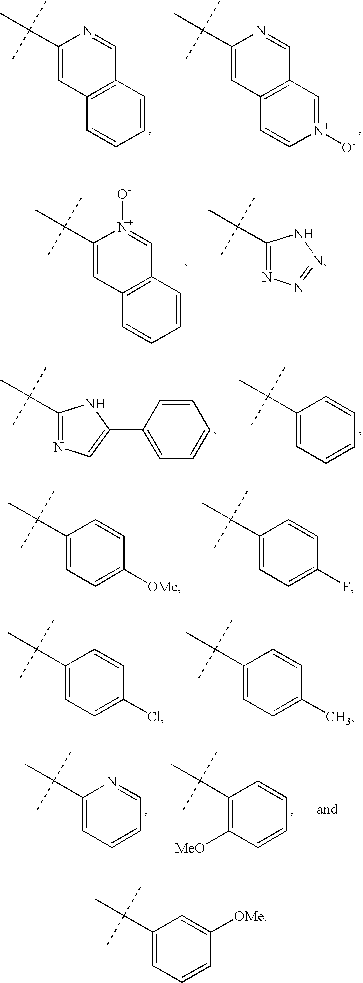

In one embodiment of any of formulae (I-CCXXXII),

Y² is halogen;

Y³ is hydrogen;

Y¹ and Y⁴ are each independently hydrogen or halogen; and

Z is selected from the group consisting of:

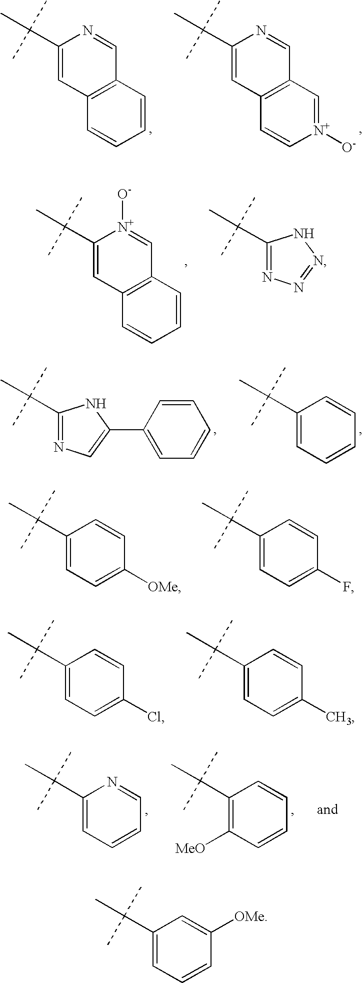

In one embodiment of any of formulae (I-CCXXXII),

Y³ is hydrogen;

Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen;

Z is selected from the group consisting of:

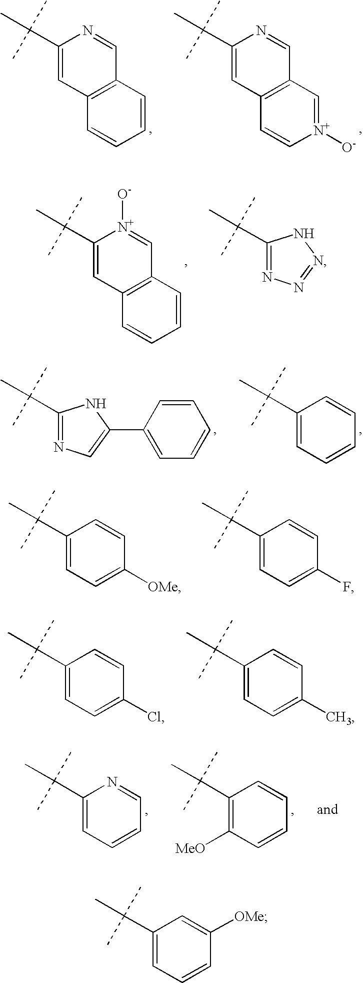

and X², X³ and X⁵ are hydrogen.

In one embodiment of any of formulae (I-CCXXXII),

Y² is halogen;

Y³ is hydrogen;

Y¹ and Y⁴ are each independently hydrogen or halogen;

Z is selected from the group consisting of:

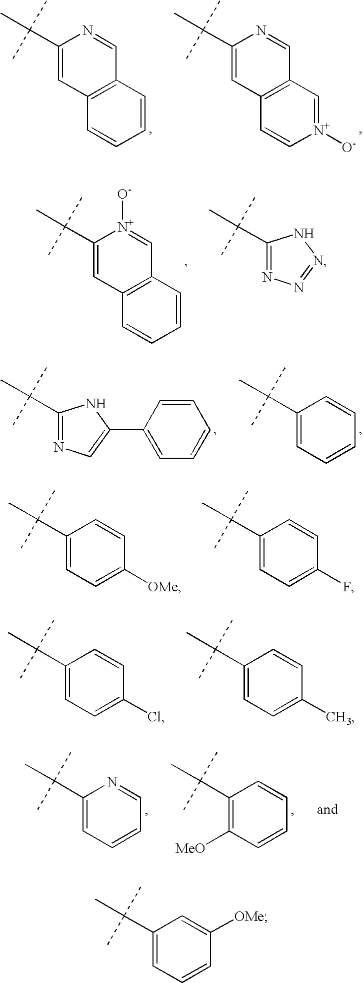

and X², X³ and X⁵ are hydrogen.

In one embodiment of any of formulae (I-CCXXXII),

Y³ is hydrogen;

Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen;

Z is selected from the group consisting of:

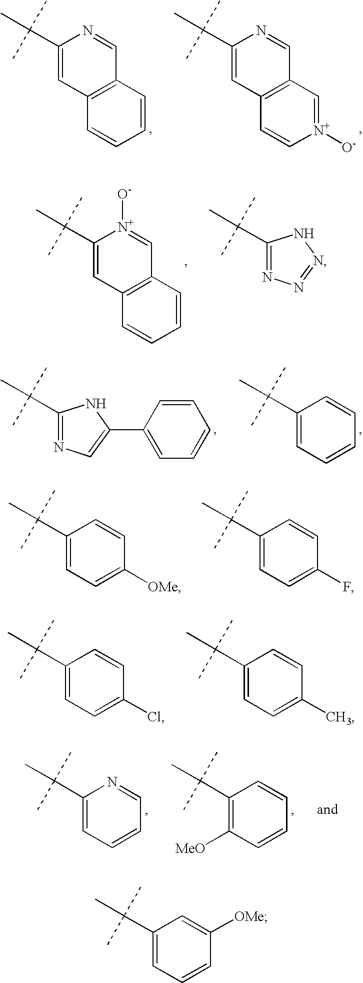

X², X³ and X⁵ are hydrogen; and

X¹ is selected from the group consisting of:

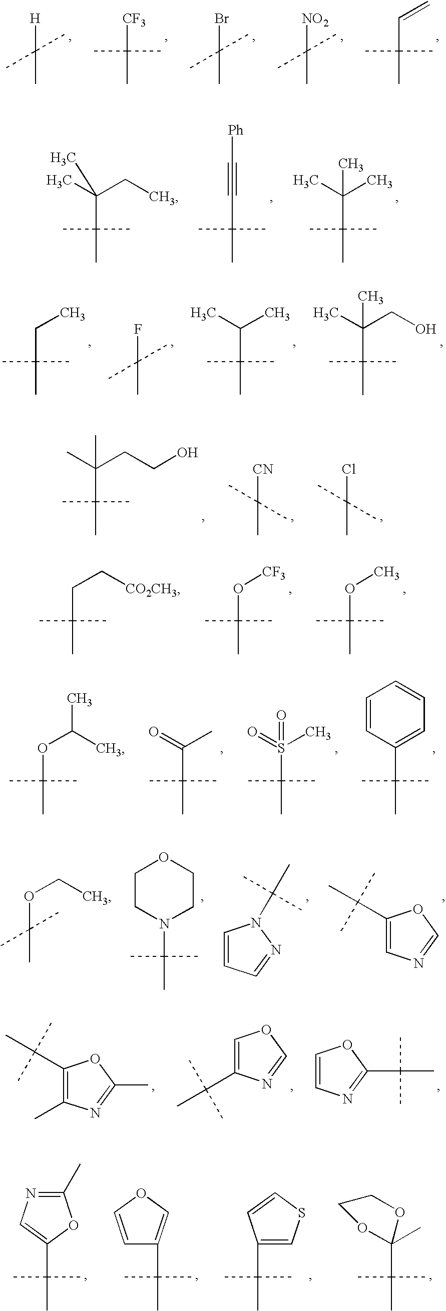

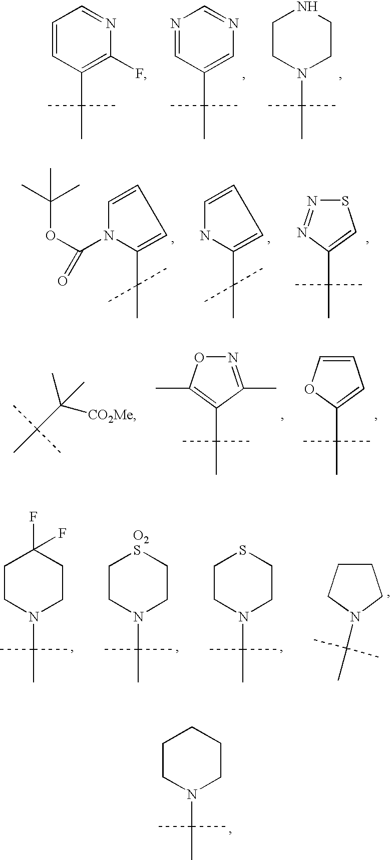

In one embodiment of any of formulae (I-CCXXXII),

Y² is halogen;

Y³ is hydrogen;

Y¹ and Y⁴ are each independently hydrogen or halogen;

Z is selected from the group consisting of:

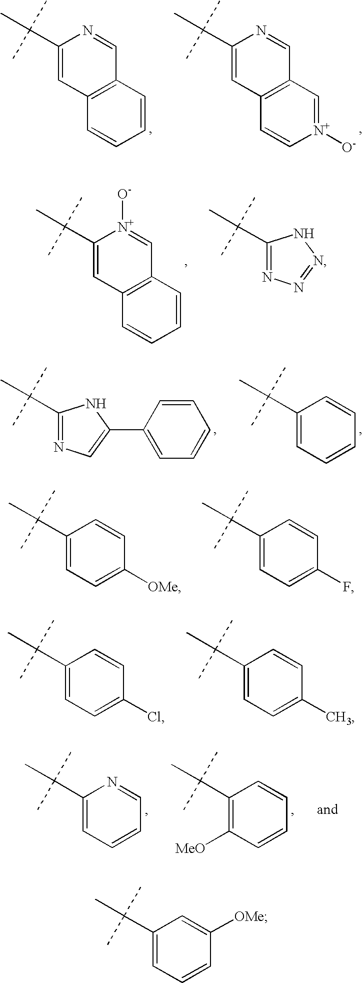

X³, X⁴ and X⁵ are hydrogen; and

X¹ is selected from the group consisting of:

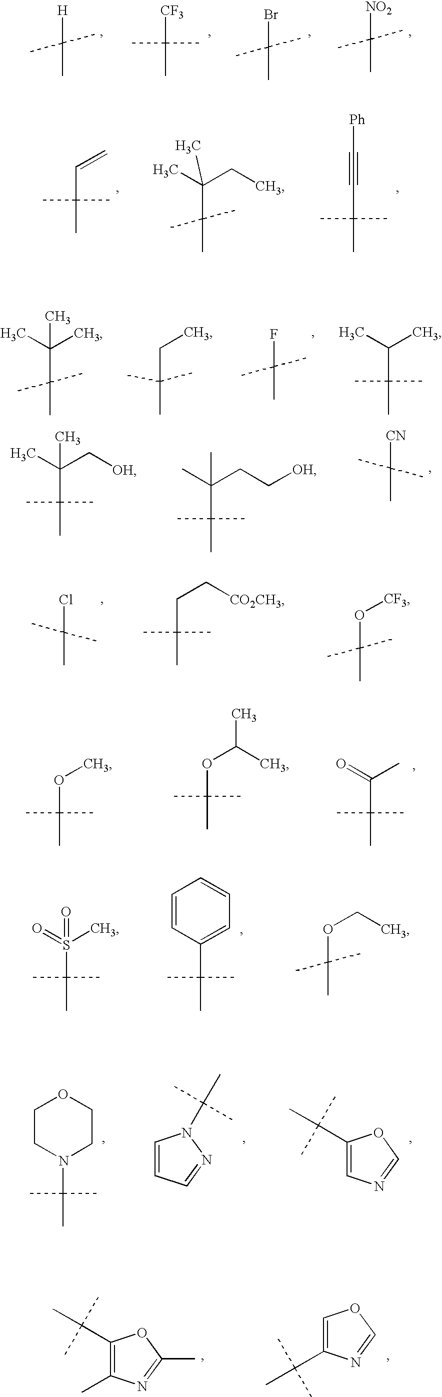

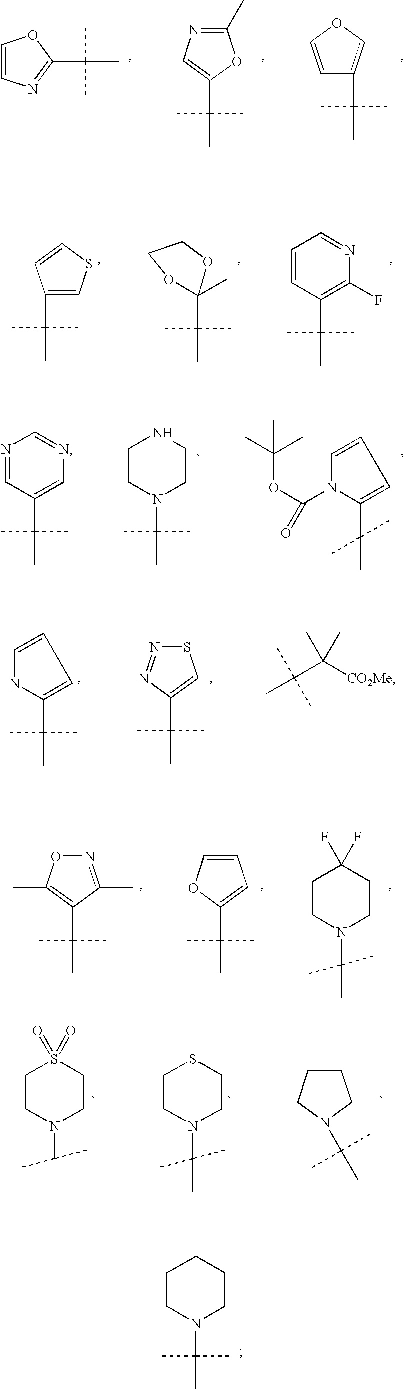

and X² is halogen, —CN, —NO₂, —OCF₃, —OCH₃, —CH₃, —CF₃, —CO₂H, —CONHCH₃, or hydrogen.

In one embodiment of any of formulae (I-CCXXXII),

Y³ is hydrogen;

Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen;

Z is selected from the group consisting of:

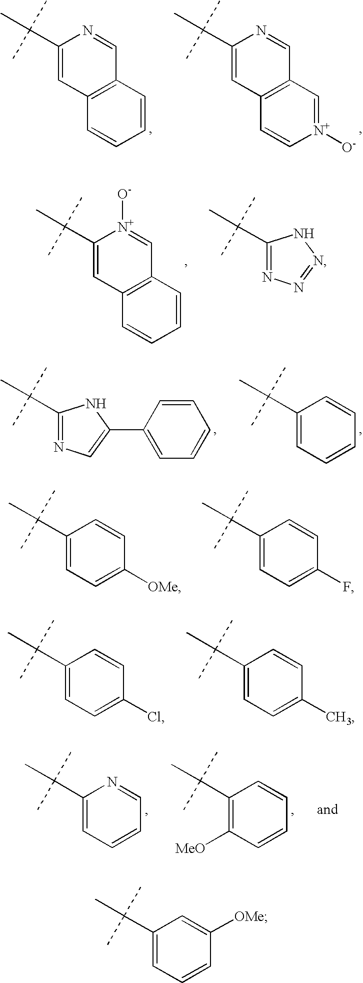

X³, X⁴ and X⁵ are hydrogen;

X² is halogen, —CN, —NO₂, —OCF₃, —OCH₃, —CH₃, —CF₃, —CO₂H, —CONHCH₃, or hydrogen; and

X¹ is selected from the group consisting of:

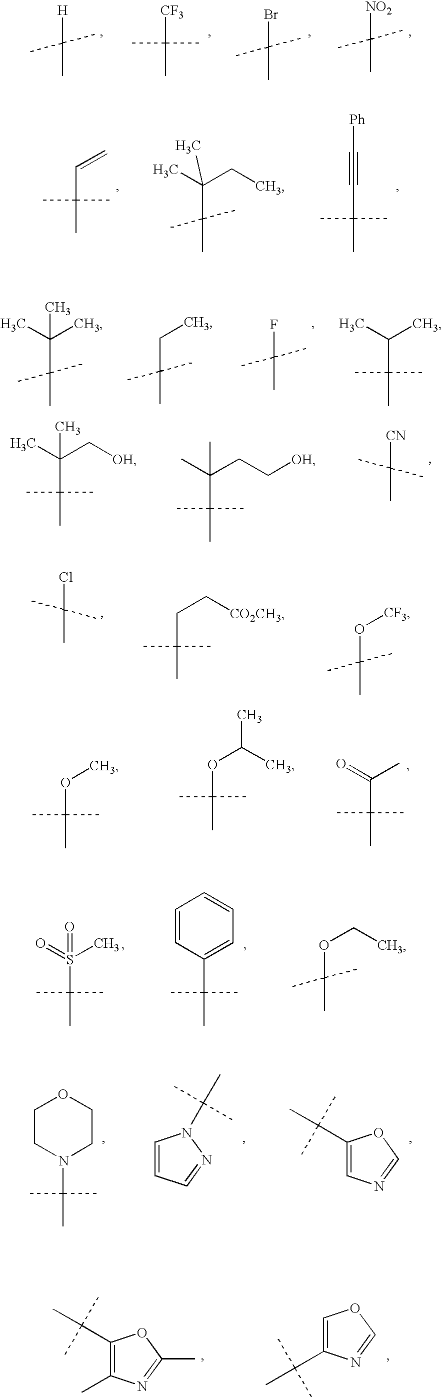

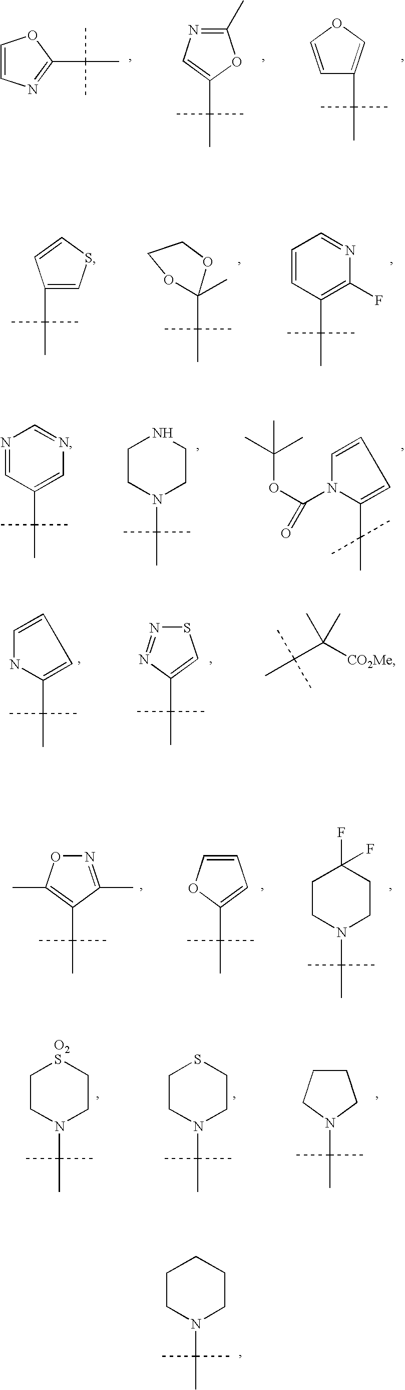

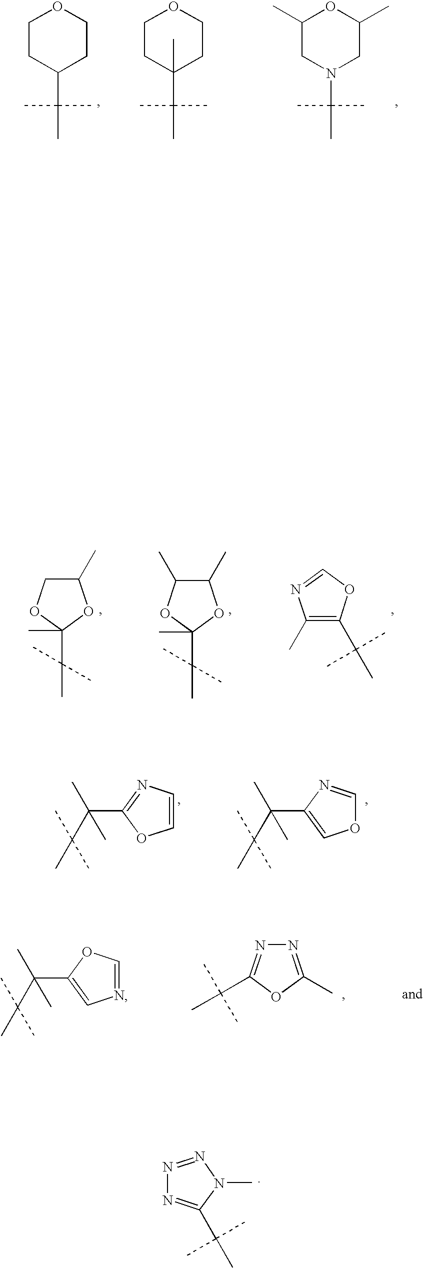

In one embodiment of any of formulae (I-CCXXXII),

Y² is halogen;

Y³ is hydrogen;

Y¹ and Y⁴ are each independently hydrogen or halogen;

Z is selected from the group consisting of:

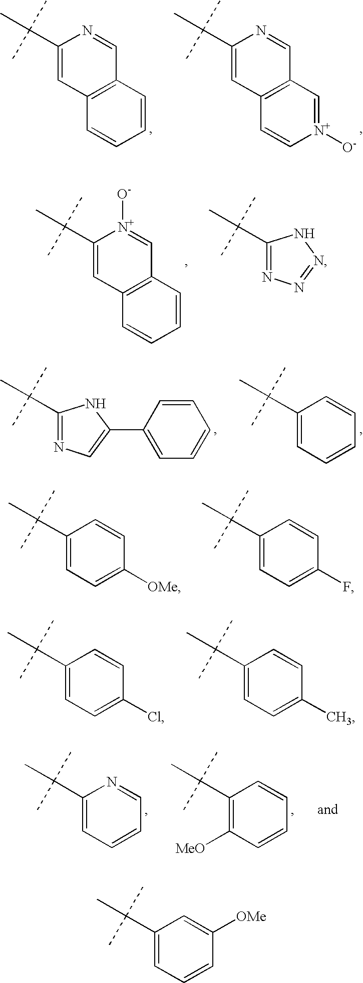

X³, X⁴ and X⁵ are hydrogen;

X² is halogen, —CN, or —CF₃; and

X¹ is selected from the group consisting of:

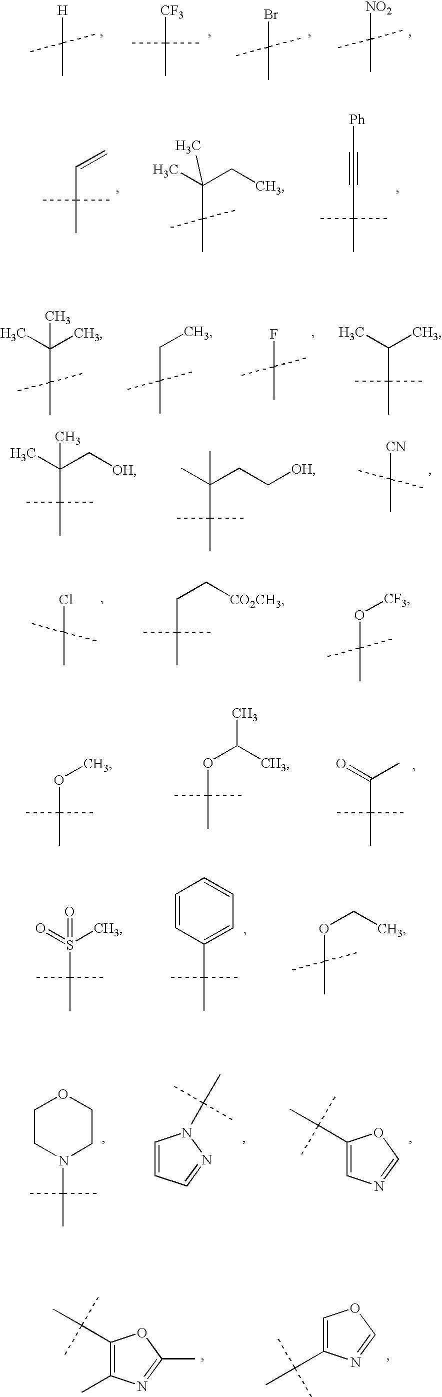

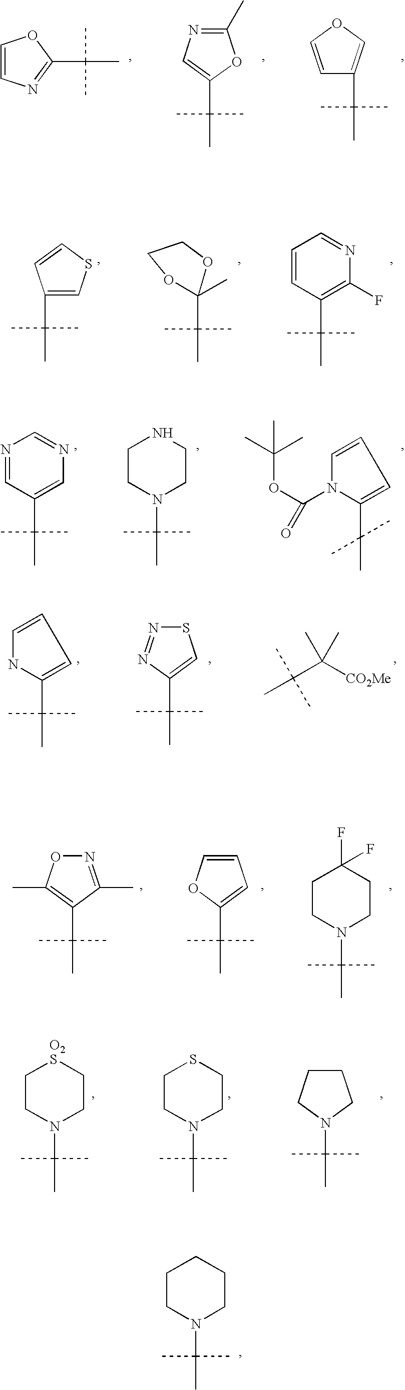

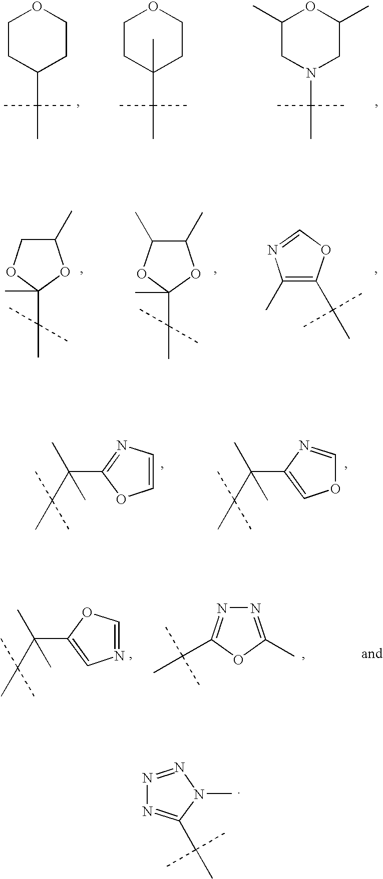

In one embodiment of any of formulae (I-CCXXXII),

Y³ is hydrogen;

Y¹ is chlorine or fluorine, with the following provisos: when Y¹ is chlorine, both Y² and Y⁴ are hydrogen; and when Y¹ is fluorine, either Y² or Y⁴ are fluorine, the other being hydrogen;

Z is selected from the group consisting of:

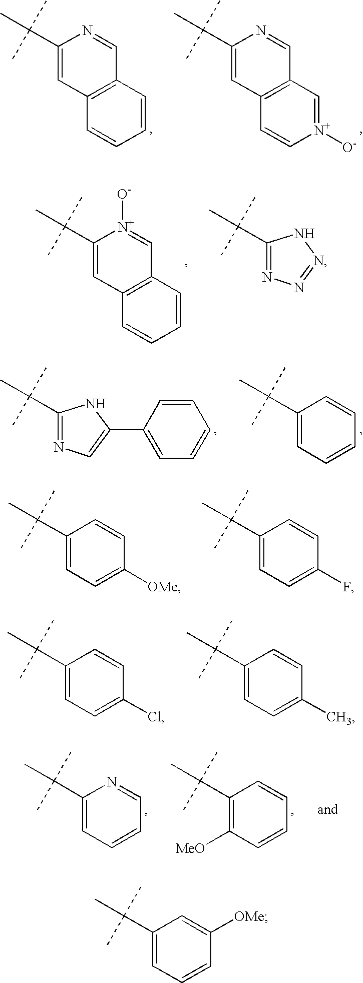

X³, X⁴ and X⁵ are hydrogen;

X³ is hydrogen; and

X¹ is selected from the group consisting of:

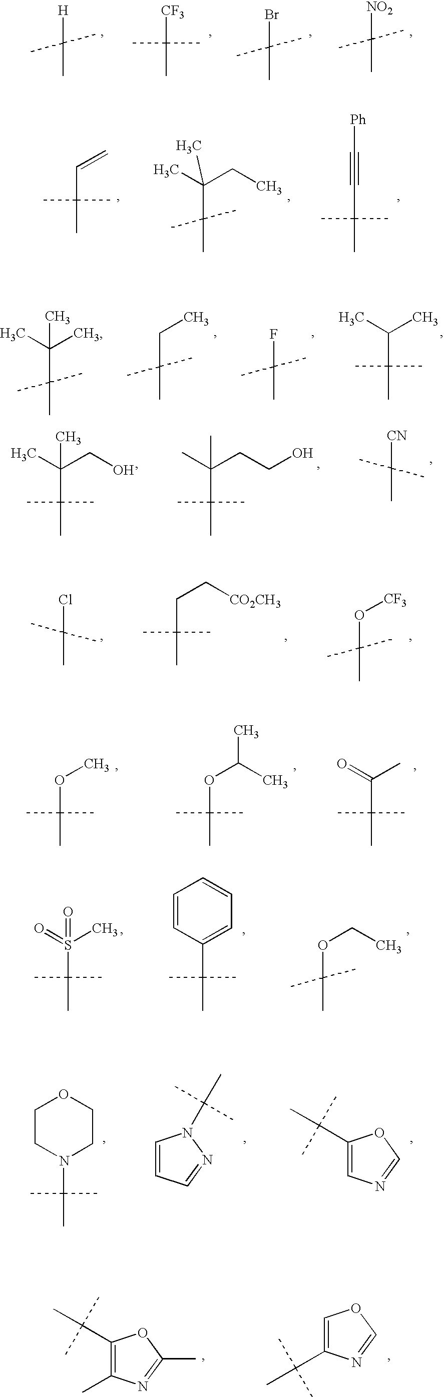

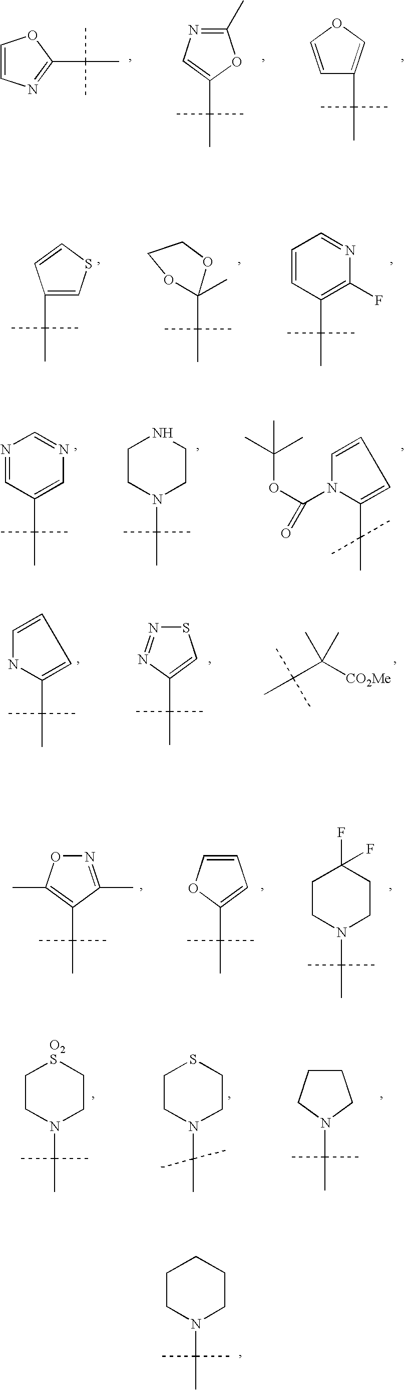

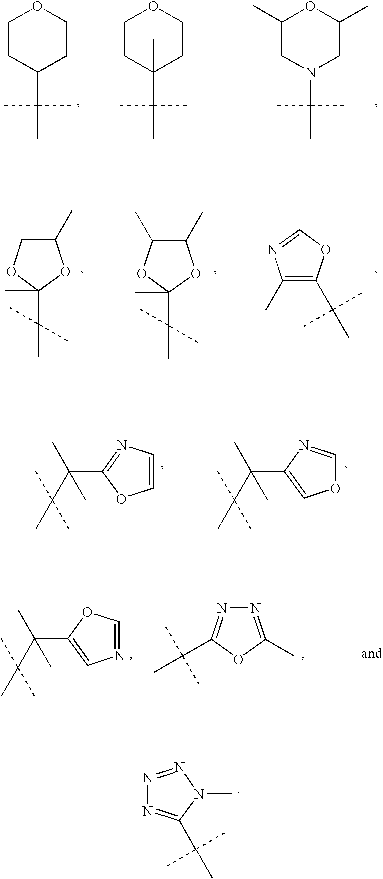

In one embodiment of any of formulae (I-CCXXXII),

Y² is halogen;

Y³ is hydrogen;

Y¹ and Y⁴ are each independently hydrogen or halogen;

Z is selected from the group consisting of:

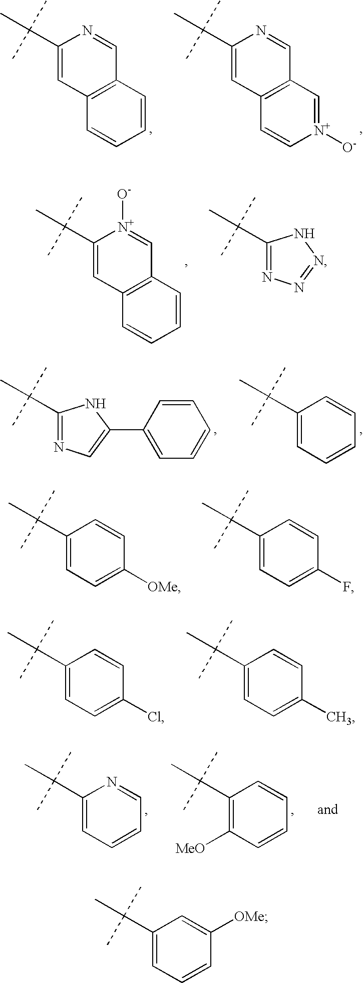

X³, X⁴ and X⁵ are hydrogen;

X² is hydrogen; and

X¹ is selected from the group consisting of:

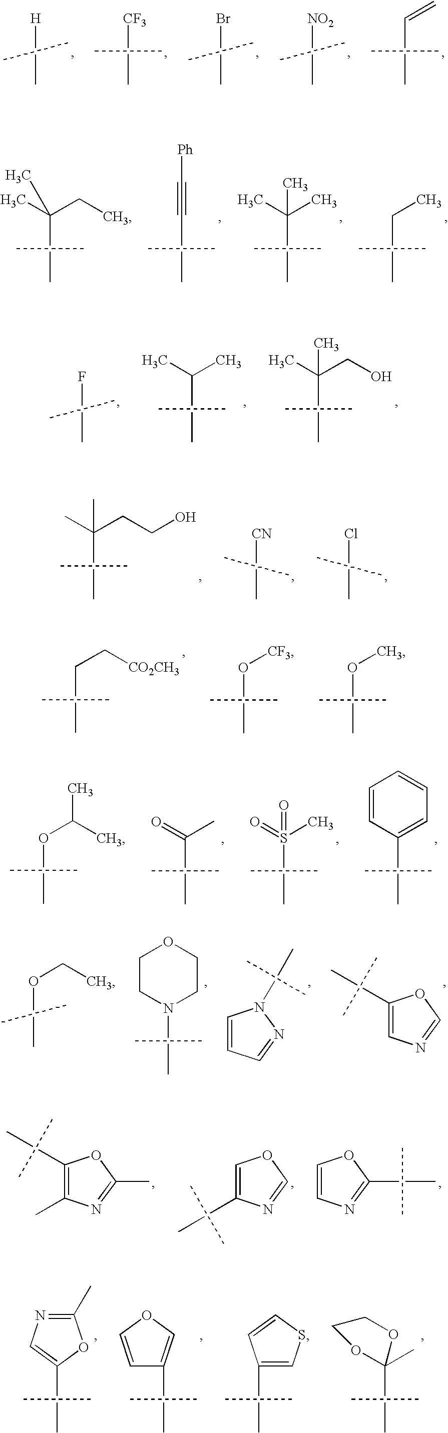

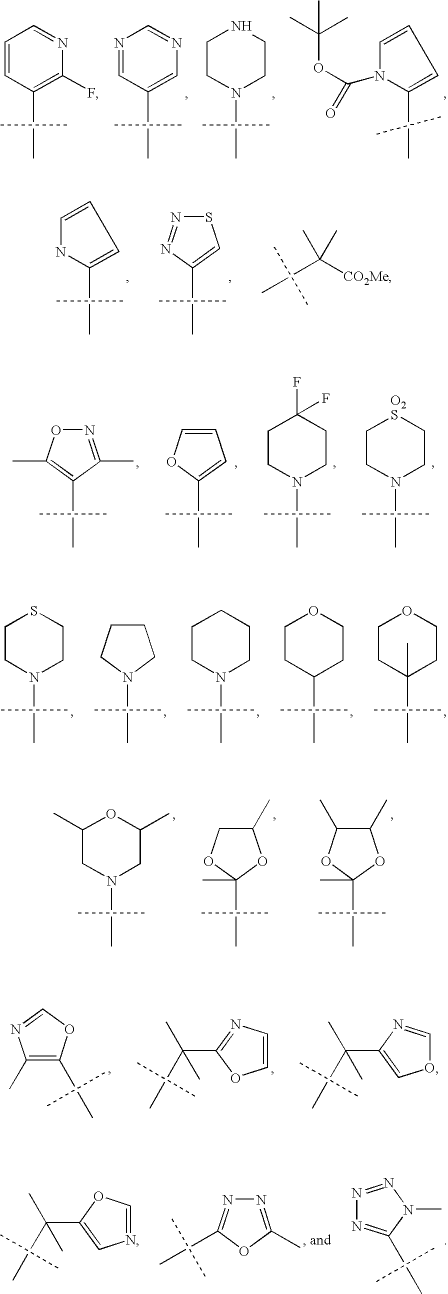

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine;

X², X³ and X⁵ are hydrogen; and

Z is selected from the group consisting of:

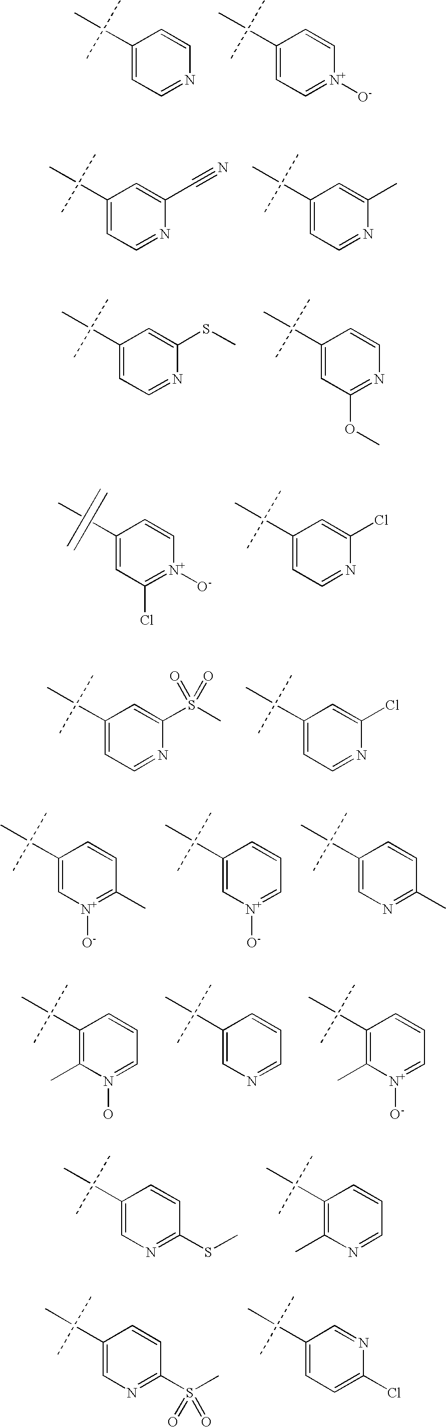

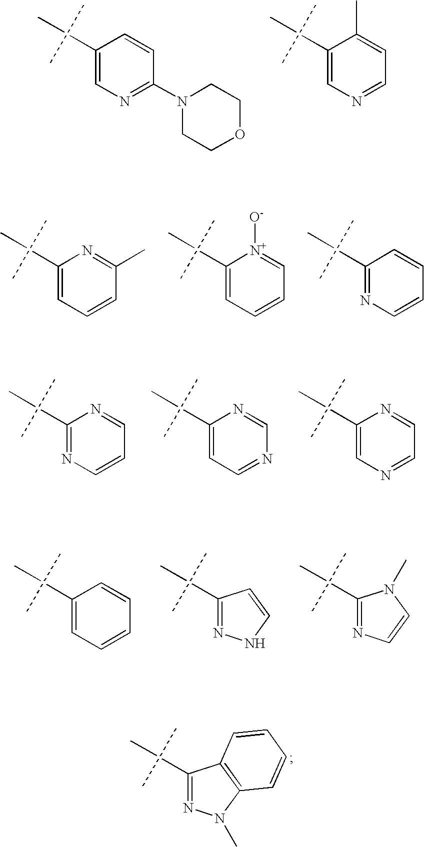

R^{c} when present is as defined for formula (I), preferably R^{c} is hydrogen or —C(O)R, more preferably COMe;

T when present is as defined for formula (I), preferably T is ═CHC(O)R⁴¹, ═CHC(O)₂R⁴¹, ═CH₂, ═NOR^{d} or ═NOMe, more preferably ═CHC(O)Me, ═CHC(O)₂Me.

In other embodiments Y² is halogen; Y³ is hydrogen; and Y¹ and Y⁴ are each independently hydrogen or halogen.

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine; and

X¹ is selected from the group consisting of:

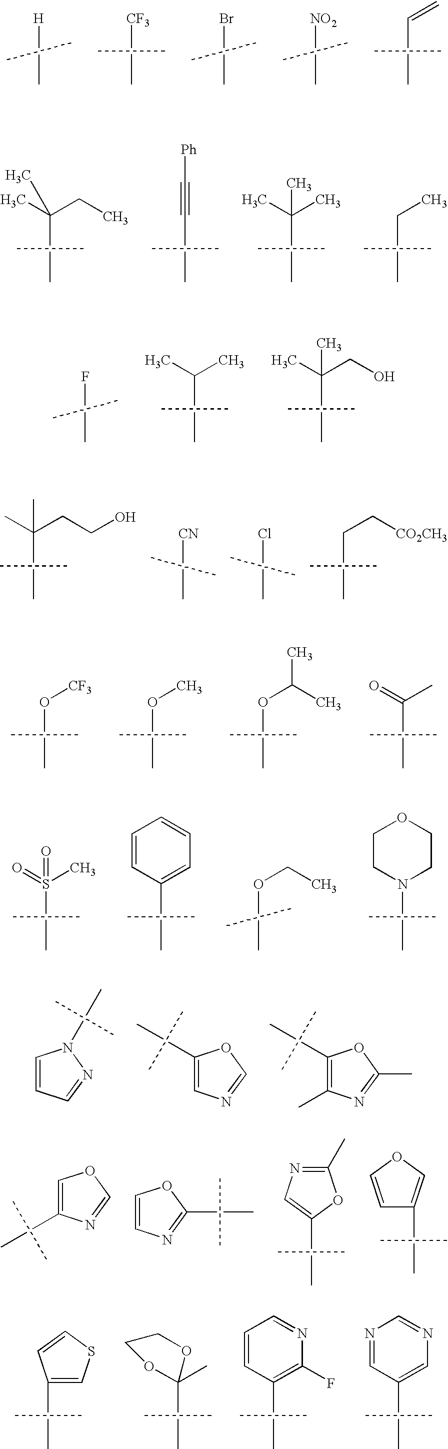

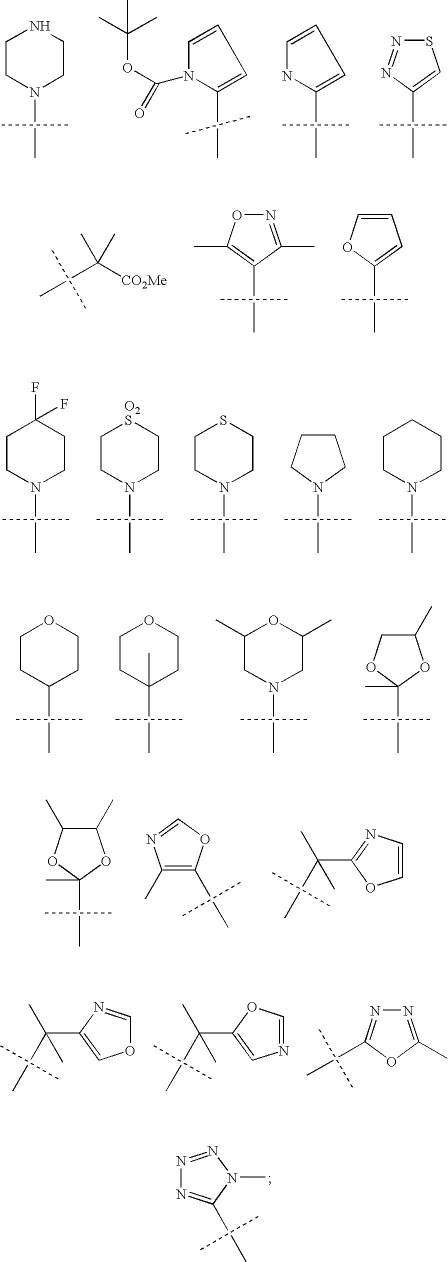

X², X³ and X⁵ are hydrogen; and

Z is selected from the group consisting of:

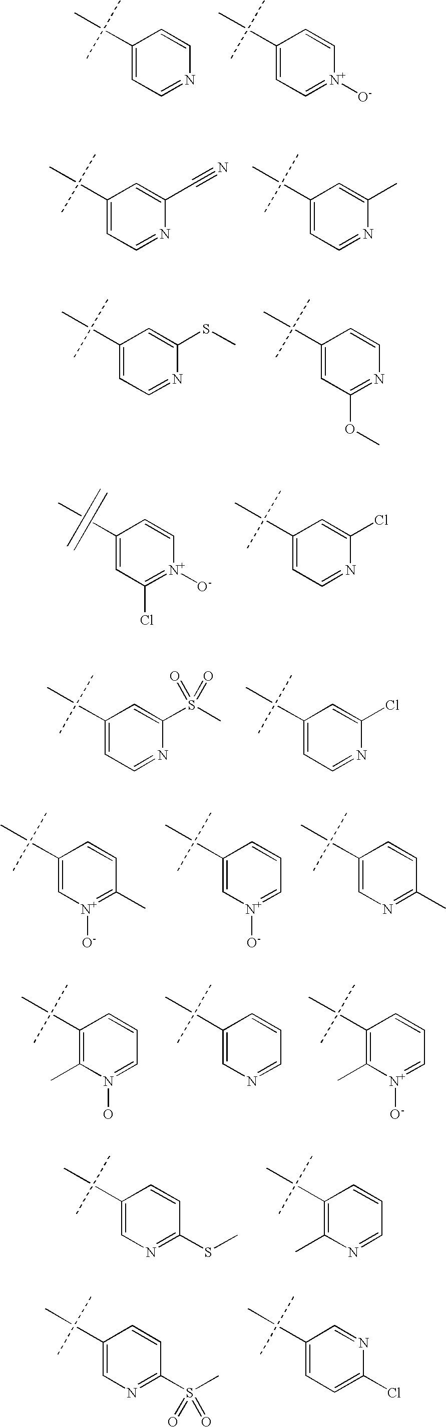

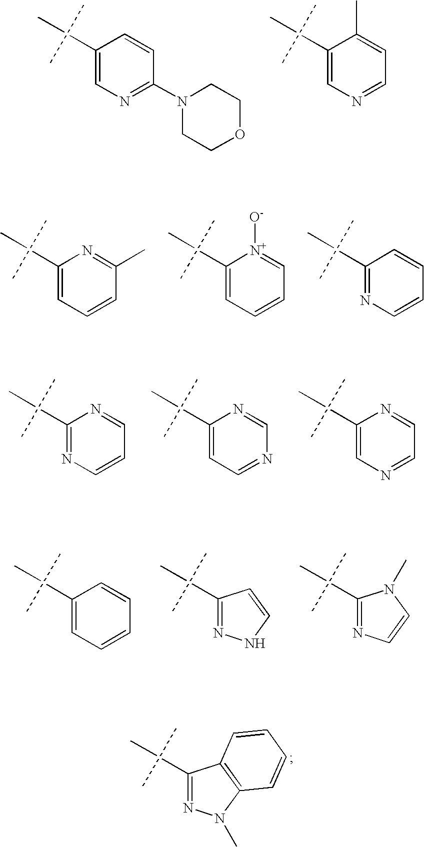

R^{c} when present is as defined for formula (I), preferably R^{c} is hydrogen or —C(O)R, more preferably COMe;

T when present is as defined for formula (I), preferably T is ═CHC(O)R⁴¹, ═CHC(O)₂R⁴¹, ═CH₂, ═NOR^{d}, or ═NOMe, more preferably ═CHC(O)Me, ═CHC(O)₂Me.

In other embodiments Y² is halogen; Y³ is hydrogen; and Y¹ and Y⁴ are each independently hydrogen or halogen.

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine;

X¹ is selected from the group consisting of:

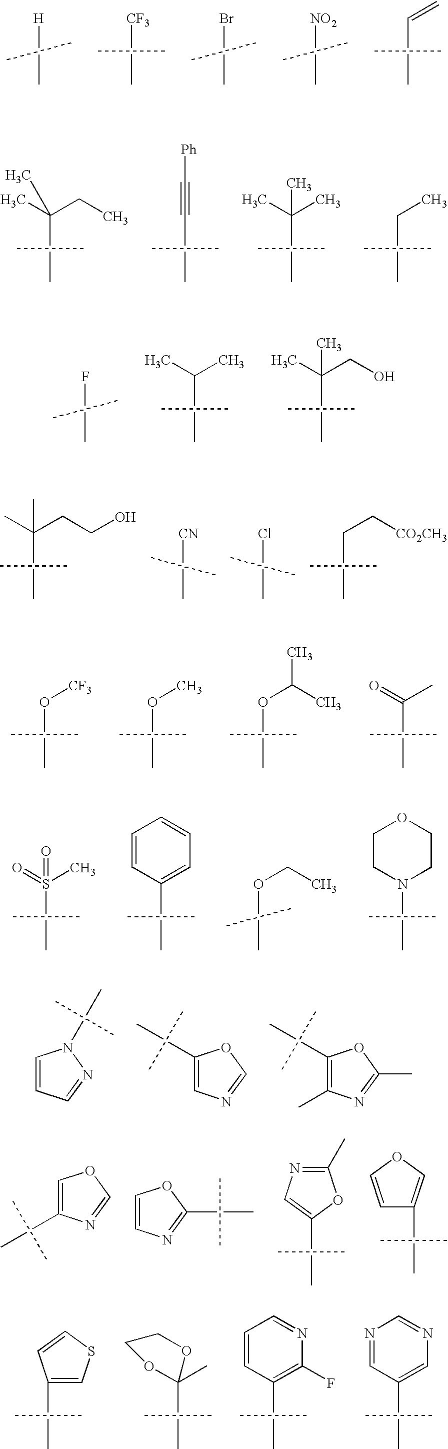

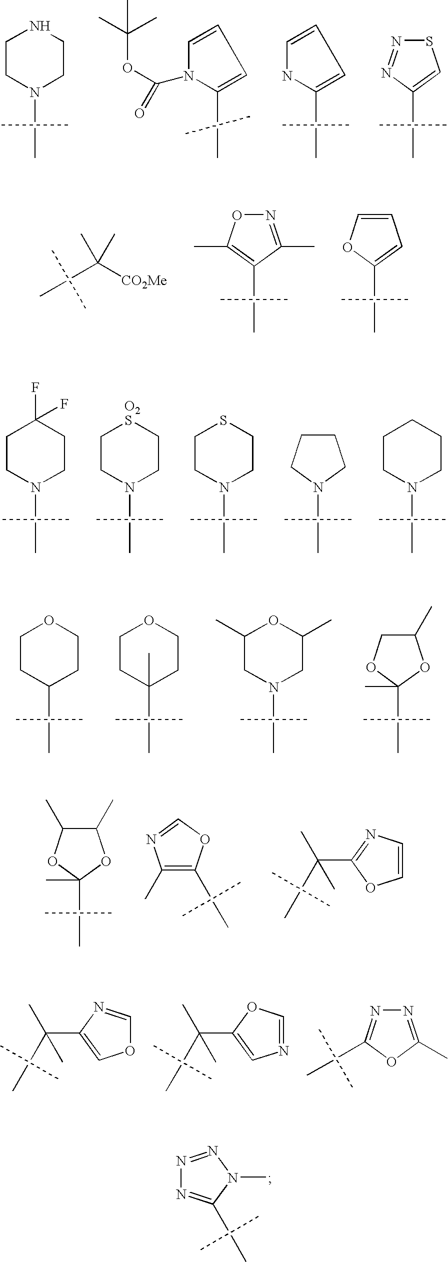

X³, X⁴ and X⁵ are hydrogen;

X² is halogen, —CN, or —CF₃;

Z is selected from the group consisting of:

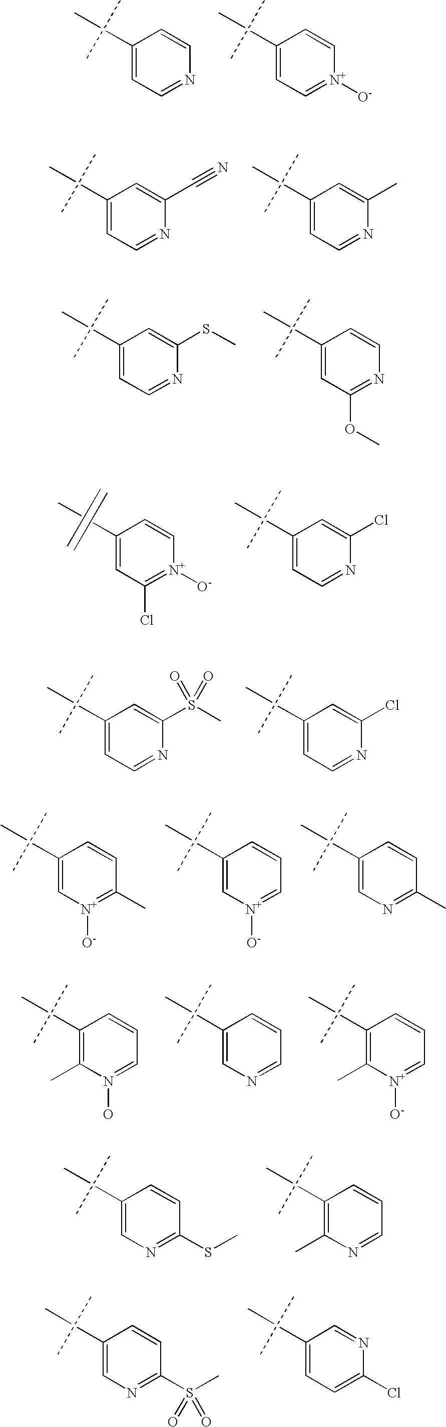

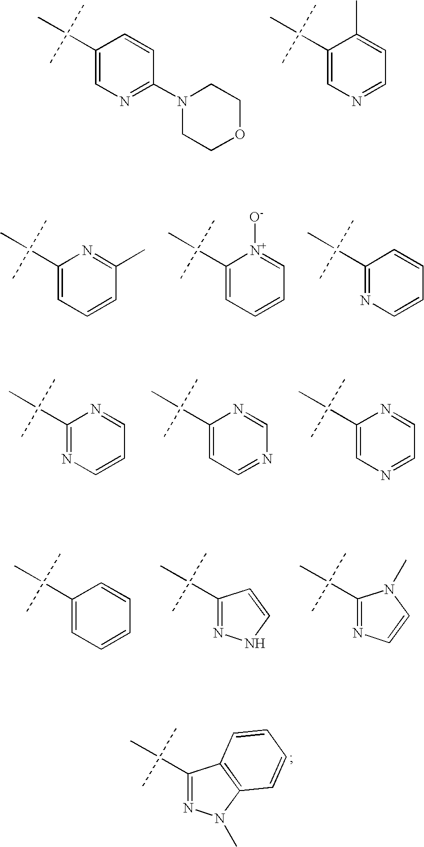

R^{c} when present is as defined for formula (I), preferably R^{c} is hydrogen or —C(O)R, more preferably —COMe;

T when present is as defined for formula (I), preferably T is ═CHC(O)R⁴¹, ═CHC(O)₂R⁴¹, ═CH₂, ═NOR⁴, or ═NOMe, more preferably ═CHC(O)Me, ═CHC(O)₂Me.

In other embodiments Y² is halogen; Y³ is hydrogen; and Y¹ and Y⁴ are each independently hydrogen or halogen.

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine;

X¹ is selected from the group consisting of:

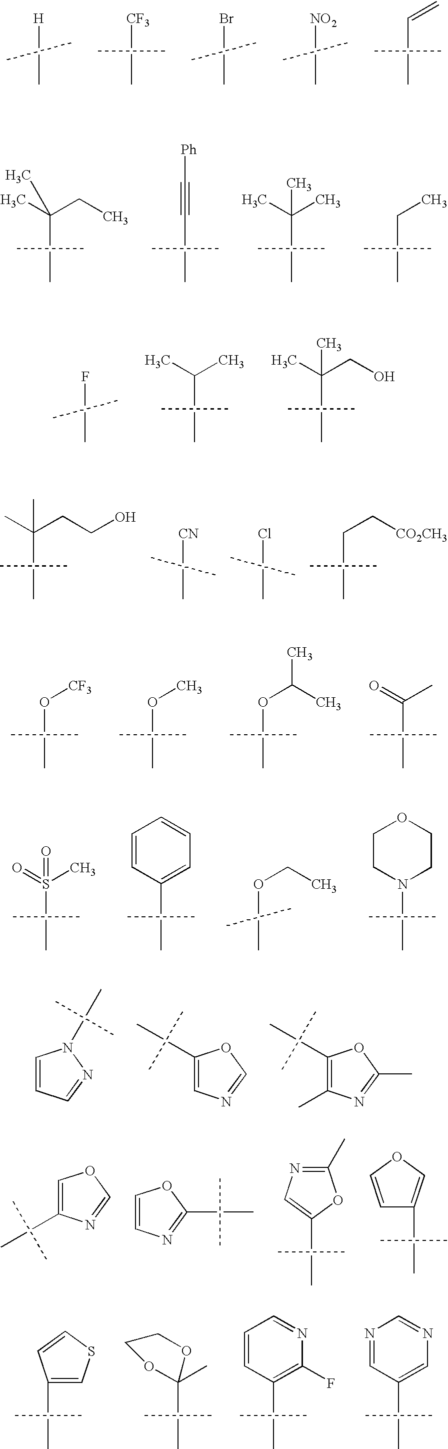

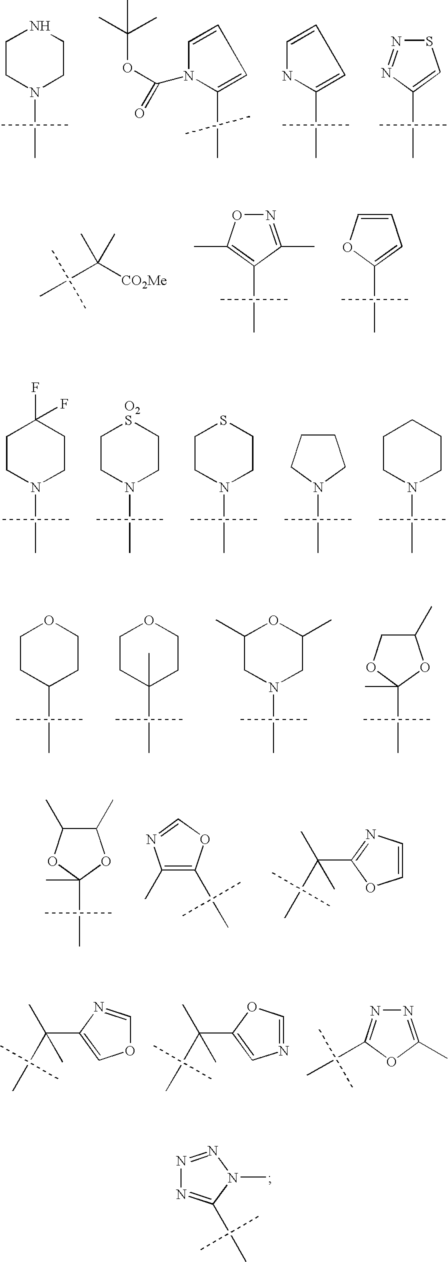

X³, X⁴ and X⁵ are hydrogen;

X² is hydrogen;

and Z is selected from the group consisting of:

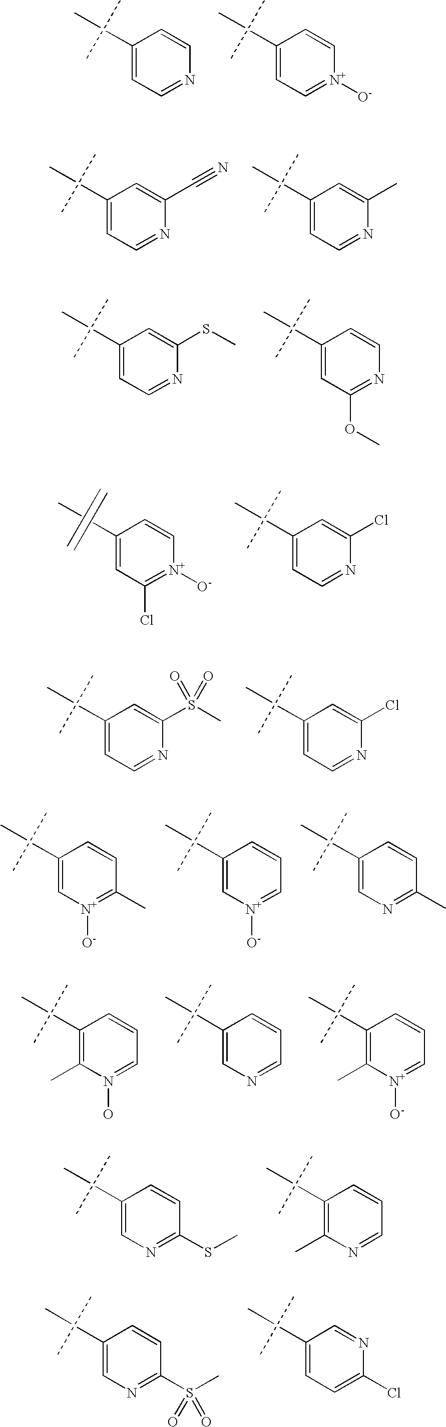

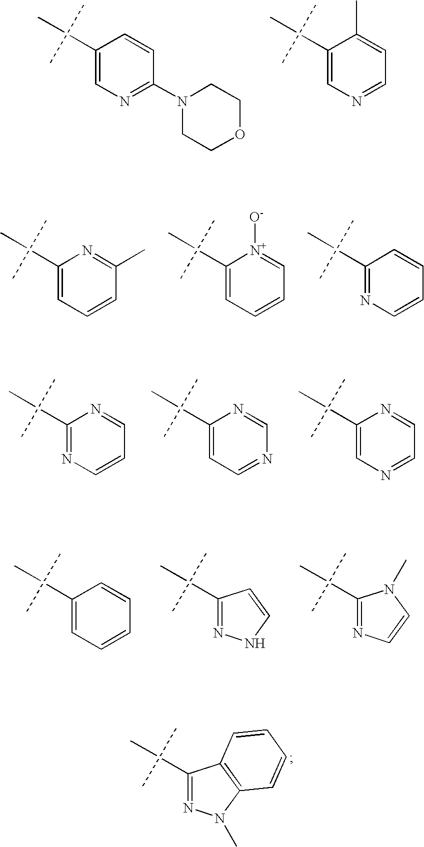

R^{c} when present is as defined for formula (I), preferably R^{c} is hydrogen or —C(O)R, more preferably —COMe;

T when present is as defined for formula (I), preferably T is ═CHC(O)R⁴¹, ═CHC(O)₂R⁴¹, ═CH₂, or ═NOR^{d}, or ═NOMe, more preferably ═CHC(O)Me, ═CHC(O)₂Me.

In other embodiments Y² is halogen; Y³ is hydrogen; and Y¹ and Y⁴ are each independently hydrogen or halogen.

In one embodiment of any of formulae (I-CCXXXII),

Y³ is hydrogen;

Y¹ is chlorine or fluorine, with the proviso that when Y¹ is chlorine, both Y² and Y⁴ are hydrogen or when Y¹ is fluorine, either Y² or Y⁴ is also fluorine;

X¹ is selected from the group consisting of:

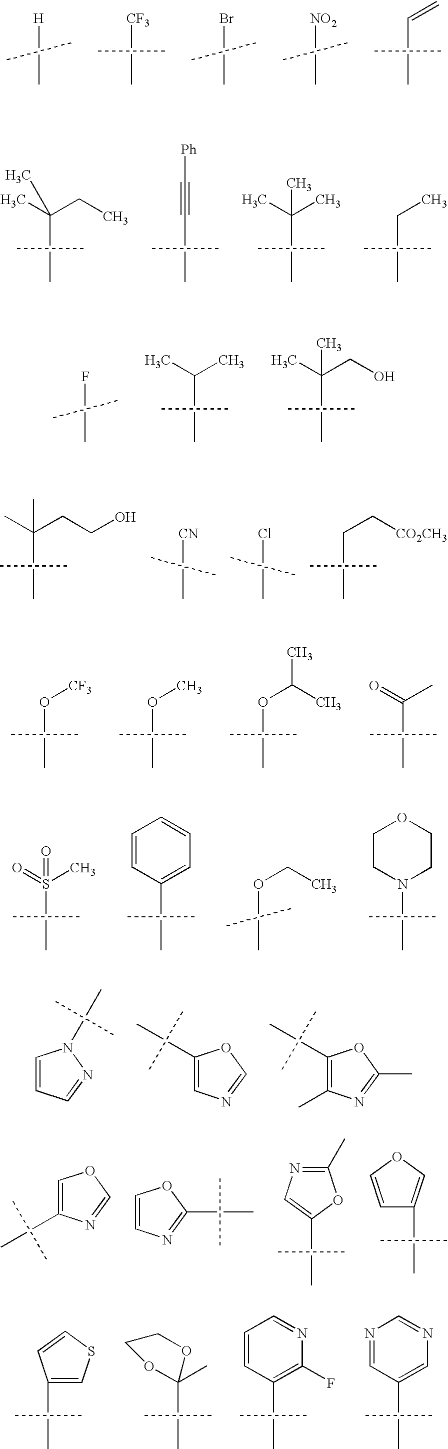

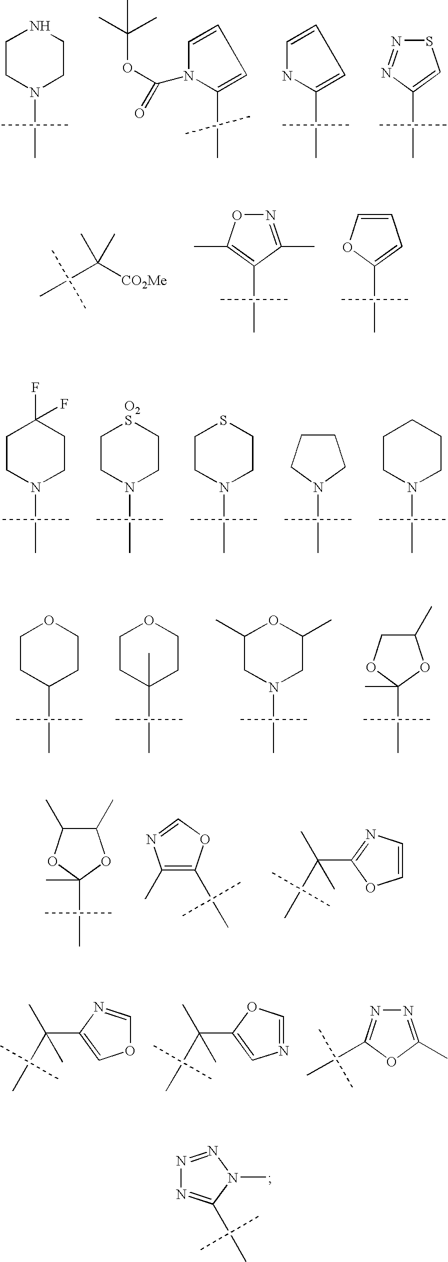

X³, X⁴ and X⁵ are hydrogen;

X² is hydrogen;

and Z is selected from the group consisting of:

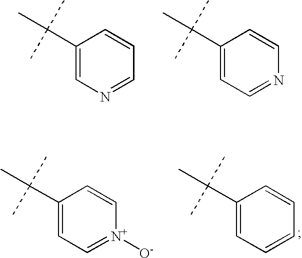

R^{c} when present is as defined for formula (I), preferably R^{c} is hydrogen or —C(O)R, more preferably —COMe;

T when present is as defined for formula (I), preferably T is ═CHC(O)R⁴¹, ═CHC(O)₂R⁴¹, ═CH₂, ═NOR^{d}, or ═NOMe, more preferably ═CHC(O)Me, ═CHC(O)₂Me.

In other embodiments Y² is halogen; Y³ is hydrogen; and Y¹ and Y⁴ are each independently hydrogen or halogen.

In one embodiment of any of formulae (I-CCXXXII), T is ═CHC(O)Me, ═CHCO₂Me, ═CH₂, ═NOEt, or ═NOMe; X³, X⁴ and X⁵ are hydrogen; X² is selected from the group consisting of hydrogen, fluorine, chlorine, —CN and trifluoromethoxy; and X¹ is selected from one of the residues shown below:

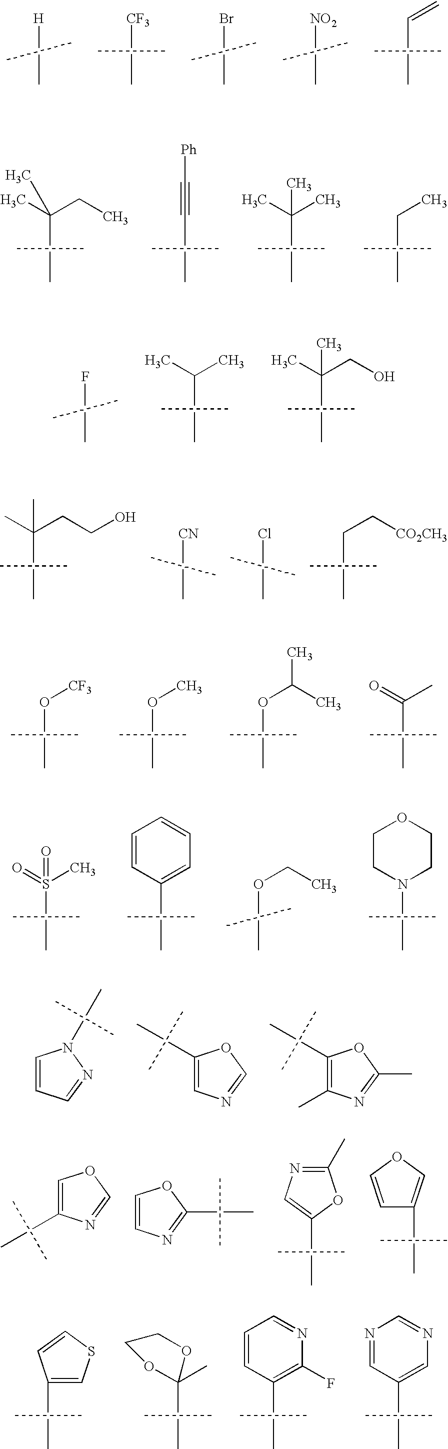

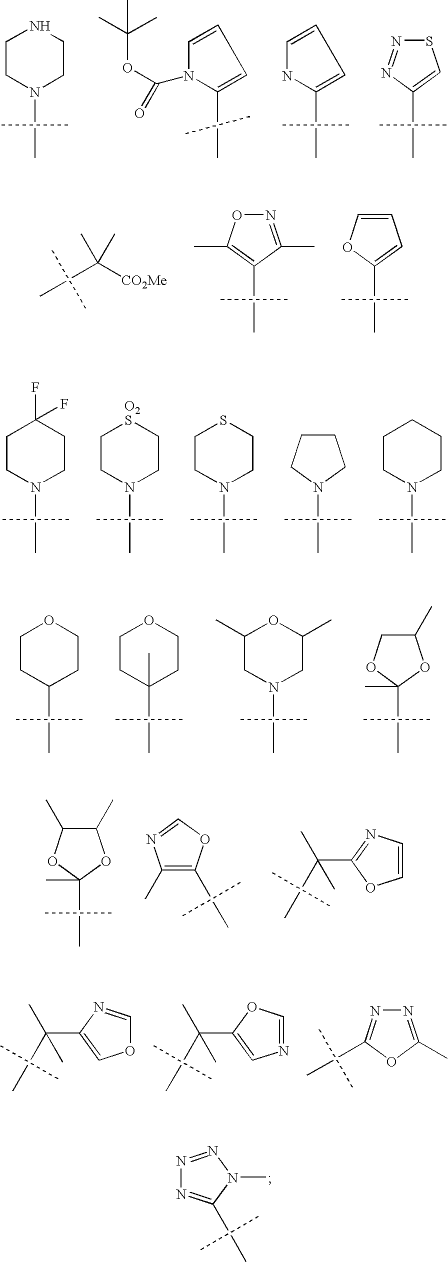

with the proviso that at least one substituent other than hydrogen is present;

Y³ is hydrogen, and Y¹ is chlorine or fluorine; where when Y¹ is chlorine, both Y² and Y⁴ are hydrogen; or alternatively, when Y¹ is fluorine, then either Y² or Y⁴ is also fluorine, with the other residue then being hydrogen; and

Z is selected from the following group consisting of:

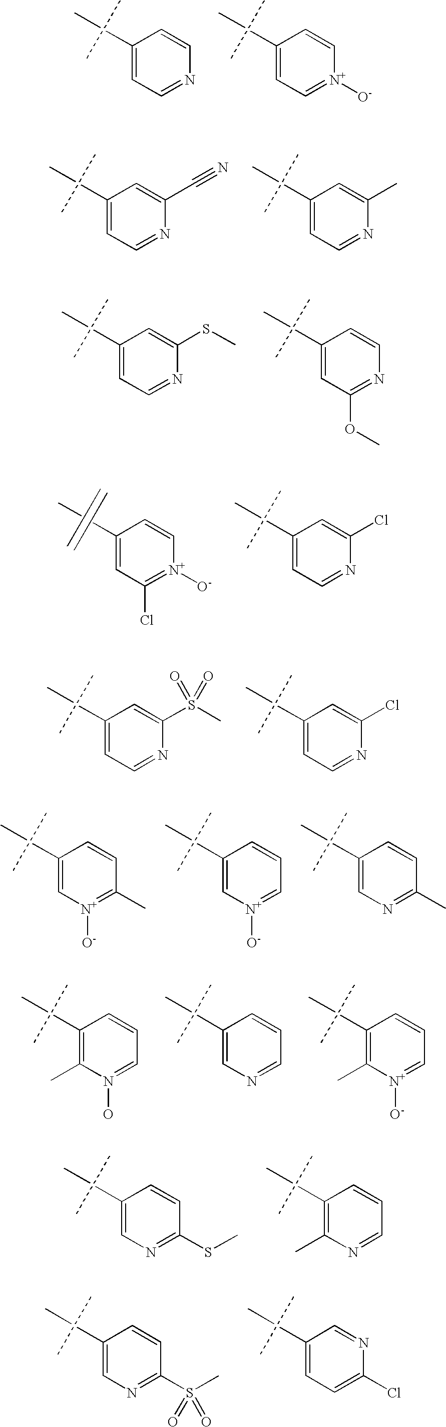

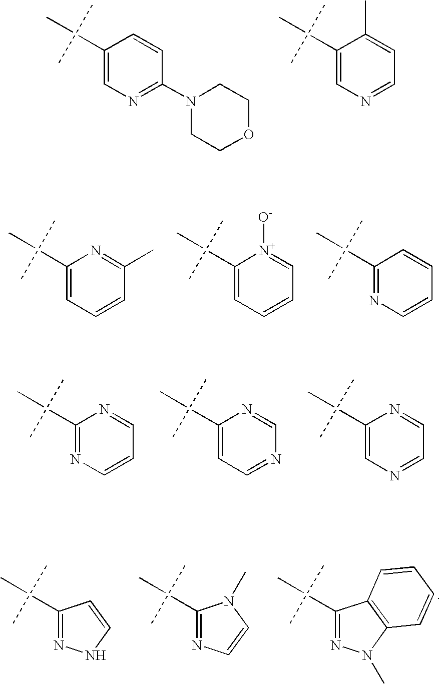

In other embodiments Y² is halogen; Y³ is hydrogen; and Y¹ and Y⁴ are each independently hydrogen or halogen.

In one embodiment of any of formulae (I-CCXXXII) where L is C=T, T is ═CHC(O)Me, ═CHCO₂Me, ═CH₂ or ═NOMe; X³, X⁴ and X⁵ are hydrogen; X² is selected from the group consisting of hydrogen, fluorine, chlorine, —CN and trifluoromethoxy; X¹ is selected from one of the residues shown below:

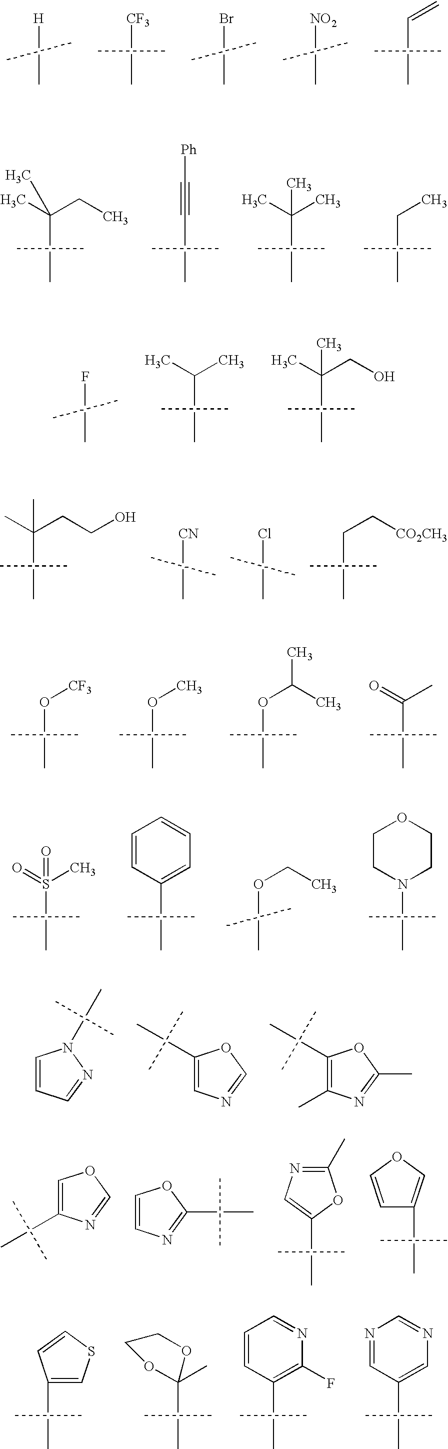

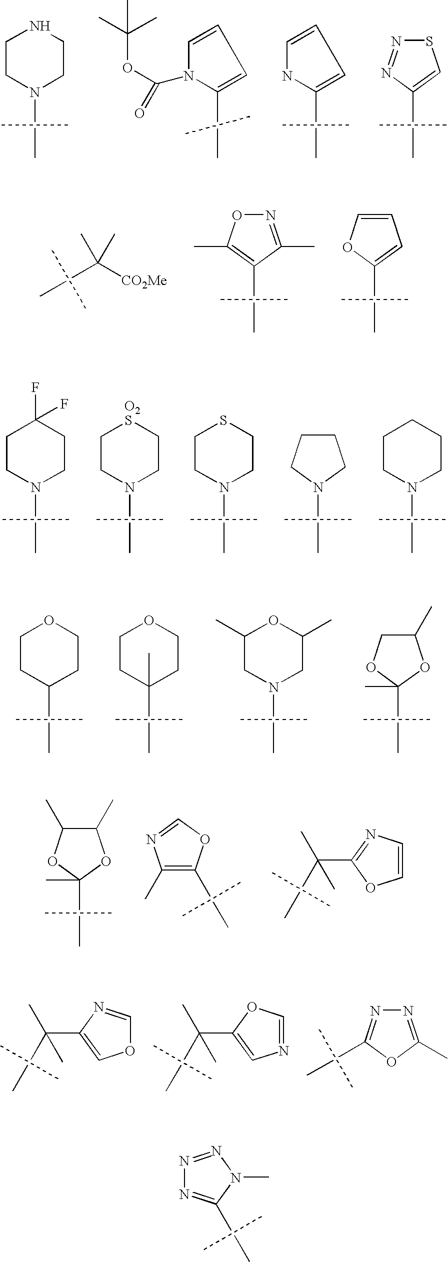

with the proviso that at least one substituent other than hydrogen is present;

Y³ is hydrogen, and Y¹ is chlorine or fluorine; where when Y¹ is chlorine, both Y² and Y⁴ are hydrogen; or alternatively, if Y¹ is fluorine, then either Y² or Y⁴ is also fluorine, with the other residue then being hydrogen; and

Z is substituted with one or two substituents selected from the group consisting of:

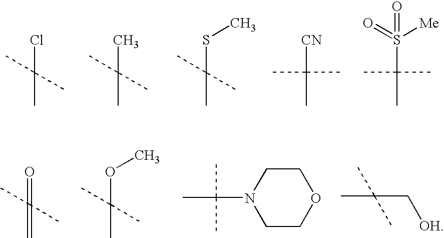

In other embodiments Y² is halogen; Y³ is hydrogen; and Y¹ and Y⁴ are each independently hydrogen or halogen.

In one highly preferred embodiment of any of formulae (I-CCXXXII), X³, X⁴ and X⁵ are hydrogen; X² is selected from the group consisting of hydrogen, fluorine, chlorine, —CN, methyl, and trifluoromethoxy and X¹ is selected from one of the residues shown below:

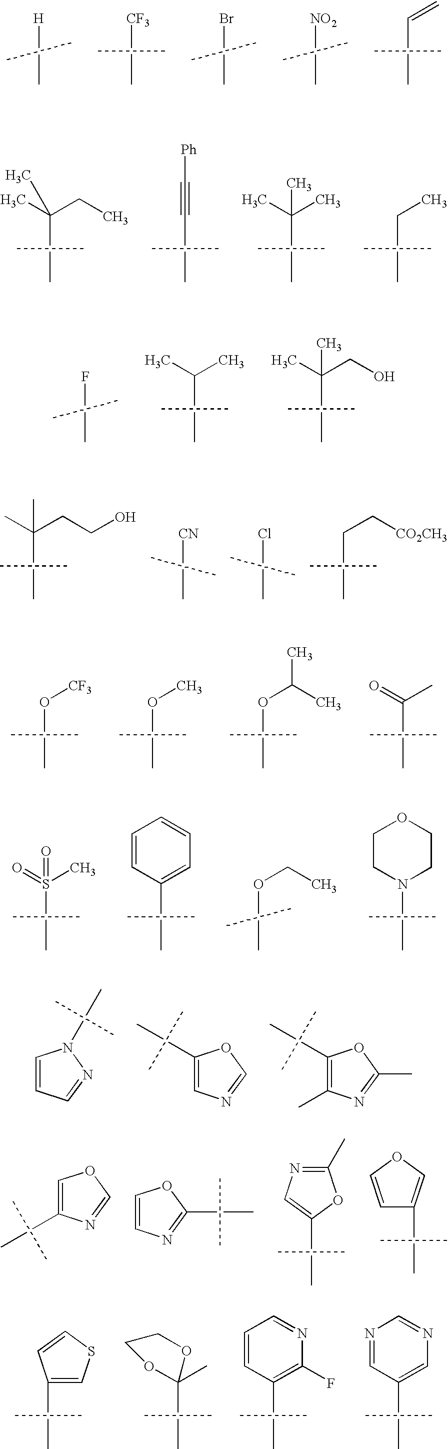

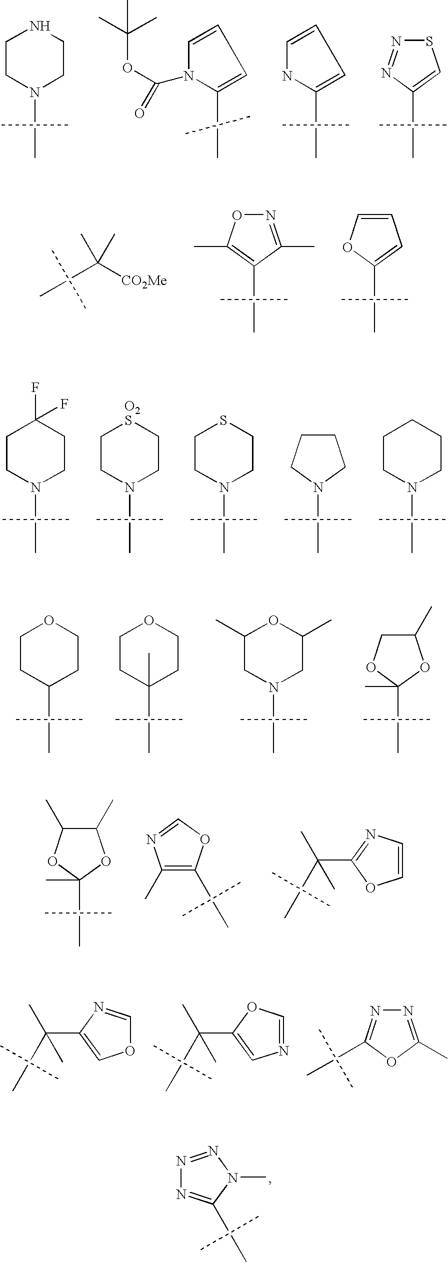

with the proviso that at least one of the substituents is other than hydrogen and that when X² is methyl, then X¹ is other than hydrogen;

when Y³ is hydrogen, Y¹ is chlorine or fluorine; when Y¹ is chlorine, both Y² and Y⁴ are hydrogen also; or when Y¹ is fluorine, either Y² or Y⁴ is also fluorine, with the other residue being hydrogen;

Z is selected from the group consisting of:

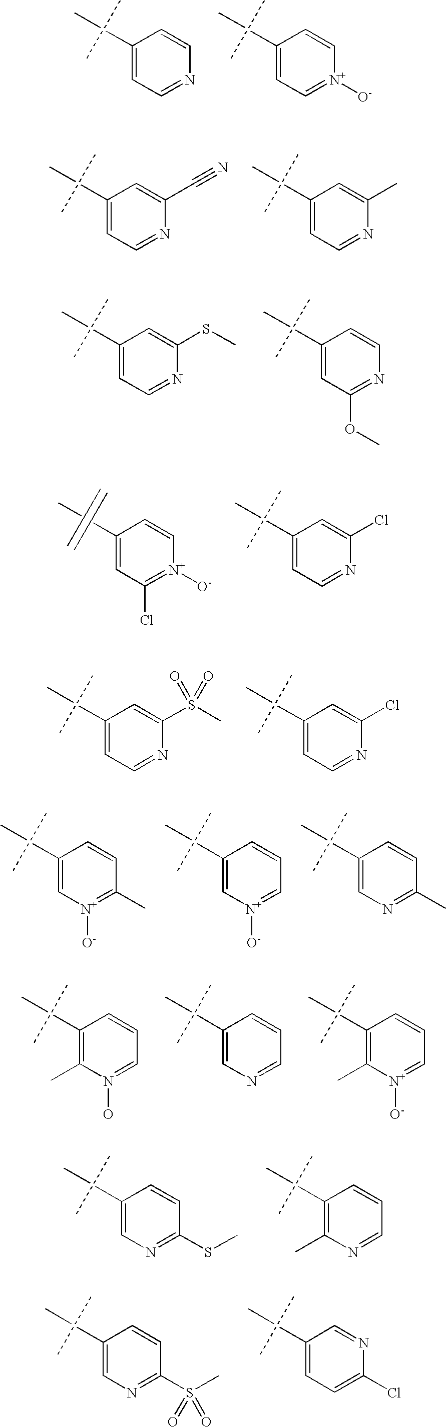

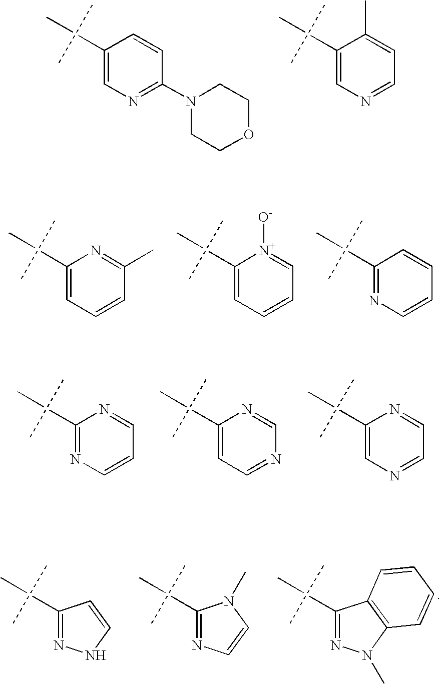

In other embodiments Y² is halogen; Y³ is hydrogen; and Y¹ and Y⁴ are each independently hydrogen or halogen.

In other highly preferred embodiments of any of formulae (I-CCXXXII) where L is —CR^{a}R^{b}— or —NR^{c}—, X³, X⁴ and X⁵ are hydrogen; X² is selected from the group consisting of hydrogen, fluorine, chlorine, —CN and trifluoromethoxy; and X¹ is selected from the group consisting of:

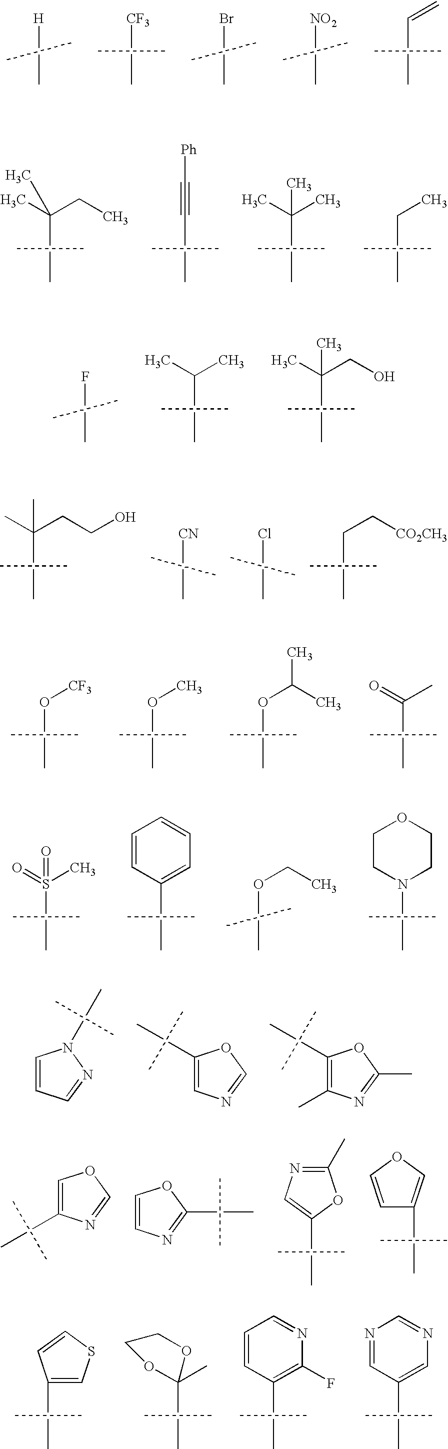

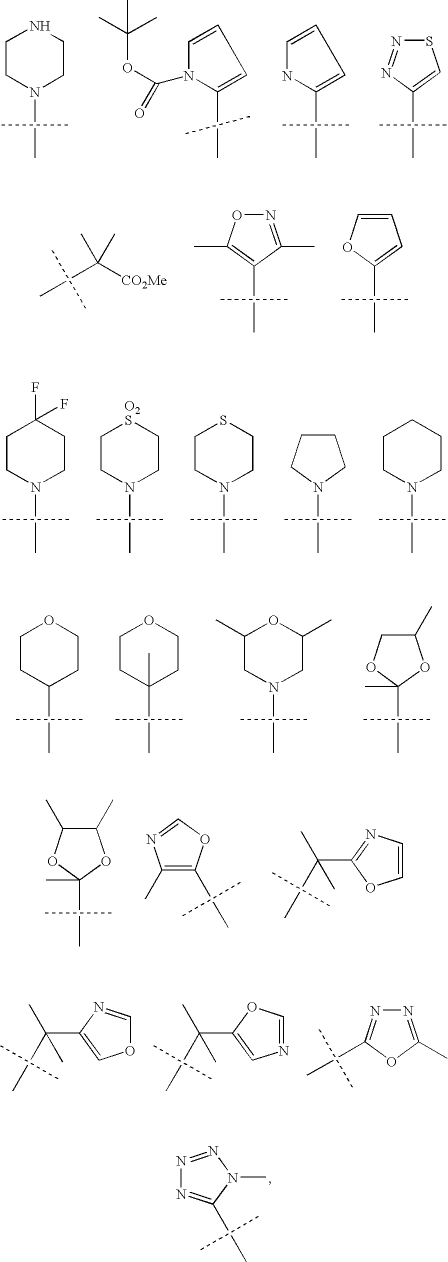

with the proviso that at least one substituent other than hydrogen is present;

when Y¹ or Y^{b} is chlorine, then Y^{a} is hydrogen; or alternatively, when Y¹ or Y^{b} is fluorine, then Y^{a} is hydrogen or fluorine; and

Z is substituted with one or two substituents selected from the group consisting of:

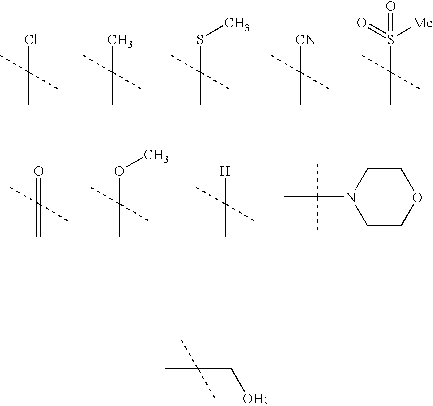

or Z is unsubstituted.

In other embodiments Y² is halogen; Y³ is hydrogen; and Y¹ and Y⁴ are each independently hydrogen or halogen.

In one highly preferred embodiment of any of formulae (I-CCXXXII), X³, X⁴ and X⁵ are hydrogen; X² is selected from the group consisting of hydrogen, fluorine, chlorine, —CN, methyl, and trifluoromethoxy; and X¹ is selected from group consisting of:

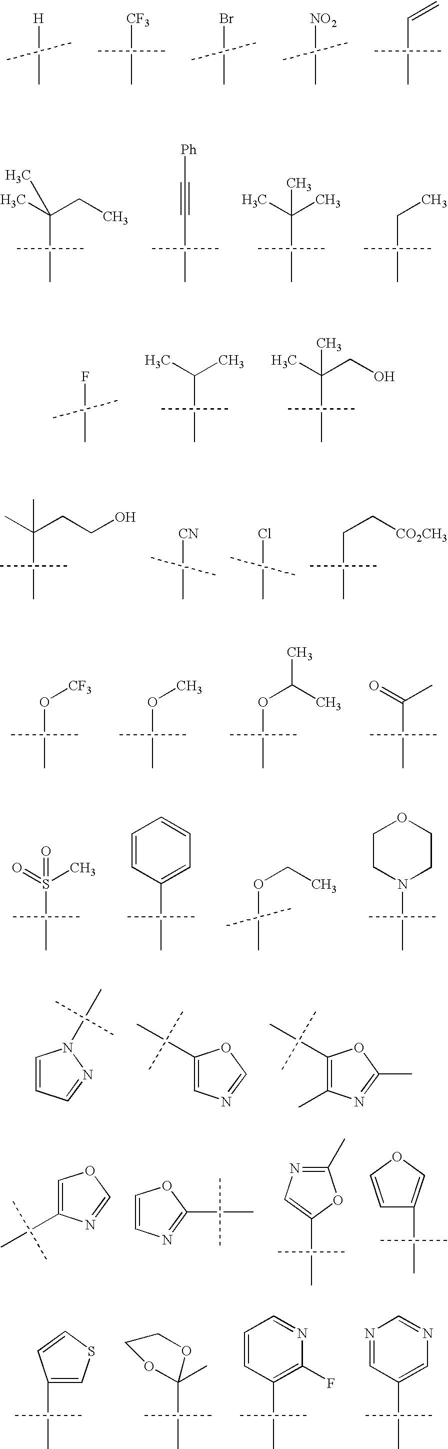

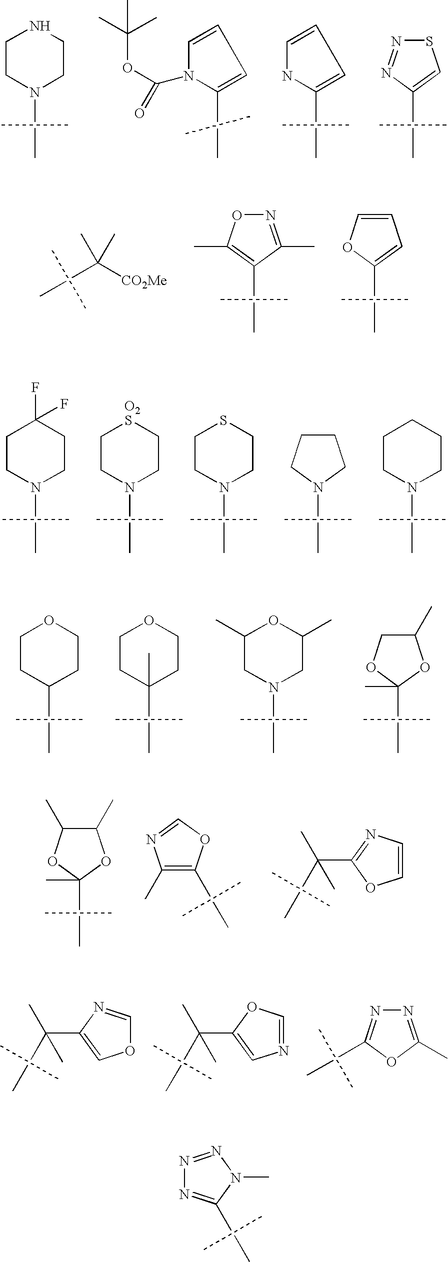

with the proviso that at least one of the substituents is other than hydrogen and that when X² is methyl, X¹ is other than hydrogen;

Y³ is hydrogen, and Y¹ is chlorine or fluorine, but when Y¹ is chlorine, both Y² and Y⁴ are hydrogen; alternatively, when Y¹ is fluorine, then either Y² or Y⁴ is also fluorine, with the other residue then being hydrogen; and

Z is selected from the group consisting of:

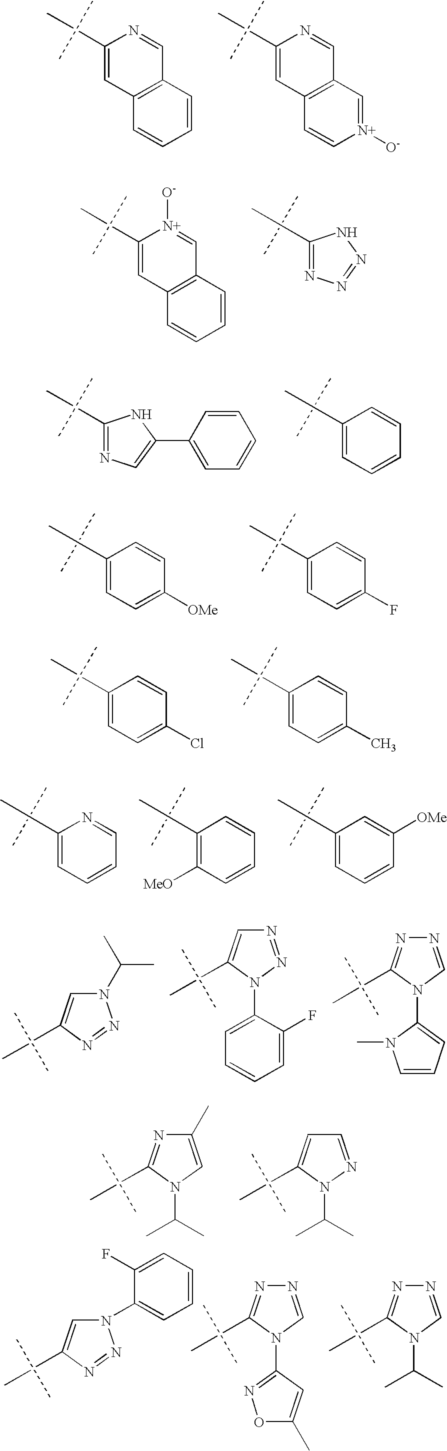

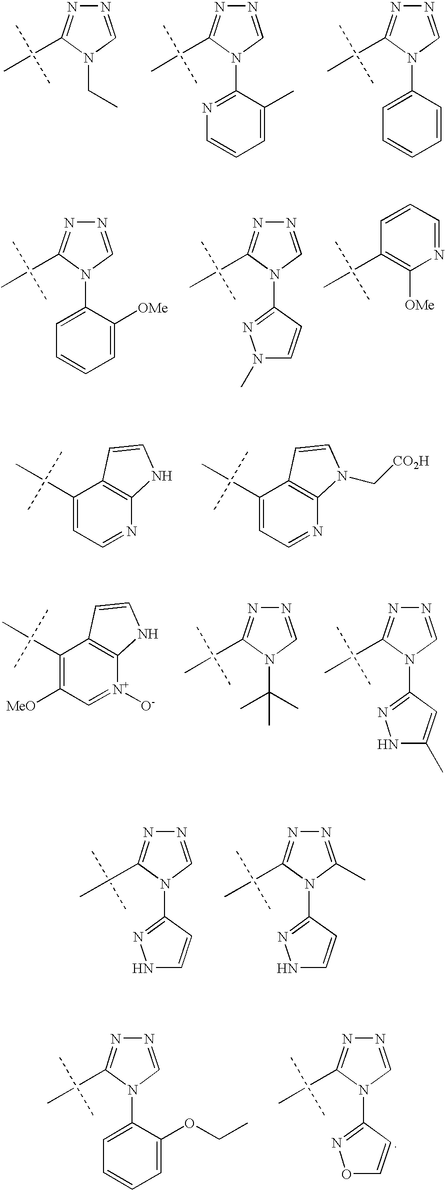

In other embodiments Y² is halogen; Y³ is hydrogen; and Y¹ and Y⁴ are each independently hydrogen or halogen.

In other highly preferred embodiments, in any of formulae (I-CCXXXII) where L is a bond, X³, X⁴ and X⁵ are hydrogen, X² is selected from the group consisting of hydrogen, fluorine, chlorine, —CN and trifluoromethoxy, and X¹ is selected from the group consisting of:

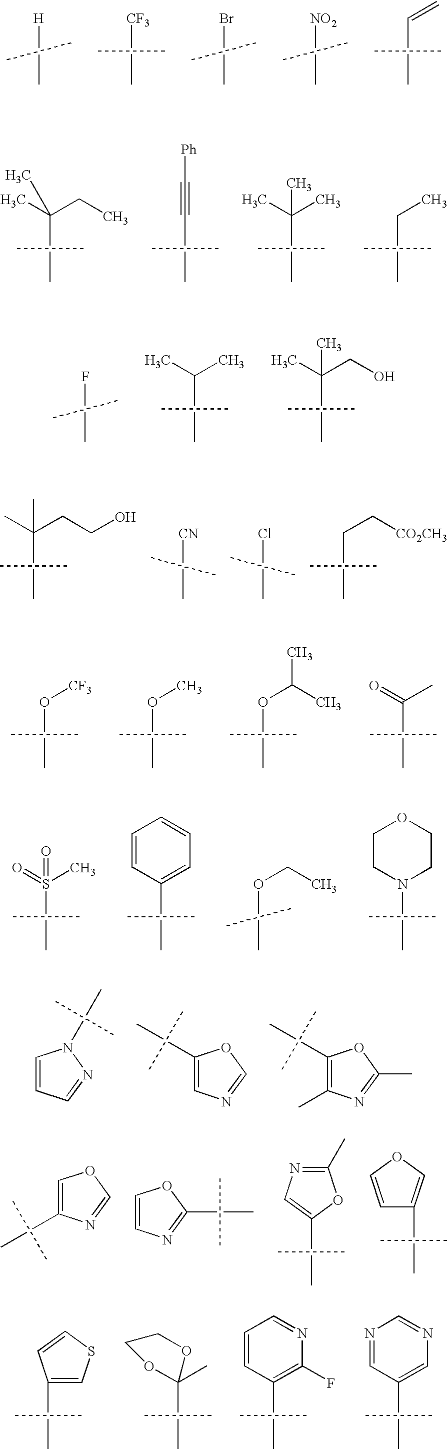

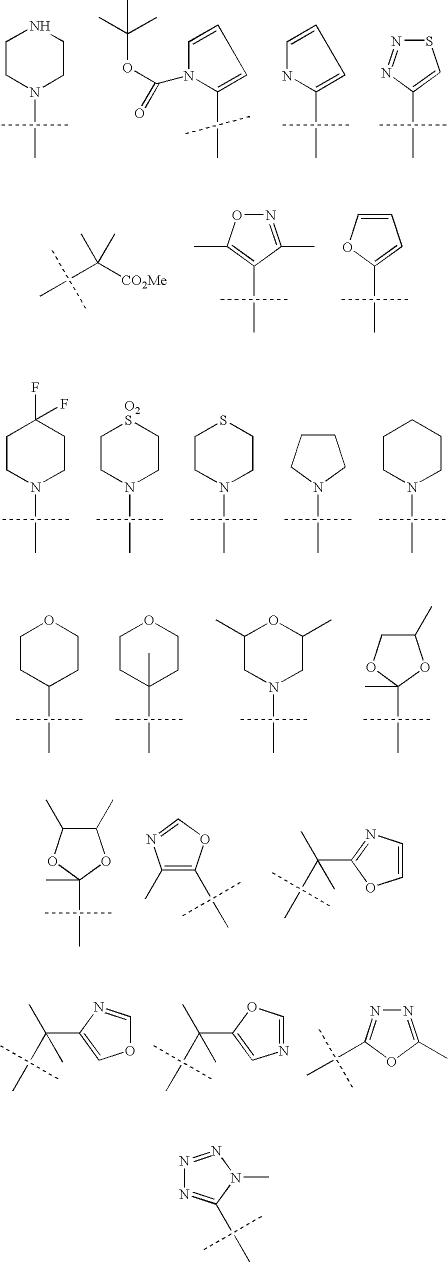

with the proviso that:

at least one of X¹ and X² is other than hydrogen;

when Y¹ or Y^{b} is chlorine, Y^{a} is hydrogen; alternatively when Y¹ or Yb is fluorine, then Y^{a} is hydrogen or fluorine; and

Z is substituted with one or two substitutents selected from the group consisting of:

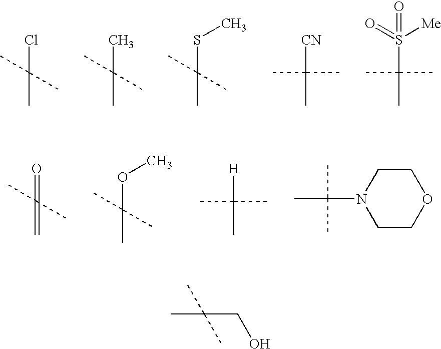

or Z is unsubstituted;

or Z is substituted with one or two substitutents selected from the group consisting of hydrogen, ═O, —OCH₃, —Cl, —F, and —CH₃.

In other embodiments Y² is halogen; Y³ is hydrogen; and Y¹ and Y⁴ are each independently hydrogen or halogen.

In one embodiment of formula (CLIVe), X¹ is selected from the group consisting of hydrogen, halogen, and substituted or unsubstituted C₁₋₈ alkyl; X² is selected from the group consisting of halogen, substituted or unsubstituted C₁₋₈ alkyl, —CN, —NO₂, and —CO₂R³; Y^{a} is hydrogen, Y¹ is selected from the group consisting of halogen, substituted or unsubstituted C₁₋₄ alkyl, —CO₂R⁶, and —OR⁶; L is —C(O)—; Z^{a} and Z^{b} are each independently selected from the group consisting of hydrogen, halogen, substituted or unsubstituted C₁₋₈ alkyl, and ═O; and R³ is hydrogen or substituted or unsubstituted C₁₋₈ alkyl, —CO₂R′, —C(O)R′, and —SiR′R″R″; where R′, R″ and R′″ each independently selected from the group consisting of hydrogen, substituted or unsubstituted C₁₋₈ alkyl, and substituted or unsubstituted aryl.

In one embodiment of formula (V), X¹ and X² are selected from the group consisting of —F, —Cl, —Br, —CN, —NO₂, —OCH₃, —OCF₃, —CH₃, —CF₃, —CO₂H, and —CONHCH₃. Y^{a} is hydrogen; Y¹ is selected from the group consisting of of —F, —Cl, —Br, —CN, —OCH₃, —CF₃, and —CH₃; L is —C(O)—; and Z is other than substituted or unsubstituted phenyl, substituted or unsubstituted pyridyl and substituted or unsubstituted pyridyl-N-oxide.

In one embodiment of formula (VI), X¹ and X² are selected from the group consisting of —F, —Cl, —Br, —CN, —NO₂, —OCH₃, —OCF₃, —CH₃, —CF₃, —CO₂H, and —CONHCH₃. Y^{a} is hydrogen; Y¹ is selected from the group consisting of of —F, —Cl, —Br, —CN, —OCH₃, —CF₃, and —CH₃; L is —C(O)—; and Z is other than substituted or unsubstituted phenyl, substituted or unsubstituted pyridyl and substituted or unsubstituted pyridyl-N-oxide.

In one embodiment of formula (V), X¹ is selected from the group consisting of hydrogen, halogen, and substituted or unsubstituted C₁₋₈ alkyl; X² is selected from the group consisting of halogen, substituted or unsubstituted C₁₋₈ alkyl, —CN, —NO₂, and —CO₂R³; Y^{a} is hydrogen, Y¹ is selected from the group consisting of halogen, substituted or unsubstituted C₁₋₄ alkyl, —CO₂R⁶, and —OR⁶; L is —C(O)— and Z is selected from the group consisting of substituted or unsubstituted imidazolyl, substituted or unsubstituted pyrrolidinyl, substituted or unsubstituted pyrrolopyridinyl, substituted or unsubstituted pyrazolyl, substituted or unsubstituted morpholinyl, substituted or unsubstituted piperidinyl, substituted or unsubstituted phenyl, substituted or unsubstituted oxazolyl, substituted or unsubstituted pyridyl, and substituted or unsubstituted tetrazolyl.

In one embodiment of formula (V), X¹ is selected from the group consisting of hydrogen, halogen, methyl, tert-butyl and —CF₃; X² is selected from the group consisting of halogen, methyl, —CF₃, —CN, —NO₂, and —CO₂R³, Y^{a} is hydrogen, Y¹ is selected from the group consisting of halogen, substituted or unsubstituted C₁₋₄ alkyl, —CO₂R⁶, and —OR⁶; L is —C(O)— and Z is selected from the group consisting of substituted or unsubstituted imidazolyl, substituted or unsubstituted pyrrolidinyl, substituted or unsubstituted pyrrolopyridinyl, substituted or unsubstituted pyrazolyl, substituted or unsubstituted morpholinyl, substituted or unsubstituted piperidinyl, substituted or unsubstituted phenyl, substituted or unsubstituted oxazolyl, substituted or unsubstituted pyridyl, and substituted or unsubstituted tetrazolyl.

In one embodiment of formula (V), when L is a bond, Z is other than substituted or unsubstituted oxazol-2-yl, substituted or unsubstituted 1,3,4-oxadiazol-2-yl, substituted or unsubstituted 1,2,4-oxadiazol-5-yl, and substituted or unsubstituted furan-2-yl.

In one embodiment of formula (VI), X¹ is selected from the group consisting of hydrogen, halogen, and substituted or unsubstituted C₁₋₈ alkyl; X² is selected from the group consisting of halogen, substituted or unsubstituted C₁₋₈ alkyl, —CN, —NO₂, and —CO₂R³; Y^{a} is hydrogen, Y¹ is selected from the group consisting of halogen, substituted or unsubstituted C₁₋₄ alkyl, —CO₂R⁶, and —OR⁶; L is —C(O)— and Z is selected from the group consisting of substituted or unsubstituted imidazolyl, substituted or unsubstituted pyrrolidinyl, substituted or unsubstituted pyrrolopyridinyl, substituted or unsubstituted pyrazolyl, substituted or unsubstituted morpholinyl, substituted or unsubstituted piperidinyl, substituted or unsubstituted phenyl, substituted or unsubstituted oxazolyl, substituted or unsubstituted pyridyl, and substituted or unsubstituted tetrazolyl.

In one embodiment of formula (VI), X¹ is selected from the group consisting of hydrogen, halogen, methyl, tert-butyl and —CF₃; X² is selected from the group consisting of halogen, methyl, —CF₃, —CN, —NO₂, and —CO₂R³; Y^{a} is hydrogen, Y¹ is selected from the group consisting of halogen, substituted or unsubstituted C₁₋₄ alkyl, —CO₂R⁶, and —OR⁶; L is —C(O)— and Z is selected from the group consisting of substituted or unsubstituted imidazolyl, substituted or unsubstituted pyrrolidinyl, substituted or unsubstituted pyrrolopyridinyl, substituted or unsubstituted pyrazolyl, substituted or unsubstituted morpholinyl, substituted or unsubstituted piperidinyl, substituted or unsubstituted phenyl, substituted or unsubstituted oxazolyl, substituted or unsubstituted pyridyl, and substituted or unsubstituted tetrazolyl.

In one embodiment of formula (VI), when L is a bond, Z is other than substituted or unsubstituted oxazol-2-yl, substituted or unsubstituted 1,3,4-oxadiazol-2-yl, substituted or unsubstituted 1,2,4-oxadiazol-5-yl, and substituted or unsubstituted furan-2-yl.

Compositions that Modulate Chemokine Activity

In another aspect, the present invention provides compositions that modulate chemokine activity, specifically CCR2 activity or CCR9 acitivity. Generally, the compositions for modulating chemokine receptor activity in humans and animals will comprise a pharmaceutically acceptable excipient or diluent and a compound having the formula provided above as any of formulae (I-III).

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this invention may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil in water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids-and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols. Additionally, the compounds can be administered via ocular delivery by means of solutions or ointments. Still further, transdermal delivery of the subject compounds can be accomplished by means of iontophoretic patches and the like.

For topical use, creams, ointments, jellies, solutions or suspensions containing the compounds of the present invention are employed. As used herein, topical application is also meant to include the use of mouth washes and gargles.

The pharmaceutical compositions and methods of the present invention may further comprise other therapeutically active compounds as noted herein, such as those applied in the treatment of the above mentioned pathological conditions.

In one embodiment, the present invention provides a composition consisting of a pharmaceutically acceptable carrier and a compound of the invention.

Methods of Treatment

Depending on the disease to be treated and the subject's condition, the compounds and compositions of the present invention may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional non toxic pharmaceutically acceptable carriers, adjuvants and vehicles appropriate for each rouse of administration. The present invention also contemplates administration of the compounds and compositions of the present invention in a depot formulation.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5, 0.5 to 5.0, or 5.0 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In still other embodiments, the present methods are directed to the treatment of allergic diseases, wherein a compound or composition of the invention is administered either alone or in combination with a second therapeutic agent, wherein said second therapeutic agent is an antihistamine. When used in combination, the practitioner can administer a combination of the compound or composition of the present invention and a second therapeutic agent. Also, the compound or composition and the second therapeutic agent can be administered sequentially, in any order.

The compounds and compositions of the present invention can be combined with other compounds and compositions having related utilities to prevent and treat the condition or disease of interest, such as inflammatory conditions and diseases, including inflammatory bowel disease, allergic diseases, psoriasis, atopic dermatitis and asthma, and those pathologies noted above. Selection of the appropriate agents for use in combination therapies can be made one of ordinary skill in the art. The combination of therapeutic agents may act synergistically to effect the treatment or prevention of the various disorders. Using this approach, one may be able to achieve therapeutic efficacy with lower dosages of each agent, thus reducing the potential for adverse side effects.

In treating, preventing, ameliorating, controlling or reducing the risk of inflammation, the compounds of the present invention may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, biological TNF sequestrants, fentanyl, ibuprofen, indomethacin, ketorolac, morphine, naproxen, phenacetin, piroxicam, a steroidal analgesic, sufentanyl, sunlindac, tenidap, and the like.

Similarly, the compounds of the present invention may be administered with a pain reliever; a potentiator such as caffeine, an H2-antagonist, simethicone, aluminum or magnesium hydroxide; a decongestant such as pseudophedrine; an antitussive such as codeine; a diuretic; a sedating or non-sedating antihistamine; a very late antigen (VLA-4) antagonist; an immunosuppressant such as cyclosporin, tacrolimus, rapamycin, EDG receptor agonists, or other FK-506 type immunosuppressants; a steroid; a non-steroidal anti-asthmatic agent such as a β2-agonist, leukotriene antagonist, or leukotriene biosynthesis inhibitor; an inhibitor of phosphodiesterase type IV (PDE-IV); a cholesterol lowering agent such as a HMG-CoA reductase inhibitor, sequestrant, or cholesterol absorption inhibitor; and an anti-diabetic agent such as insulin, α-glucosidase inhibitors or glitazones.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

Methods of Treating or Preventing CCR2-mediated Conditions or Diseases

In yet another aspect, the present invention provides methods of treating or preventing a CCR2-mediated condition or disease by administering to a subject having such a condition or disease a therapeutically effective amount of any compound of formula (I) above. Compounds for use in the present methods include those compounds according to formula (I), those provided above as embodiments, those specifically exemplified in the Examples below, and those provided with specific structures herein. The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the phrase “CCR2-mediated condition or disease” and related phrases and terms refer to a condition or disease characterized by inappropriate, i.e., less than or greater than normal, CCR2 functional activity. Inappropriate CCR2 functional activity might arise as the result of CCR2 expression in cells which normally do not express CCR2, increased CCR2 expression (leading to, e.g., inflammatory and immunoregulatory disorders and diseases) or decreased CCR2 expression. Inappropriate CCR2 functional activity might also arise as the result of MCP-1 secretion by cells which normally do not secrete MCP-1, increased MCP-1 expression (leading to, e.g., inflammatory and immunoregulatory disorders and diseases) or decreased MCP-1 expression. A CCR2-mediated condition or disease may be completely or partially mediated by inappropriate CCR2 functional activity. However, a CCR2-mediated condition or disease is one in which modulation of CCR2 results in some effect on the underlying condition or disease (e.g., a CCR2 antagonist results in some improvement in patient well being in at least some patients). Furthermore, MCP-2, 3 and 4 are also CCR2 ligands.

In one embodiment, the present invention provides a method of treating a CCR2-mediated condition or disease involving administering to a subject a safe and effective amount of the compound or composition of the invention.

In one embodiment, the present invention provides a method of treating a CCR2-mediated condition or disease involving administering to a subject a safe and effective amount of the compound or composition of the invention, where the CCR2-mediated condition or disease is atherosclerosis.

In one embodiment, the present invention provides a method of treating a CCR2-mediated condition or disease involving administering to a subject a safe and effective amount of the compound or composition of the invention, where the CCR2-mediated condition or disease is restenosis.

In one embodiment, the present invention provides a method of treating a CCR2-mediated condition or disease involving administering to a subject a safe and effective amount of the compound or composition of the invention, where the CCR2-mediated condition or disease is multiple sclerosis.

In one embodiment, the present invention provides a method of treating a CCR2-mediated condition or disease involving administering to a subject a safe and effective amount of the compound or composition of the invention, where the CCR2-mediated condition or disease is selected from the group consisting of inflammatory bowel disease, renal fibrosis, rheumatoid arthritis, obesity and noninsulin-dependent diabetes.

In one embodiment, the present invention provides a method of treating a CCR2-mediated condition or disease involving administering to a subject a safe and effective amount of the compound or composition of the invention, where the CCR2-mediated condition or disease is type 2 diabetes.

In one embodiment, the present invention provides a method of treating a CCR2-mediated condition or disease involving administering to a subject a safe and effective amount of the compound or composition of the invention, where the CCR2-mediated condition or disease is selected from the group consisting of chronic obstructive pulmonary disease, idiopathic pulmonary fibrosis and idiopathic pneumonia syndrome.

In one embodiment, the present invention provides a method of treating a CCR2-mediated condition or disease involving administering to a subject a safe and effective amount of the compound or composition of the invention, where the administering is oral, parenteral, rectal, transdermal, sublingual, nasal or topical.

In one embodiment, the present invention provides a method of treating a CCR2-mediated condition or disease involving administering to a subject a safe and effective amount of the compound or composition of the invention, where the compound is administered in combination with an anti-inflammatory or analgesic agent.

In one embodiment, the present invention provides a method of treating a CCR2-mediated condition or disease involving administering to a subject a safe and effective amount of the compound or composition of the invention, where an anti-inflammatory or analgesic agent is also administered.

In one embodiment, the present invention provides a method of modulating CCR2 function in a cell, where the CCR2 function in the cell is modulated by contacting the cell with a CCR2 modulating amount of the compound of the present invention.

In one embodiment, the present invention provides a method of treating a CCR2-mediated condition or disease involving administering to a subject a safe and effective amount of the compound or composition of the invention, where the disease is selected from the group consisting of pulmonary fibrosis, transplantation rejection, graft-versus-host disease and cancer.

In yet other embodiments, the present methods are directed to the treatment of psoriasis wherein a compound or composition of the invention is used alone or in combination with a second therapeutic agent such as a corticosteroid, a lubricant, a keratolytic agent, a vitamin D₃ derivative, PUVA and anthralin.

In other embodiments, the present methods are directed to the treatment of atopic dermatitis using a compound or composition of the invention either alone or in combination with a second therapeutic agent such as a lubricant and a corticosteroid.

In further embodiments, the present methods are directed to the treatment of asthma using a compound or composition of the invention either alone or in combination with a second therapeutic agent such as a β2-agonist and a corticosteroid.

Methods of Treating or Preventing CCR9-mediated Conditions or Diseases

In yet another aspect, the present invention provides methods of treating or preventing a CCR9-mediated condition or disease by administering to a subject having such a condition or disease a therapeutically effective amount of any compound of formula (I) above. Compounds for use in the present methods include those compounds according to formula (I), those provided above as embodiments, those specifically exemplified in the Examples below, and those provided with specific structures herein. The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the phrase “CCR9-mediated condition or disease” and related phrases and terms refer to a condition or disease characterized by inappropriate, i.e., less than or greater than normal, CCR9 functional activity. Inappropriate CCR9 functional activity might arise as the result of CCR9 expression in cells which normally do not express CCR9, increased CCR9 expression (leading to, e.g., inflammatory and immunoregulatory disorders and diseases) or decreased CCR9 expression. Inappropriate CCR9 functional activity might also arise as the result of TECK secretion by cells which normally do not secrete TECK, increased TECK expression (leading to, e.g., inflammatory and immunoregulatory disorders and diseases) or decreased TECK expression. A CCR9-mediated condition or disease may be completely or partially mediated by inappropriate CCR9 functional activity. However, a CCR9-mediated condition or disease is one in which modulation of CCR9 results in some effect on the underlying condition or disease (e.g., a CCR9 antagonist results in some improvement in patient well being in at least some patients).

The term “therapeutically effective amount” means the amount of the subject compound that will elicit the biological or medical response of a cell, tissue, system, or animal, such as a human, that is being sought by the researcher, veterinarian, medical doctor or other treatment provider.

Diseases and conditions associated with inflammation, immune disorders, infection and cancer can be treated or prevented with the present compounds, compositions, and methods. In one group of embodiments, diseases or conditions, including chronic diseases, of humans or other species can be treated with inhibitors of CCR9 function. These diseases or conditions include: (1) allergic diseases such as systemic anaphylaxis or hypersensitivity responses, drug allergies, insect sting allergies and food allergies, (2) inflammatory bowel diseases, such as Crohn's disease, ulcerative colitis, ileitis and enteritis, (3) vaginitis, (4) psoriasis and inflammatory dermatoses such as dermatitis, eczema, atopic dermatitis, allergic contact dermatitis, urticaria and pruritus, (5) vasculitis, (6) spondyloarthropathies, (7) scleroderma, (8) asthma and respiratory allergic diseases such as allergic asthma, allergic rhinitis, hypersensitivity lung diseases and the like, (9) autoimmune diseases, such as fibromyalagia, scleroderma, ankylosing spondylitis, juvenile RA, Still's disease, polyarticular juvenile RA, pauciarticular juvenile RA, polymyalgia rheumatica, rheumatoid arthritis, psoriatic arthritis, osteoarthritis, polyarticular arthritis, multiple sclerosis, systemic lupus erythematosus, type I diabetes, type II diabetes, glomerulonephritis, and the like, (10) graft rejection (including allograft rejection), (11) graft-v-host disease (including both acute and chronic), (12) other diseases in which undesired inflammatory responses are to be inhibited, such as atherosclerosis, myositis, neurodegenerative diseases (e.g., Alzheimer's disease), encephalitis, meningitis, hepatitis, nephritis, sepsis, sarcoidosis, allergic conjunctivitis, otitis, chronic obstructive pulmonary disease, sinusitis, Behcet's syndrome and gout, (13) immune mediated food allergies such as Coeliac (Celiac) disease (14) pulmonary fibrosis and other fibrotic diseases, and (15) irritable bowel syndrome.

In another group of embodiments, diseases or conditions can be treated with modulators and agonists of CCR9 function. Examples of diseases to be treated by modulating CCR9 function include cancers, cardiovascular diseases, diseases in which angiogenesis or neovascularization play a role (neoplastic diseases, retinopathy and macular degeneration), infectious diseases (viral infections, e.g., HIV infection, and bacterial infections) and immunosuppressive diseases such as organ transplant conditions and skin transplant conditions. The term “organ transplant conditions” is means to include bone marrow transplant conditions and solid organ (e.g., kidney, liver, lung, heart, pancreas or combination thereof) transplant conditions.

Preferably, the present methods are directed to the treatment of diseases or conditions selected from inflammatory bowel disease including Crohn's disease and Ulcerative Colitis, allergic diseases, psoriasis, atopic dermatitis and asthma, autoimmune disease such as rheumatoid arthritis and immune-mediated food allergies such as Coelaic disease.

In yet other embodiments, the present methods are directed to the treatment of psoriasis where a compound or composition of the invention is used alone or in combination with a second therapeutic agent such as a corticosteroid, a lubricant, a keratolytic agent, a vitamin D₃ derivative, PUVA and anthralin.

In other embodiments, the present methods are directed to the treatment of atopic dermatitis using a compound or composition of the invention either alone or in combination with a second therapeutic agent such as a lubricant and a corticosteroid.

In further embodiments, the present methods are directed to the treatment of asthma using a compound or composition of the invention either alone or in combination with a second therapeutic agent such as a β—2-agonist and a corticosteroid.

Preparation of Modulators

The following examples are offered to illustrate, but not to limit, the claimed invention.

Additionally, those skilled in the art will recognize that the molecules claimed in this patent may be synthesized using a variety of standard organic chemistry transformations.

Certain general reaction types employed widely to synthesize target compounds in this invention are summarized in the examples. Specifically, generic procedures for sulfonamide formation, pyridine N-oxide formation and 2-aminophenyl-arylmethanone synthesis via Friedel-Crafts type approaches are given, but numerous other standard chemistries are described within and were employed routinely.

While not intended to be exhaustive, representative synthetic organic transformations which can be used to prepare compounds of the invention are included below.

These representative transformations include: standard functional group manipulations; reductions such as nitro to amino; oxidations of functional groups including alcohols and pyridines; aryl substitutions via IPSO or other mechanisms for the introduction of a variety of groups including nitrile, methyl and halogen; protecting group introductions and removals; Grignard formation and reaction with an electrophile; metal-mediated cross couplings including but not limited to Stille, Ullmann, Harwig, Buckvald, Suzuki and Sonigashira reactions and variants thereof; halogenations and other electrophilic aromatic substitution reactions; diazonium salt formations and reactions of these species; etherifications; cyclative condensations, dehydrations, oxidations and reductions leading to heteroaryl groups; aryl metallations and transmetallations and reaction of the ensuing aryl-metal species with an electrophile such as an acid chloride or Weinreb amide; amidations; esterifications; nuclephilic substitution reactions; alkylations; acylations; sulfonamide formation; chiorosulfonylations; ester and related hydrolyses; Wittig, Peterson, Julia, Grubb olefinations and standard olefinations; and the like.

Certain molecules claimed in this patent can exist in different enantiomeric and diastereomeric forms and all such variants of these compounds are within the scope of the invention.

In the descriptions of the syntheses that follow, some precursors were obtained from commercial sources. These commercial sources include Aldrich Chemical Co., Acros Organics, Ryan Scientific Incorporated, Oakwood Products Incorporated, Lancaster Chemicals, Sigma Chemical Co., Lancaster Chemical Co., TCI-America, Alfa Aesar, Davos Chemicals, and GFS Chemicals.

Compounds of the invention, including those listed in the table of activities, can be prepared by the methods and approaches described in the following experimental section, and by the use of standard organic chemistry transformations that are well known to those skilled in the art. Examples of approaches that may be taken to synthesize these compounds are shown below. Nonetheless, one skilled in the art will recognize that alternative methods may be employed to synthesize the target compounds of this invention, and that the approaches described within the body of this document are not exhaustive, but do provide broadly applicable and practical routes to compounds of interest.

### Examples

Exemplary compounds used in the method of the invention and in pharmaceutical compositions of the invention include but are not limited to the compounds listed in Table 2. Pharmaceutically acceptable salts of the compounds listed in Table 2 are also useful in the method of the invention and in pharmaceutical compositions of the invention.

Reagents and solvents used below can be obtained from commercial sources such as Aldrich Chemical Co. (Milwaukee, Wis., USA). ¹H-NMR were recorded on a Varian Mercury 400 MHz NMR spectrometer. Significant peaks are tabulated in the order: multiplicity (br, broad; s, singlet; d, doublet; t, triplet; q, quartet; m, multiplet) and number of protons. Mass spectrometry results are reported as the ratio of mass over charge, followed by the relative abundance of each ion (in parenthesis). In tables; a single m/e value is reported for the M+H (or, as noted, M−H) ion containing the most common atomic isotopes. Isotope patterns correspond to the expected formula in all cases. Electrospray ionization (ESI) mass spectrometry analysis was conducted on a. Hewlett-Packard MSD electrospray mass spectrometer using the HP1100 HPLC for sample delivery. Normally the analyte was dissolved in methanol at 0.1 mg/mL and 1 microliter was infused with the delivery solvent into the mass spectrometer, which scanned from 100 to 1500 daltons. All compounds could be analyzed in the positive ESI mode, using acetonitrile/water with 1% formic acid as the delivery solvent. The compounds provided below could also be analyzed in the negative ESI mode, using 2 mM NH₄OAc in acetonitrile/water as the delivery system.

Compounds within the scope of this invention can be synthesized as described below, using a variety of reactions known to the skilled artisan. A sample of useful routes to both the benzophenone and heteroaryl derived subunits and to fully elaborated sulfonamide molecules of formulae (I, III, VI and VIII) within this claim are provided below.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

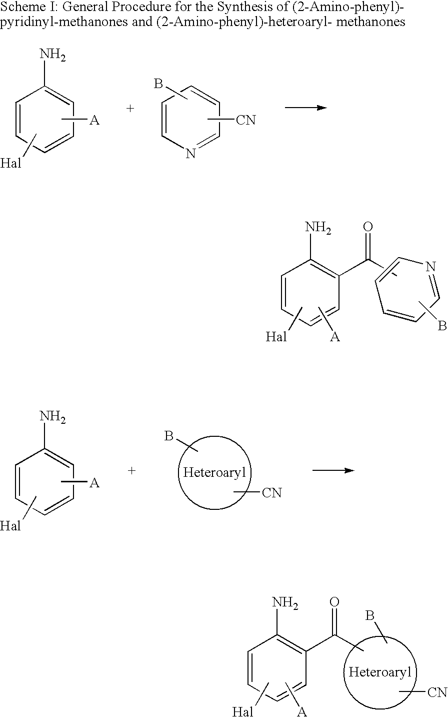

To 12.5 mL 1 M BCl₃ (12 mmol, 1.2 eq.) in methylene chloride stirred at 0° C. was added a solution of the desired haloaniline (10 mmol, 1.0 eq.) in 15 mL of TCE drop wise over 20 minutes. After 10 minutes the desired cyanopyridine (11 mmol, 1.1 eq.) was added followed by AlCl₃ (15 mmol, 1.5 eq.). The reaction was brought to RT, stirred for an hour then heated at 80-90° C. until all of the DCM was distilled off. The reaction mixture was then refluxed at 160° C. for 4 hours, cooled to RT and stirred overnight. 10 mL 3 M HCl were carefully added and the mixture was refluxed at 120° C. for 2-3 hours while reaction progress was monitored by LC/MS. The crude reaction was cooled to RT and 100 mL water were added. The crude mixture was extracted with DCM (2×50 mL), the aqueous layer was set aside and the organic layer was back extracted with 50 mL 1 M HCl (aq.). All aqueous layers were combined, brought to pH 12 with 3 M NaOH (aq.) and extracted with DCM (4×50 mL). The DCM layer was dried on Na₂SO₄, filtered and concentrated by rotary evaporation. The crude product was washed liberally with Et₂O and dried under vacuum, and further purified by conventional techniques such as column chromatography when necessary.

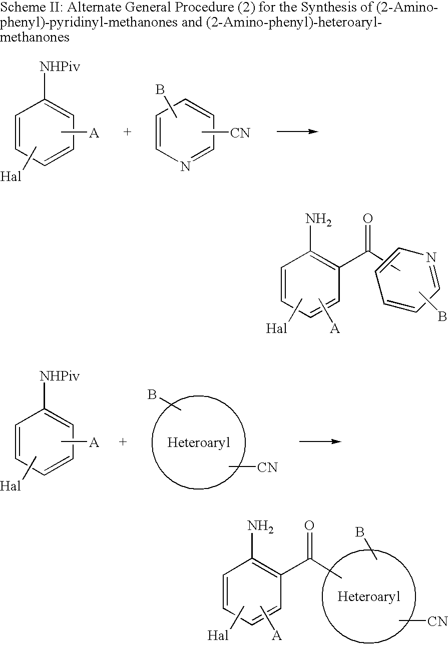

To a solution of desired aniline (40 mmol) in 25 mL pyridine was added 5.3 mL (43.1 mmol) of pivaloyl chloride and the reaction mixture stirred overnight at room temperature. The mixture was poured into vigorously stirring 6M HCl, and the solids were collected by vacuum filtration, washed well with H₂O, and dried in vacuo.

2,2-Dimethyl propanamide protected aniline (0.0095 mol) in dry THF (40 mL) was cooled to −5° C. n-Butyl lithium (24 mL, 1.2 M, 0.0284 mol) was added dropwise and the reaction stirred at the same temperature for 2 h. The reaction mixture was cooled to −70° C. and to this was added the desired aryl or heteroaryl carboxylic acid (0.0142 mol), dissolved in dry THF (10 mL), dropwise The mixture was stirred at room temperature for 18 h, quenched with water and extracted with ethyl acetate. The extract was washed with brine solution and concentrated. The product was purified by column using 5-10% of ethyl acetate in pet ether as eluent.

Removal of the pivaloyl protecting group form the amino ketone (0.4 g, 0.0013 mol) in 2 mL of methanol was effected via addition of potassium hydroxide (0.48g, 0.00857 mol) in 1.2 mL of water. The reaction mixture was heated at 70° C. for 6 h, diluted with water and extracted with ethyl acetate. The extract was washed with water, brine and concentrated. The crude material was purified by column chromatography.

Alternatively, 6N HCl (10 mL) and intermediate pivaloyl protected aminoketone (1.2 g, 3.755 mmol) were heated at 90° C. overnight, cooled to room temperature, the reaction mixture basified by adding saturated sodium bicarbonate solution and extracted with ether. The organic layer was washed with brine, dried over sodium sulfate and concentrated. The product was purified by column chromatography.

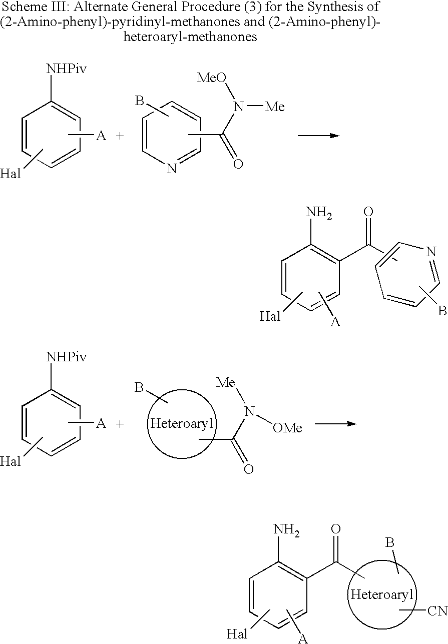

Trimethylacetyl chloride was added drop wise to a solution of desired aniline in dry pyridine and the reaction was stirred under nitrogen overnight. About half of the pyridine was removed by rotary evaporation, then the mixture was treated with 6M hydrochloric acid and extracted with ethyl acetate. The extracts were washed with saturated aqueous NaHCO₃ and with water, then were dried (MgSO₄), filtered and concentrated by rotary evaporation.

EDC and desired heteroaryl carboxylic acid were stirred in acetonitrile-THF with N,O-dimethylhydroxylamine hydrochloride and triethylamine. After stirring overnight at ambient temperature, the resulting reaction mixture was added to ice water and extracted with ethyl acetate (3×100 mL). The extracts were dried, filtered, and concentrated.

To a stirred solution of the pivaloyl protected intermediate in dry THF was added 2.5M n-butyllithium in hexane at −40° C. and the mixture was stirred at 0° C. for 2 h. A solution of the Weinreb amide in dry THF was added dropwise and the reaction was stirred at ambient temp overnight. The mixture was diluted with water and extracted with ethyl acetate and the organic layer was dried (MgSO₄), filtered and concentrated, to yield, after purification by HPLC or column chromatography, the pivaloyl protected aminoketone intermediate.

Deprotection with 70% sulfuric acid was carried out at 75° C. and progress monitored by LC/MS. The reaction was allowed to cool to ambient temperature, and was washed with ether-hexane. The acidic aqueous layer was cooled in an ice bath and aqueous NaOH was added drop wise to basify the mixture. The product was extracted with ethyl acetate and the extracts were washed with saturated aqueous NaHCO3 (2×100 mL), with saturated aqueous sodium chloride, dried (MgSO₄), filtered and concentrated, yielding the desired (2-Amino-phenyl)-heteroaryl-methanone.

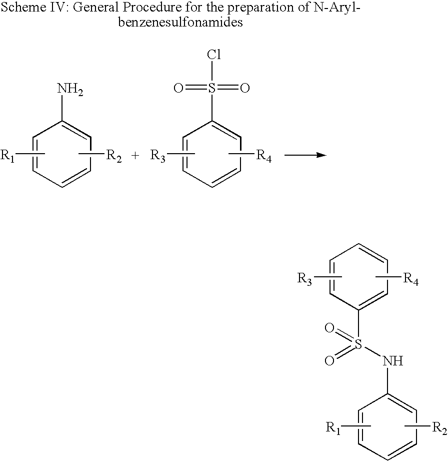

To the desired aniline (0.5 mmol) dissolved in pyridine and cooled in an ice-water bath was added a solution of an aryl sulfonyl chloride (0.5 mmol) dissolved in cold pyridine. The reaction mixture was then heated to 60° C. with gentle shaking for 16 h. Evaporation of the solvent with standard workup followed by either flash chromatography or reversed phase HPLC yielded the corresponding N-aryl-benzenesulfonamides.

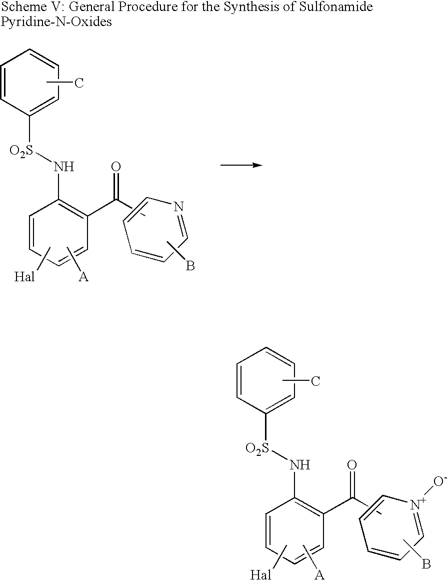

The desired N-aryl-benzenesulfonamide (250 μmol) was dissolved in _{CH2Cl2} (2 mL) and m-CPBA (1.0-1.5 eq) was then added. The reaction was shaken at RT and monitored by LC-MS. Additional m-CPBA was added as needed in aliquots until the reaction was complete. In most cases the reaction required 15-24 h reaction time. Standard workup led to the required products.

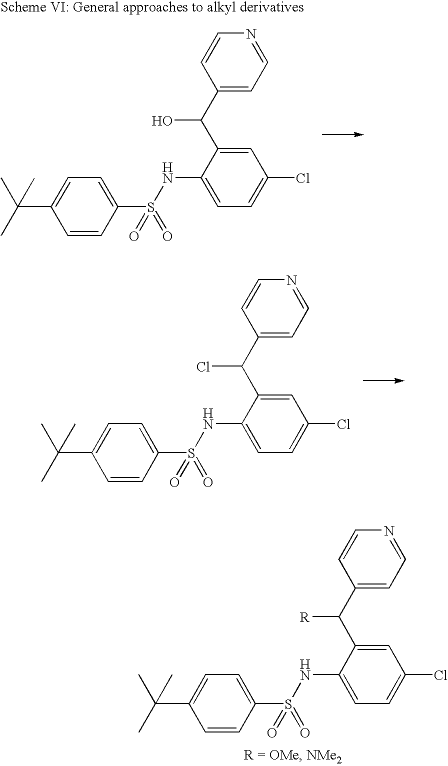

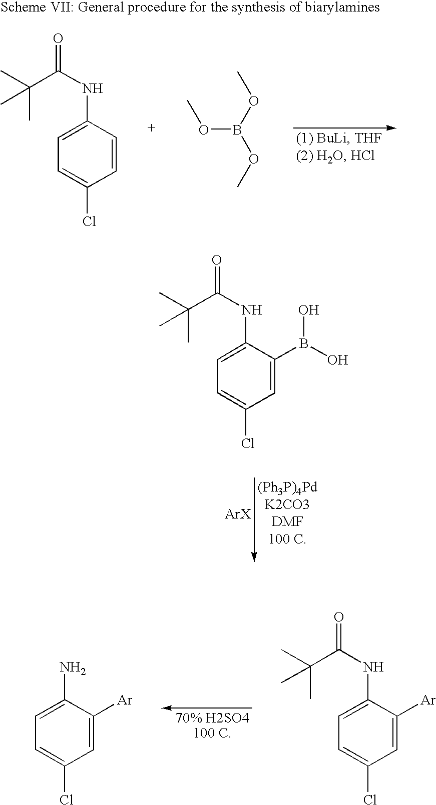

N-(4-Chloro-phenyl)-2,2-dimethyl-propionamide (2.11 g, 10 mmol) was dissolved in anhydrous THF (25 mL) and cooled to 0° C., then n-butyl lithium (22 mmol, 8.8 mL of 2.5 M solution in hexanes) was added dropwise over 30 minutes. The reaction mixture was stirred at this temperature for an additional 2 h and was then cooled to −78° C. A solution of trimethylborate (3.11 g, 30 mmol) in anhydrous THF was added and the mixture was allowed to warm to room temperature and stirred overnight. After concentrating by rotary evaporation the crude product was acidified with 3N

HCl. The crystalline solid was collected by filtration, washed with water and dried to afford 1.2 g of the boronic acid MS: MS (M+H⁺): 256. The boronic acid (0.5 mmol), the appropriate aryl halide (0.5 mmol), tetrakis(triphenylphosophine)palladium(0) (0.025 mmol) and K₂CO₃ (1 mmol) were suspended DMF (5 mL) and heated at 100° C. overnight. After cooling to room temperature the reaction mixture was diluted with ether (20 mL) and washed twice with 10 mL portions of water. The organic extract was concentrated and product was purified by flash chromatography on silica gel column using 5-20% ethylacetate/hexane solvent mixture. The pivaloyl protected biaryl was suspended in 70% sulfuric acid and refluxed overnight. After cooling to room temperature the reaction was cautiously added to cold concentrated aqueous NaOH solution to achieve neutral pH and extracted with ethyl acetate. The organic layer was dried over anhydrous magnesium sulfate and the solvent was evaporated to afford product.

### 4-Chloro-2-pyridin-2-yl-phenylamine

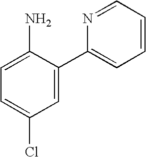

The title compound was prepared according to the general procedure described in Scheme XI using 2-chloropyridine as the aryl halide component. MS: (M+H)/z=205.

### 4-Chloro-2-isoquinolin-1-yl-phenylamine

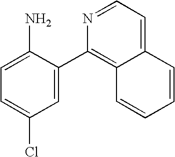

The title compound was prepared according to the general procedure described in Scheme XI using 1-chloroisoquinoline as the aryl halide component. MS: (M+H)/z=255.

### 4-Chloro-2-isoquinolin-3-yl-phenylamine

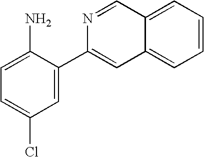

The title compound was prepared according to the general procedure described in Scheme XI using 3-bromoisoquinoline as the aryl halide component. MS: (M+H)/z=255.

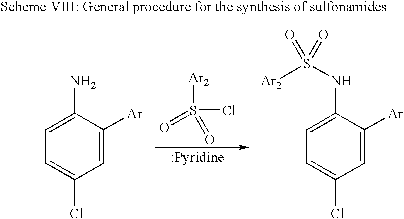

To the desired aniline (0.5 mmol) dissolved in pyridine and cooled in an ice-water bath was added a solution of an aryl sulfonyl chloride (0.5 mmol) dissolved in cold pyridine. The reaction mixture was then heated to 60° C. with gentle shaking for 16 h. Evaporation of the solvent with standard workup followed by either flash chromatography or reverse phase HPLC yielded the corresponding N-aryl-benzenesulfonamide.

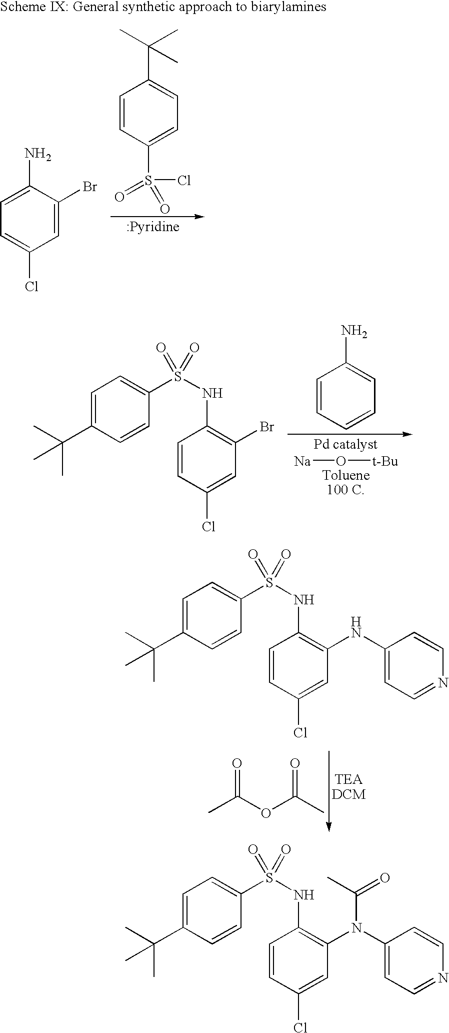

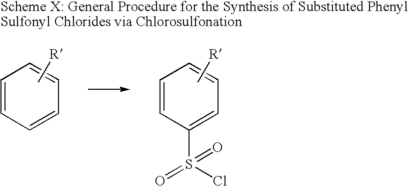

The desired benzene derivative (1.4 mmol) was dissolved in CHCl₃ (15 mL) at 0° C., and to this was added chlorosulfonic acid (4.2 mmol). After 30 minutes, the reaction mixture was warmed to room temperature, and additional chlorosulfonic acid (4.2 mmol) was added. After a further hour, the reaction mixture was cooled to 0° C., and crushed ice added to the reaction. The reaction mixture was partitioned between 1M pH 7 phosphate buffer and ether, and the ether layer washed with saturated aqueous NaCl, dried over Na₂SO₄, filtered, and concentrated in vacuo to yield crude product.

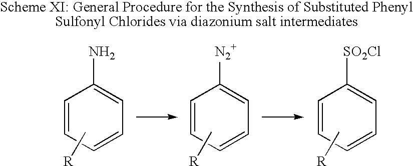

The desired aniline (0.0848 mol) was added slowly to concentrated HCl (109.2 mL), the reaction stirred at room temperature for 15 min, then cooled to 0° C., sodium nitrite (6.2 g, 0.1103 mol in 26 mL of water) added dropwise and the reaction stirred for 15 min.

Separately, distilled water (0.198 mL) was cooled to 0° C. and thionyl chloride (42.9 g, 0.3605 mol) was added dropwise, the mixture warmed to and stirred at room temperature for 17 h, then re-cooled to 0° C., and copper (I) chloride (0.120 g) added in small portions with further stirring for 30 mins to yield a yellowish green solution (Solution A).

This Cu(I) solution (A) was added dropwise to the aniline/HCl solution at −5° C. , and stirring continued at 0° C. for 75 min. The reaction mixture was diluted with chloroform. The organic layer was separated, washed with brine, dried over sodium sulphate and concentrated in vacuo.

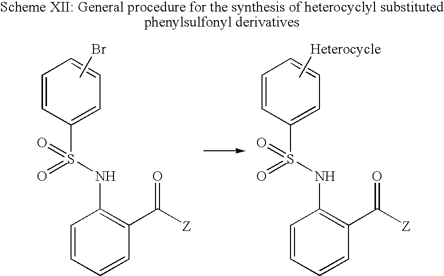

The desired bromobenzenesulfonamide derivative (0.22 mmol) was dissolved in anhydrous dioxane (6 mL), and to this solution was added potassium phosphate tribasic monohydrate (1.32 mmol), rac-2,2′-bis(diphenylphosphino)-1,1′-binaphthyl (0.032 mmol), followed by the desired heterocycle (1.1 mmol). The mixture was purged under nitrogen, and Pd(dba)₃ (0.01 mmol) was added. The reaction mixture was heated overnight at 90° C., cooled, water (5 mL) added, and extracted with ethyl acetate 3 times. The combined organic layer was washed with brine, dried over magnesium sulfate, filtered and concentrated. The crude reaction mixture was purified using HPLC.

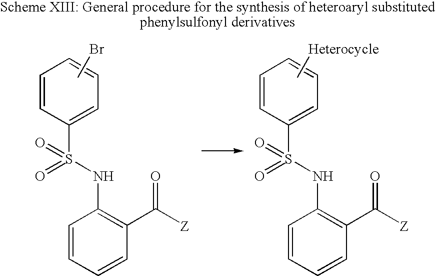

The desired bromobenzenesulfonamide derivative (0.25 g, 0.55 mmol) was dissolved in 2.5 mL of anhydrous dimethylformamide. To this solution was added 0.14 g (1.3 mmol) sodium carbonate, suitable heteroaryl-3-boronic acid (0.68 mmol), and Pd(PPh₃)₄ 19 mg (0.014 mmol). The reaction mixture was heated overnight at 80° C. under a nitrogen atmosphere. The mixture was quenched with water and extracted 3 times with dichloromethane. The organic layer was dried over magnesium sulfate, filtered, and concentrated, and the crude product purified by flash column chromatography.

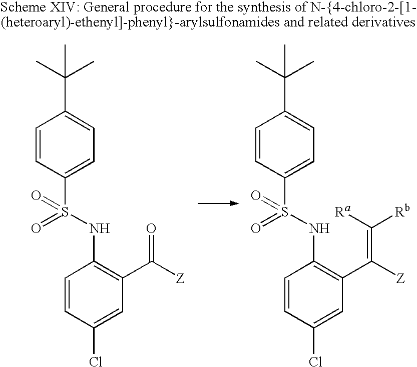

To a magnetically stirred suspension of arylketone (1.0 mmol) in dry THF (30 mL) cooled to −78° C. was added dropwise a solution of 1.4 M Grignard reagent in toluene/THF (1.4 mL) and the reaction was slowly warmed to 0° C. The reaction was monitored by LCMS, and after 8 h was quenched with water and concentrated to give crude alcohol intermediate that was purified by preparative HPLC or column chromatography.

To this alcohol (0.080 mmol) in dry toluene (10 mL) was added 5.0 mg of para-toluenesulfonic acid monohydrate and the reaction was heated at reflux under nitrogen. The reaction was monitored by LCMS, and after 72 h was concentrated to give crude olefin product, which was purified by preparative HPLC or column chromatography.

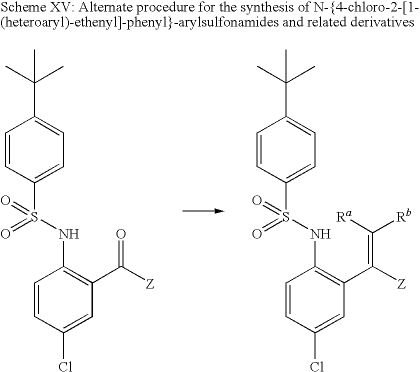

General olefination procedures can be employed to affect the conversion of aryl ketones to alkene derivatives. Non-exhaustive standard organic chemistry transformations that can be employed include (a) the Wittig olefination (b) Peterson olefiniation (c) Lombardo reaction (d) Metal mediated carbene insertion reactions (e) dehydration reactions of an intermediate alcohols catalysed or enabled by an inorganinc or organic acid or other standard dehydrating reagents (f) elimination of an active intermediate such as a mesylate or tosylate.

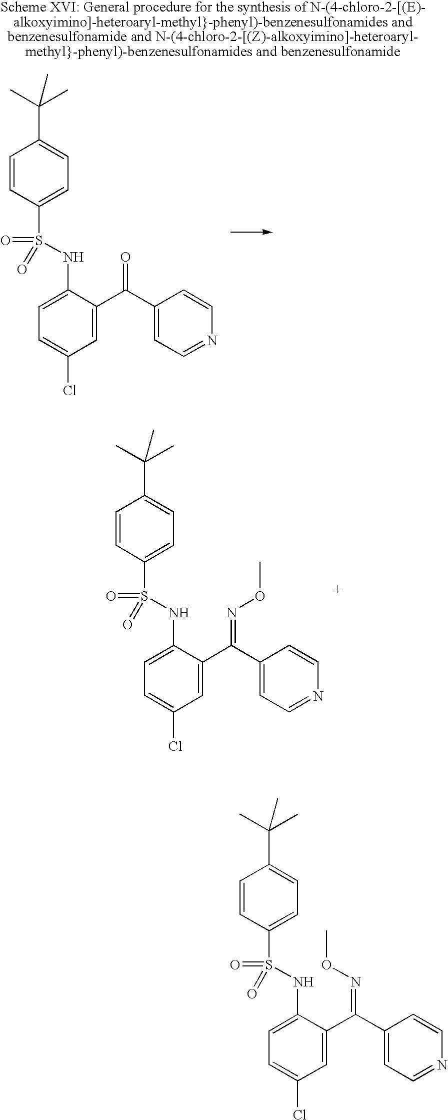

Aryl ketone (1 mmol), O-alkylhydroxylamine hydrochloride (835 mg, 10 mmol), anhydrous pyridine (1.6 mL) and absolute ethanol (5 mL) were placed in a vial. The vial was capped, placed in a 70° C. sand-bath and shaken for 20 h. After cooling to room temperature the crude reaction mixture was diluted with ethyl acetate (20 mL) and washed twice with water (20 mL). The organic layer was concentrated and the isomeric products were isolated by preparative HPLC.

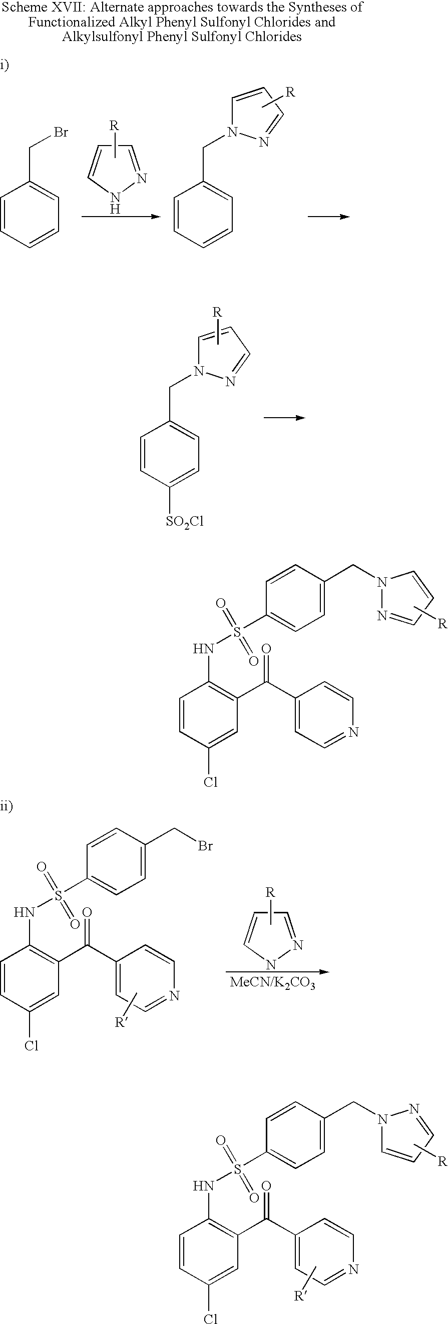

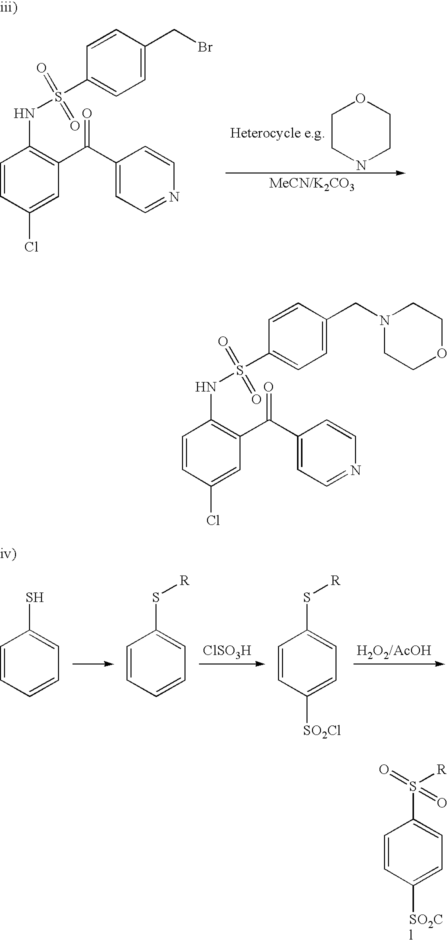

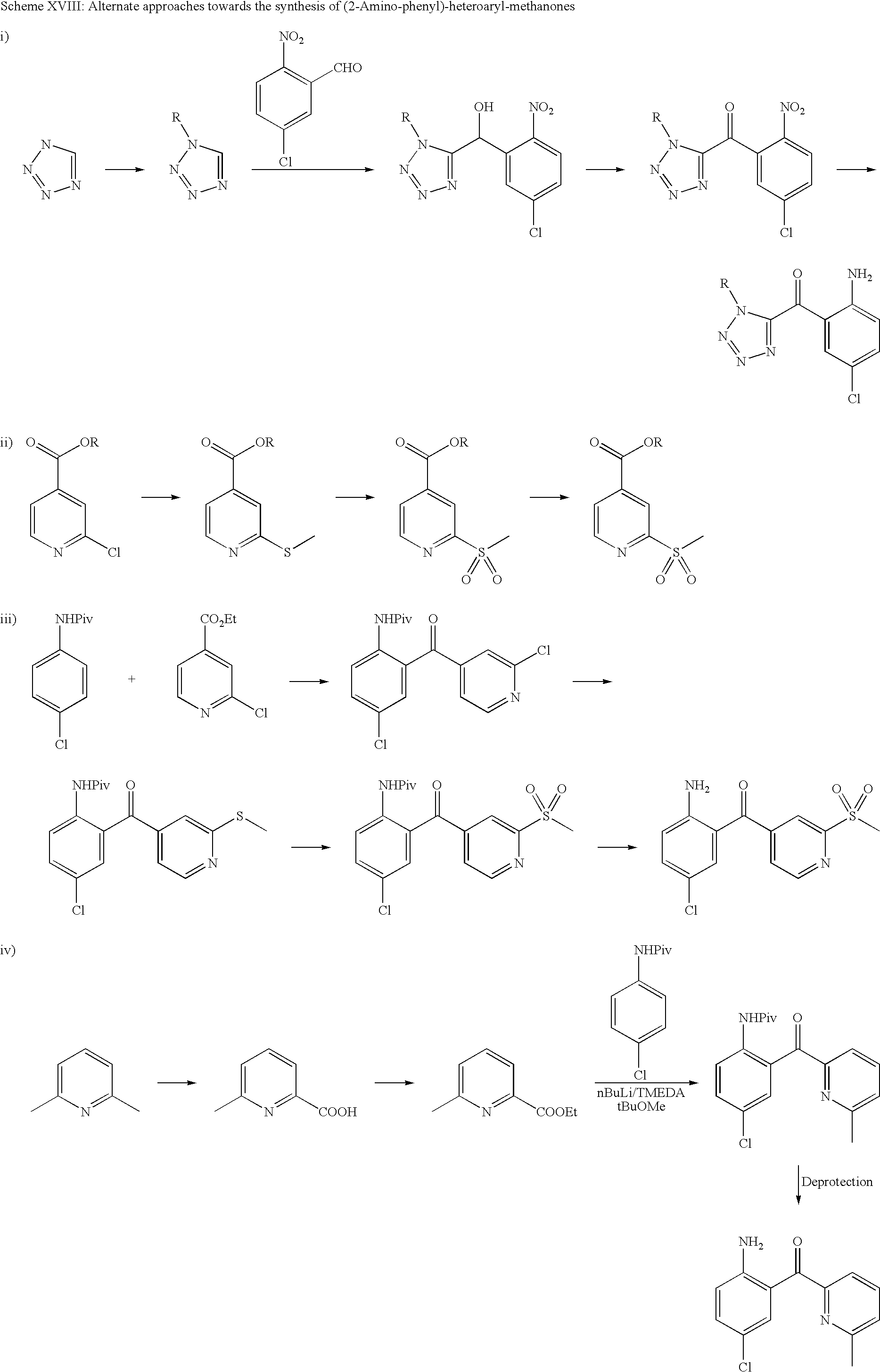

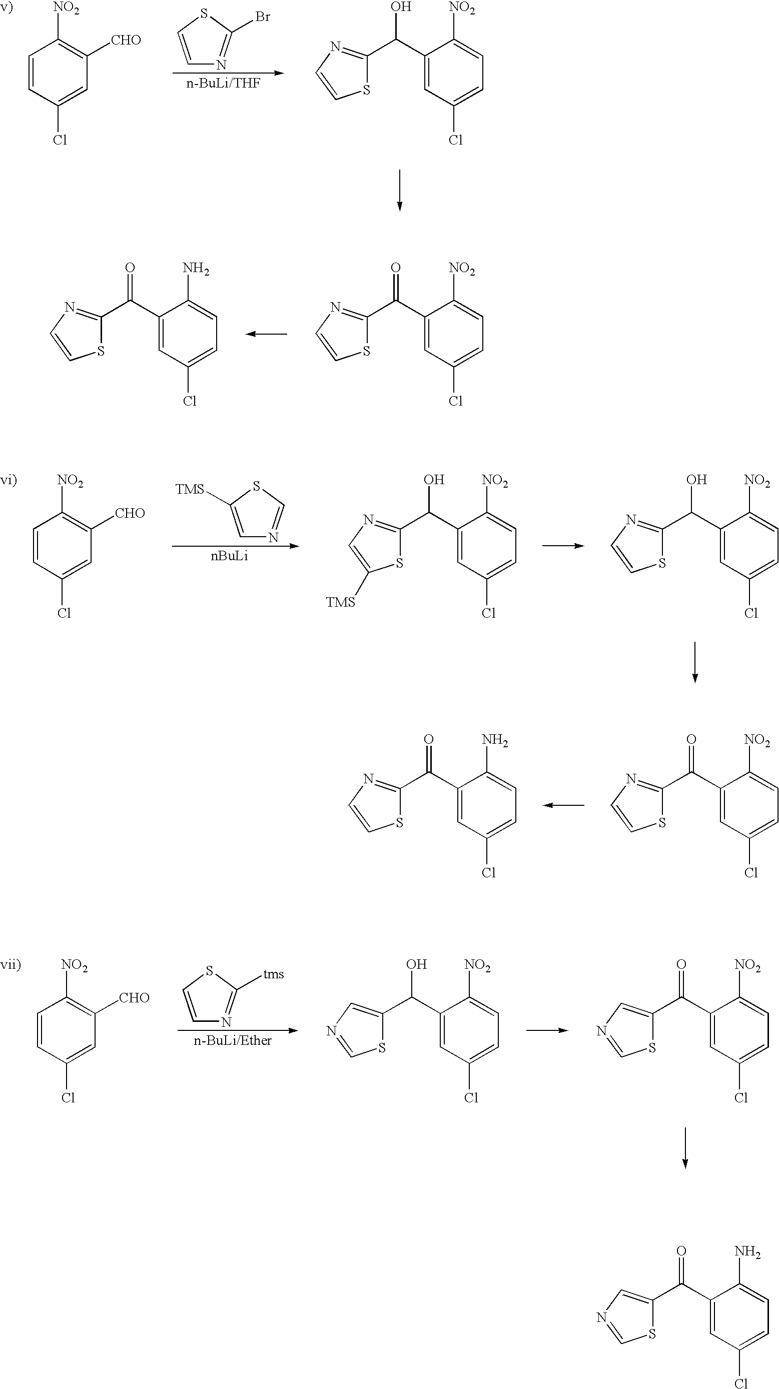

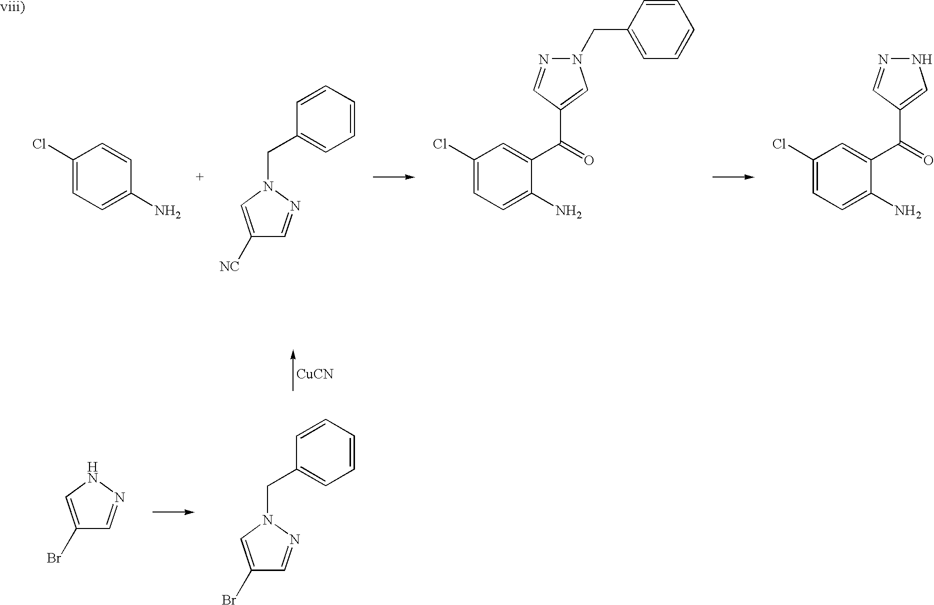

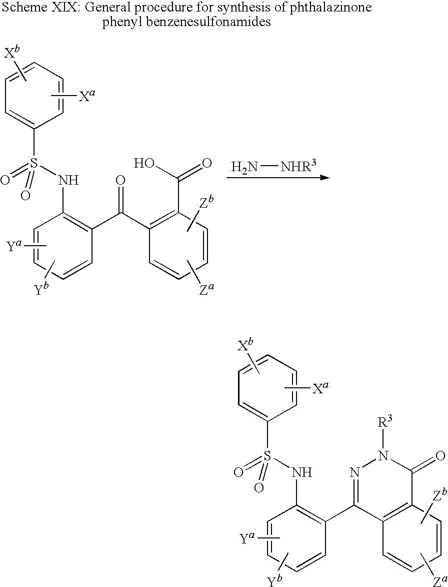

To a solution of the keto arylsulfonamide in a suitable solvent such as ethanol is added hydrazine or an alkyl hydrazine. The resulting mixture is heated to reflux to form the phthalazinone ring. After the reaction is complete, mixture is cooled to room temperature, diluted with an organic solvent, washed with dilute aqueous acid, dried and concentrated under reduced pressure. The residue is purified by preparative HPLC to afford the phthalazinone phenyl benzenesulfonamide.

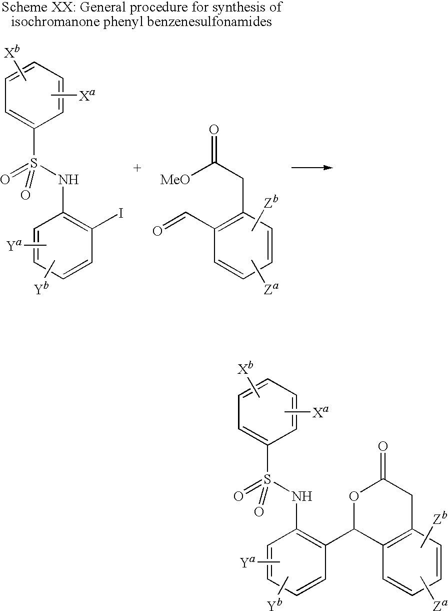

To a stirred soloution of the iodo-sulfonamide in an appropriate solvent such as THF at 0° C. was added dropwise, a solution of isopropyl magnesium bromide. After the iodo/magnesium exchange is complete (about 1 hour), the aldehyde (2-3 eq.) is added. The resulting mixture is stirred at 0° C. and then warmed to room temperature. The reaction mixture is quenched with aqueous NH₄Cl and extracted with EtOAc. The combined organic extracts are washed with brine, dried (Na₂SO₄), filtered, and concentrated under reduced pressure to afford the isochromanone phenyl benzenesulfonamide.

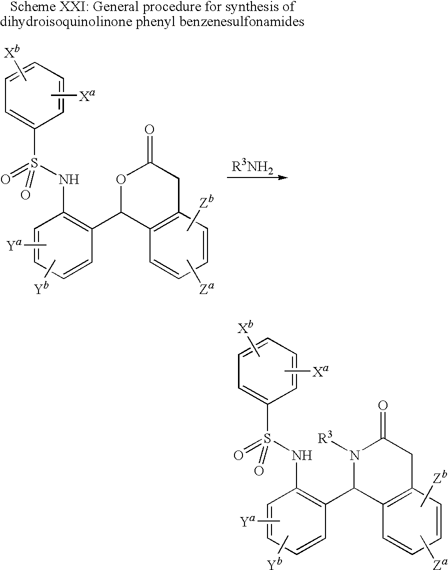

To a solution of the isochromanone phenyl benzenesulfonamide and an appropriate primary amine in pyridine is added a solution of KMnO₄ (2 eq.) in 1 N NaOH. The mixture is heated at 80° C. and monitored by LCMS. After reaction completion, the mixture is diluted with water, cooled by means of an ice-water bath and acidified to pH 1 with concentrated HCl. The resulting keto acid is extracted with EtOAc and purified via flash chromatography on silica gel. The product containing fraction are combined and the solvent removed under reduced pressure. The residue is dissolved in dry THF containing 0.1% acetic acid and treated with sodium triacetoxyborohydride (3 eq.). The reaction is stirred at room temperature overnight and the mixture is diluted with water, extracted with EtOAc. The combined organic layers are concentrated under reduced pressure and purified via flash chromatography to afford the dihydroisoquinolinone phenyl benzenesulfonamide.

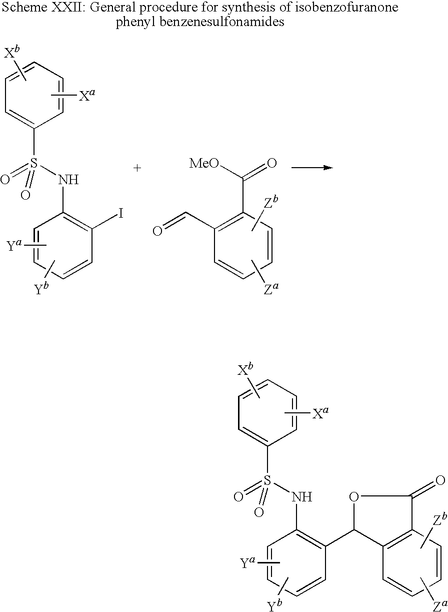

To a stirred soloution of the iodo-sulfonamide in an appropriate solvent such as THF at 0° C. was added dropwise, a solution of isopropyl magnesium bromide. After the iodo/magnesium exchange is complete (about 1 hour), the aldehyde (2-3 eq.) is added. The resulting mixture is stirred at 0° C. and then warmed to room temperature. The reaction mixture is quenched with aqueous NH₄Cl and extracted with EtOAc. The combined organic extracts are washed with brine, dried (Na₂SO₄), filtered, and concentrated under reduced pressure to afford the isobenzofuranone phenyl benzenesulfonamide.

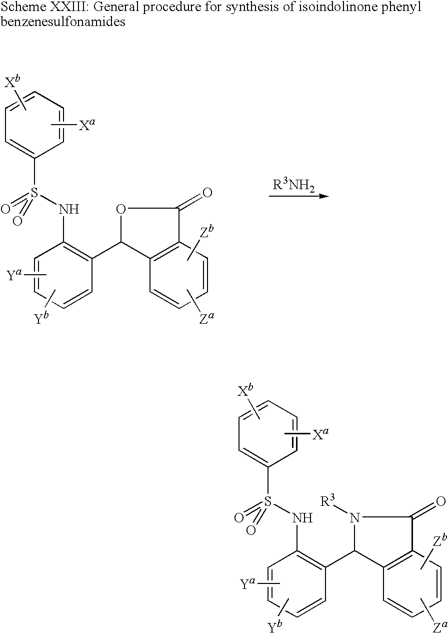

A solution of the isobenzofuranone phenyl benzenesulfonamide and an appropriate primary amine are suspended ethanol, heated at 80° C., and monitored by TCL or LCMS for reaction completion. After reaction completion, the mixture is cooled, diluted with water, and extracted with EtOAc. The combined organic layers are concentrated under reduced pressure and purified via flash chromatography to afford the isoindolinone phenyl benzenesulfonamide.

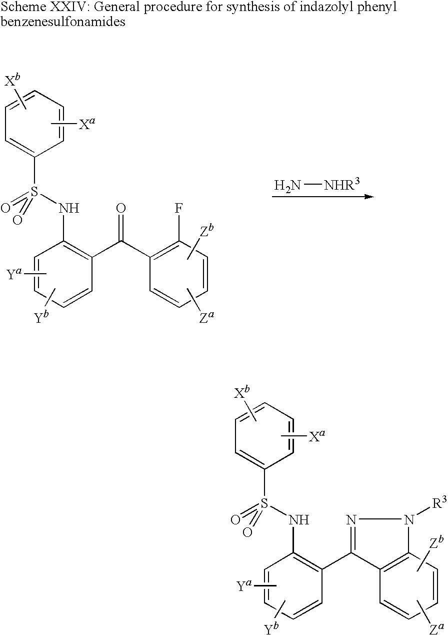

To a solution of the fluoro aminobenzophenone benzenesulfonamide in a suitable solvent is added hydrazine or an alkyl hydrazine (such as methyl hydrazone). The resulting mixture is heated to reflux to form the phthalazinone ring. The resulting mixture is cooled to room temperature, diluted with an organic solvent, washed with dilute aqueous acid, washed with dilute aqueous base, dried and concentrated under reduced pressure. The residue is purified by preparative HPLC to afford the indazolyl phenyl benzenesulfonamides.

### Example 1

### 4-Chloro-N-(5-chloro-2-phenoxy-phenyl)-3-trifluoromethyl-benzenesulfonamide

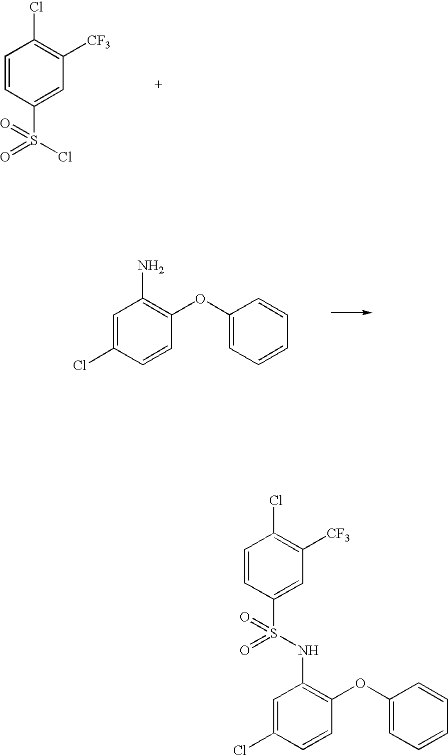

To a solution of 5-chloro-2-phenoxy-phenylamine (75 mg, 0.34 mmol) in anhydrous pyridine (0.5 mL) was added drop wise a solution of 4-chloro-3-trifluoromethyl-benzenesulfonyl chloride (95 mg, 0.341 mmol) in pyridine (0.5 mL). The resulting mixture was stirred at room temperature for 2 h. The reaction mixture was separated by preparative HPLC (20→80% gradient of ACN-water) and pure product fractions were lyophilized to provide pure product as a solid. ¹H NMR (400 MHz, CDCl₃) δ 8.04 (d, J=2.0 Hz, 1H), 7.80 (dd, J=8.4, 2.0 Hz, 1H), 7.70 (d, J=2.4 Hz, 1H), 7.51 (d, J=8.4 Hz, 1H), 7.27-7.23 (m, 2H), 7.14-7.10 (m, 1H), 7.05-7.02 (m, 2H), 6.66 (d, J=8.4 Hz, 1H), 6.60-6.56 (m, 2H). MS m/z: 484.0 (M+Na).

### Example 2

### 3,4-Dichloro-N-[5-chloro-2-(morpholine-4-carbonyl)phenyl]benzenesulfonamide

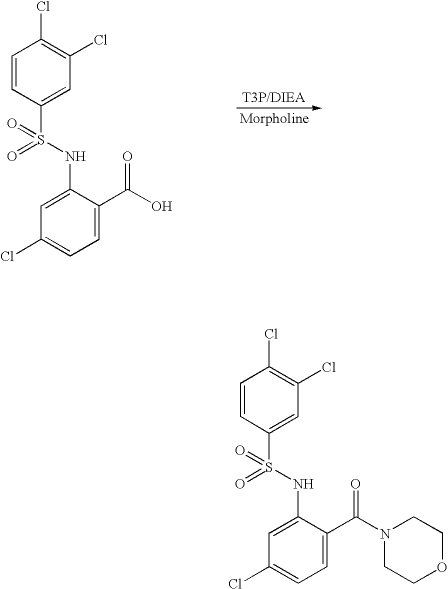

To a mixture of 4-chloro-2-(3,4-dichlorobenzenesulfonylamino)-benzoic acid (21 mg), morpholine (10 mg) and N,N-diisopropylethylamine (0.019 mL) in CH₂Cl₂ (1.5 mL) was added 1-propanephosphonic acid cyclic anhydride (50% in ethyl acetate, 0.024 mL). After four hours the reaction mixture was directly purified via flash column (65% ethyl acetate in hexane) to afford 14 mg of 3,4-dichloro-N-[5-chloro-2-(morpholine-4-carbonyl)phenyl]benzenesulfonamide as a pure white powder. ¹H-NMR (400 MHz, CDCl₃): δ 7.86 (d, 1H), 7.67-7.60 (m, 2H), 7.36 (m, 1H), 7.14 (m, 1H), 6.72 (m, 1H), 3.71 (m, 4H), 3.12 (m, 4H). MS: (M+H)/z=451.2.

### Example 3

### 3,4-Dichloro-N-[2-(2-oxazolyl-5 chlorophenyl]benzenesulfonamide

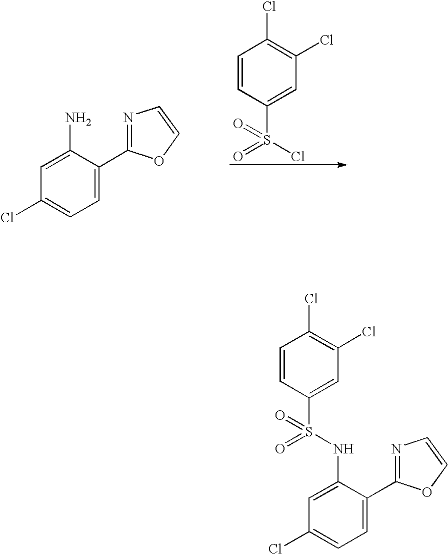

To an ice-water cooled solution of 2-(4-chloro-2-aminophenyl)-1,3-oxazole (97 mg, 0.5 mmol) in pyridine (1.0 mL) was added 3,4-dichlorobenzenesulfonylchloride (123 mg, 0.5 mmol) in pyridine (0.5 mL). The mixture was stirred at room temperature for 3 hours. The mixture was directly purified via Prep HPLC to give 3,4-dichloro-N-[2-(2-oxazolyl-5 chlorophenyl]benzenesulfonamide as a white powder. ¹H-NMR (400 MHz, CDCl₃): δ 7.94 (s, 1H), 7.85 (d, J=1.6 Hz, 1H), 7.75 (s, 1H), 7.70 (s, 1H), 7.64 (m, 1H), 7.48 (m, 1H), 7.30 (s, 1H), 7.10 (d, 1H). MS: (M+H)/z=405.

### Example 4

### 4-Chloro-N-[5-chloro-2-(pyridine-4-carbonyl)-phenyl]-3-trifluoromethylbenzenesulfonamide

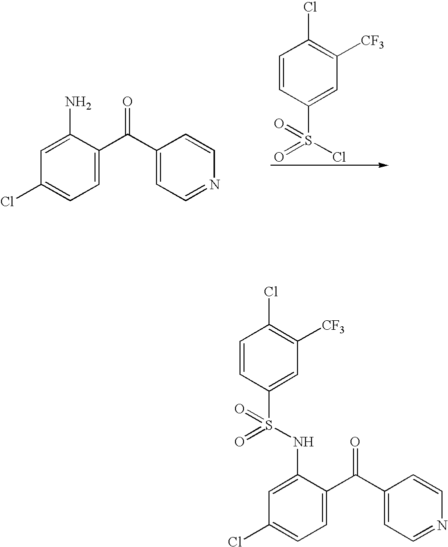

To an ice-water cooled solution of 2-amino-4-chloro-phenyl-pyridin-4-yl-methanone (40 mg, 0.17 mmol) in pyridine (1.0 mL) was added 4-chloro-3-trifluoromethylbenzenesulfonylchloride (48 mg, 0.17 mmol) in pyridine (0.5 mL). The mixture was stirred at room temperature for 3 hours. The mixture was diluted with ethyl acetate, washed with water and dried. After concentration the residue was purified via flash column (50% ethyl acetate in hexane) to give 4-chloro-N[5-chloro-2-(pyridine-4-carbonyl)-phenyl]-3-trifluoromethylbenzenesulfonamide as a white powder. ¹H-NMR (400 MHz, CDCl₃): δ 8.73 (d, 2H), 7.95 (d, 1H), 7.80 (m, 1H), 7.67 (m, 1H), 7.52-7.49(m, 2H), 7.45 (m, 1H), 7.37 (m, 1H) MS: (M+H)/z=475.0.

### Example 5

### 4-Chloro-N-[5-chloro-2-pyridin-2-yl-phenyl]-3-trifluoromethylbenzenesulfonamide

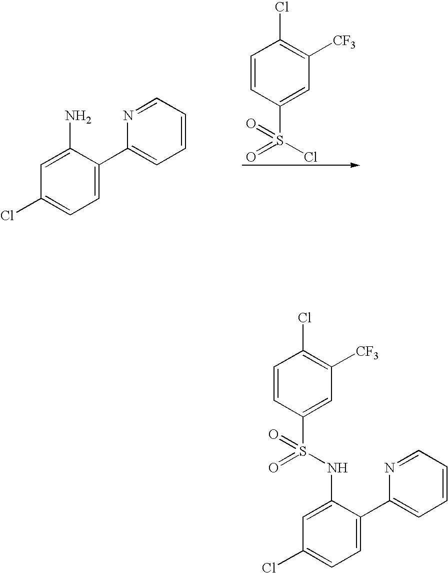

To an ice-water cooled solution of 5-chloro-2-(2-pyridin-yl)-aniline (42 mg, 0.20 mmol) in pyridine (1.0 mL) was added 4-chloro-3-trifluoromethylbenzenesulfonylchloride (57 mg, 0.20 mmol) in pyridine (0.5 mL). The mixture was stirred at room temperature for 3 hours. The mixture was diluted with ethyl acetate, washed with water and dried. After concentration the residue was purified via flash. column (30% ethyl acetate in hexane) to give 4-chloro-N-[5-chloro-2-pyridin-2-yl-phenyl]-3-trifluoromethylbenzenesulfonamide as a white powder. ¹H-NMR (400 MHz, CDCl₃): δ 11.98 (s, 1H), 8.60 (s, 1H), 7.76-7.62 (m, 3H), 7.45-7.25 (m, 6H). MS: (M+H)/z=447.2.

### Example 6

### 4-Chloro-3-trifluoromethyl-N-[5-chloro-2-(morpholine-4-carbonyl)-phenyl]benzenesulfonamide

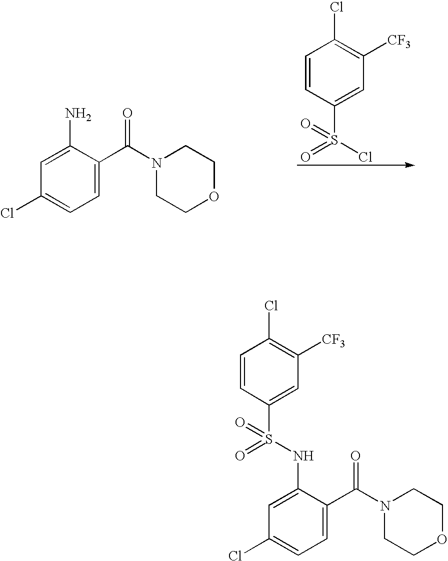

To an ice-water cooled solution of (2-amino-4-chloro-phenyl)-morpholin-4-yl-methanone (170 mg, 0.71 mmol) in pyridine (1.0 mL) was added 4-chloro-3-trifluoromethylbenzenesulfonylchloride (191 mg, 0.71 mmol) in pyridine (0.5 mL). The mixture was stirred at room temperature for 3 hours. The mixture was diluted with ethyl acetate, washed with water and dried. After concentration the residue was purified via flash column (60% ethyl acetate in hexane) to give 113 mg of 4-chloro-3-trifluoromethyl-N-[5-chloro-2-(morpholine-4-carbonyl)-phenyl]benzenesulfonamide as a white powder. ¹H-NMR (400 MHz, CDCl₃): δ 8.80 (s, 1H), 8.15 (s, 1H), 7.96 (d, 1H), 7.65-7.60 (m, 2H), 7.25-7.10 (m, 2H), 3.78-3.10 (m, 8H). MS: (M+H)/z=483.0.

### Example 7

### 4-Chloro-3-trifluoromethyl-N-[5-chloro-2-(piperidine-1-carbonyl)-phenyl]benzenesulfonamide

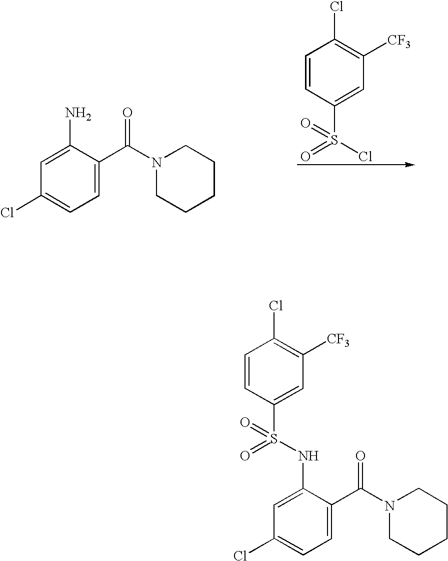

To an ice-water cooled solution of (2-amino-4-chloro-phenyl)-piperidin-1-yl-methanone (170 mg, 0.71 mmol) in pyridine (1.0 mL) was added 4-chloro-3-trifluoromethylbenzenesulfonylchloride (191 mg, 0.71 mmol) in pyridine (0.5 mL). The mixture was stirred at room temperature for 3 hours. The mixture was diluted with ethyl acetate, washed with water and dried. After concentration the residue was purified via flash column (60% ethyl acetate in hexane) to give of 4-chloro-3-trifluoromethyl-N-[5-chloro-2-(piperidine-1-carbonyl)-phenyl]benzenesulfonamide as a white powder. ¹H-NMR (400 MHz, CDCl₃): δ 8.88 (s, 1H), 8.13 (s, 1H), 7.94 (m, 1H), 7.67-7.55 (m, 2H), 7.08 (m, 2H), 3.60-3.21 (m, 4H), 1.79-1.32 (m, 6H). MS: (M+H)/z =481.1.

### Example 8

### 4-Chloro-3-trifluoromethyl-N-[5-chloro-2-(pyrrolidine-1-carbonyl)-phenyl]benzenesulfonamide

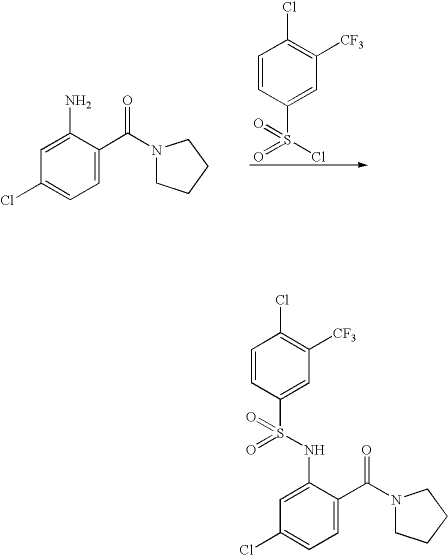

To an ice-water cooled solution of (2-amino-4-chloro-phenyl)-piperidin-1-yl-methanone (165 mg, 0.71 mmol) in pyridine (1.0 mL) was added 4-chloro-3-trifluoromethylbenzenesulfonylchloride (191 mg, 0.71 mmol) in pyridine (0.5 mL). The mixture was stirred at room temperature for 3 hours. The mixture was diluted with ethyl acetate, washed with water and dried. After concentration the residue was purified via flash column (60% ethyl acetate in hexane) to give 4-chloro-3-trifluoromethyl-N-[5-chloro-2-(pyrrolidine-1-carbonyl)-phenyl]benzenesulfonamide as a white powder. ¹H-NMR (400 MHz, CDCl₃): δ 8.88 (s, 1H), 8.13 (s, 1H), 7.94 (m, 1H), 7.67-7.55 (m, 2H), 7.08 (m, 2H), 3.60-3.21 (m, 4H), 1.79-1.32 (m, 4H). MS: (M+H)/z=467.0.

### Example 9

### 1-[4-chloro-3-trifluoromethyl-benzenesulfonylamino)-benzoyl]-pyrrolidine-2-carboxylic acid methyl ester

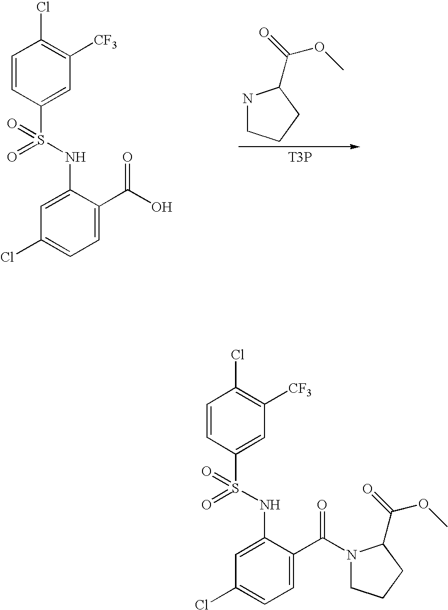

To a mixture of 4-chloro-2-(4-chloro-3-trifluoromethylbenzenesulfonylamino)-benzoic acid (20.3 mg), methyl proline hydrochloride (25 mg) and N,N-diisopropylethylamine (0.052 mL) in CH₂Cl₂ (1.0 mL) was added 1-propanephosphonic acid cyclic anhydride (50% in ethyl acetate, 0.045 mL). After four hours the reaction mixture was directly purified via flash column (65% ethyl acetate in hexane) to afford 7.0 mg of 1-[4-chloro-3-trifluoromethyl-benzenesulfonylamino)-benzoyl]-pyrrolidine-2-carboxylic acid methyl ester as a white powder. ¹H-NMR (400 MHz, CDCl₃): δ 9.04 (m, 1H), 8.27 (m, 1H), 7.90 (m, 1H), 7.83 (m, 1H), 7.52 (m, 1H), 7.12-7.05 (m, 2H), 4.97 (m, 1H), 3.82 (s, 3H), 3.20 (m, 2H), 2.18-1.85 (m, 4H). MS: (M+H)/z=525.0.

### Example 10

### 1-[4-chloro-3-trifluoromethyl-benzenesulfonylamino)-benzoyl]-pyrrolidine-2-carboxylic acid

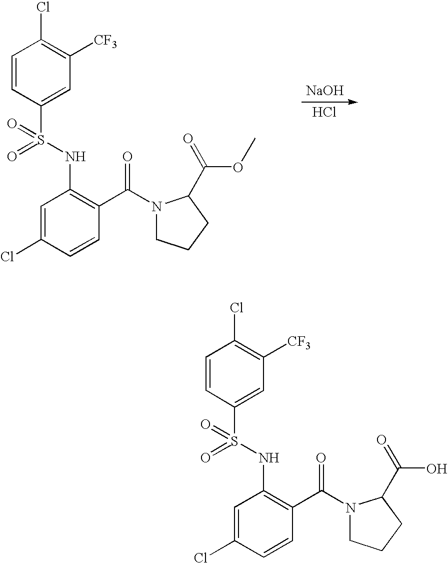

A mixture of 1-[4-chloro-3-trifluoromethyl-benzenesulfonylamino)-benzoyl]-pyrrolidine-2-carboxylic acid methyl ester (15 mg), 2N NaOH (1.5 mL) and methanol (1.5 mL) was stirred at rt overnight. To the solution mixture was added slowly 2M HCl until white solid precipitated (pH=2). Filtration followed by vacuum drying afforded 1-[4-chloro-3-trifluoromethyl-benzenesulfonylamino)-benzoyl]-pyrrolidine-2-carboxylic acid as a white solid. ¹H-NMR (400 MHz, CDCl₃): δ 9.00 (s, 1H), 8.11 (s, 1H), 7.92 (m, 1H), 7.67 (s, 1H), 7.58 (d, 1H), 7.21 (d, 1H), 7.09 (m, 1H), 4.65-4.61 (m, 1H), 3.28-3.26 (m, 2H), 2.36-2.28 (m, 1H), 2.19-2.12 (m, 1H), 1.98-1.90 (m, 1H), 1.87-1.80 (m, 1H). MS: (M+Na)/z=511.0.

### Example 11

### 1-[4-Chloro-2-(4-chloro-3-trifluoromethyl-benzenesulfonylamino)-benzoyl]-pyrrolidine-2-carboxylic acid methylamide

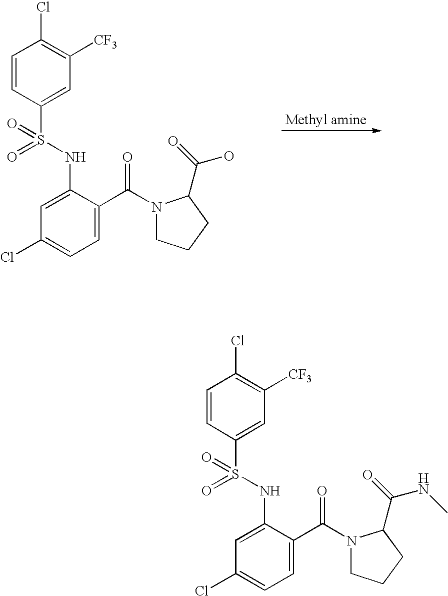

To a mixture of 4-chloro-2-(4-chloro-3-trifluoromethylbenzenesulfonylamino)-benzoic acid (21 mg), 2N methyl amine in THF (0.15 mL) and N,N-diisopropylethylamine (0.104 mL) in CH₂Cl₂ (2 mL) was added 1-propanephosphonic acid cyclic anhydride (50% in ethyl acetate, 0.08 mL). After four hours the reaction mixture was directly purified via flash column (8% methanol in CH₂Cl₂) to afford 1-[4-Chloro-2-(4-chloro-3-trifluoromethyl-benzenesulfonylamino)-benzoyl]-pyrrolidine-2-carboxylic acid methylamide as a white powder. ¹H-NMR (400 MHz, CDCl₃): δ 8.27 (m, 1H), 7.90 (m, 1H), 7.83 (m, 1H), 7.52 (m, 1H), 7.12-7.05 (m, 2H), 4.97 (m, 1H), 3.20 (m, 2H), 3.12(s, 3H), 2.18-1.85 (m, 4H). MS: (M+H)/z=524.

### Example 12

### 1-[4-Chloro-2-(4-chloro-3-trifluoromethyl-benzenesulfonylamino)-benzoyl]-pyrrolidine-2-carboxylic acid dimethylamide

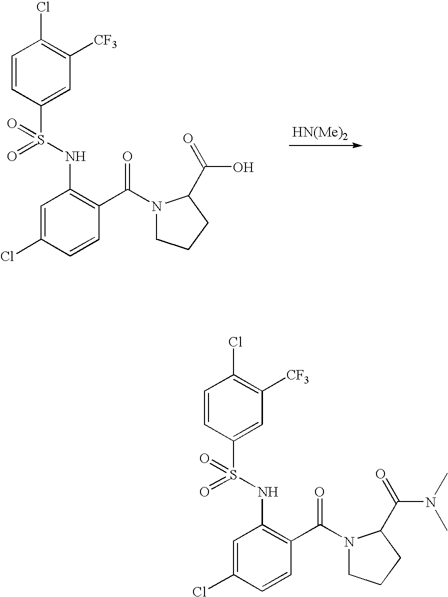

To a mixture of 4-chloro-2-(4-chloro-3-trifluoromethylbenzenesulfonylamino)-benzoic acid (21 mg), dimethyl amine hydrochloride (12 mg) and N,N-diisopropylethylamine (0.104 mL) in CH₂Cl₂ (2 mL) was added of 1-propanephosphonic acid cyclic anhydride (50% in ethyl acetate, 0.08 mL). After four hours the reaction mixture was directly purified via flash column (8% methanol in CH₂Cl₂) to afford 1-[4-chloro-2-(4-chloro-3-trifluoromethyl-benzenesulfonylamino)-benzoyl]-pyrrolidine-2-carboxylic acid dimethylamide as a white powder. ¹H-NMR (400 MHz, CDCl₃): δ 8.27 (m, 1H), 7.90 (m, 1H), 7.83 (m, 1H), 7.52 (m, 1H), 7.12-7.05 (m, 2H), 4.97 (m, 1H), 3.20 (m, 2H), 3.14(s, 3H), 3.09 (s, 3H), 2.18-1.85 (m, 4H). MS: (M+H)/z=538.

### Example 13

### 4-Chloro-N-[5-chloro-2-(1,1-dioxo-thiomorpholine-4-carbonyl)-phenyl]-3-trifluoromethyl-benzenesulfonamide

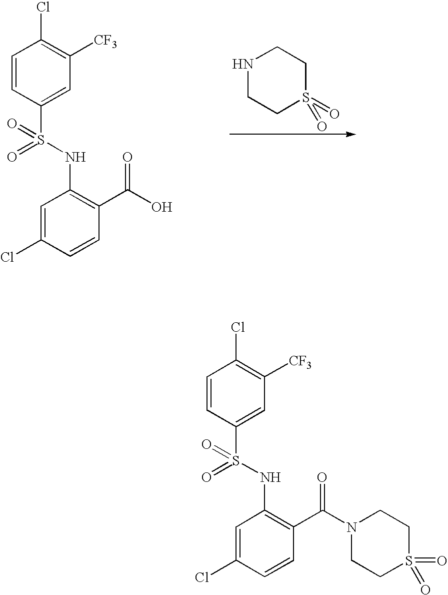

To a mixture of 4-chloro-2-(4-chloro-3-trifluoromethylbenzenesulfonylamino)-benzoic acid (50 mg), thiomorpholine 1,1-dioxide (81 mg) and N,N-diisopropylethylamine (0.125 mL) in CH₂Cl₂ (3 mL) was added 1-propanephosphonic acid cyclic anhydride (50% in ethyl acetate, 0.15 mL). After four hours the reaction mixture was directly purified via Prep HPLC (20-80% acetonitrile in water) to afford 4-chloro-N-[5-chloro-2-(1,1-dioxo-thiomorpholine-4-carbonyl)-phenyl]-3-trifluoromethyl-benzenesulfonamide as a white powder. ¹H-NMR (400 MHz, CDCl₃): δ 8.36 (m, 1H), 8.18 (m, 1H), 8.00 (m, 1H), 7.69 (d, J=7.4 Hz, 1H), 7.40 (m, 1H), 7.19-7.22 (m, 2H), 4.05 (m, 4H), 3.09 (m, 4H). MS: (M+H)/z=531.

### Example 14

### 4-chloro-N-[4,5-difluoro-2-(pyridine-3-carbonyl)-phenyl]-3-trifluoromethyl-benzenesulfonamide

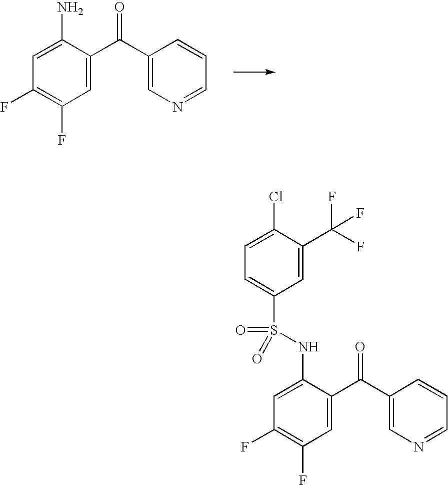

To a solution of the starting aminoketone (47 mg, 0.20 mmol) in dry pyridine (0.5 mL) was added dropwise a solution of 4-chloro-3-trifluoromethyl)benzenesulfonyl chloride (57 mg, 0.20 mmol) in dry pyridine (0.3 mL). The reaction was shaken overnight at room temperature. The reaction was separated by preparative HPLC (20→80% gradient of acetonitrile-water) and the pure product fractions were lyophilized to provide 4-chloro-N-[4,5-difluoro-2-(pyridine-3-carbonyl)-phenyl]-3-trifluoromethyl-benzenesulfonamide as a light yellow solid. ¹H-NMR (400 MHz, CDCl₃): δ 10.20 (br s, 1H), 8.79 (dd, 1H, J=4.8 Hz, J=1.4 Hz), 8.61 (d, 1H, J=1.9 Hz), 7.98 (m, 1H), 7.78 (dm, 1H, J=8.4 Hz), 7.72 (dm, 1H, J=7.7 Hz), 7.57 (m, 1H), 7.39 (m, 2H), 7.19 (m, 1H). MS m/z: 477.3 (M+1).

### Example 15

### 4-Chloro-N-[4-chloro-2-(oxazole-4-carbonyl)-phenyl]-3-trifluoromethyl-benzenesulfonamide

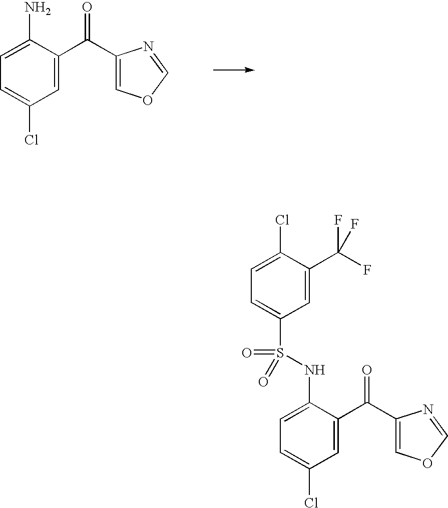

To a solution of the starting aminoketone (44 mg, 0.20 mmol) in dry pyridine (0.5 mL) was added dropwise a solution of 4-chloro-3-trifluoromethyl)benzenesulfonyl chloride (57 mg, 0.20 mmol) in dry pyridine (0.3 mL). The reaction was shaken overnight at room temperature. The reaction was separated by preparative HPLC (20→80% gradient of ACN-water) and the pure product fractions were lyophilized to provide 4-chloro-N-[4-chloro-2-(oxazole-4-carbonyl)-phenyl]-3-trifluoromethyl-benzenesulfonamide as a light yellow solid: ¹H-NMR (400 MHz, CDCl₃): δ 10.22 (s, 1H), 8.34 (d, 1H, J=2.2 Hz), 8.27 (d, 1H, J=1.1 Hz), 8.00 (d, 1H, J=2.2 Hz), 7.94 (d, 1H, J=1.1 Hz), 7.72 (dd, 1H, J=8.4 Hz, J=2.2 Hz), 7.53 (dd, 1H, J=8.8 Hz, J=2.2 Hz), 7.46 (d, 1H, J=8.4 Hz). MS m/z: 465.0 (M+1).

### Example 16

### 4-Chloro-N-[4-chloro-2-(1-methyl-1H-pyrazole-3-carbonyl)-phenyl]-3-trifluoromethyl-benzenesulfonamide

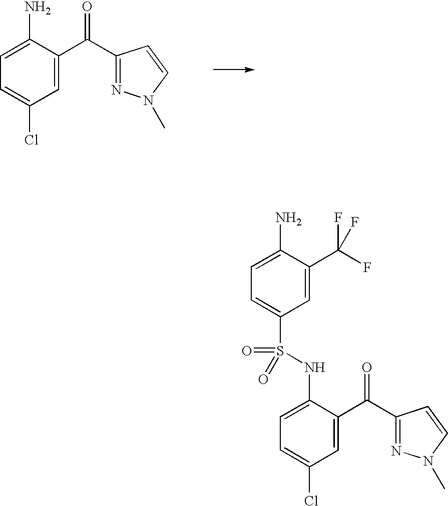

To a solution of the starting aminoketone (44 mg, 0.20 mmol) in dry pyridine (0.5 mL) was added dropwise a solution of 4-chloro-3-trifluoromethyl)benzenesulfonyl chloride (57 mg, 0.20 mmol) in dry pyridine (0.3 mL). The reaction was shaken overnight at room temperature. The reaction was separated by preparative HPLC (20 80% gradient of ACN-water) and the pure product fractions were lyophilized to provide 4-chloro-N-[4-chloro-2-(1-methyl-1H-pyrazole-3-carbonyl)-phenyl]-3-trifluoromethyl-benzenesulfonamide as a light yellow solid: ¹H-NMR (400 MHz, CDCl₃): δ 7.95 (m, 1H), 7.90 (m, 1H), 8.50 (m, 4H), 7.25 (m, 2H), 7.15 (m, 1H), 3.99 (s, 3H). MS m/z: 478.0 (M+1).

### Example 17

### N-(2-Benzoyl-5-chloro-phenyl)-4-chloro-benzenesulfonamide

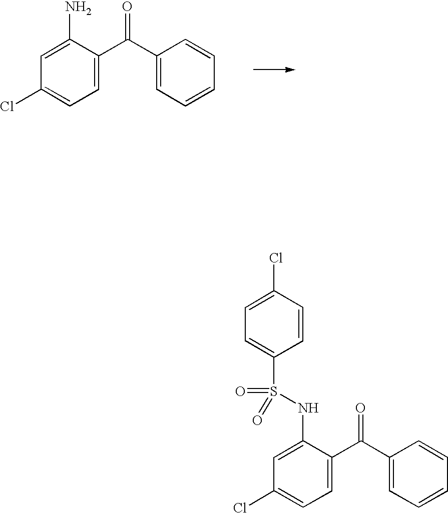

To a solution of the starting aminoketone (35 mg, 0.15 mmol) in dry pyridine (0.5 mL) was added dropwise a solution of 4-chlorobenzenesulfonyl chloride (33 mg, 0.16 mmol) in dry pyridine (0.25 mL). The reaction was shaken and heated (45° C.) overnight (complete by LCMS). The product was separated by preparative HPLC (20→80% gradient of ACN-water) and the pure product fractions were lyophilized to provide N-(2-benzoyl-5-chloro-phenyl)-4-chloro-benzenesulfonamide as a light yellow solid: ¹H-NMR (400 MHz, CDCl₃): δ 10.59 (s, 1H), 8.88 (m, 2H), 7.80 (m, 3H), 7.57 (m, 2H), 7.43 (d, 2H, J=8.5 Hz), 7.27 (d, 1H, J=8.4 Hz), 7.07 (dd, 1H, J=8.4 Hz, J=1.8 Hz). MS m/z 406.5 (M+1).

### Example 18

### N-(2-Benzoyl-5-chloro-phenyl)-3-trifluoromethyl-benzenesulfonamide

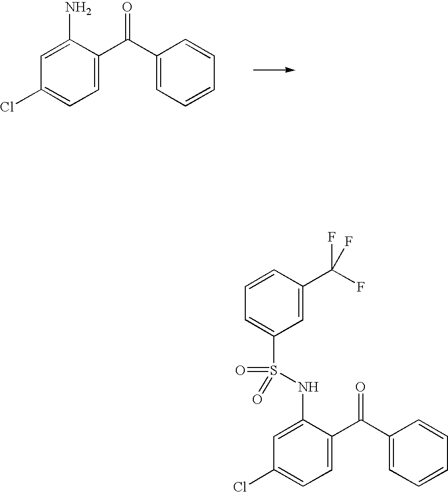

To a solution of the starting aminoketone (35 mg, 0.15 mmol) in dry pyridine (0.5 mL) was added dropwise a solution of 3-(trifluoromethyl)benzenesulfonyl chloride (5.0 mg, 0.0 mmol) in dry pyridine (0.25 mL). The reaction was shaken and heated overnight at room temperature (complete by LCMS). The product was separated by preparative HPLC (20→80% gradient of ACN-water) and the pure product fractions were lyophilized to provide N-(2-Benzoyl-5-chloro-phenyl)-3-trifluoromethyl-benzenesulfonamide as a light yellow solid. ¹H-NMR (400 MHz, CDCl₃): δ 10.67 (s, 1H), 8.83 (m, 2H), 8.09 (br s, 1H), 8.07 (d, 1H, J=7.7 Hz), 7.82 (d, 1H, J=2.2 Hz), 7.79 (d, 1H, J=8.1 Hz), 7.62 (t, 1H, J=7.7 Hz), 7.48 (m, 7.62 (d, 1H, J=8.4 Hz), 7.62 (dd, 1H, J=8.4 Hz, J=2.2 Hz). MS m/z 440.5 (M+1).

### Example 19

### N-(2-Benzoyl-5-chloro-phenyl)-3,4-difluoro-benzenesulfonamide

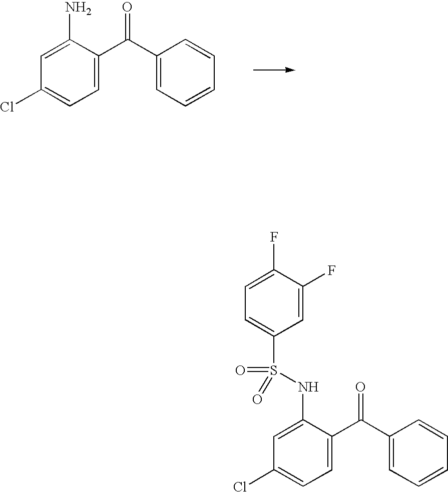

To a solution of the starting aminoketone (35 mg, 0.15 mmol) in dry pyridine (0.5 mL) was added dropwise a solution of 3,4-difluorobenzenesulfonyl chloride (35 mg, 0.15 mmol) in dry pyridine (0.3 mL). The reaction was stirred and heated for 3 h then overnight at room temperature (complete by LCMS). The product was separated by preparative HPLC (20→80% gradient of acetonitrile-water) and the pure product fractions were lyophilized to provide N-(2-benzoyl-5-chloro-phenyl)-3,4-difluoro-benzenesulfonamide as a light yellow solid. ¹H-NMR (400 MHz, CDCl₃): δ 10.75 (s, 1H), 8.87 (d, 2H, J=6.2 Hz), 7.79 (d, 1H, J=2.2 Hz), 7.72 (m, 2H), 7.56 (m, 2H), 7.32 (d, 1H, J=8.4 Hz), 7.29 (m, 1H), 7.09 (dd, 1H, J=8.4 Hz, J=1.8 Hz). MS m/z 409.1 (M+1).

### Example 20

### N-(2-Benzoyl-5-chloro-phenyl)-3-cyano-4-fluoro-benzenesulfonamide

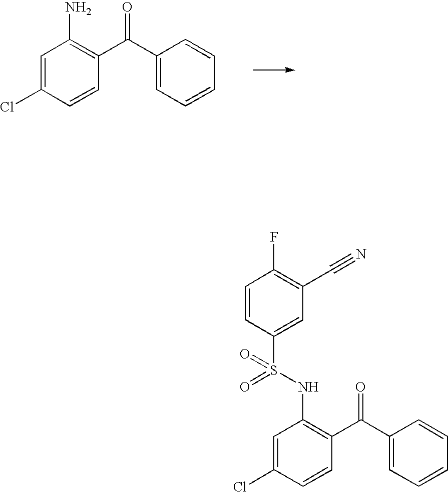

To a solution of the starting aminoketone (22 mg, 0.10 mmol) in dry pyridine (0.5 mL) was added dropwise a solution of 4-fluoro-3-cyano-benzenesulfonyl chloride (28.0 mg, 0.10 mmol) in dry pyridine (0.5 mL). The reaction was stirred overnight at room temperature (complete by LCMS). The product was separated by preparative HPLC (20→80% gradient of acetonitrile-water) and the pure product fractions were lyophilized to provide N-(2-benzoyl-5-chloro-phenyl)-3-cyano-4-fluoro-benzenesulfonamide as a light yellow solid. ¹H-NMR (400 MHz, CDCl₃): δ 10.80 (br s, 1H), 8.81 (m, 2H), 8.14 (m, 2H), 7.77 (m, 1H), 7.42 (d, 1H, J=8.4 Hz), 7.32 (m, 3H), 7.12 (d, 1H, J=8.4 Hz). MS m/z 416.1 (M+1).

### Example 21

### 4-Chloro-N-[5-chloro-2-(hydroxy-phenyl-methyl)-phenyl]-3-trifluoromethyl-benzenesulfonamide

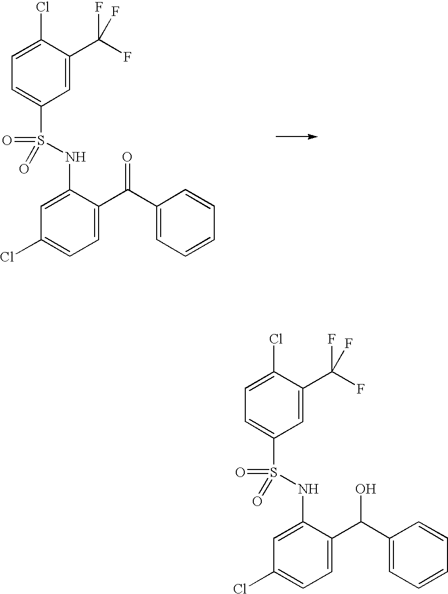

To a solution of the starting ketone (59 mg, 0.12 mmol) in dry THF (1.0 mL) and methanol (1.0 mL) was added sodium borohydride (4.0 mg, 0.10 mmol). The reaction was stirred for 0.5 h at room temperature (complete by LCMS), then was quenched with 1 drop of 2N HCl. The product was separated by preparative HPLC (20→80% gradient of acetonitrile-water) and the pure product fractions were lyophilized to provide 4-chloro-N-[5-chloro-2-(hydroxy-phenyl-methyl)-phenyl]-3-trifluoromethyl-benzenesulfonamide as a white solid. ¹H-NMR (400 MHz, CDCl₃): δ 8.63 (m, 2H), 7.98 (d, 1H, J=2.2 Hz), 7.83 (m, 3H), 7.64 (d, 1H, J=8.4 Hz), 7.27 (m, 2H), 7.15 (d, 1H, J=8.1 Hz), 6.82 (dm, 1H, J=2.2 Hz), 6.21 (m, 1H). MS m/z 477.0 (M+1).

### Example 22

### 4-Chloro-N-(3-chloro-phenyl)-3-trifluoromethyl-benzenesulfonamide

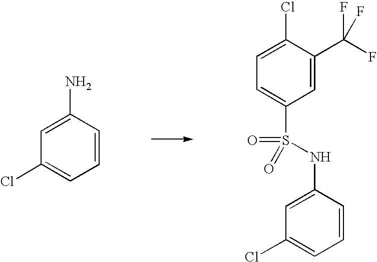

To a solution of 3-chloroaniline (64 mg, 0.50 mmol) in dry pyridine (0.5 mL) was added dropwise a solution of 4-chloro-3-trifluoromethyl)benzenesulfonyl chloride (139 mg, 0.50 mmol) in dry pyridine (0.5 mL). The reaction was stirred overnight at room temperature (complete by LCMS). The reaction was added to a mixture of ice and dilute HCl, and the aqueous mixture was extracted with ethyl acetate (3 X 30 mL). The extracts were dried (MgSO₄), filtered and concentrated. The syrup was chromatographed on silica gel using EtOAc-hexane (5:95, 10:90, 15:85) and pure product fractions were concentrated to provide 4-chloro-N-(3-chloro-phenyl)-3-trifluoromethyl-benzenesulfonamide as a white crystalline solid. ¹H-NMR (400 MHz, CDCl₃): δ 8.07 (d, 1H, J=2.2 Hz), 7.82 (dd, 1H, J=8.4 Hz, J=2.2 Hz), 7.60 (d, 1H, J=8.4 Hz), 7.21 (t, 1H, J=7.8 Hz), 7.15 (dm, 1H, J=7.8 Hz), 7.11 (t, 1H, J=2.0 Hz), 6.95 (ddd, 1H, J=7.8 Hz, J=2.0 Hz, J=1.1 Hz), 6.65 (s, 1H). MS m/z 371.1 (M+1).

### Example 23

### 4-Chloro-N-(4-chloro-phenyl)-3-trifluoromethyl-benzenesulfonamide

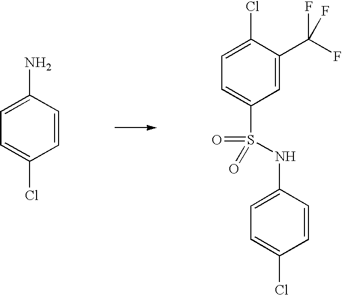

To a solution of 4-chloroaniline (64 mg, 0.50 mmol) in dry pyridine (0.5 mL) was added dropwise a solution of 4-chloro-3-trifluoromethyl)benzenesulfonyl chloride (139 mg, 0.50 mmol) in dry pyridine (0.5 mL). The reaction was stirred overnight at room temperature (complete by LCMS). The reaction was added to a mixture of ice and dilute HCl, and the aqueous mixture was extracted with ethyl acetate (3×30 mL). The extracts were dried (MgSO₄), filtered and concentrated. The syrup was chromatographed on silica gel using EtOAc-hexane (5:95, 10:90, 15:85) and pure product fractions were concentrated to provide 4-chloro-N-(4-chloro-phenyl)-3-trifluoromethyl-benzenesulfonamide as a white crystalline solid. ¹H-NMR (400 MHz, CDCl₃): δ 8.05 (d, 1H, J=2.2 Hz), 7.75 (dd, 1H, J=8.4 Hz, J=2.2 Hz), 7.58 (d, 1H, J=8.4 Hz), 7.25 (m, 2H), 7.01 (m, 2H), 6.60 (s, 1H). MS m/z 371.1 (M+1).

### Example 24

### 3,4-Dichloro-N-[4-chloro-2-(4-fluoro-benzoyl)-phenyl]-benzenesulfonamide

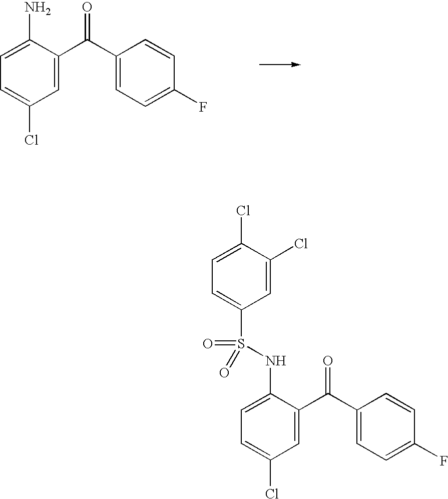

To a solution of the starting aminoketone (50 mg, 0.20 mmol) in dry pyridine (0.5 mL) was added dropwise a solution of 3,4-dichlorobenzenesulfonyl chloride (52 mg, 0.21 mmol) in dry pyridine. The reaction was shaken overnight at room temperature. The reaction was separated by preparative HPLC (20→80% gradient of acetonitrile-water) and the pure product fractions were lyophilized to provide 3,4-dichloro-N-[4-chloro-2-(4-fluoro-benzoyl)-phenyl]-benzenesulfonamide as a light yellow solid. ¹H-NMR (400 MHz, CDCl₃): δ 9.40 (br s, 1H), 7.69 (d, 1H, J=8.8 Hz), 7.62 (d, 1H, J=1.8 Hz), 7.51 (dd, 1H, J=8.8 Hz, J=2.6 Hz), 7.42 (m, 3H), 7.31 (d, 1H, J=2.6 Hz), 7.24 (d, 1H, J=8.4 Hz), 7.11 (t, 2H, J=8.4 Hz). MS m/z: 458.2 (M+1).

### Example 25

### 3,4-Dichloro-N-[4-chloro-2-(2-fluoro-benzoyl)-phenyl]-benzenesulfonamide

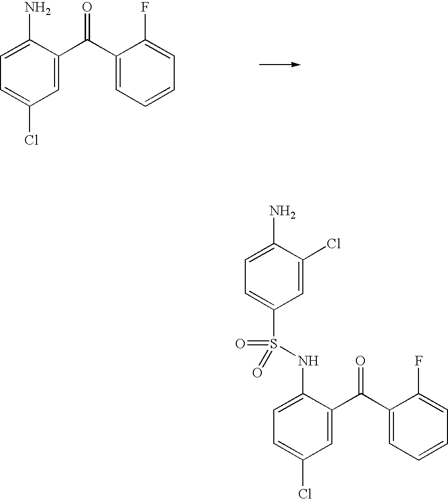

To a solution of the starting aminoketone (50 mg, 0.20 mmol) in dry pyridine (0.5 mL) was added dropwise a solution of 3,4-dichlorobenzenesulfonyl chloride (52 mg, 0.21 mmol) in dry pyridine. The reaction was shaken overnight at room temperature. The reaction was separated by preparative HPLC (20→80% gradient of acetonitrile-water) and the pure product fractions were lyophilized to provide 3,4-dichloro-N-[4-chloro-2-(2-fluoro-benzoyl)-phenyl]-benzenesulfonamide as a light yellow solid. ¹H-NMR (400 MHz, CDCl₃): δ 10.38 (s, 1H), 7.81 (d, 1H, J=2.2 Hz), 7.72 (d, 1H, J=8.8 Hz), 7.57 (m, 2H), 7.50 (dd, 1H, J=8.8 Hz, J=2.2 Hz), 7.43 (d, 1H, J=8.4 Hz), 7.33 (m, 2H), 7.27 (dd, 1H, J=7.7 Hz, J=0.7 Hz), 7.13 (tm, 1H, J=8.8 Hz). MS m/z: 458.3 (M+1).

### Example 26

### N-(2-Benzoyl-phenyl)-3,4-dichloro-benzenesulfonamide

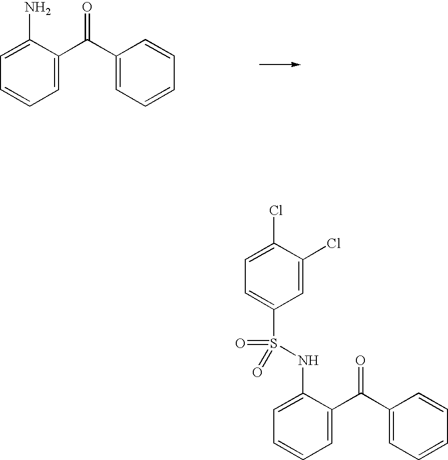

To a solution of the starting 2-aminobenzophenone (78 mg, 0.39 mmol) in dry pyridine (0.5 mL) was added dropwise a solution of 3,4-dichlorobenzenesulfonyl chloride (99 mg, 0.40 mmol) in dry pyridine. The reaction was shaken overnight at room temperature. The reaction was separated by preparative HPLC (20→80% gradient of acetonitrile-water) and the pure product fractions were treated with aqueous sodium bicarbonate and extracted with ethyl acetate. The extracts were dried over magnesium sulfate, filtered, and concentrated to yield N-(2-benzoyl-phenyl)-3,4-dichloro-benzenesulfonamide as a waxy crystalline solid. ¹H-NMR (400 MHz, CDCl₃): δ 7.76 (d, 1H, J=8.0 Hz), 7.66 (d, 1H, J=2.2 Hz), 7.57 (m, 2H), 7.46 (dd, 1H, J=8.4 Hz, J=2.2 Hz), 7.43 (d, 1H, J=8.4 Hz), 7.40 (m, 5H), 7.25 (d, 1H, J=8.4 Hz), 7.18 (td, 1H, J=7.3 Hz, J=1.1 Hz). MS m/z: 406.2 (M+1).

This compound was prepared according to the following literature procedure: Zhou et al.; Bioorganic & Med. Chem., 9, 2061-2071 (2001).

Measuring Efficacy of Chemokine Modulators

In Vitro Assays

A variety of assays can be used to evaluate the compounds provided herein, including signaling assays, migration assays, ligand binding assays, and other assays of cellular response. Chemokine receptor signaling assays can be used to measure the ability of a compound, such as a potential CCR2 antagonist, to block CCR2 ligand- (e.g. MCP-1)-induced signaling or a potential CCR9 antagonist, to block CCR9 ligand- (e.g. TECK)-induced signaling. A migration assay can be used to measure the ability of a compound of interest, such as a possible chemokine antagonist, to block chemokine-mediated cell migration in vitro. The latter is believed to resemble chemokine-induced cell migration in vivo. A ligand binding assay can be used to measure the ability of a compound, such as a potential CCR2 antagonist, to block the interaction of MCP-1 with its receptor or a potential CCR9 antagonist, to block the interaction of TECK with its receptor.

In a suitable assay, a chemokine protein (whether isolated or recombinant) is used which has at least one property, activity, or functional characteristic of a mammalian chemokine protein. The property can be a binding property (to, for example, a ligand or inhibitor), a signaling activity (e.g., activation of a mammalian G protein, induction of rapid and transient increase in the concentration of cytosolic free calcium ion), cellular response function (e.g., stimulation of chemotaxis or inflammatory mediator release by leukocytes), and the like.

The assay can be a cell-based assay that utilizes cells stably or transiently transfected with a vector or expression cassette having a nucleic acid sequence that encodes the chemokine receptor. Cell lines naturally expressing the chemokine can also be used. The cells are maintained under conditions appropriate for expression of the receptor and are contacted with a putative agent under conditions appropriate for binding to occur. Binding can be detected using standard techniques. For example, the extent of binding can be determined relative to a suitable control (for example, relative to background in the absence of a putative agent, or relative to a known ligand). Optionally, a cellular fraction, such as a membrane fraction, containing the receptor can be used in lieu of whole cells.

Detection of binding or complex formation can be detected directly or indirectly. For example, the putative agent can be labeled with a suitable label (e.g., fluorescent label, chemiluminescent label, isotope label, enzyme label, and the like) and binding can be determined by detection of the label. Specific and/or competitive binding can be assessed by competition or displacement studies, using unlabeled agent or a ligand (e.g., MCP-1 or TECK) as a competitor.

Binding inhibition assays can be used to evaluate the present compounds. In these assays, the compounds are evaluated as inhibitors of ligand binding using, for example, MCP-1 or TECK. In one embodiment, the CCR2 receptor is contacted with a ligand such as MCP-1 and a measure of ligand binding is made. The receptor is then contacted with a test agent in the presence of a ligand (e.g., MCP-1) and a second measurement of binding is made. In another embodiment, the CCR9 receptor is contacted with a ligand such as TECK and a measure of ligand binding is made. The receptor is then contacted with a test agent in the presence of a ligand (e.g., TECK) and a second measurement of binding is made. A reduction in the extent of ligand binding is indicative of inhibition of binding by the test agent. The binding inhibition assays can be carried out using whole cells which express the chemokine, or a membrane fraction from cells which express the chemokine.

The binding of a G protein coupled receptor by, for example, an agonist, can result in a signaling event by the receptor. Accordingly, signaling assays can also be used to evaluate the compounds of the present invention and induction of signaling function by an agent can be monitored using any suitable method. For example, G protein activity, such as hydrolysis of GTP to GDP, or later signaling events triggered by receptor binding can be assayed by known methods (see, for example, PCT/US97/15915; Neote et al., Cell, 72:415425 (1993); Van Riper et al., J. Exp. Med., 177:851-856 (1993) and Dahinden et al., J. Exp. Med., 179:751-756 (1994)).

Chemotaxis assays can also be used to assess receptor function and evaluate the compounds provided herein. These assays are based on the functional migration of cells in vitro or in vivo induced by an agent, and can be used to assess the binding and/or effect on chemotaxis of ligands, inhibitors, or agonists. A variety of chemotaxis assays are known in the art, and any suitable assay can be used to evaluate the compounds of the present invention. Examples of suitable assays include those described in PCT/US97/15915; Springer et al., WO 94/20142; Berman et al., Immunol. Invest., 17:625-677 (1988); and Kavanaugh et al., J. Immunol., 146:4149-4156 (1991)).

Calcium signaling assays measure calcium concentration over time, preferably before and after receptor binding. These assays can be used to quantify the generation of a receptor-signaling mediator, Ca⁺⁺, following receptor binding (or absence thereof). These assays are useful in determining the ability of a compound, such as those of the present invention, to generate the receptor signaling mediator by binding to a receptor of interest. Also, these assays are useful in determining the ability of a compound, such as those of the present invention, to inhibit generation of the receptor signaling mediator by interfering with binding between a receptor of interest and a ligand.

In calcium signaling assays used to determine the ability of a compound to interfere with binding between a chemokine receptor and a known chemokine ligand, chemokine receptor-expressing cells (CCR2-expressing cells such as THP-1 cells or CCR9-expressing cells such as T cell line MOLT-4 cells) are first incubated with a compound of interest, such as a potential chemokine antagonist, at increasing concentrations. The cell number can be from 10⁵ to 5×10⁵ cells per well in a 96-well microtiter plate. The concentration of the compound being tested may range from 0 to 100 μM. After a period of incubation (which can range from 5 to 60 minutes), the treated cells are placed in a Fluorometric Imaging Plate Reader (FLIPR®) (available from Molecular Devices Corp., Sunnyvale, Calif.) according to the manufacturer's instruction. The FLIPR® system is well known to those skilled in the art as a standard method of performing assays. The cells are then stimulated with an appropriate amount of the chemokine ligand (MCP-1 for CCR2 or TECK for CCR9) at 5-100 nM final concentration, and the signal of intracellular calcium increase (also called calcium flux) is recorded. The efficacy of a compound as an inhibitor of binding between the chemokine and the ligand can be calculated as an IC₅₀ (the concentration needed to cause 50% inhibition in signaling) or IC₉₀ (at 90% inhibition).

In vitro cell migration assays can be performed (but are not limited to this format) using the 96-well microchamber (called ChemoTX™). The ChemoTX™ system is well known to those skilled in the art as a type of chemotactic/cell migration instrument. In this assay, CCR2-expressing cells (such as THP-1) or CCR9-expressing cells (such as MOLT-4) are first incubated with a compound of interest; such as a possible CCR2 or CCR9 antagonist, respectively, at increasing concentrations. Typically, fifty thousand cells per well are used, but the amount can range from 10³-10⁶ cells per well. The chemokine ligand (for example, CCR2 ligand MCP-1, typically at 0.1 nM (but can range from 5-100 nM), or CCR9 ligand TECK, typically at 50 nM (but can range from 5-100 nM)), is placed at the lower chamber and the migration apparatus is assembled. Twenty microliters of test compound-treated cells are then placed onto the membrane. Migration is allowed to take place at 37° C. for a period of time, typically 1.5 hours for CCR2 or 2.5 hours for CCR9. At the end of the incubation, the number of cells that migrated across the membrane into the lower chamber is then quantified. The efficacy of a compound as an inhibitor of chemokine-mediated cell migration is calculated as an IC₅₀ (the concentration needed to reduce cell migration by 50%) or IC₉₀ (for 90% inhibition).

In vivo Efficacy Models for Human IBD

T cell infiltration into the small intestine and colon have been linked to the pathogenesis of human inflammatory bowel diseases which include Coeliac disease, Crohn's disease and ulcerative colitis. Blocking trafficking of relevant T cell populations to the intestine is believed to be an effective approach to treat human IBD. CCR9 is expressed on gut-homing T cells in peripheral blood, elevated in patients with small bowel inflammation such as Crohn's disease and Coeliac disease. CCR9 ligand TECK is expressed in the small intestine. It is thus believed that this ligand-receptor pair plays a role in IBD development by mediating migration of T cells to the intestine. Several animal models exist and can be used for evaluating compounds of interest, such as potential CCR9 antagonists, for an ability to affect such T cell migration and/or condition or disease, which might allow efficacy predictions of antagonists in humans.

Animal Models with Pathology Similar to Human Ulcerative Colitis

A murine model described by Panwala and coworkers (Panwala et al., J Immunol., 161(10):5733-44 (1998)) involves genetic deletion of the murine multi-drug resistant gene (MDR). MDR knockout mice (MDR−/−) are susceptible to developing a severe, spontaneous intestinal inflammation when maintained under specific pathogen-free facility conditions. The intestinal inflammation seen in MDR−/− mice has a pathology similar to that of human inflammatory bowel disease (IBD) and is defined by Th1 type T cells infiltration into the lamina propria of the large intestine.

Another murine model was described by Davidson et al., J. Exp. Med., 184(1):241-51(1986). In this model, the murine IL-10 gene was deleted and mice rendered deficient in the production of interleukin 10 (IL-10−/−). These mice develop a chronic inflammatory bowel disease (IBD) that predominates in the colon and shares histopathological features with human IBD.

Another murine model for IBD has been described by Powrie et al., Int Immunol., 5(11):1461-71 (1993), in which a subset of CD4+ T cells (called CD45RB(high)) from immunocompetent mice are purified and adoptively transferred into immunodeficient mice (such as C.B-17 scid mice). The animal restored with the CD45RBhighCD4+ T cell population developed a lethal wasting disease with severe mononuclear cell infiltrates in the colon, pathologically similar with human IBD.

Murine Models with Oathology Similar to Human Crohn's Disease

The TNF ARE(−/−) model. The role of TNF in Crohn's disease in human has been demonstrated more recently by success of treatment using anti-TNF alpha antibody by Targan et al., N. Engl. J. Med., 337(15):1029-35 (1997). Mice with aberrant production of TNF-alpha due to genetic alteration in the TNF gene (ARE−/−) develop Crohn's-like inflammatory bowel diseases (see Kontoyiannis et al., Immunity, 10(3):387-98 (1999)).

The SAMP/yit model. This is model described by Kosiewicz et al., J. Clin. Invest., 107(6):695-702 (2001). The mouse strain, SAMP/Yit, spontaneously develops a chronic inflammation localized to the terminal ileum. The resulting ileitis is characterized by massive infiltration of activated T lymphocytes into the lamina propria, and bears a remarkable resemblance to human Crohn's disease.

Examples of in vitro Assays

Reagents

THP-1 cells and MOLT-4 cells were obtained from the American Type Culture Collection (Manassas, Va.) and cultured in RPMI tissue culture medium supplemented with 10% fetal calf serum (FCS) in a humidified 5% CO₂ incubator at 37° C. Recombinant human chemokine proteins MCP-1 and TECK were obtained from R&D Systems (Minneapolis, Minn.). ¹²⁵I-labeled MCP-1 protein was obtained from Amersham (Piscataway, N.J.). ChemoTX® chemotaxis microchambers were purchased from Neuro Probe (Gaithersburg, Md.). CyQUANT® cell proliferation kits were purchased from Molecular Probes (Eugene, Oreg.). Calcium indicator dye Fluo-4 AM was purchased from Molecular Devices (Mountain View, Calif.).

Conventional Migration Assay

Conventional-migration assay was used to determine the efficacy of potential receptor antagonists in blocking migration mediated through chemokines (such as CCR2 or CCR9). This assay was routinely performed using the ChemoTX® microchamber system with a 5-μm pore-sized polycarbonate membrane. To begin such an assay, chemokine expressing cells (such as THP-1 cells for CCR2 assay or MOLT-4 cells for CCR9 assay) were harvested by centrifugation of cell suspension at 1000 RPM on a GS-6R Beckman centrifuge. The cell pellet was resuspended in chemotaxis buffer (HBSS with 0.1% BSA) at 10×10⁶cells/mL for CCR2 assay (5 ×10⁶cells/mL for CCR9 assay). Test compounds at desired concentrations were prepared from 10 mM stock solutions by serial dilutions in chemotaxis buffer. An equal volume of cells and compounds were mixed and incubated at room temperature for 15 minutes. Afterwards, 20 μL of the mixture was transferred onto the porous membrane of a migration microchamber, with 29 μL of chemokine ligand (0.1 nM chemokine MCP-1 protein for CCR2 assay or 50 nm chemokine TECK protein for CCR9 assay) placed at the lower chamber. Following an incubation at 37° C. (90-minute for CCR2; 150-minute for CCR9), during which cells migrated against the chemokine gradient, the assay was terminated by removing the cell drops from atop the filter. To quantify cells migrated across the membrane, 5 μL of 7X CyQUANT® solution was added to each well in the lower chamber, and the fluorescence signal measured on a Spectrafluor Plus fluorescence plate reader (TECAN, Durham, N.C.). The degree of inhibition was determined by comparing migration signals between compound-treated and untreated cells. IC₅₀ calculation was further performed by non-linear squares regression analysis using Graphpad Prism (Graphpad Software, San Diego, Calif.).

BiRAM Assay

The primary screen to identify chemokine antagonists was carried out using BiRAM assay (WO 02101350, US2004023286), which detects potential hits by their ability to activate cell migration under inhibitory chemokine concentration. To begin such an assay, chemokine expressing cells (such as THP-1 cells for CCR2 assay or MOLT-4 cells for CCR9 assay) were harvested by centrifugation of cell suspension at 1000 RPM on a GS-6R Beckman centrifuge. The cell pellet was resuspended in chemotaxis buffer (HBSS/0.1% BSA) at 10×10⁶ cells/mL for CCR2 assay (5×10⁶ cells/mL for CCR9 assay). Twenty-five microliters of cells was mixed with an equal volume of a test compound diluted to 20 μM in the same buffer. Twenty microliters of the mixture was transferred onto the filter in the upper chemotaxis chamber, with 29 μL of chemokine solution containing chemokine ligand (100 nM chemokine MCP-1 and MIP-1α protein for CCR2 assay or 500 nm chemokine TECK protein for CCR9 assay) was placed in the lower chamber. Following an incubation at 37° C. (90-minute for CCR2; 150-minute for CCR9), the assay was terminated by removing the cell drops from atop the filter. To quantify cells migrated across the membrane, 5 μL of 7X CyQUANT® solution was added to each well in the lower chamber, and the fluorescence signal measured on a Spectrafluor Plus fluorescence plate reader (TECAN, Durham, N.C.).

For selection of potential hits, the level of migration activation was calculated as a RAM index-the ratio between the signal of a particular well and the median signal of the whole plate. Compounds with a RAM index of greater than 1.5 for CCR2 assay (1.8 for CCR9 assay) were regarded as RAM positive, and were selected for IC₅₀ determinations in conventional functional assays.

Calcium Flux Assay

Calcium flux assay measures an increase in intracellular calcium following ligand-induced receptor activation. In the screen of chemokine antagonists, it was used as a secondary assay carried out on a FLIPR® machine (Molecular Devices, Mountain View, Calif.). To begin an assay, chemokine expressing cells (such as THP-1 cells for CCR2 assay or MOLT-4 cells for CCR9 assay) were harvested by centrifugation of cell suspension, and resuspended to 1.5×10⁶ cells/mL in HBSS (with 1% fetal calf serum). Cells were then labeled with a calcium indicator dye Fluo-4 AM for 45 minutes at 37° C. with gentle shaking. Following incubation, cells were pelletted, washed once with HBSS and resuspended in the same buffer at a density of 1.6×10⁶ cells/mL. One hundred microliters of labeled cells were mixed with 10 μL of test compound at the appropriate concentrations on an assay plate. Chemokine protein (MCP-1 at a final concentration of 0.1 nM for CCR2 assay or TECK at a final concentration of 25 nM for CCR9 assay) to activate the receptor. The degree of inhibition was determined by comparing calcium signals between compound-treated and untreated cells. IC₅₀ calculations were further performed by non-linear squares regression analysis using Graphpad Prism (Graphpad Software, San Diego, Calif.).

Ligand Binding Assay

Ligand binding assay was used to determine the ability of potential CCR2 antagonists to block the interaction between CCR2 and its ligand MCP-1. CCR2 expressing THP-1 cells were centrifuged and resuspended in assay buffer (20 mM HEPES pH 7.1, 140 mM NaCl, 1 mM CaCl₂, 5 mM MgCl₂, and with 0.2% bovine serum albumin) to a concentration of 2.2×10⁵ cells/mL. Binding assays were set up as follows. First, 0.09 mL of cells (1×10⁵ THP-1 cells/well) was added to the assay plates containing the compounds, giving a final concentration of ˜2-10 μM each compound for screening (or part of a dose response for compound IC₅₀ determinations). Then 0.09 mL of ¹²⁵I labeled MCP-1 (obtained from Amersham; Piscataway, NJ) diluted in assay buffer to a final concentration of ·50 pM, yielding ·30,000 cpm per well, was added, the plates sealed and incubated for approximately 3 hours at 4° C. on a shaker platform. Reactions were aspirated onto GF/B glass filters pre-soaked in 0.3% polyethyleneimine (PEI) solution, on a vacuum cell harvester (Packard Instruments; Meriden, Conn.). Scintillation fluid (50 μL; Microscint 20, Packard Instruments) was added to each well, the plates were sealed and radioactivity measured in a Top Count scintillation counter (Packard Instruments). Control wells containing either diluent only (for total counts) or excess MCP-1 (1 μg/mL, for non-specific binding) were used to calculate the percent of total inhibition for compound. The computer program Prism from GraphPad, Inc. (San Diego, Calif.) was used to calculate IC₅₀ values. IC₅₀ values are those concentrations required to reduce the binding of labeled MCP-1 to the receptor by 50%.

Discovery of Chemokine Antagonists

The discovery of chemokine antagonists was carried out in two steps: First, BiRAM assay was used to screen a compound library in a high-throughput manner. The assay detected compounds by their ability to cause a positive migration signal under BiRAM condition. Secondly, BiRAM positive compounds were tested to determine their IC₅₀ values using the conventional migration, calcium flux assays and ligand binding assays.

For instance, in a screen of approximately 100,000 compounds, 2000 individual wells representing approximately 2% of total compounds showed a desired RAM index (greater than 1.5 for CCR2, greater than 1.8 for CCR9). These compounds were cheery-picked and retested in duplicate wells by RAM assay. A total of 156 compounds were confirmed BiRAM positives.

Since a BiRAM positive signal indicates only the presence of a receptor antagonist and not how strongly it blocks receptor functions, the BiRAM positive compounds were further tested for potency in conventional migration, calcium flux and ligand binding assays. IC₅₀ determinations on this subset discovered several compounds with an IC₅₀ less than 1 pM and that did not inhibit other chemokine receptors examined at significant levels.

In vivo Efficacy

A 17-day study of type II collagen-induced arthritis was conducted to evaluate the effects of a modulator on arthritis-induced clinical ankle swelling. Rat collagen-induced arthritis is an experimental model of polyarthritis that has been widely used for preclinical testing of numerous anti-arthritic agents (see Trentham et al., J. Exp. Med. 146(3):857-868 (1977), Bendele et al., Toxicologic Pathol. 27:134-142 (1999), Bendele et al., Arthritis Rheum. 42:498-506 (1999)). The hallmarks of this model are reliable onset and progression of robust, easily measurable polyarticular inflammation, marked cartilage destruction in association with pannus formation and mild to moderate bone resorption and periosteal bone proliferation.

Female Lewis rats (approximately 0.2 kilograms) are anesthetized with isoflurane and injected with Freund's Incomplete Adjuvant containing 2 mg/mL bovine type II collagen at the base of the tail and two sites on the back on days 0 and 6 of this 17-day study. The test modulator is dosed daily by sub-cutaneous injection from day 9 to day 17 at a dose of 100 mg/kg and a volume of 1 mL/kg in the following vehicle (24.5% Cremaphore EL, 24.5% common oil, 1% Benzylalcohol and 50% Distilled water). Caliper measurements of the ankle joint diameter are taken daily, and reducing joint swelling is taken as a measure of efficacy.

The MDR1a-knockout mice, which lack the P-glycoprotein gene, spontaneously develop colitis under specific pathogen-free condition. The pathology in these animals has been characterized as Th1-type T cell-mediated inflammation similar to ulcerative colitis in humans. Disease normally begins to develop at around 8-10 weeks after birth. However the ages at which disease emerges and the ultimate penetrance level often vary considerably among different animal facilities.

In a study using the MDR1 a-knockout mice, a CCR9 antagonist is evaluated by prophylactic administration for its ability to delay disease onset. Female mice (n=34) are dosed with 50 mg/kg twice a day by subcutaneous injections for 14 consecutive weeks starting at age 10 weeks. The study is evaluated for IBD-associated growth retardation.

Evaluation of a Test Modulator in a Rat Model of Thioglycollate-Induced Peritoneal Inflammation

A 2-day study of thioglycollate-induced inflammation is conducted to evaluate the effects of the test modulator. The hallmarks of this model are reliable onset and progression of robust, easily measurable inflammatory cellular infiltrate. For the induction of inflammatory peritonitis in Lewis rats, Brewer-Thioglycollate (1.0 mL, 4% solution in distilled water) is injected intra peritoneal (i.p.). Before this injection, the treatment group received test modulator or vehicle and the control group received the same volume of PBS as i.p. injection. After 2 days, a peritoneal lavage is performed with ice-cold PBS containing 1 mM EDTA. The recovered cells are counted with a cell counter (Coulter Counter; Coulter Pharmaceutical, Palo Alto, Calif.) and monocytes/macrophages were identified by flow cytometry using light-scatter properties.

The inhibition of the number of inflammatory macrophages elicited following tioglycollate injection is evaluated.

Evaluation of a Test Modulator in a Mouse Model of Bacterial Infection

A 1-day study of streptococcus pneumoniae infection is conducted to evaluate the effects of the test modulator. The model measures bacterial infection and spread in an animal following pulmonary infection with live bacterial cultures, measured by inflammatory cellular infiltrate, and assessment of bacterial burden. C57/B6 mice are inoculated intra nasally with LD50 400 CFU at day 0. Groups are either test modulator or vehicle control treated 1 day prior to bacterial inoculation and twice daily throughout the study. Bacterial burden is measured at 24 hours by plating serial dilutions of homogenized lung tissue on agar plates and counting colonies.

Pharmacologics to be Used in Conjunction with CCR2 Compounds

Pharmacological agents that can be used in conjunction with the CCR2 antagonists of the current invention include those used for the treatments of atherosclerosis, restenosis, multiple sclerosis, pulmonary fibrosis, inflammatory bowel disease, rheumatoid arthritis, graft-versus-host disease, renal fibrosis, psoriasis, transplantation rejection, obesity, diabetes, hypercholesterolemia and cancer.

In the tables below, structures and activity are provided for representative compounds described herein. Activity is provided as follows for either or both of the chemotaxis assay and/or calcium mobilization assays, described above.

TABLE 2
Compounds with activity in one of the chemotaxis, binding or
calcium mobilization assays, with IC₅₀ < 500 nM

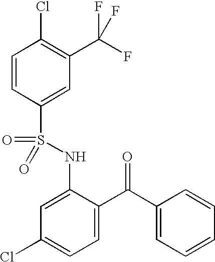

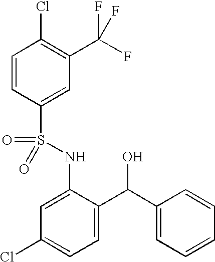

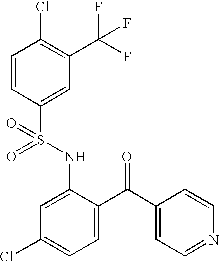

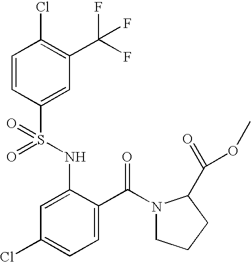

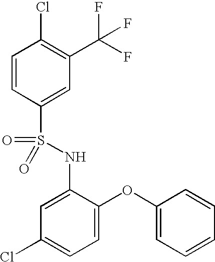

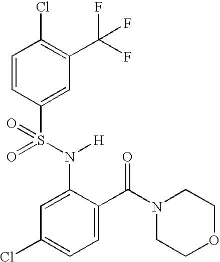

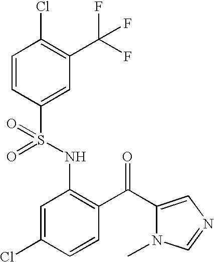

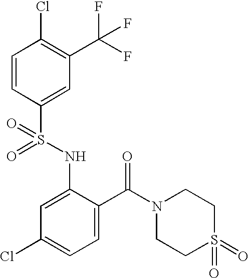

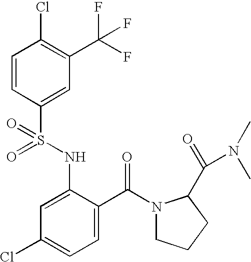

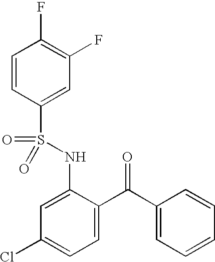

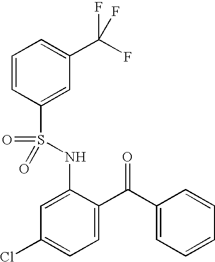

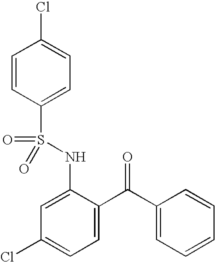

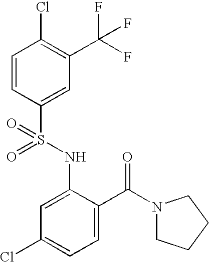

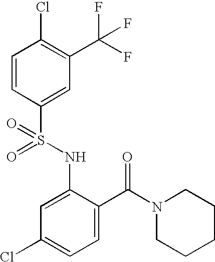

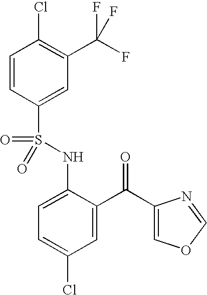

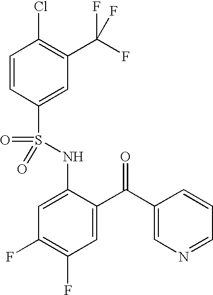

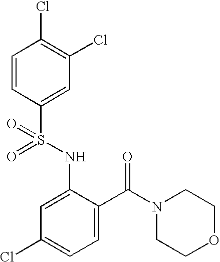

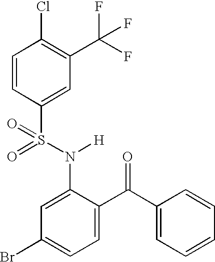

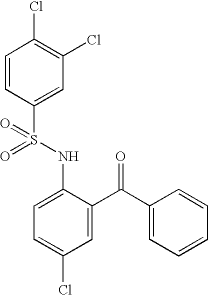

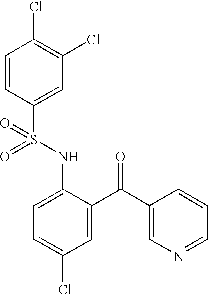

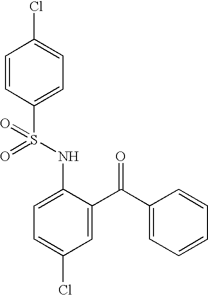

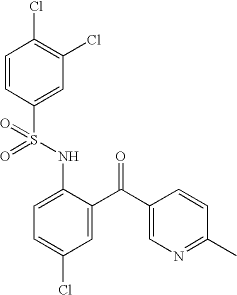

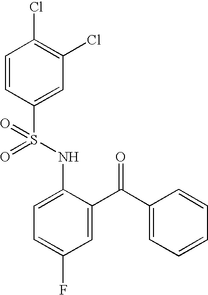

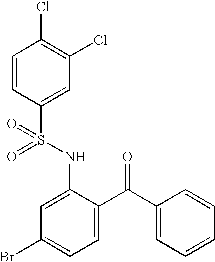

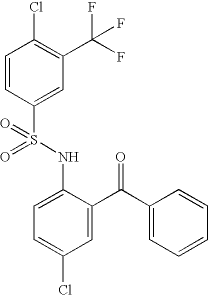

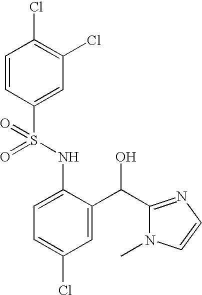

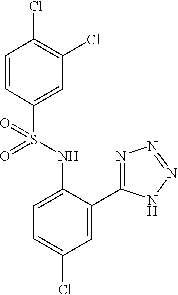

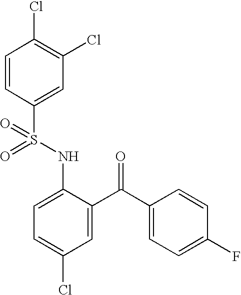

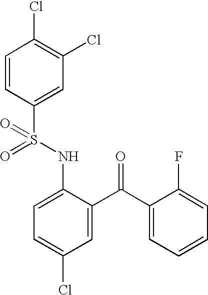

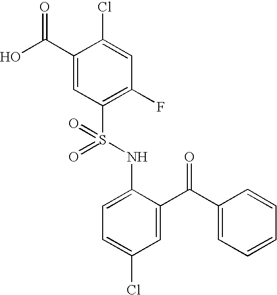

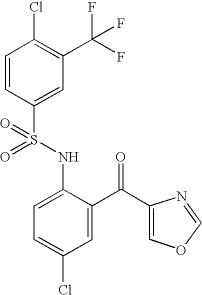

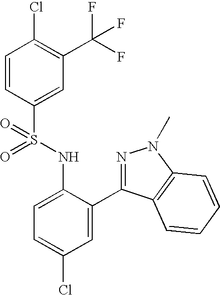

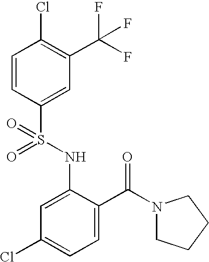

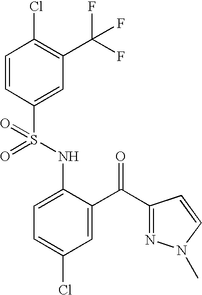

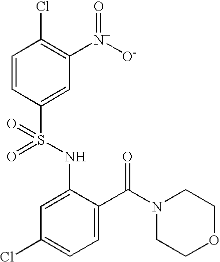

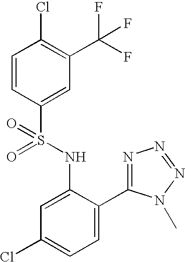

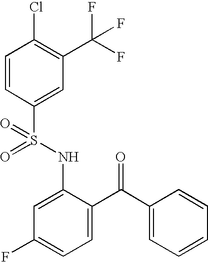

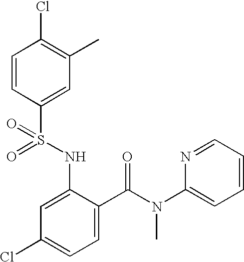

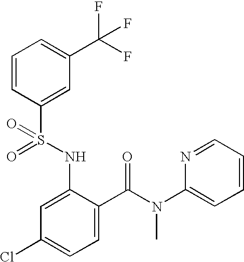

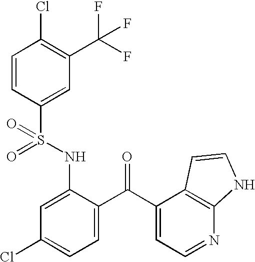

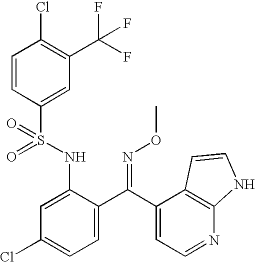

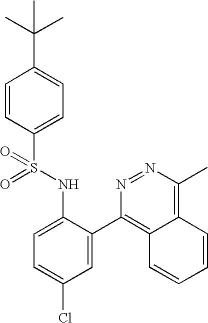

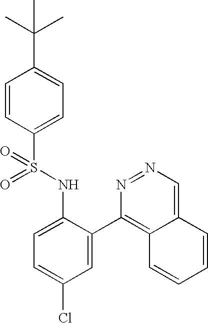

TABLE 3
Compounds with activity in one of the chemotaxis, binding or
calcium mobilization assays, with 500 nM < IC₅₀ < 5000 nM

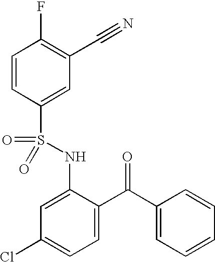

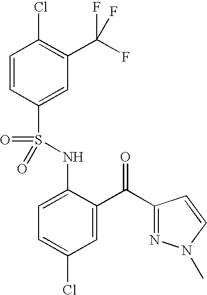

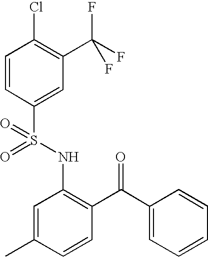

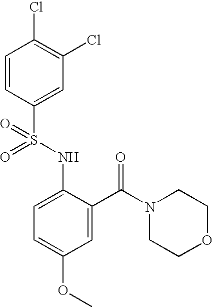

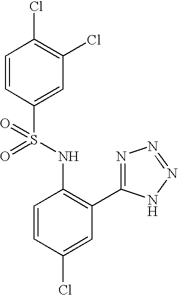

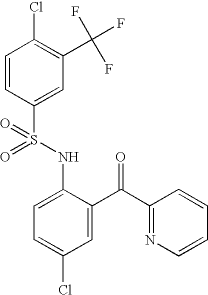

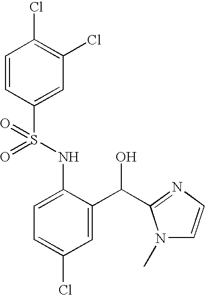

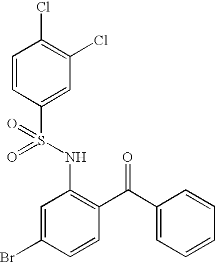

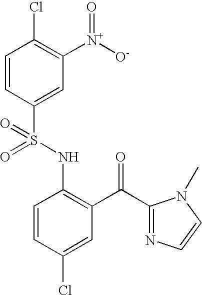

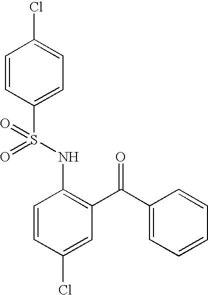

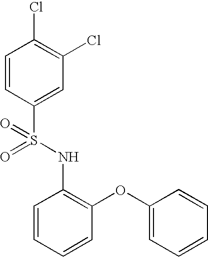

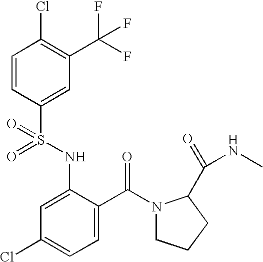

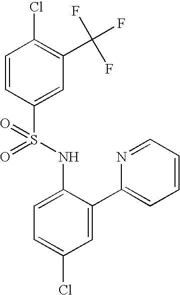

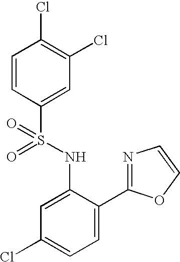

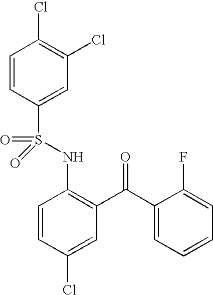

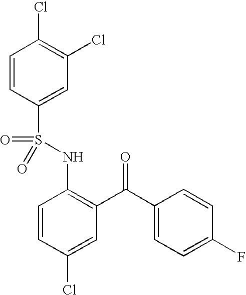

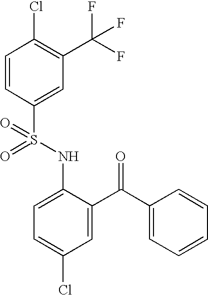

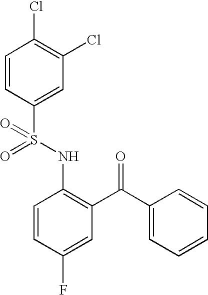

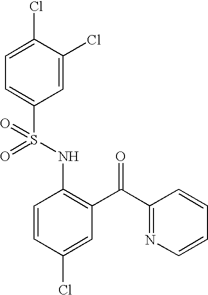

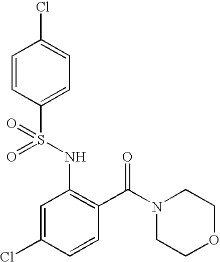

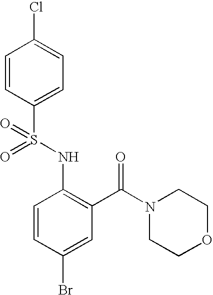

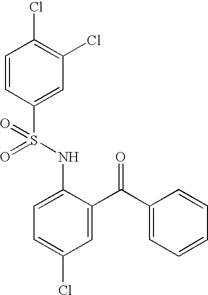

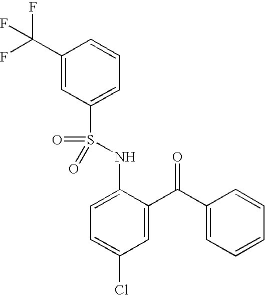

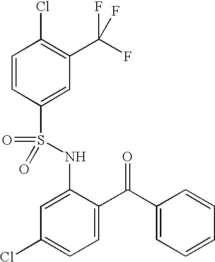

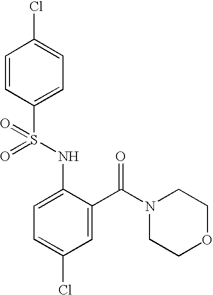

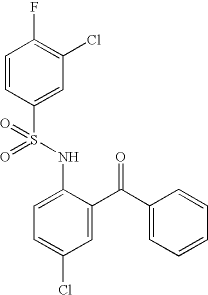

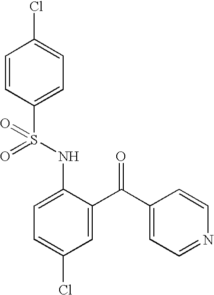

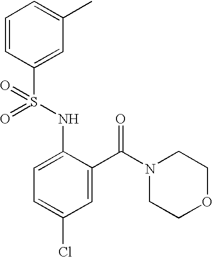

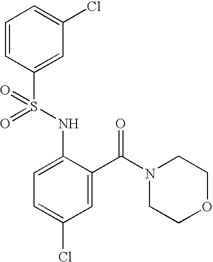

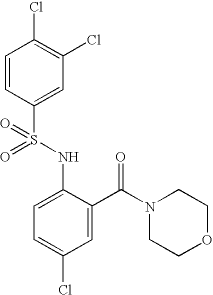

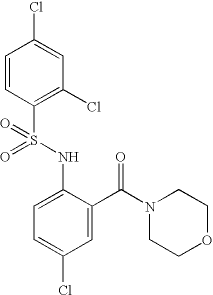

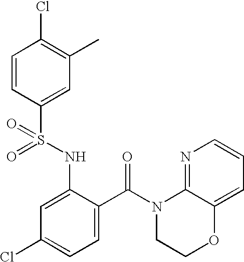

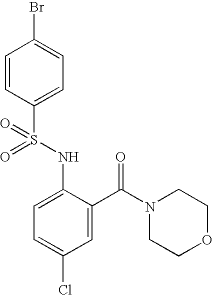

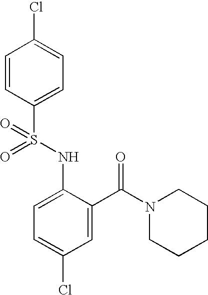

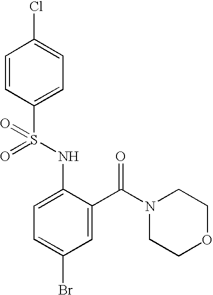

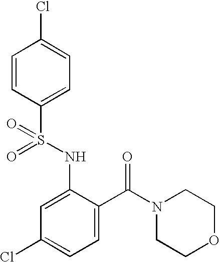

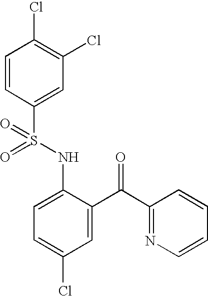

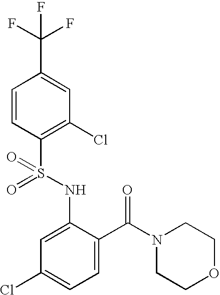

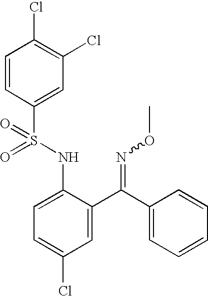

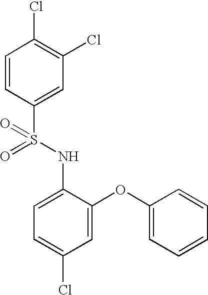

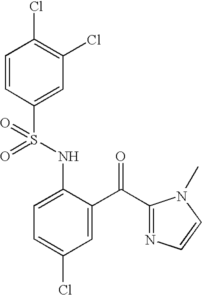

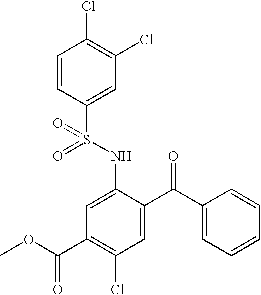

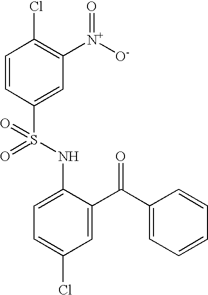

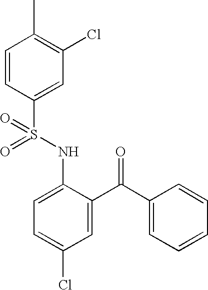

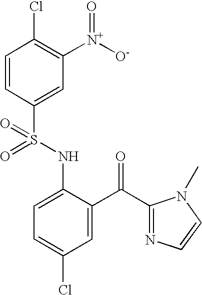

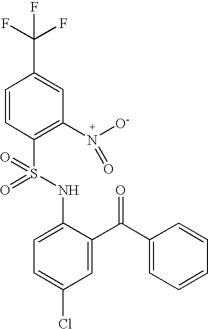

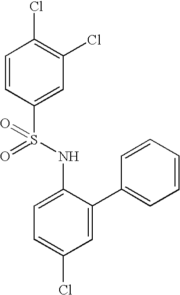

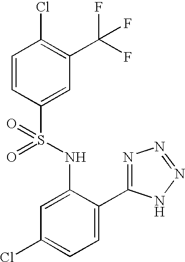

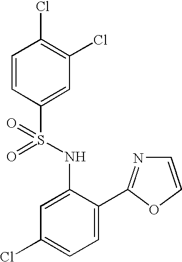

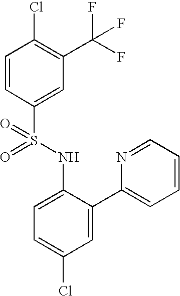

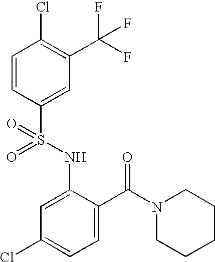

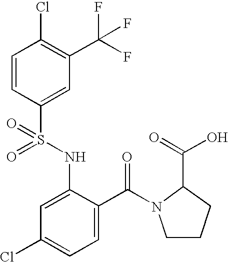

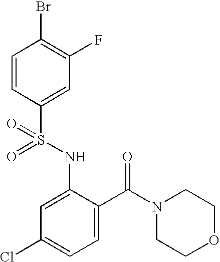

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-38. (canceled)

39. A compound of the formula (a), or a pharmaceutically acceptable salt thereof: 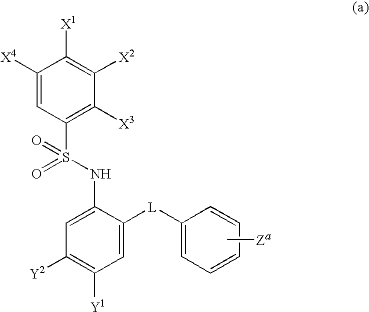
where X¹ is halogen; X² is halogen, or substituted alkyl; and X³ and X⁴ are each independently hydrogen, halogen, substituted or unsubstituted C₁₋₈ alkyl, —CN, —NO₂, —CO₂H, or C₁₋₈ haloalkyl;
where one of Y¹ and Y² is halogen, and the other is hydrogen;
where Z^{a} is 1-4 substituents selected from the group consisting of hydrogen, halogen, and substituted or unsubstituted C₁₋₈ alkyl; where the substituents may together with the atom(s) to which they are attached, form a substituted or unsubstituted 5-, 6-, or 7-membered ring; and
where L is selected from the group consisting of —C(O)—, —O—, and —C(—OH)—.

40. A composition comprising a pharmaceutically acceptable carrier and the compound or the pharmaceutically acceptable salt thereof according to claim 39.

41. A compound of the formula (b), or a pharmaceutically acceptable salt thereof: 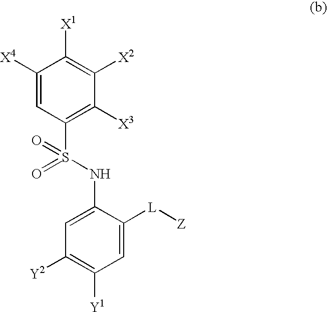
where X¹ is halogen; X² is halogen, or substituted alkyl; and X³ and X⁴ are each independently hydrogen, halogen, substituted or unsubstituted C₁₋₈ alkyl, —CN, —NO₂, —CO₂H, or C₁₋₈ haloalkyl;
where one of Y¹ and Y² is halogen, and the other is hydrogen;
where Z is substituted or unsubstituted pyridinyl; where the substituents may together with the atom(s) to which they are attached, form a substituted or unsubstituted 5-, 6-, or 7-membered ring; and
where L is selected from the group consisting of —C(O)—, —O—, and —C(—OH)—.

42. A composition comprising a pharmaceutically acceptable carrier and the compound or the pharmaceutically acceptable salt thereof according to claim 41.

43. A compound of the formula (c), or a pharmaceutically acceptable salt thereof: 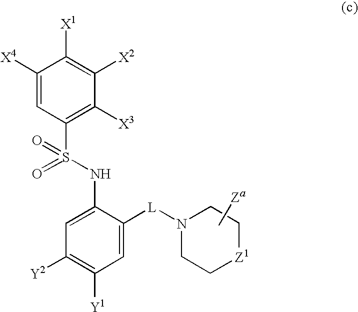
where X¹ is halogen; X² is halogen, or substituted alkyl; and X³ and X⁴ are each independently hydrogen, halogen, substituted or unsubstituted C₁₋₈ alkyl, —CN, —NO₂, —CO₂H, or C₁₋₈ haloalkyl;
where one of Y¹ and Y² is halogen, and the other is hydrogen;
where Z¹ is —CH₂—, —O— or —SO₂—;
where Z^{a} is 1-4 substituents selected from the group consisting of hydrogen, halogen, and substituted or unsubstituted C₁₋₈ alkyl; where the substituents may together with the atom(s) to which they are attached, form a substituted or unsubstituted 5-, 6-, or 7-membered ring; and
where L is —C(O)—.

44. A composition comprising a pharmaceutically acceptable carrier and the compound or the pharmaceutically acceptable salt thereof according to claim 43.

45. A compound or a pharmaceutically acceptable salt thereof, wherein the compound is selected from the group consisting of:

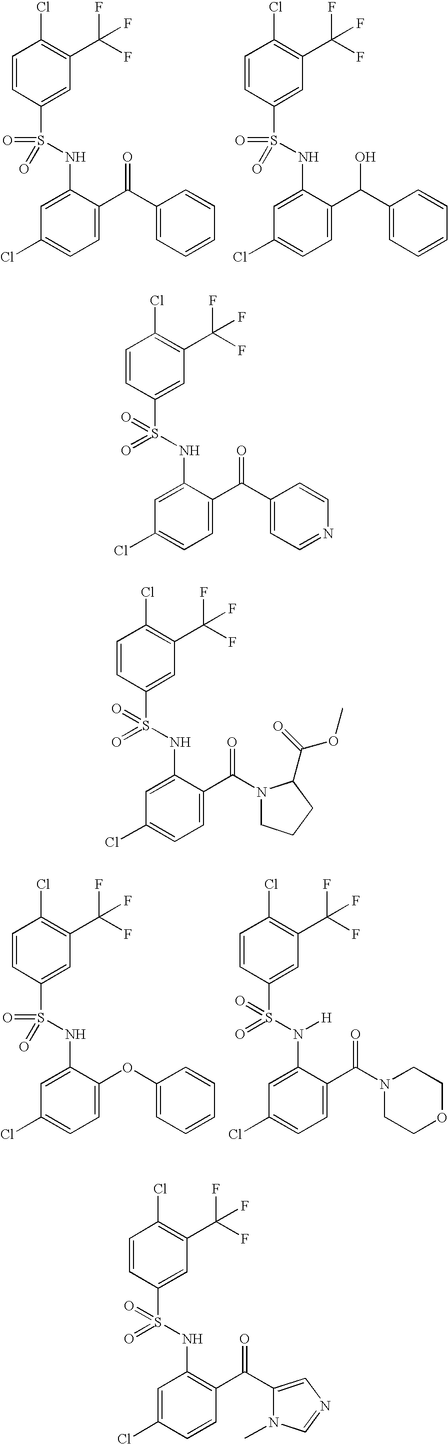

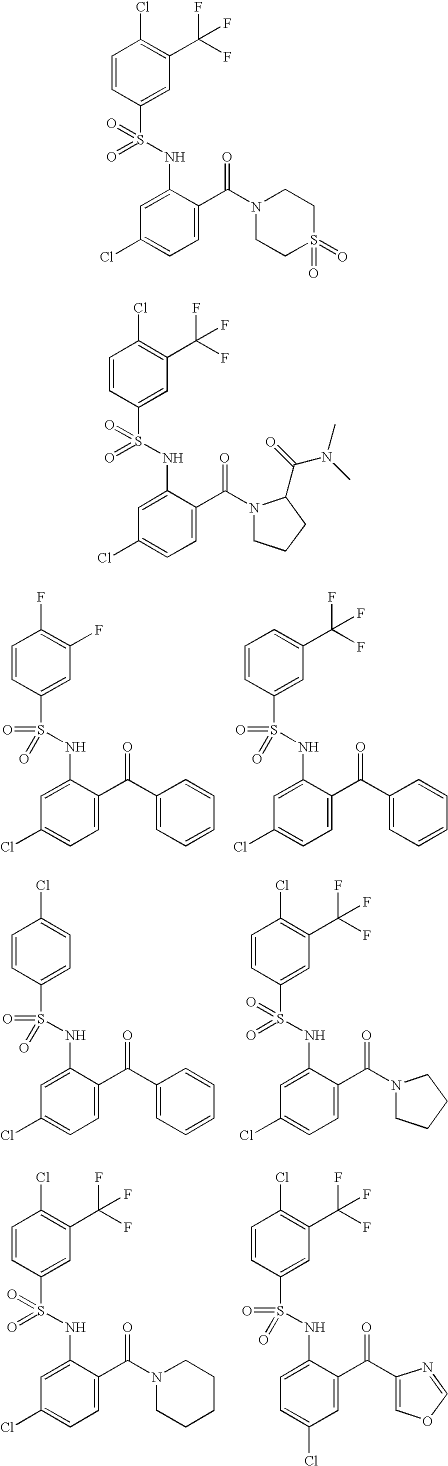

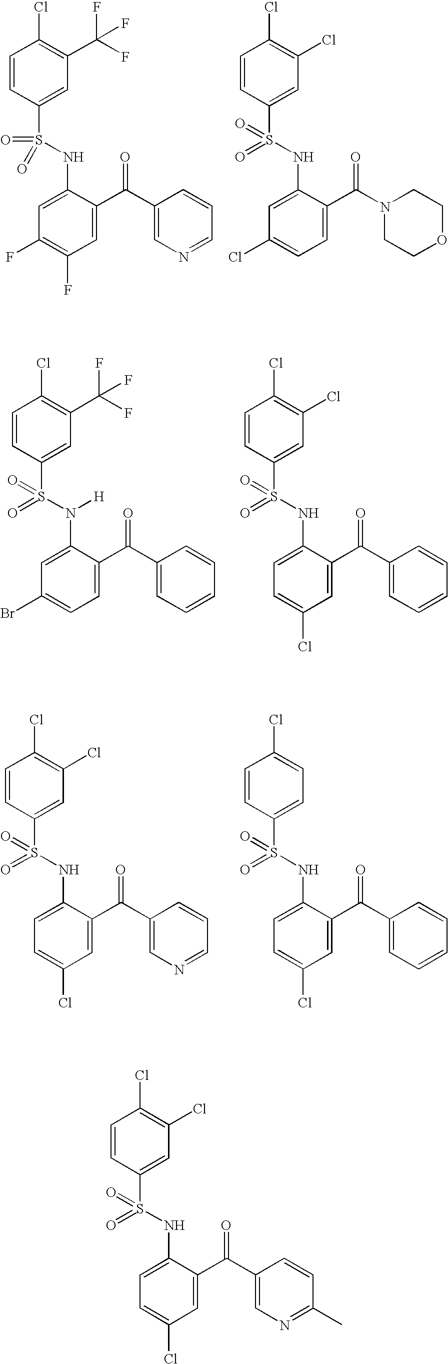

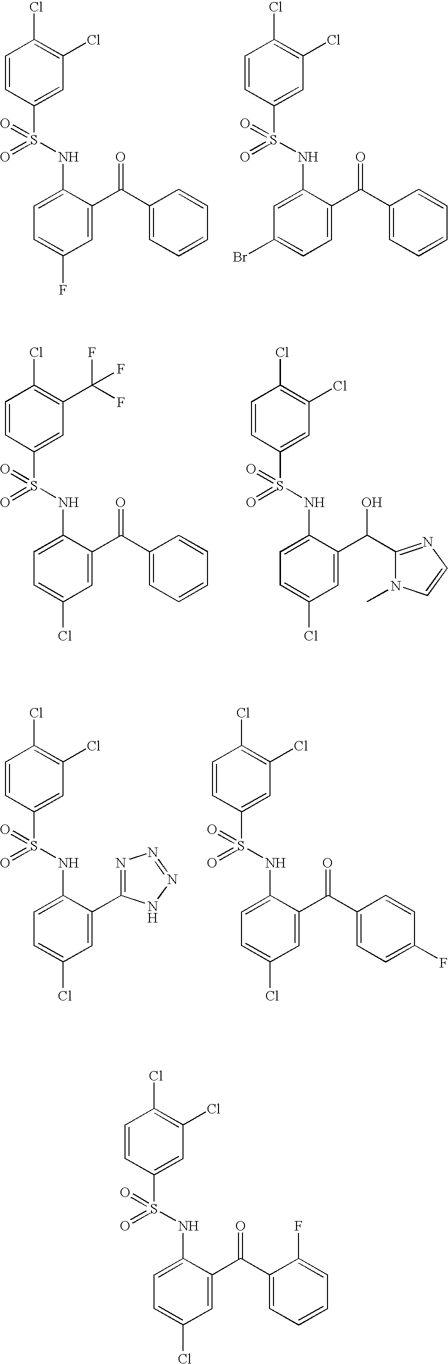

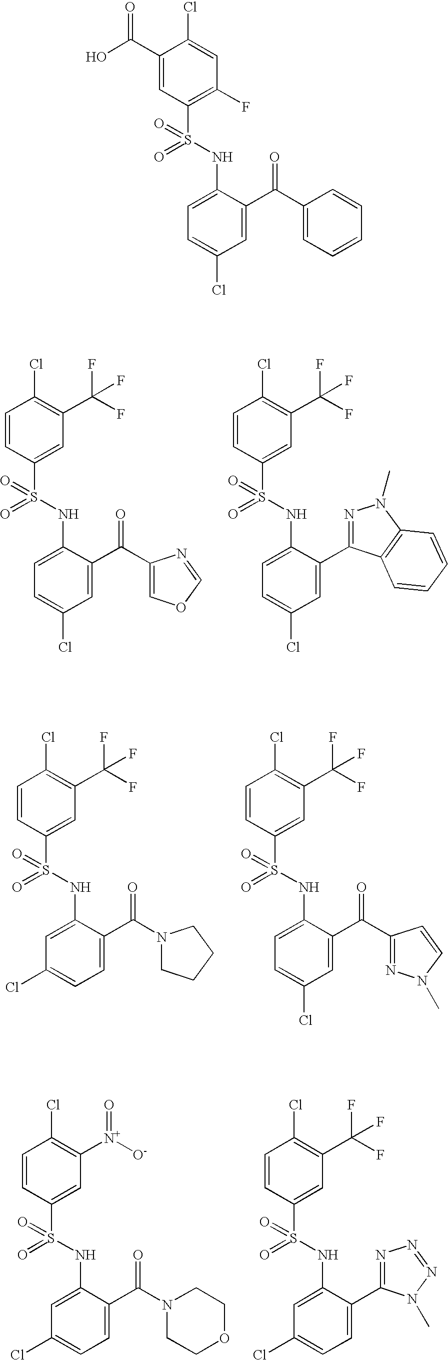

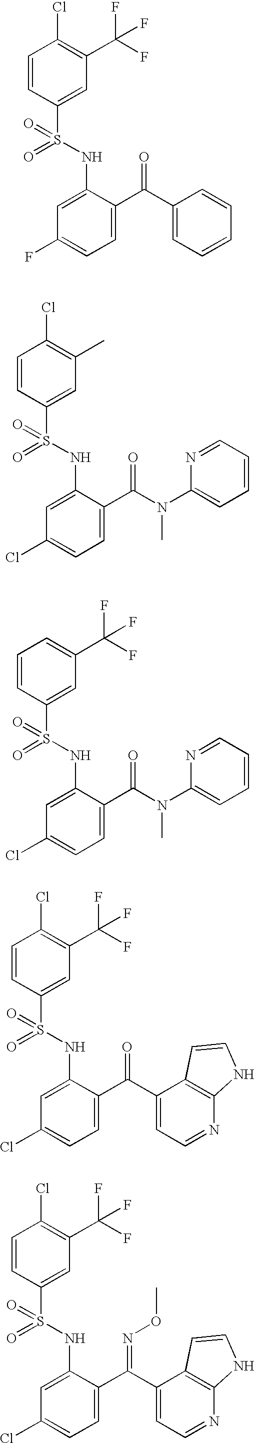

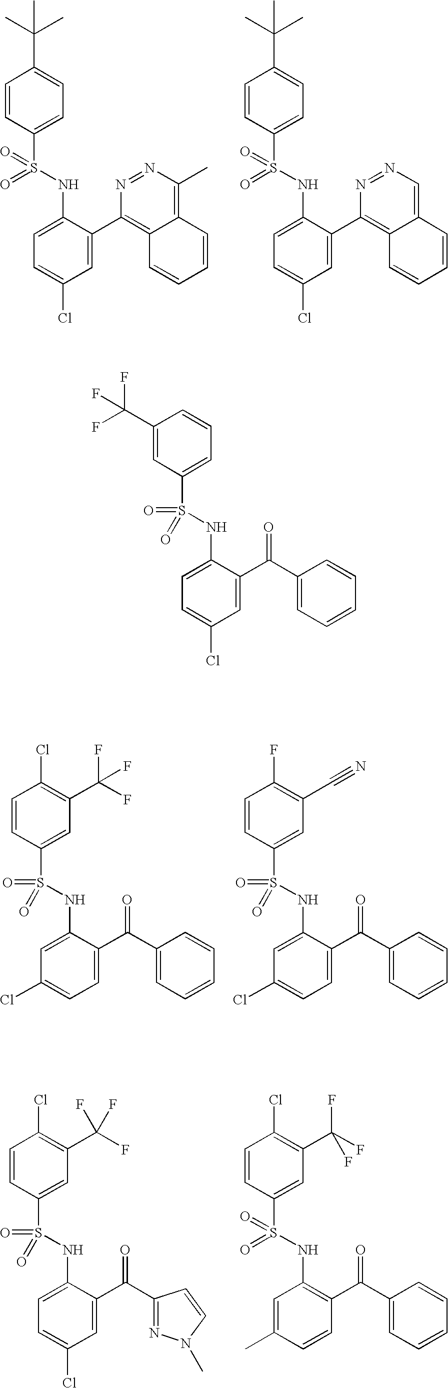

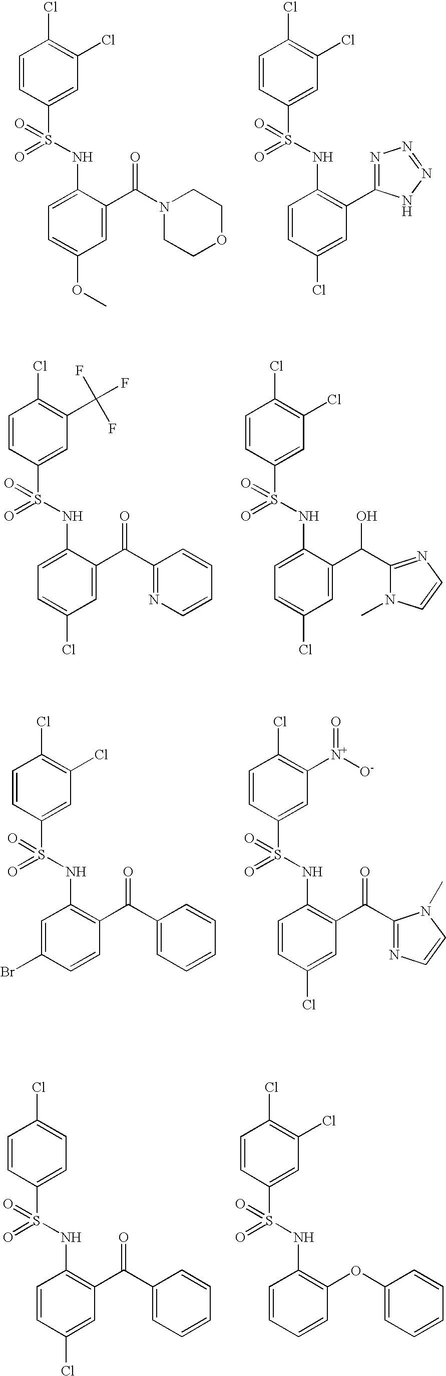

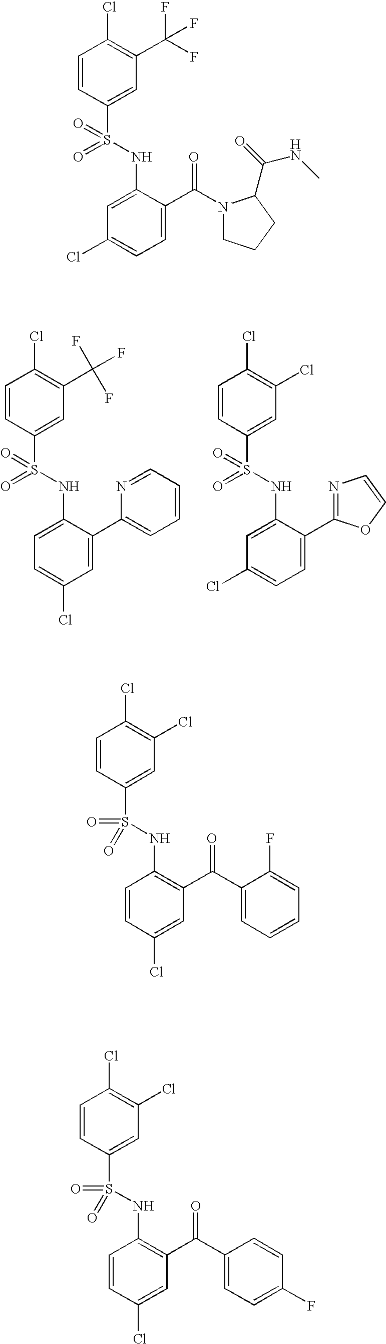

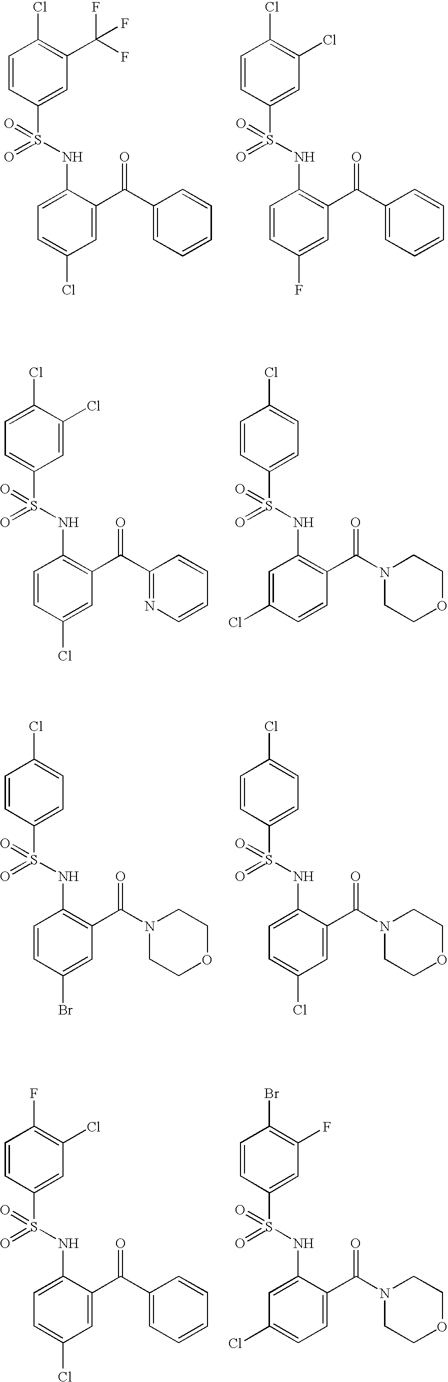

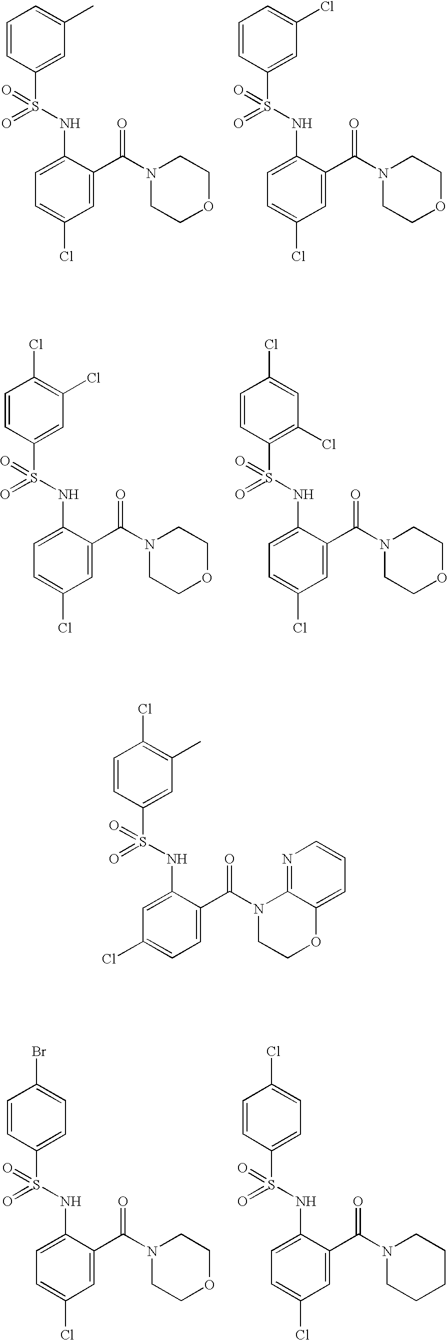

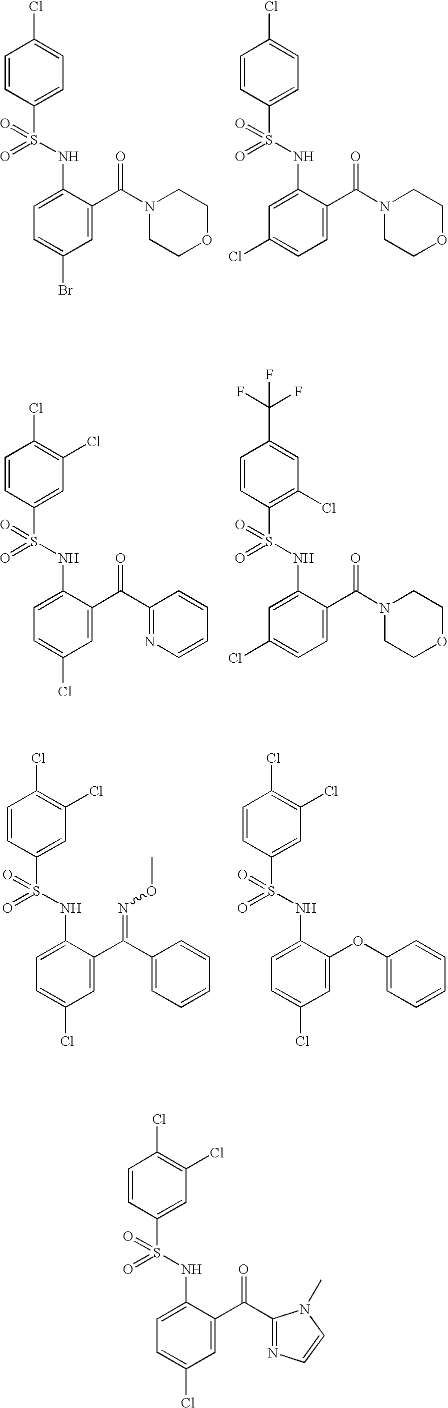

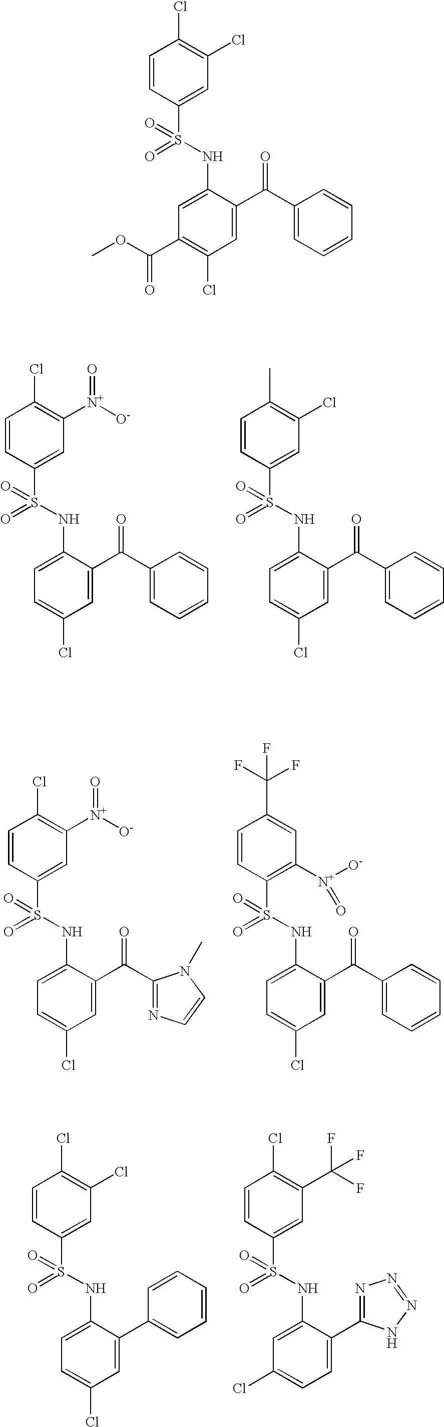

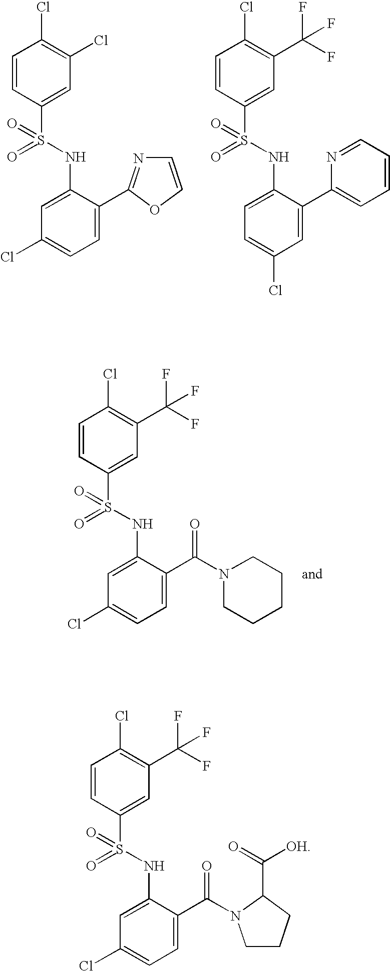

46. A composition comprising a pharmaceutically acceptable carrier and the compound or the pharmaceutically acceptable salt thereof according to claim 45.

47. A method for treating a CCR2-mediated condition or disease comprising administering to a subject an effective amount of the compound or the pharmaceutically acceptable thereof according to claim 45.
